SEQUENCE LISTING (27 sequences)

SEQ ID NO: 1 - DNA, 2368 bp, Homo sapiens
tcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattga

SEQ ID NO: 2 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 3 - DNA, 2368 bp, Homo sapiens
tcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgaaactttcacatcaacctctttcaagtgctgccctggctgaaggagaaactccaagatgaggatttgggttttctatgaggggtttcctgctggacaggggcgtgggattga

SEQ ID NO: 4 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARNFHINLFQVLPWLKEKLQDEDLGFL

SEQ ID NO: 5 - DNA, 2368 bp, Homo sapiens
tcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcaggcagcattggggccagcgacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgaaactttcacatcaacctctttcaagtgctgccctggctgaaggagaaactccaagatgaggatttgggttttctatgaggggtttcctgctggacaggggcgtgggattga

SEQ ID NO: 6 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASDFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARNFHINLFQVLPWLKEKLQDEDLGFL

SEQ ID NO: 7 - DNA, 2368 bp, Homo sapiens
tcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacaggcggcagcgatcgtcctggacccttcaggctccatgaacatctacctggtgctagatggatcaggcagcattggggccagcgacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctatgaggggtttcctgctggacaggggcgtgggattga

SEQ ID NO: 8 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQAAAIVLDPSGSMNIYLVLDGSGSIGASDFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 9 - DNA, 2428 bp, Mus sp.
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccggaacttccacatcaacctcttccaggtgctgccctggctaaaggacaagctcaaagatgaggatttgggttttctatgaagagcttcctgcagggagagtgtgaggacag

SEQ ID NO: 10 - protein, 761 aa, Mus sp.
MESPQLCLVLLVLGFSSGGVSATPVLEARPQVSCSLEGVEIKGGSFQLLQGGQALEYLCPSGFYPYPVQTRTCRSTGSWSDLQTRDQKIVQKAECRAIRCPRPQDFENGEFWPRSPFYNLSDQISFQCYDGYVLRGSANRTCQENGRWDGQTAICDDGAGYCPNPGIPIGTRKVGSQYRLEDIVTYHCSRGLVLRGSQKRKCQEGGSWSGTEPSCQDSFMYDSPQEVAEAFLSSLTETIEGADAEDGHSPGEQQKRKIVLDPSGSMNIYLVLDGSDSIGSSNFTGAKRCLTNLIEKVASYGVRPRYGLLTYATVPKVLVRVSDERSSDADWVTEKLNQISYEDHKLKSGTNTKRALQAVYSMMSWAGDAPPEGWNRTRHVIIIMTDGLHNMGGNPVTVIQDIRALLDIGRDPKNPREDYLDVYVFGVGPLVDSVNINALASKKDNEHHVFKVKDMEDLENVFYQMIDETKSLSLCGMVWEHKKGNDYHKQPWQAKISVTRPLKGHETCMGAVVSEYFVLTAAHCFMVDDQKHSIKVSVGGQRRDLEIEEVLFHPKYNINGKKAEGIPEFYDYDVALVKLKNKLKYGQTLRPICLPCTEGTTRALRLPQTATCKQHKEQLLPVKDVKALFVSEQGKSLTRKEVYIKNGDKKASCERDATKAQGYEKVKDASEVVTPRFLCTGGVDPYADPNTCKGDSGGPLIVHKRSRFIQVGVISWGVVDVCRDQRRQQLVPSYARNFHINLFQVLPWLKDKLKDEDLGFL

SEQ ID NO: 11 - DNA, 2428 bp, Mus sp.
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcaggcagcatcggaagcagcgacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccggaacttccacatcaacctcttccaggtgctgccctggctaaaggacaagctcaaagatgaggatttgggttttctatgaagagcttcctgcagggagagtgtgaggacag

SEQ ID NO: 12 - protein, 761 aa, Mus sp.
MESPQLCLVLLVLGFSSGGVSATPVLEARPQVSCSLEGVEIKGGSFQLLQGGQALEYLCPSGFYPYPVQTRTCRSTGSWSDLQTRDQKIVQKAECRAIRCPRPQDFENGEFWPRSPFYNLSDQISFQCYDGYVLRGSANRTCQENGRWDGQTAICDDGAGYCPNPGIPIGTRKVGSQYRLEDIVTYHCSRGLVLRGSQKRKCQEGGSWSGTEPSCQDSFMYDSPQEVAEAFLSSLTETIEGADAEDGHSPGEQQKRKIVLDPSGSMNIYLVLDGSGSIGSSDFTGAKRCLTNLIEKVASYGVRPRYGLLTYATVPKVLVRVSDERSSDADWVTEKLNQISYEDHKLKSGTNTKRALQAVYSMMSWAGDAPPEGWNRTRHVIIIMTDGLHNMGGNPVTVIQDIRALLDIGRDPKNPREDYLDVYVFGVGPLVDSVNINALASKKDNEHHVFKVKDMEDLENVFYQMIDETKSLSLCGMVWEHKKGNDYHKQPWQAKISVTRPLKGHETCMGAVVSEYFVLTAAHCFMVDDQKHSIKVSVGGQRRDLEIEEVLFHPKYNINGKKAEGIPEFYDYDVALVKLKNKLKYGQTLRPICLPCTEGTTRALRLPQTATCKQHKEQLLPVKDVKALFVSEQGKSLTRKEVYIKNGDKKASCERDATKAQGYEKVKDASEVVTPRFLCTGGVDPYADPNTCKGDSGGPLIVHKRSRFIQVGVISWGVVDVCRDQRRQQLVPSYARNFHINLFQVLPWLKDKLKDEDLGFL

SEQ ID NO: 13 - DNA, 2428 bp, Mus sp.
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcaggcggcagcgattgtcctagacccctcgggctccatgaatatctacctggtgctagatggatcaggcagcatcggaagcagcgacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctatgaagagcttcctgcagggagagtgtgaggacag

SEQ ID NO: 14 - protein, 761 aa, Mus sp.
MESPQLCLVLLVLGFSSGGVSATPVLEARPQVSCSLEGVEIKGGSFQLLQGGQALEYLCPSGFYPYPVQTRTCRSTGSWSDLQTRDQKIVQKAECRAIRCPRPQDFENGEFWPRSPFYNLSDQISFQCYDGYVLRGSANRTCQENGRWDGQTAICDDGAGYCPNPGIPIGTRKVGSQYRLEDIVTYHCSRGLVLRGSQKRKCQEGGSWSGTEPSCQDSFMYDSPQEVAEAFLSSLTETIEGADAEDGHSPGEQQAAAIVLDPSGSMNIYLVLDGSGSIGSSDFTGAKRCLTNLIEKVASYGVRPRYGLLTYATVPKVLVRVSDERSSDADWVTEKLNQISYEDHKLKSGTNTKRALQAVYSMMSWAGDAPPEGWNRTRHVIIIMTDGLHNMGGNPVTVIQDIRALLDIGRDPKNPREDYLDVYVFGVGPLVDSVNINALASKKDNEHHVFKVKDMEDLENVFYQMIDETKSLSLCGMVWEHKKGNDYHKQPWQAKISVTRPLKGHETCMGAVVSEYFVLTAAHCFMVDDQKHSIKVSVGGQRRDLEIEEVLFHPKYNINGKKAEGIPEFYDYDVALVKLKNKLKYGQTLRPICLPCTEGTTRALRLPQTATCKQHKEQLLPVKDVKALFVSEQGKSLTRKEVYIKNGDKKASCERDATKAQGYEKVKDASEVVTPRFLCTGGVDPYADPNTCKGDSGGPLIVHKRSRFIQVGVISWGVVDVCRDQRRQQLVPSYARDFHINLFQVLPWLKDKLKDEDLGFL

SEQ ID NO: 15 - DNA, 2428 bp, Mus sp.
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacag

SEQ ID NO: 16 - protein, 761 aa, Mus sp.
MESPQLCLVLLVLGFSSGGVSATPVLEARPQVSCSLEGVEIKGGSFQLLQGGQALEYLCPSGFYPYPVQTRTCRSTGSWSDLQTRDQKIVQKAECRAIRCPRPQDFENGEFWPRSPFYNLSDQISFQCYDGYVLRGSANRTCQENGRWDGQTAICDDGAGYCPNPGIPIGTRKVGSQYRLEDIVTYHCSRGLVLRGSQKRKCQEGGSWSGTEPSCQDSFMYDSPQEVAEAFLSSLTETIEGADAEDGHSPGEQQKRKIVLDPSGSMNIYLVLDGSDSIGSSNFTGAKRCLTNLIEKVASYGVRPRYGLLTYATVPKVLVRVSDERSSDADWVTEKLNQISYEDHKLKSGTNTKRALQAVYSMMSWAGDAPPEGWNRTRHVIIIMTDGLHNMGGNPVTVIQDIRALLDIGRDPKNPREDYLDVYVFGVGPLVDSVNINALASKKDNEHHVFKVKDMEDLENVFYQMIDETKSLSLCGMVWEHKKGNDYHKQPWQAKISVTRPLKGHETCMGAVVSEYFVLTAAHCFMVDDQKHSIKVSVGGQRRDLEIEEVLFHPKYNINGKKAEGIPEFYDYDVALVKLKNKLKYGQTLRPICLPCTEGTTRALRLPQTATCKQHKEQLLPVKDVKALFVSEQGKSLTRKEVYIKNGDKKASCERDATKAQGYEKVKDASEVVTPRFLCTGGVDPYADPNTCKGDSGGPLIVHKRSRFIQVGVISWGVVDVCRDQRRQQLVPSYARDFHINLFQVLPWLKDKLKDEDLGFL

SEQ ID NO: 17 - DNA, 35 bp, Artificial Sequence
  Description of Artificial Sequence Synthetic primer
ctagctagctcctgccccaggcccagcttctctcc

SEQ ID NO: 18 - DNA, 29 bp, Artificial Sequence
  Description of Artificial Sequence Synthetic primer
ctagctagctcaatcccacgcccctgtcc

SEQ ID NO: 19 - DNA, 7912 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgagctccaccgcggtggcggccgctctagaactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaatctagaactagtgtaagtatcaaggttacaagacaggtttaaggagaccaatagaaactgggcttgtcgagacagagaagactcttgcgtttctgataggcacctattggtcttactgacatccactttgcctttctctccacagactagtggatctcgagatgaagcggagagagctggagaagaaactgaggaaagtgcgcgtgacacctcaacaggacaagtactataccatcggcaacctgcagtgggccatccgcatgatcaacctgatgggcatcaagtgcgtgtgcgacgaggaatgcagcgccgctgaggtcgccctgatcatcacccagtttagcgccctcgacctggagaactcccctatccgcggcaaggaagaggtggccatcaagaataccctgaaggtgttttggagcctgctggccggatacaagcctgagagcaccgagaccgccctgggatactgggaagccttcacctacagagagagggaagctagagccgacaaggagggagagatcaagagcatctaccctagcctgacccagaacacccagaacaagaaacagaccagcaatcagacaaacacccagagcctgcccgctatcaccacacaggatggcacccctcgcttcgaccccgacctgatgaagcagctgaagatctggtccgatgccacagagcgcaatggagtggacctgcatgccgtgaacatcctgggagtgatcacagccaacctggtgcaagaagagatcaagctcctgctgaatagcacacccaagtggcgcctggacgtgcagctgatcgagagcaaagtgagagagaaggagaacgcccaccgcacctggaagcagcatcaccctgaggctcccaagacagacgagatcattggaaagggactgagctccgccgagcaggctaccctgatcagcgtggagtgcagagagaccttccgccagtgggtgctgcaggctgccatggaggtcgcccaggctaagcacgccacacccggacctatcaacatccatcaaggccctaaggaaccctacaccgacttcatcaaccgcctggtggctgccctggaaggaatggccgctcccgagaccacaaaggagtacctcctgcagcacctgagcatcgaccacgccaacgaggactgtcagtccatcctgcgccctctgggacccaacacacctatggagaagaaactggaggcctgtcgcgtggtgggaagccagaagagcaagatgcagttcctggtggccgctatgaaggaaatggggatccagtctcctattccagccgtgctgcctcacacacccgaagcctacgcctcccaaacctcagggcccgaggatggtaggagatgttacggatgtgggaagacaggacatttgaagaggaattgtaaacagcaaaaatgctaccattgtggcaaacctggccaccaagcaagaaactgcaggtcaaaaaacgggaagtgctcctctgccccttatgggcagaggagccaaccacagaacaattttcaccagagcaacatgagttctgtgaccccatctgcaccccctcttatattagattagacaaacagccttttataaaggtgttcattggcggccgctgggtgaagggactggtggactcgggcgctgacgaggtggtgctgaagaacatccactgggaccgcatcaaaggctaccctggaacacccatcaagcagatcggcgtgaacggcgtgaacgtggctaagcgcaaaacacatgtggagtggagattcaaagacaagaccggcatcattgacgtcctcttcagcgacacacctgtgaacctgtttggcagaagcctgctcagatccatcgtgacctgctttaccctgctggtgcacaccgagaagatcgagccactgcctgtgaaggtgcgcggccctggacctaaggtgccacaatggcccctgaccaaggagaaataccaggccctgaaggagatcgtgaaggacctgctggccgagggaaagatcagcgaagctgcctgggacaacccttacaacacacccgtgttcgtgatcaagaagaaaggcaccggccgctggcgcatgctgatggacttccgcgagctgaataagatcaccgtgaaaggccaagagttcagcacaggactcccttatccacccggcatcaaggagtgtgagcacctgaccgccatcgacatcaaggacgcctacttcaccatccctctgcacgaggacttcagacccttcacagccttcagcgtggtcccagtgaaccgcgagggccccatcgagcgcttccagtggaacgtcctgcctcaaggctgggtgtgctcccctgccatctaccagaccacaacccagaagatcattgagaacatcaagaagagccatcccgacgtgatgctgtatcagtacatggatgacctcctgattggcagcaatcgcgatgaccacaagcagatcgtgcaggagatcagagacaagctgggcagctatggcttcaagacacccgacgagaaagtgcaggaagagcgcgtgaagtggatcggcttcgagctgacacctaagaaatggagattccagcctaggcaactgaagatcaagaacccactgaccgtgaacgaactccagcagctggtcggcaactgtgtgtgggtgcagcccgaggtgaagatccctctgtacccactgaccgatctgctccgcgacaagaccaacctgcaggaaaagatccagctgacacccgaggccatcaagtgcgtggaagagttcaacctgaagctgaaagatcccgagtggaaggacagaattcgcgaaggagccgagctggtgatcaagatccaaatggtccctcgcggcatcgtgttcgacctgctgcaagacggcaatcctatctggggaggcgtgaaaggactgaactacgaccacagcaacaagatcaagaagatcctgcgcaccatgaacgagctgaaccgcaccgtggtgatcatgaccggacgcgaagctagctttctcctgcctggatccagcgaggattgggaggccgccctgcagaaggaagagagcctgacccaaatctttcccgtgaagttctaccgccatagctgtagatggacaagcatctgtggacccgtccgcgagaacctgaccacctactataccgacggcgggaagaaaggaaagacagctgccgcagtgtactggtgtgaaggaagaactaagagcaaagtgttccctggaaccaatcaacaggctgagctgaaggcaatctgcatggctctgctggacggacctcccaagatgaacatcatcaccgacagccgctacgcttatgagggcatgagagaggaacctgagacctgggctcgcgagggcatctggctggagattgcaaagatcctgccattcaagcaatacgtcggagtgggctgggtccctgctcacaaaggcattggaggcaataccgaggctgacgaaggagtgaagaaagccctggagcaaatggcaccatgttcccctcccgaggctatcctgctcaaacctggcgagaagcaaaacctggagaccggcatctacatgcaaggcctgagacctcagagcttcctgccccgcgctgacctccctgtcgcaatcactggcaccatggtggactccgagctgcagctccaactgctgaacatcggcaccgagcacattcgcatccagaaggacgaggtgttcatgacatgcttcctggagaacatccctagcgccaccgaagaccacgagagatggcacacatccccagacatcctggtccgccagttccacctgcccaagcgcatcgccaaggagatcgtcgcccgctgccaggagtgcaagagaaccacaacctccccagtgcgcggcaccaaccctagaggacgcttcctgtggcagatggacaacacacactggaacaaaaccatcatttgggtcgcagtggagactaacagcggactggtggaggctcaggtgattcccgaagagaccgcactgcaagtggccctgtgtatcctccagctgatccaacgctacaccgtcctgcacctgcacagcgacaacggaccctgcttcacagctcaccgcatcgagaacctgtgcaagtacctgggcatcaccaagacaaccggcattccctacaatcctcagagccaaggagtcgtggaaagagcccatcgcgacctgaaggacagactggctgcctatcaaggcgactgcgagaccgtggaagctgcactgagcctcgccctggtcagcctgaacaagaagagaggaggcatcggcggacacacaccctacgagatctatctggagagcgagcacaccaagtatcaggaccaactggagcagcaattcagcaagcagaagatcgagaaatggtgctacgtccgcaacagacgcaaggagtggaagggcccttacaaggtgctgtgggatggcgacggagctgcagtgatcgaggaagagggcaagaccgctctgtatccccaccggcacatgcgcttcatcccacctcccgacagcgatatccaggacggctccagctgatctagagtcgccctttaagctgcaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctggggggatcctctaggatcccccgggctgcaggaattcgatatcaagcttatcgataccgtcgacctcgagggggggcccggtaccacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 4026 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgagctccaccgcggtggcggccgctctagaactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaatctagaactagtgtaagtatcaaggttacaagacaggtttaaggagaccaatagaaactgggcttgtcgagacagagaagactcttgcgtttctgataggcacctattggtcttactgacatccactttgcctttctctccacagactagtggatcgatccgccaccatggaccaggacctggacagagctgagagaggcgagagaggcggcggaagcgaggaactgctgcaggaagagatcaacgagggacggctgacagctagagaggccctgcagacctggatcaacaatgatagccctcggtacgtgaagaaactgagacagggacagcccgaactgcccaccagccctggaggcggaggcggaagaggacacagagctaggaagctgcctggcgagcggagacctggattctggaagagcctgcgggagctggtggagcagaacagacggaagcaggagagacggctgagcggactggacagacggattcagcagctggaggacctggtgagacacatgagcctgggcagccctgatcctagcacacctagcgccagcgtcctgtccgtgaatcctcccgctcagacacctctgggacacctgccacctagaagctacttcaagctgaaacgggtggactgtggcgctggctgggacctgagaaccacagctgctcccggcctgcctatctgcgagctggactggattcagggcaccaagtgaggatcgatcccccgggctgcaggaattcgatatcaagcttatcgataccgtcgacctcgagaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctgctcgagggggggcccggtaccacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 21 - DNA, 5007 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgagctccaccgcggtggcggccgctctagaactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaatctagaactagtgtaagtatcaaggttacaagacaggtttaaggagaccaatagaaactgggcttgtcgagacagagaagactcttgcgtttctgataggcacctattggtcttactgacatccactttgcctttctctccacagactagtggatcagcttgccaccatggtgtccgccatcgtgctgtacgtgctgctggccgctgccgcacacagcgccttcgccgccgagcactgcaacgcccagatgaaaaccggcccctacaagatcaagaacctggacatcaccccccccaaggagaccctgcagaaggacgtggagatcaccatcgtggagaccgactacaacgagaacgtgatcatcggctacaagggctactaccaggcctacgcctacaacggcggcagcctggaccccaacaccagagtggaggagaccatgaaaaccctgaatgtgggcaaggaagatctgctgatgtggagcatcaggcagcagtgcgaagtgggcgaggagctgatcgacagatggggcagcgacagcgacgactgcttcagagacaacgagggcagaggccagtgggtgaagggcaaggagctggtgaagcggcagaacaacaaccacttcgcccaccacacctgcaacaagagctggagatgcggcatcagcaccagcaagatgtacagccggctggagtgccaggatgacaccgatgagtgccaggtgtacatcctggacgccgagggcaaccctatcaacgtgaccgtggacaccgtgctgcacagagatggcgtgtccatgatcctgaagcagaagagcaccttcaccaccaggcagatcaaggccgcctgcctgctgatcaaggacgacaagaacaaccccgagagcgtgaccagagagcactgcctgatcgacaacgacatctacgacctgagcaagaacacctggaactgcaagttcaaccggtgcatcaagcggaaagtggagcacagagtgaagaagagaccccccacctggaggcacaatgtgcgggccaagtacaccgagggcgataccgccaccaagggcgacctgatgcacatccaggaagaactgatgtacgagaacgacctgctgaagatgaacatcgagctgatgcacgcccacatcaacaagctgaacaacatgctgcacgacctgatcgtgtccgtggccaaagtggacgagaggctgatcggcaacctgatgaacaacagcgtgtccagcaccttcctgagcgacgacaccttcctgctgatgccctgcaccaacccccctgcccacaccagcaactgctacaacaacagcatctacaaggagggccggtgggtggccaacaccgacagcagccagtgcatcgacttctccaactataaggagctggccatcgacgacgatgtggagttctggatccccaccatcggcaacaccacctaccacgacagctggaaggacgccagcggctggagcttcatcgcccagcagaagtccaacctgatcaccaccatggagaacaccaagttcggcggagtgggcaccagcctgagcgatatcaccagcatggccgagggcgagctggccgccaagctgaccagcttcatgttcggccacgtggtgaacttcgtgatcatcctgatcgtgatcctgttcctgtactgcatgatccggaaccggaaccggcagtactgatagtctaggatcccccgggctgcaggaattcgatatcaagcttatcgataccgtcgacctcgagaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctgctcgagggggggcccggtaccacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 22 - DNA, 6329 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggatcccgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataaaccatatcttcactctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaacagttggcgcccaacgtggggctgagtaagagagactcggctcgagtaaaagaagacccagctcgaacgagaagactccggacaggtgagtagttgcgtgttttccccggggtgaagagaagggagttagaaaagaagcttcgtaaggttagggtgacaccccaacaggataaatattatactatagggaatcttcaatgggccattagaatcacgtgatgcatcgataaataaaaaaagagggggaatagggggccatacaccatatgaaatatacctagaatcagaacataccaaataccaagaccaactagaacaacaattttcaaaacaaaaaattgaaaagtggtgttacgtaaggaacagaagaaaggaatggaaaggaccctacaaagtgttgtgggacggagacggggcagcagtaatagaggagaattcgtggattcttgtaaaggtccccagctatgggtttgtggtagtaaatgacacagatacaccaccatccctccgcatccgaaagcctcgagcagtcggactagcaatattcctgcttgtgctggctatcatggccatcacatcctccttggtggcagctacaacgctcgtgaaccagcacacgacggctaaggttgtggagagggttgtgcaaaatgtgtcatatattgctcaaacccaggaccaattcacccacctgttcaggaatataaacaacagattaaatgtcctacaccatagagtttcatacttggagtgtacacttaatgggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccacttaaggcctgcagctagcaccggttgtacaagtcaagcggccaaccctccctagatctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgccttctaggaaactcctttgggacatcttccgccacgctcctattttaaacttaaaagggtggactgtggggcagggtgggacctcaggacaacagcagcccccggacttcccatatgtgtttatttgtgaaatttgtgatgctattgctttatttgtaatctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaacagatatatactgtcaacatcccatttggtagcttatgttctagacaagattctcaacaaattcttccccctgaatgtttatttaaaaaaaaaaaacaactactagggctctgtgcatatgtaagtgagatccttattagcaggagaacagcaataagatattattacattacaatattatatcctagggtattataatgcaaggccattatcacatacttggctaacagggtccatactgttgtaatgtattaaaaccagactgagtaataaaattgacaacaatataatcatcatctttgttataggtgggggcatttttcagatgaggtctcagagcacctgccaagcatggacctcgagggggggcccggtacccagcttttgttccctttagtgagggttaattgcgcgcttggcgtaatcatggtcatagctgtttcctgtgtgaaattgttatccgctcacaattccacacaacatacgagccgggagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 23 - DNA, 7068 bp, Artificial Sequence
  Description of Artificial Sequence Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccggcatcccgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataaaccatatcttcactctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaacagttggcgcccaacgtggggctgagtaagagagactcggctcgagtaaaagaagacccagctcgaacgagaagactccggacaggtgagtagttgcgtgttttccccggggtgaagagaagggagttagaaaagaagcttcgtaaggttagggtgacaccccaacaggataaatattatactatagggaatcttcaatgggccattagaatcacgtgatgcatcgataaataaaaaaagagggggaatagggggccatacaccatatgaaatatacctagaatcagaacataccaaataccaagaccaactagaacaacaattttcaaaacaaaaaattgaaaagtggtgttacgtaaggaacagaagaaaggaatggaaaggaccctacaaagtgttgtgggacggagacggggcagcagtaatagaggagaattcgtggattcttgtaaaggtccccagctatgggtttgtggtagtaaatgacacagatacaccaccatccctccgcatccgaaagcctcgagcagtcggactagcaatattcctgcttgtgctggctatcatggccatcacatcctccttggtggcagctacaacgctcgtgaaccagcacacgacggctaaggttgtggagagggttgtgcaaaatgtgtcatatattgctcaaacccaggaccaattcacccacctgttcaggaatataaacaacagattaaatgtcctacaccatagagtttcatacttggagtgtacacttaatgggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccacttaaggcccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagtcgacctgcagctagcaccggttgtacaagtcaagcggccaaccctccctagatctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgccttctaggaaactcctttgggacatcttccgccacgctcctattttaaacttaaaagggtggactgtggggcagggtgggacctcaggacaacagcagcccccggacttcccatatgtgtttatttgtgaaatttgtgatgctattgctttatttgtaatctgtacttcagctcgtgtagctcattagctccgagctccccaacctacagcctgagaggcactggctcggttgggtagccagcctttcgggtaataaaggcttgttggcattcggcatctacccgtgcctcctgtcttgtcttactcgagcgaacccacaactccgtcctgctgagctcacagctcgcggggcggtgaagaacacccaacagatatatactgtcaacatcccatttggtagcttatgttctagacaagattctcaacaaattcttccccctgaatgtttatttaaaaaaaaaaaacaactactagggctctgtgcatatgtaagtgagatccttattagcaggagaacagcaataagatattattacattacaatattatatcctagggtattataatgcaaggccattatcacatacttggctaacagggtccatactgttgtaatgtattaaaaccagactgagtaataaaattgacaacaatataatcatcatctttgttataggtgggggcatttttcagatgaggtctcagagcacctgccaagcatggacctcgagggggggcccggtacccagcttttgttccctttagtgagggttaattgcgcgcttggcgtaatcatggtcatagctgtttcctgtgtgaaattgttatccgctcacaattccacacaacatacgagccgggagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 24 - DNA, 24 bp, Artificial Sequence
  Description of Artificial Sequence Synthetic primer
acaccaccatccctccgcatccga

SEQ ID NO: 25 - DNA, 24 bp, Artificial Sequence
  Description of Artificial Sequence Synthetic primer
tgggtttgtggtagtaaatgacac

SEQ ID NO: 26 - DNA, 20 bp, Artificial Sequence
  Description of Artificial Sequence Synthetic primer
tggttcacgagcgttgtagc

SEQ ID NO: 27 - protein, 253 aa, Homo sapiens
MHSWERLAVLVLLGAAACAAPPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

(19) United States
(12) Patent Application Publication
(10) Pub. No.: US 2010/0120665 A1
(43) Pub. Date: May 13, 2010

(54) TREATMENT OF DISEASES CHARACTERIZED BY INFLAMMATION

(75) Inventors: Michael Kaleko, Rockville, MD (US); Tianci Luo, Clarksville, MD (US)

Correspondence Address: Wellstat Management Company. LLC, LEGAL DEPARTMENT, 930 CLOPPER ROAD, GAITHERSBURG, MD, 20878, US

(73) Assignee: ADVANCED VISION THERAPIES, INC., Gaithersburg, MD (US)

(21) Appl. No.: 12/529,177

(22) PCT Filed: Feb. 29, 2008
(86) PCT No.: PCT/US08/55498

§ 371 Date: Aug. 28, 2009

Related U.S. Application Data
(60) Provisional application No. 60/892,395, filed on Mar. 1, 2007.
(60) Provisional application No. 60/985,024, filed on Nov. 2, 2007.

(51) Int. Cl. A61K 38/16; A61P 9/10; A61P 19/02; A61P 27/00; A61K 31/7088; C12N 15/63; C07K 14/00

(52) U.S. Cl. 514/8; 514/12; 514/44 R; 435/320.1; 530/350

(57) ABSTRACT

The invention provides, in part, methods, nucleic acids, vectors, proteins and binding molecules that can be used to modulate a pathway such as a complement pathway. These methods and compositions can be utilized, inter alia, for the study and/or treatment of various conditions or diseases related to a complement pathway.

### RELATED APPLICATIONS

This application claims the benefit of U.S. Provisional Application Nos. 60/892,395, filed Mar. 1, 2007 and 60/985,024, filed Nov. 2, 2007, the disclosures of which are incorporated herein by reference in their entireties.

### BACKGROUND

The complement system is a critical component of the innate and adaptive immune system (reviewed by Volanakis, 1998). Complement plays an important role in microbial killing, and is essential for the transport and clearance of immune complexes. Many of the activation products of the complement system are also associated with proinflammatory or immunoregulatory functions. The complement system consists of plasma and membrane-associated proteins that are organized in three enzymatic-activation cascades: the classical, the lectin, and the alternative pathways. All three pathways can lead to the formation of the terminal complement complex (TCC) and an array of biologically active products.

In some cases, complement activation is initiated either by specific antibodies recognizing and binding to a variety of pathogens and foreign molecules, and/or by direct interaction of complement proteins with foreign substances. On activation, these pathways result in the formation of unstable protease complexes, the C3-convertases. The classical pathway C3-convertase, C4b2a, and the alternative pathway C3-convertase, C3bBb, are both able to cleave the α chain of C3 generating C3b. C3b has the potential to bind covalently to biological surfaces. C3b binding leads to opsonization for phagocytosis by polymorphonuclear cells and macrophages. When additional C3b is available, the C3-convertases can function as C5-convertases, cleaving C5 and initiating the assembly of the TCC, or membrane attack complex (MAC), which mediates cellular lysis by insertion of pore-forming protein complexes into targeted cell membranes.

The precise function of the complement system depends on tight regulation, as activation of the complement cascade leads to the production of a number of proteins that contribute to inflammation. This is beneficial when contributing to a host defense, but can be detrimental if activated on self tissue. Typically, activation of C3 in the blood is kept at a low level, and C3b deposition is limited to the surface of pathogens.

To regulate the complement system, a number of complement regulatory proteins function to restrict complement activation. These proteins interact with C3 or C4 derivatives and are encoded by closely linked genes that comprise the Regulator of Complement Activation (RCA) gene cluster on human chromosome 1q32 (Diaz-Guillen et al., 1999).

Complement factor H(CFH or 1H) a plasma protein encoded by one of the RCA genes, is a soluble activation inhibitor of the alternative complement pathway (Muller-Eberhard et al., 1980; Zipfel et al., 2002; Rodriguez de Cordoba et al., 2004). CFH prevents binding of factor B to C3b, displays decay-accelerating activity for dissociation of the C3bBb complex, acts as a cofactor for the cleavage of C3b by factor I, and blocks the generation of C5b6-9, also known as membrane attack complex (MAC) (Whaley and Ruddy, 1976; Weiler et al., 1976; Pangburn et al., 1977). CFH binds to and interacts with multiple ligands including C3b, heparin, bacterial surface proteins, the acute phase protein, C-reactive protein (CRP), adrenomedullin and cell surface receptors (Zipfel et al., 2002).

Human CFH is a member of a protein family composed of seven structurally and immunologically related, multidomain, multifunctional serum proteins. These include CFH and factor H-like protein 1 (FHL-1) and five factor H-related proteins (FHR-1 to FHR-5). Each of these proteins is composed exclusively of short consensus repeats (SCRs) or complement control modules, each encoded by a separate exon. CHF, 150 kDa, is composed of 20 SCRs. FHL-1, 43 kDa, and composed of seven SCR domains, is derived from CFH by alternative splicing (Estaller et al., 1991; Sim et al., 1993). FHL-1, like CFH, functions as a complement regulator and displays cofactor and decay-accelerating activity (Zipfel and Sherka, 1999). In addition, FHL-1 has unique functions including acting as an adhesion protein due to the presence of an exposed RGD domain in SCR4 (Hellwage et al., 1997). CFH is present in human plasma at 500 μg/ml. In contrast, the FHL-1 plasma concentration is 10-50 μg/ml. The five FHR proteins are each derived from a separate gene in the RCA gene cluster. Although members of this family differ in the number of SCRs, the individual SCR domains display a high degree of homology to each other.

The organization of CFH and related proteins into multiple, individually folded protein domains suggests a structure/function relationship. The complement regulatory domains of CFH and FHL-1 are located within the four amino terminal SCRs. Three C3-binding domains are located in CFH. The N-terminal domain (SCRs 1-4) binds to intact C3b (Gordon et al., 1995; Kuhn et al., 1995; 1996), the middle domain (SCRs 12-14) binds to the C3c fragment, and the C-terminal domain (SCR 19-20) binds to C3d (Jokiranta et al., 2000). The C-terminal domain (SCR 19-20) also blocks the lytic function of the C5b6-9 TCC (Zipfel et al., 2002). This function is absent in FHL-1. CFH and FHL-1 contain overlapping binding sites for heparin, C-reactive protein (CRP), and M-protein located in SCR7 (Giannakis et al., 2003).

Proteins in the family are predominantly synthesized in the liver, although CFH has also been demonstrated to be expressed in a wide variety of cell types, such as peripheral blood lymphocytes, myoblasts, fibroblasts, neurons, and glia cells (Friese et al., 1999). More recently, CFH sequences were identified in an expressed sequence tag library derived from human retinal pigment epithelial (RPE) cells and choroid (Wistow et al., 2002), and immunohistochemical staining identified CFH in choroid vessels and in an area bordering the RPE (Klein et al., 2005).

A CFH polymorphism is linked to an increased risk of age related macular degeneration (AMD) (Edwards et al., 2005; Klein et al., 2005; Haines et al., 2005). This polymorphism, a tyrosine to histidine change at amino acid 402 (tyr402his or Y402H), accounts for about 50% of the attributable risk of AMD. The Y→H polymorphism identified in the recent Science articles is located in SCR7 (Edwards et al., 2005; Klein et al., 2005; Haines et al., 2005).

CRP is known to activate the classic complement pathway, and inhibits the deposition of C5b6-9 through the direct binding of CFH (Mold et al., 1999). CFH binding to heparin and/or CRP could potentially be altered by the replacement of a neutral tyrosine with a positively charged histidine (Rodriguez de Cordoba et al., 2004). Elevated serum levels of CRP were observed in AMD patients compared to controls in a clinical study (Seddon et al., 2004). Furthermore, nutritional supplementation with zinc slows down the progression of AMD and biochemical studies have shown that CFH function is sensitive to zinc concentration (AREDS Research Group, 2001; Blom et al., 2003). Therefore, altered binding of CFH to CRP or heparin on retinal surfaces caused by the tyr402his substitution could affect the level of inflammation in the outer retina (Edwards et al., 2005; Klein et al., 2005; Haines et al., 2005).

CFH deficiencies have been described both in humans and animals. They are caused by mutations that result in truncations or amino acid substitutions that impair CFH function (Ault et al., 1997; Sanchez-Corral et al., 2002; Hegasy et al., 2003). Lack of CFH in plasma causes uncontrolled activation of the alternative complement pathway with consumption of C3 and other terminal complement components (Thompson & Winterborn, 1981; Ault et al., 1997). Dysfunctional CFH molecules in humans have been associated with two different renal diseases, membranoproliferative glomerulonephritis (MPGN) and atypical hemolytic uremic syndrome (Wyatt et al., 1982; Ault, 2000; Zipfel, 2001). Drusen of identical composition to that found in AMD are found in the eyes of patients with MPGN type II. This drusen normally appears in early adulthood at the time of the appearance of the kidney disease, significantly earlier than in AMD (Mullins et al., 2001; Colville et al., 2003). Furthermore, animals with CFH deficiencies develop renal disease with features of MPGN (Hogasen et al., 1995; Pickering et al., 2002). In the pig, CFH deficiency results in a progressive glomerulonephritis, similar to human MPGN type II that leads to renal failure (Hogasen et al., 1995). Similarly, the CFH knockout mouse spontaneously develops a glomerulonephritis that also resembles human MPGN type II (Pickering et al., 2002). Drusen composed of complement and immunoglobulin deposition were detected in the Ccl-2-deficient (chemokine ligand 2) or Ccr-2-deficient (Ccl-2 receptor) mouse, a possible model of AMD, indicating that rodents can develop drusen (Ambati et al., 2003).

Complement activation has been implicated in several diverse human diseases, including atherosclerosis and Alzheimer's disease. Vitronectin, an abundant component of drusen, is also a component of extracellular deposits associated with atherosclerosis (Niculescu et al., 1989), amyloidosis (Dahlback et al., 1993), elastosis (Dahlback et al., 1988), and MPGN type II (Jansen et al., 1993). Vitronectin is a multifunctional protein that functions in cell adhesion, maintenance of hemostasis, and inhibition of complement-induced cell lysis (Preissner, 1991). Furthermore, atherosclerotic plaques share a number of other constituents with drusen, such as complement components and apoliproprotein E. An association between advanced AMD and atherosclerosis of carotid arteries was reported in an epidemiological study (Vingerling et al., 1995) and another study identified a significant correlation between elastotic degeneration of nonsolar-exposed dermis and choroidal neovascularization in AMD patients (Blumenkranz et al., 1986). Finally, amyloid β peptide, a major constituent of neuritic plaques in Alzheimer's disease, is also found in drusen (Johnson et al., 2002). Amyloid β peptide has been implicated as a primary activator of complement (Bradt et al., 1998).

While the complement system mediates such manifestations of inflammation, a number of stimuli can trigger activation of the complement system. For example, TGFβ molecules induce expression of certain complement factors, while suppressing expression of other complement factors.

Age-related macular degeneration (AMD) is the most common cause of decreased vision in individuals over 65 years of age in the developed world. Dry AMD is characterized by a progressive degeneration of the macula causing central field visual loss. Approximately 25% of individuals between 65-74 have some degree of dry AMD, while the incidence increases to 40% between the ages of 75-84 (Hamdi & Kenney, 2003). In the US, an estimated 10 million people have decreased vision due to AMD, and with the increasing age of the population, 21 million people in the U.S. are at risk (Hamdi & Kenney, 2003). A more acute debilitating AMD includes florid neovascularization and extravasation in the retina, known as wet AMD. There is currently no effective therapy for AMD.

Like many other chronic diseases, AMD is caused by a combination of genetic and environmental risk factors. These risk factors include age, smoking, and family history (AREDS Research Group, 2000). A heritable component is manifest as an autosomal dominant trait in a significant proportion of affected individuals (Gorin et al., 1999).

A characteristic of AMD is the accumulation of drusen, located between the basal lamina of the retinal pigment epithelium (RPE) and the inner layer of Bruch's membrane (Pauleikhoff et al., 1990; Bressler et al., 1990). Drusen, as well as other age-related changes that occur proximal to Bruch's membrane, contribute to the dysfunction and degeneration of the RPE and retina by inducing ischemia as well as restricting the exchange of nutrient and waste products between the retina and choroid (reviewed by Bird, 1992). Several studies have indicated immune-mediated processes in the development of AMD. Importantly, autoantibodies were detected in the sera of AMD patients (Penfold et al., 1990), as predicted by the hypothesis that immune and inflammatory-mediated processes are involved in the development and/or removal of drusen.

Comprehensive analysis of the molecular composition of human drusen, as well as of the RPE cells that flank or overlie drusen, demonstrated immunoreactivity to immunoglobulins and components of the complement system that are associated with immune complex deposition (Johnson et al., 2000). Drusen also contains multifunctional proteins such as vitronectin (Hagemen et al., 1999) and apolipoprotein E (Anderson et al., 2001) that play a role in immune system modulation. In addition, molecules involved in the acute phase response to inflammation, such as amyloid P component and α₁-antitrypsin, have also been identified in drusen (Mullins et al., 2000), as well as proteins involved in coagulation and fibrinolysis (factor X, thrombin, and fibrinogen) (Mullins et al., 2000). Drusen formation and associated RPE pathology were suggested to contribute to a chronic inflammatory response that activates the complement cascade (Hageman et al., 2001; Johnson et al., 2001).

One other form of an optic disorder arising from AMD and resulting in perturbations of the retina is Geographic Atrophy, which leads to death of patches of rod and cone cells, as well as of the RPE cells.

Citation or discussion of a reference herein shall not be construed as an admission that such is prior art to the present invention.

### SUMMARY OF THE INVENTION

The present invention, in part, provides methods and compositions for modulating an immunological pathway. The present invention relates, in part, to the delivery of a molecule(s) to a cell. The cell may be in vitro or in vivo. Some embodiments of the invention relate to modulating a complement pathway, e.g., a classical, lectin or alternative pathway.

The present invention, in part, provides methods of modulating (e.g., inhibiting) complement pathways and/or complement related diseases. Inter alia, the inventors describe herein compositions and methods for modulating complement pathways. Modulation of complement pathways can be used to modulate a disease state in an animal, to study underlying mechanisms of a disease, as control arms of a study related to complement, and for the production of different components and/or end products in a complement pathway. Some embodiments of the invention can be used to study an immunological pathway, to study associated disease states, to develop treatments for a disease state(s), to create disease states in an animal (e.g., to develop a model in an animal such as a mouse or rat) or in vitro, or for screening drugs. Some embodiments of the invention involve modulating a classical complement pathway; an alternative complement pathway or a lectin complement pathway.

Provided herein are various compositions and/methods for modulating an immunological pathway, such as a complement pathway.

The inventors provide herein, inter alia, compositions/molecules for inhibiting a complement pathway. For example, the inventors have found that certain analogs of factor B attenuate complement activation by maintaining the complex of C3bB with factor D. Accordingly, provided herein are examples of factor B analogs that are shown to inhibit complement activity. Also provided are analogs of factor D that would similarly attenuate complement activation. These analogs may provide an advantage of attenuating but not completely blocking complement activation.

In some embodiments, molecule(s) are delivered to determine their effect, or lack thereof, e.g., on a part of the eye. In some embodiments, molecules are delivered to provide a beneficial or therapeutic effect, e.g., in a human. In some embodiments, molecules (e.g., a protein) of the invention are used to study a biological pathway or disease state, to screen drugs, or as controls in studies and/or assays. In some embodiments, a molecule(s) modulates, enhances, mediates, or inhibits a complement pathway, e.g., a classical pathway, a lectin pathway, or an alternative pathway. In some embodiments, a molecule of the invention is a peptide, a protein, a complement inhibitory factor or a nucleic acid.

Additionally, the invention provides various methods for delivering molecules of the invention, e.g., to an eye or to particular parts/area of an eye. The methods of the invention also contemplate delivering a molecule(s) one or multiple times, e.g., as described herein.

Some embodiments of the invention provide methods and/or compositions for studying, inhibiting, stabilizing, exasperating (e.g., to produce a disease model in an animal), curing, treating, preventing, diminishing the severity of, shortening the course of, ameliorating, or altering the pathology, signs or symptoms of a disease or condition. In some embodiments, a disease or condition is a complement-mediated, complement-associated, complement-related or complement-dependent disease or condition. In some embodiments, a disease or condition is a blinding ocular disease, a disease arising from inflammation, early age related macular degeneration, age-related macular degeneration (AMD), wet AMD, glaucoma, uveitis, geography atrophy, diabetic proliferative retinopathy and others as described herein.

The invention also provides methods and compositions for blocking, inhibiting, enhancing and/or modulating (i) a reaction involving C3b and factor B; (ii) a reaction involving C3bB and factor D; (iii) a reaction involving C3b, factor B and factor D; (iv) cleavage of factor B (e.g., by factor D); (v) cleavage of C3 (e.g., by factor B); (vi) dissociation of factor D from C3bBD and/or (vii) a complement pathway, e.g., alternative complement pathway.

Some embodiments of the invention provide methods of treating, ameliorating, or preventing a factor B-mediated disease in a subject, e.g., by inhibiting the synthesis, cleavage or activity of factor B.

The present invention also provides proteins including mutants of or variants of components of a complement pathway, such as factor B, e.g., as described herein. Some embodiments of the invention provide a factor B variant with one or more of the following characteristics: reduced ability to cleave C3, tighter binding to factor D, tighter binding to C3b or reduced ability to be cleaved by factor D.

The invention includes (i) molecules that bind to both factors C3b and D e.g., fB3, a bispecific antibody, etc; (ii) complement protein analogs with increased binding (as compared to their native form) to both factors C3b and D; (iii) complement protein analogs with increased binding (as compared to their native form) to factor D; and (iv) complement protein analogs with increased binding (as compared to their native form) to C3bB complex. The invention also includes methods of inhibiting a complement pathway using the molecules of the invention, such as those of i-iv, described in this paragraph.

Some embodiments of the invention provide methods of treating a complement-mediated disease comprising administering to a patient in need of treatment a pharmaceutical composition comprising a molecule that inhibits complement activity and/or a vector that comprises a transgene that codes for the molecule and/or a cell comprising the vector expressing the molecule. In some embodiments, the molecule is a complement factor analog.

In some embodiments, a complement factor comprises one or more altered functions. In some embodiments, the altered complement factor comprises diminished protease activity. In some embodiments, the complement factor is factor B. In some embodiments, a complement factor B analog comprises increased C3b binding affinity as compared to the unaltered complement factor. In some cases this is accomplished by an alteration in the C3b binding domain such as a substitution of an aspartic acid, an asparagine or both. In some embodiments, the aspartic acid is replaced with glycine, alanine or asparagine. In some embodiments, the asparagine is replaced with glycine, alanine, or aspartic acid. In some embodiments, this aspartic acid corresponds to amino acid 279 of SEQ ID NO:2 and this asparagine corresponds to amino acid 285 of SEQ ID NO:2.

In some embodiments, the factor B comprises an alteration in the active site of the serine protease domain. In some embodiments, a serine protease domain comprises or consists of the amino acids corresponding to 739 to 746 of SEQ ID NO:2. In some cases, an alteration comprises substitution of an aspartic acid, e.g., with serine, tyrosine, glycine, alanine, glutamic acid or asparagine. In some embodiments, the substituted aspartic acid corresponds to amino acid 740 of SEQ ID NO:2. In some embodiments, a complement factor B analog comprises SEQ ID NO:4 or comprises amino acids 26-764 of SEQ ID NO:4.

In some embodiments, a complement factor B analog has diminished ability to be cleaved by factor D as compared to the native factor B. In some embodiments, a factor B analog comprises an alteration in the factor D cleavage site. In some embodiments, the alteration comprises substitution of at least one lysine, an arginine or both, e.g., with alanine for each. In some embodiments, the at least one lysine corresponds to amino acid 258 or 260 of SEQ ID NO:2 and said arginine corresponds to amino acid 259 of SEQ ID NO:2.

The invention also provides factor D analogs with diminished proteolytic activity as compared to a native complement factor D and/or increased C3bBb binding affinity as compared to a native complement factor D. In some embodiments, a complement factor D analog comprises a reduced ability to cleave factor B as compared to a native complement factor D. In some embodiments, a complement factor D analog comprises an alteration in the serine protease catalytic domain of fD. In some embodiments, an alteration in the serine protease catalytic domain of fD comprises: (i) a substitution or deletion of an amino acid corresponding to His66, Asp114, or Ser208 of SEQ ID NO:27 (human fD); or (ii) an insertion of at least one amino acid next to the His66, Asp114, or Ser208 of SEQ ID NO:27. In some embodiments, the amino acid corresponding to the His66 is substituted with at least one neutral amino acid, at least one negatively charged amino acid or at least one nonpolar amino acid. In some embodiments, the amino acid corresponding to the Asp114 is substituted with at least one positively charged or at least one nonpolar amino acid. In some embodiments, the amino acid corresponding to the Ser 208 is substituted with at least one charged or at least one nonpolar amino acid. In some embodiments, a complement factor D analog comprises one or more additional amino acids at the N-terminus as compared to a wild-type factor D. In some embodiments, the one or more additional amino acids comprise glycine and arginine.

Some embodiments of the invention provide methods of treating a complement-mediated disease comprising administering to a patient in need of treatment a pharmaceutical composition comprising: (i) a complement factor D analog that inhibits or reduces complement activity; (ii) a vector that encodes the complement factor D analog; or (iii) cells containing the vector that encodes the complement factor D analog.

In some embodiments, a molecule that inhibits complement activity is a molecule that binds a complement factor. In some embodiments, this molecule comprises at least one complementary determining region of an antibody that binds a complement factor. In some embodiments, the molecule is an antibody or fragment thereof that binds the complement factor. In some embodiments, an antibody is a human, humanized, chimeric, murine, chicken or rabbit antibody. In some embodiments, a binding molecule is an aptamer that binds the complement factor. In some embodiments, a binding molecule of the invention binds factor B, factor C3b or factor D.

In some embodiments, a complement mediated disease is a disease of the eye. In some embodiments, a pharmaceutical composition of the invention is delivered/administered to the eye, e.g., via intravitreal injection, subretinal injection, injection into the anterior chamber of the eye, injection or application locally on the cornea, subconjunctival injection, subtenon injection, or by eyedrops.

The invention also provides a factor B analog comprising diminished protease activity and altered C3b binding affinity. Some embodiments of the invention provide a complement factor D analog comprising diminished protease activity.

Some embodiments of the invention provide a method of treating a disease in a mammal comprising administering to the mammal a pharmaceutical composition comprising a molecule that inhibits or reduces complement activity. In some instances, the molecule is a protein or a nucleic acid. In some embodiments, the pharmaceutical composition comprises a vector that encodes the molecule. In some embodiments, the protein is an analog of a complement pathway component, e.g., a factor D or factor B analog. In some embodiments, an analog is a human factor B1, B2 or B3 or a mouse factor B1, B2 or B3.

Some methods of the invention relate to methods of treatment or prevention of a complement-mediated disease or disorder, for example, wherein the disease is drusen formation, macular degeneration, AMD, atherosclerosis, diabetic retinopathy, vitreoretinopathy, corneal inflammation, airway hyperresponsiveness, immune-related diseases, autoimmune-related diseases, lupus nephritis, systemic lupus erythematosus (SLE), arthritis, rheumatologic diseases, anti-phospholipid antibody syndrome, intestinal and renal I/R injury, asthma, atypical hemolytic-uremic syndrome, Type II membranoproliferative glomerulonephritis, non-proliferative glomerulonephritis, fetal loss, glaucoma, uveitis, ocular hypertension, brain injury, stroke, post-traumatic organ damage, post infarction organ damage, vasculitis, ischemic-reperfusion injury, trauma of heart and lung bypass procedures, for example, as used in open heart surgery, cerebrovascular accident, Alzheimer's disease, transplant rejection, infections, sepsis, septic shock, Sjögren's syndrome, myasthenia gravis, antibody-mediated skin diseases, Type I and Type II diabetes mellitus, thyroiditis, idiopathic thrombocytopenic purpura and hemolytic anemia, neuropathies, multiple sclerosis, cardiopulmonary bypass injury, polyarteritis nodosa, Henoch Schonlein purpura, serum sickness, Goodpasture's disease, systemic necrotizing vasculitis, post streptococcal glomerulonephritis, idiopathic pulmonary fibrosis, membranous glomerulonephritis, myocardial infarction, acute shock lung syndrome, adult respiratory distress syndrome, reperfusion, rejection and/or a complement mediated disease.

Some methods of the invention comprise administration of one or more of Factor H, Factor H-like 1, MCP, DAF, CD59 or a soluble form of MCP either alone or prior to, subsequent to or concurrently with a complement factor analog of the invention.

In some embodiments, a catalytic antibody is used to inhibit complement activity. In some embodiments, a catalytic antibody acts as a protease, such as by cleaving factor B, factor D, factor Bb, factor C3 and/or factor C3b complement protein.

In some embodiments, an RNA that inhibits expression of a complement protein is used, e.g., wherein the RNA is a ribozyme, an antisense oligonucleotide, a siRNA, a miRNA or an RNAi and, e.g., wherein the complement protein is C3, fB, fD, C5, C6, C7, C8, or C9. In some embodiments, a pharmaceutical composition comprises RNA that inhibits expression of at least two different complement proteins.

Some embodiments of the invention provide an isolated molecule that binds to both factors C3b and D, wherein the molecule is not a native factor B, an fB1, fB2 or fB3. Some embodiments of the invention provide a complement protein analog with increased binding to both factors C3b and D, as compared to their native form, wherein the complement protein analog is not an fB1, fB2 or fB3. Some embodiments of the invention provide a complement protein analog with increased binding to factor D, as compared to their native form, wherein the complement protein analog is not an fB1, fB2 or fB3. Some embodiments of the invention provide a complement protein analog with increased binding to C3bB complex, as compared to their native form, wherein the complement protein analog is not an fB1, fB2 or fB3. In some embodiments, a complement protein analog has increased binding of at least 2-fold, e.g., as measured by immunoprecipitation.

In some embodiments, a vector is a retroviral vector, a lentiviral vector, an adenoviral vector, an AAV vector, a Herpes viral vector, a Hepatitis viral vector, such as a Hepatitis B or Hepatitis D vector, an SV40 vector and an EBV vector. In some embodiments, a lentivirus is HIV, EIAV, SIV, FIV or BIV. In some embodiments, a viral vector comprises decay accelerating factor. The invention also provides a cell that produces a viral vector of the invention.

In some embodiments of the invention, an anti-inflammatory is administered prior to, concurrently with, and/or after the administration of a pharmaceutical composition of the invention. In some embodiments, an anti-inflammatory is administered in the same solution and/or same syringe as the pharmaceutical composition. In some embodiments, an anti-inflammatory is administered to the eye. Anti-inflammatories include, but are not limited to, dexamethasone, dexamethasone sodium metasulfobenzoate, dexamethasone sodium phosphate, rapamycin, FK506, fluorometholone, bromfenac, pranoprofen, RESTASIS™, a cyclosporine ophthalmic emulsion, naproxen, glucocorticoids, ketorolac, ibuprofen, tolmetin, non-steroidal anti-inflammatory drugs, steroidal anti-inflammatory drugs, diclofenac, flurbiprofen, indomethacin, and suprofen.

In some embodiments, complement activity is inhibited by administering to a mammal a first molecule that inhibits complement activity and a vector that encodes a second molecule that inhibits complement activity. In some case, the first and second molecules are different. In some cases, the first molecule is administered prior to, concurrently with, and/or after administration of the vector. In some cases, the first molecule and the vector are administered in the same solution and/or same syringe. Sometimes the first molecule, the vector or both may be administered to the eye.

Some embodiments of the invention provide, a complement factor D analog comprising diminished proteolytic activity as compared to a native complement factor D.

In some embodiments of the invention, a molecule of the invention or a vector encoding said molecule is administered about or at least once every: week; month; 2 months; 3 months; 6 months; 9 months; year; 18 months; 2 years; 30 months; 3 years; 5 years; or 10 years to an individual, e.g., administered to the eye. In some embodiments, a molecule of the invention or a vector encoding said molecule is administered once and inhibits complement activity for a day or longer, for a month of longer, or for a protracted period of time up to the life of the individual. In other embodiments, the molecule of the invention or vector encoding said molecule is administered not more than once: a week; a month; every 2 months; every 3 months; every 6 months; every 9 months; every year; every 18 months; every 2 years; every 30 months; every 3 years; every 5 years; or every 10 years to an individual, e.g., administered to the eye.

Some embodiments of the invention provide a vector construct or viral vector carrying a nucleic acid encoding a molecule of the invention (e.g., a therapeutic molecule), such as a complement inhibitory factor, such as decay accelerating factor (DAF) or a complement factor B analog that lacks or has less of a biological function as compared to the wild type molecule, or a binding molecule (e.g., an aptamer, antibody, antibody-like or antibody-derived molecule) that specifically binds to a molecule involved in a complement function, pathway or activity. In some embodiments, transformations utilizing a vector construct or viral vector of the invention provide sustained delivery to and/or expression of a molecule from a cell, e.g., in the retina.

The present invention additionally provides methods for transforming (in vivo or in vitro) a cell, a particular cell type(s) or a population of cells. Some methods of the invention can be used to transform cells including, but not limited to, retinal cells and/or RPE cells. Some methods of the invention can be used to transform cells of the sclera, cornea, iris, ciliary body, choroid, conjunctiva, tenons capsule, retina, subretinal tissue, extraocular adipose, muscle (e.g., extraocular muscle) and/or fascial tissue. However, the invention is not limited to transforming certain cell types. In some embodiments, the cell is in a particular organ or compartment within an animal, such as brain, an eye, spinal cord, a joint, within the circulatory system, and/or the blood. In some embodiments, a molecule is delivered to a cell and subsequently, the cell is introduced into an animal.

Some embodiments of the invention provide methods for detecting efficacy or measuring efficacy comprising detecting and/or measuring complement activity and/or a complement pathway component and/or its activity, e.g., after administration of a composition of the invention. In some embodiments, this can be measured continually or periodically to monitor a disease state and/or be used in the process of determining further treatment methods (related to those described herein or other methods).

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” The use of the term/phrase “and/or” when used with a list means one or more of the listed items may be utilized, e.g., it is not limited to one or all of the elements.

Additional features and advantages are described in the following Detailed Description.

### BRIEF DESCRIPTION OF THE DRAWINGS

For the purpose of illustrating the invention, there are depicted in the drawings certain embodiments of the invention. However, the invention is not limited to the precise arrangements and instrumentalities of embodiments depicted in the drawings.

FIG. 1 shows examples depicting the progression of AMD. Early AMD is characterized by the deposition of drusen beneath the retinal pigment epithelial (RPE) cell layer. Drusen is visualized as pale or white spots in the middle panel.

FIG. 2 shows examples of four constructs for making BIV (bovine immunodeficiency virus)-based vectors. RSV is a Rous sarcoma virus (RSV) promoter. CMV is a cytomegalovirus (CMV) enhancer juxtaposed to the R region of the 5′ long terminal repeat (LTR). SIN indicates deletions in the enhancer and promoter from the U3 region of the 3′ LTR to yield a self-inactivating vector. φ is the packaging signal that directs packaging of vector RNA into the viral particles. Heterologous Gene refers to an expression cassette that includes both a promoter and a coding region encoding a molecule, e.g., a therapeutic molecule. Gp64 is a baculovirus gp64 envelope gene.

FIG. 3 shows expression of a green fluorescent protein (GFP) in BIV vector-transduced rat retina. 3×10⁵ transducing units (tu) in 3 μl were administered via subretinal injection. One month later, the rat was sacrificed, the retina was harvested, and slits were cut into the retina to prepare a flat whole mount. FIG. 3A: The grey outline depicts the edges of the retina. The lighter area shows transduction and GFP expression at and about the injection site. FIG. 3B shows that the immunohistochemical staining for GFP is predominantly in the RPE layer.

FIG. 4 shows expression of GFP in BIV vector-transduced mouse retinas. FIG. 4A depicts transduction and expression in an adult mouse retina two weeks after subretinal injection of 1×10⁵ tu in 1 μl. FIG. 4B depicts transduction and expression in a newborn mouse two weeks after intravitreal injection of 1×10⁵ tu in 1 μl. FIG. 4C is a high power view of FIG. 4B.

FIG. 5 shows a flat mount of rabbit retina one month after ocular administration of a BIV GFP vector showing high level expression of GFP in the RPE cells.

FIG. 6 shows expression of GFP in monkey RPE cells ten weeks after administration of a BIV vector encoding GFP.

FIG. 7 shows that a BIV vector can efficiently transduce primary human RPE cells. The left panel shows staining for RPE65, a 65 KD RPE specific protein. The central and right panels show bright field and fluorescent views of the cells transduced with GFP vector.

FIG. 8 shows that a BIV vector encoding an endostatin demonstrated efficacy dampening angiogenesis in an animal model of florid neovascularization. The top left panel depicts an untreated eye. The bottom two left panels depict eyes treated with the control vector and those on the right depict eyes treated with the BIV endostatin vector. The top panels are fluorescein angiographs. The middle panels are histological sections of the retinas. The bottom panels are cross-sections of the entire eyes.

FIG. 9 shows the curves of vector titer and BSA level in the elution fractions from a Sephacryl S 500-HR column. For this study, the culture medium from which the vector was purified was supplemented with 2% FBS. Details are described in Example 30.

FIG. 10 shows that a BIV vector expressing T2-TrpRS inhibited neovascularization in the laser injury model. FIG. 10 shows the average size of the neovascular areas from the two cohorts. The areas of neovascularization were significantly smaller in the T2-TrpRS (a carboxyl-terminal fragment of tryptophan tRNA synthetase) vector-treated eyes.

FIG. 11 depicts the classical and lectin complement pathways. The classical pathway is initiated through C1 while the lectin pathway is initiated through mannose binding lectin (MBL). C4bC2a is a protease that cleaves C3 to C3a and C3b and is termed the C3 convertase. Similarly, C4bC2aC3b cleaves C5 to C5a and C5b and is termed the C5 convertase. C3a, C4a, and C5a have inflammatory properties and attract phagocytotic cells. C5b6-9 forms the membrane attack complex (MAC), which creates membrane pores that kill infectious agents but can also damage host cells. MASP is mannan-binding lectin associated serine protease.

FIG. 12 depicts the alternative complement pathway. This pathway is constitutively active at a low level through spontaneous cleavage of C3. In the presence of an appropriate surface, C3b binds to complement factor B (fB). This complex is then cleaved by complement factor D (fD) to yield C3bBb. Spontaneous dissociation (“decay”) of this complex within minutes leads to its inactivation, whereas stabilization by properdin generates a complex that cleaves C3; that is, a C3 convertase. Several of the factors that attenuate the complement pathways do so by accelerating the decay of the C3 and C5 convertases (see Table One, below). Please note: C3b participates in the C3 convertase to generate additional C3b thereby creating a positive feedback loop as shown by the large arrow. C3bBb is a C3 convertase. C3bBbC3b is a C5 convertase.

FIG. 13 shows expression of vector-derived fB in human retinal cells. ARPE cells (an RPE derived cell line) were transduced with BIV-based vectors encoding fB constructs, see Example 8. Medium from each of the transduced cell populations was subjected to Western analysis and probed for fB. Lane 1, 100 ng of purified human plasma-derived fB; Lanes 2-5, 40 μl of media from cells transduced with vectors encoding human wild type fB, fB3, fB2, and fB1, respectively. All of the lanes are from the same gel.

FIG. 14 shows structure of a single chain antibody that can be subcloned, e.g., as a Heterologous Gene in a transfer vector construct, e.g., of FIG. 2, or expressed from a cell. Leader is a leader sequence to direct secretion; V_{L} and V_{H} are the variable light and heavy chains or CDR containing portions thereof, respectively, connected by a linker, e.g., which are known in the art. In some embodiments, a heavy chain sequence can be placed upstream of the light chain sequence.

FIG. 15 shows results from a hemolytic assay of vector-derived human wild type fB and fB dominant negatives. The positive control is Erab (rabbit erythrocytes) mixed with distilled water to produce 100% hemolysis. Purified fB Protein represents hemolysis by human fB-depleted serum supplemented with 500 ng of purified human plasma-derived fB. The Negative Control represents hemolysis by fB-depleted human serum alone. The five right-hand bars represent hemolysis by fB-depleted serum with the addition of culture medium from cells transduced with vectors encoding GFP, wild type human fB, fB1, fB2, or fB3.

FIG. 16 shows lentiviral vector gene transfer to rat aorta and mouse brain. FIG. 16A demonstrates transduction of a section of rat aorta with an HIV-derived lentiviral vector. FIG. 16B demonstrates gene transfer to mouse brain using a BIV GFP vector. The related methods are described in Example 14.

FIG. 17 shows results of a hemolytic activity assay to assess alternative complement pathway activity using various factor B mutants. The Y-axis displays the relative hemolytic activity as measured by the hemoglobin level released into the supernatant after lysis of the erythrocytes. X-axis from left to right: Positive control with 100% lysis, red blood cells (RBC) lysed in water; WT (wild-type) hfB, factor B-depleted human serum supplemented with 500 ng of purified plasma derived human factor B protein (catalog# A408, Quidel, San Diego, Calif.); Negative control, the RBCs were incubated in isotonic saline (no red blood cell lysis); Wild type hfB vector, factor B-depleted human serum supplemented with culture medium of the cells transduced with BIV vector encoding wild type factor B; GFP vector, factor B-depleted human serum supplemented with culture medium of the cells transduced with BIV vector encoding GFP; Mutant hfB1 vector, factor B-depleted human serum supplemented with culture medium of the cells transduced with BIV vector encoding mutant fB1; Mutant hfB2 vector, factor B-depleted human serum supplemented with culture medium of the cells transduced with BIV vector encoding mutant fB2; Mutant hfB3 vector, factor B-depleted human serum supplemented with culture medium of the cells transduced with BIV vector encoding mutant fB3; GFP vector+WT fB 1:1, factor B-depleted human serum supplemented with a mixture of culture medium of the cells transduced with BIV vector encoding GFP and culture medium of the cells transduced with BIV vector encoding wild type factor B at 1 to 1 ratio; Mutant hfB1+WT fB 1:1, factor B-depleted human serum supplemented with a mixture of culture medium of the cells transduced with BIV vector encoding mutant factor B1 and culture medium of the cells transduced with BIV vector encoding wild type factor B at 1 to 1 ratio; Mutant hfB2+WT fB 1:1, factor B-depleted human serum supplemented with a mixture of culture medium of the cells transduced with BIV vector encoding mutant factor B2 and culture medium of the cells transduced with BIV vector encoding wild type factor B at 1 to 1 ratio; Mutant hfB3+WT fB 1:1, factor B-depleted human serum supplemented with a mixture of culture medium of the cells transduced with BIV vector encoding mutant factor B3 and culture medium of the cells transduced with BIV vector encoding wild type factor B at 1 to 1 ratio; GFP vector+WT fB 2:1, factor B-depleted human serum supplemented with a mixture of culture medium of the cells transduced with BIV vector encoding GFP and culture medium of the cells transduced with BIV vector encoding wild type human factor B at 2 to 1 ratio; Mutant hfB1+WT fB 2:1, factor B-depleted human serum supplemented with a mixture of culture medium of the cells transduced with BIV vector encoding mutant factor B1 and culture medium of the cells transduced with BIV vector encoding wild type factor B at 2 to 1 ratio; Mutant hfB2+WT fB 2:1, factor B-depleted human serum supplemented with a mixture of culture medium of the cells transduced with BIV vector encoding mutant factor B2 and culture medium of the cells transduced with BIV vector encoding wild type factor B at 2 to 1 ratio; Mutant hfB3+WT fB 2:1, factor B-depleted human serum supplemented with a mixture of culture medium of the cells transduced with BIV vector encoding mutant factor B3 and culture medium of the cells transduced with BIV vector encoding wild type factor B at 2 to 1 ratio.

FIG. 18 shows results of a hemolytic activity assay to assess alternative complement pathway activity. Y-axis displays the relative hemolytic activity as measured by the hemoglobin level released to the supernatant after lysis of erythrocytes. X-axis from left to right: Positive control with 100% lysis, RBC lysed in water; Blank, the RBC was incubated in isotonic saline (no red blood cell lysis); Four-fold diluted mouse serum (50 ul) was added to the following samples (40 ul each): GFP vector, culture medium of the cells transduced with BIV vector encoding GFP mixed with culture medium of the cells transduced with BIV vector encoding wild type mouse factor B at 1 to 1 ratio; Mutant mfB1 vector, culture medium of the cells transduced with BIV vector encoding mouse mutant fB1 mixed culture medium of the cells transduced with BIV vector encoding wild type mouse factor B at 1 to 1 ratio; Mutant mfB2 vector, culture medium of the cells transduced with BIV vector encoding mouse mutant fB2 mixed with culture medium of the cells transduced with BIV vector encoding wild type mouse factor B at 1 to 1 ratio; Mutant mfB3 vector, culture medium of the cells transduced with BIV vector encoding mouse mutant fB3 mixed with culture medium of the cells transduced with BIV vector encoding wild type mouse factor B at 1 to 1 ratio; GFP vector, culture medium of the cells transduced with BIV vector encoding GFP mixed with culture medium of the cells transduced with BIV vector encoding wild type mouse factor B at 2 to 1 ratio; Mutant mfB1 vector, culture medium of the cells transduced with BIV vector encoding mouse mutant fB1 mixed with culture medium of the cells transduced with BIV vector encoding wild type mouse factor B at 2 to 1 ratio; Mutant mfB2 vector, culture medium of the cells transduced with BIV vector encoding mouse mutant fB2 mixed with culture medium of the cells transduced with BIV vector encoding wild type mouse factor B at 2 to 1 ratio; Mutant mfB3 vector, culture medium of the cells transduced with BIV vector encoding mouse mutant fB3 mixed culture medium of the cells transduced with BIV vector encoding wild type mouse factor B at 2 to 1 ratio; GFP vector, culture medium of the cells transduced with BIV vector encoding GFP mixed with culture medium of the cells transduced with BIV vector encoding wild type mouse factor B at 1 to 1 ratio; Mutant hfB1 vector, culture medium of the cells transduced with BIV vector encoding human mutant fB1 mixed with culture medium of the cells transduced with BIV vector encoding wild type mouse factor B at 1 to 1 ratio; Mutant hfB2 vector, culture medium of the cells transduced with BIV vector encoding human mutant fB2 mixed with culture medium of the cells transduced with BIV vector encoding wild type mouse factor B at 1 to 1 ratio; Mutant hfB3 vector, culture medium of the cells transduced with BIV vector encoding human mutant fB3 mixed with culture medium of the cells transduced with BIV vector encoding wild type mouse factor B at 1 to 1 ratio; GFP vector, culture medium of the cells transduced with BIV vector encoding GFP mixed with culture medium of the cells transduced with BIV vector encoding wild type mouse factor B at 2 to 1 ratio; Mutant hfB1 vector, culture medium of the cells transduced with BIV vector encoding human mutant fB1 mixed with culture medium of the cells transduced with BIV vector encoding wild type mouse factor B at 2 to 1 ratio; Mutant hfB2 vector, culture medium of the cells transduced with BIV vector encoding human mutant fB2 mixed with culture medium of the cells transduced with BIV vector encoding wild type mouse factor B at 2 to 1 ratio; Mutant hfB3 vector, culture medium of the cells transduced with BIV vector encoding human mutant fB3 mixed with culture medium of the cells transduced with BIV vector encoding wild type mouse factor B at 2 to 1 ratio.

FIG. 19 shows a hemolytic activity assay to assess alternative complement pathway activity. Y-axis displays the relative hemolytic activity as measured by the hemoglobin level released to the supernatant after lysis of erythrocytes. X-axis from left to right: Positive control with 100% lysis, RBC lysed in water; Blank, RBC incubated in isotonic buffer (no lysis of red blood cells); GFP, culture medium of the cells transduced with BIV GFP vector; WT hfB vector, factor B-depleted human serum supplemented with culture medium of the cells transduced with BIV vector encoding wild type factor B; Mutant hfB 1 vector, factor B-depleted human serum supplemented with culture medium of cells transduced with BIV vector encoding mutant fB1; Mutant hfB2 vector, factor B-depleted human serum supplemented with culture medium of cells transduced with BIV vector encoding mutant fB2; Mutant hfB3 vector, factor B-depleted human serum supplemented with culture medium of cells transduced with BIV vector encoding mutant fB3; GFP vector+1:1 wWT hfB, factor B-depleted human serum supplemented with a mixture of culture medium of the cells transduced with BIV vector encoding GFP and culture medium of the cells transduced with BIV vector encoding wild type human factor B at 1 to 1 ratio; Mutant mfB1+1:1 wWT hfB, factor B-depleted human serum supplemented with a mixture of culture medium of the cells transduced with BIV vector encoding mouse mutant factor B1 and culture medium of the cells transduced with BIV vector encoding wild type human factor B at 1 to 1 ratio; Mutant mfB2+1:1 wWT hfB, factor B-depleted human serum supplemented with a mixture of culture medium of the cells transduced with BIV vector encoding mouse mutant factor B2 and culture medium of the cells transduced with BIV vector encoding human wild type factor B at 1 to 1 ratio; Mutant mfB3+1:1 wWT hfB, factor B-depleted human serum supplemented with a mixture of culture medium of the cells transduced with BIV vector encoding mouse mutant factor B3 and culture medium of the cells transduced with BIV vector encoding human wild type factor B at 1 to 1 ratio; GFP vector+2:1 wWT MB, factor B-depleted human serum supplemented with a mixture of culture medium of the cells transduced with BIV vector encoding GFP and culture medium of the cells transduced with BIV vector encoding wild type human factor B at 2 to 1 ratio; Mutant mfB1+2:1 wWT hfB, factor B-depleted human serum supplemented with a mixture of culture medium of the cells transduced with BIV vector encoding mouse mutant factor B1 and culture medium of the cells transduced with BIV vector encoding wild type human factor B at 2 to 1 ratio; Mutant mfB2+2:1 wWT hfB, factor B-depleted human serum supplemented with a mixture of culture medium of the cells transduced with BIV vector encoding mouse mutant factor B2 and culture medium of the cells transduced with BIV vector encoding human wild type factor B at 2 to 1 ratio; Mutant mfB3+2:1 wWT hfB, factor B-depleted human serum supplemented with a mixture of culture medium of the cells transduced with BIV vector encoding mouse mutant factor B3 and culture medium of the cells transduced with BIV vector encoding human wild type factor B at 2 to 1 ratio; GFP vector+4:1 wWT MB, factor B-depleted human serum supplemented with a mixture of culture medium of the cells transduced with BIV vector encoding GFP and culture medium of the cells transduced with BIV vector encoding wild type human factor B at 4 to 1 ratio; Mutant mfB1+4:1 wWT hfB, factor B-depleted human serum supplemented with a mixture of culture medium of the cells transduced with BIV vector encoding mouse mutant factor B1 and culture medium of the cells transduced with BIV vector encoding wild type human factor B at 4 to 1 ratio; Mutant mfB2+4:1 wWT hfB, factor B-depleted human serum supplemented with a mixture of culture medium of the cells transduced with BIV vector encoding mouse mutant factor B2 and culture medium of the cells transduced with BIV vector encoding human wild type factor B at 4 to 1 ratio; Mutant mfB3+4:1 wWT hfB, factor B-depleted human serum supplemented with a mixture of culture medium of the cells transduced with BIV vector encoding mouse mutant factor B3 and culture medium of the cells transduced with BIV vector encoding human wild type factor B at 4 to 1 ratio.

FIG. 20 shows a hemolytic activity assay to assess alternative complement pathway activity. Y-axis displays the relative hemolytic activity as measured by the hemoglobin level released to the supernatant after lysis of erythrocytes. X-axis from left to right: Positive control with 100% lysis, RBC lysed in water; Negative control, RBC incubated in isotonic buffer (no lysis of red blood cells); GFP vector, culture medium of the cells transduced with BIV vector encoding GFP (40 ul) mixed with two-fold diluted pig serum (50 ul); Wild type hfB vector, culture medium of the cells transduced with BIV vector encoding wild type factor B (40 ul) mixed with two-fold diluted pig serum (50 ul); Mutant hfB1 vector, culture medium of the cells transduced with BIV vector encoding human mutant fB1 (40 ul) mixed with two-fold diluted pig serum (50 ul); Mutant hfB2 vector, culture medium of the cells transduced with BIV vector encoding human mutant fB2 (40 ul) mixed with two-fold diluted pig serum (50 ul); Mutant hfB3 vector, culture medium of the cells transduced with BIV vector encoding human mutant fB3 (40 ul) mixed with two-fold diluted pig serum (50 ul); GFP vector, culture medium of the cells transduced with BIV vector encoding GFP (40 ul) mixed with four-fold diluted pig serum (50 ul); Wild type hfB vector, culture medium of the cells transduced with BIV vector encoding wild type factor B (40 ul) mixed with four-fold diluted pig serum (50 ul); Mutant hfB1 vector, culture medium of the cells transduced with BIV vector encoding human mutant fB1 (40 ul) mixed with four-fold diluted pig serum (50 ul); Mutant hfB2 vector, culture medium of the cells transduced with BIV vector encoding human mutant fB2 (40 ul) mixed with four-fold diluted pig serum (50 ul); Mutant hfB3 vector, culture medium of the cells transduced with BIV vector encoding human mutant fB3 (40 ul) mixed with four-fold diluted pig serum (50 ul); GFP vector, culture medium of the cells transduced with BIV vector encoding GFP (40 ul) mixed with six-fold diluted pig serum (50 ul); Wild type hfB vector, culture medium of the cells transduced with BIV vector encoding wild type factor B (40 ul) mixed with six-fold diluted pig serum (50 ul); Mutant hfB1 vector, culture medium of the cells transduced with BIV vector encoding human mutant fB1 (40 ul) mixed with six-fold diluted pig serum (50 ul); Mutant hfB2 vector, culture medium of the cells transduced with BIV vector encoding human mutant fB2 (40 ul) mixed with six-fold diluted pig serum (50 ul); Mutant hfB3 vector, culture medium of the cells transduced with BIV vector encoding human mutant fB3 (40 ul) mixed with six-fold diluted pig serum (50 ul).

FIG. 21 shows C3b-dependent human factor B cleavage by factor D. Western blot detection of full-length factor B or factor B cleavage products, Bb and Ba. Lane 1, Purified human factor B protein as positive control; Lane 2, Culture medium of the cells transduced by BIV vector encoding GFP incubated with factor D; Lane 3, Culture medium of the cells transduced by BIV vector encoding human wild type factor B incubated with factor D; Lane 4, Culture medium of the cells transduced by BIV vector encoding human mutant factor B1 incubated with factor D; Lane 5, Culture medium of the cells transduced by BIV vector encoding human mutant factor B2 incubated with factor D; Lane 6, Culture medium of the cells transduced by BIV vector encoding human mutant factor B3 incubated with factor D; Lane 7, Molecular weight marker; Lane 8, Culture medium of the cells transduced by BIV vector encoding GFP incubated with C3b and factor D; Lane 9, Culture medium of the cells transduced by BIV vector encoding human wild type factor B incubated with C3b and factor D; Lane 10, Culture medium of the cells transduced by BIV vector encoding human mutant factor B1 incubated with C3b and factor D; Lane 11, Culture medium of the cells transduced by BIV vector encoding human mutant factor B2 incubated with C3b and factor D; Lane 12, Culture medium of the cells transduced by BIV vector encoding human mutant factor B3 incubated with C3b and factor D. The reaction mixture for each sample is described in Table two.

FIG. 22 shows a factor B and C3b binding assay. C3b, factor D, and factor B (wt or mutants) together were incubated in a reaction mixture. The reaction mixture was immunoprecipitated with polyclonal anti-factor B antiserum, separated by SDS-PAGE, and analyzed by Western Blot analysis with anti-C3b polyclonal antiserum. Lane 1, purified C3b protein as a positive control (the lower band is C3 beta-chain co-purified with C3b); Lane 2, culture medium of the cells transduced with BIV vector encoding GFP incubated with C3b and factor D; Lane 3, culture medium of the cells transduced with BIV vector encoding human wild type factor B incubated with C3b and factor D; Lane 4, culture medium of the cells transduced with BIV vector encoding human mutant factor B3 incubated with C3b and factor D; Lane 5, culture medium of the cells transduced with BIV vector encoding human mutant factor B2 incubated with C3b and factor D; and Lane 6, culture medium of the cells transduced with BIV vector encoding human mutant factor B1 incubated with C3b and factor D.

FIG. 23 shows an assay for human complement factor B and factor D binding. Details of the experiment are found in Example 21, below. Lane 1, negative control, the reaction was performed in the absence of C3b, factor D, and factor B; Lane 2, culture medium of the cells transduced with BIV vector encoding GFP incubated with C3b and factor D; Lane 3, culture medium of the cells transduced with BIV vector encoding human wild type factor B incubated with C3b and factor D; Lane 4, molecular weight markers; Lane 5, culture medium of the cells transduced with BIV vector encoding human mutant factor B3 incubated with C3b and factor D; Lane 6, culture medium of the cells transduced with BIV vector encoding human mutant factor B2 incubated with C3b and factor D; Lane 7, culture medium of the cells transduced with BIV vector encoding human mutant factor B1 incubated with C3b and factor D; and Lane 8, purified human factor B as a positive control.

FIG. 24 shows inhibition of human alternative complement pathway activity by an anti-human factor B monoclonal antibody using a hemolytic activity assay to assess alternative complement pathway activity. The Y-axis displays the relative hemolytic activity as measured by the hemoglobin level released to the supernatant after lysis of erythrocytes. The X-axis from left to right: Positive control with 100% lysis, RBC lysed in water; Purified hfB protein, factor B-depleted human serum supplemented with 500 ng of purified human factor B protein; Negative control, the RBC was incubated in isotonic saline (no red blood cell lysis); Anti-MB mAb 0.4 μg; factor B-depleted human serum supplemented with a mixture of anti-hfB mAb (0.4 μg) and 500 ng of purified human factor B protein; Anti-hfB mAb 0.8 μg; factor B-depleted human serum supplemented with a mixture of anti-hfB mAb (0.8 μg) and 500 ng of purified human factor B protein; Anti-hfB mAb 1.6 μg, factor B-depleted human serum supplemented with a mixture of anti-MB mAb (1.6 μg) and 500 ng of purified human factor B protein; Anti-hfB mAb (2.4 μg); factor B-depleted human serum supplemented with a mixture of anti-MB mAb (2.4 μg) and 500 ng of purified human factor B protein; Control mouse IgG 0.4 μg, factor B-depleted human serum supplemented with a mixture of control mouse IgG (0.4 μg) and 500 ng of purified human factor B protein; Control mouse IgG 0.8 μg, factor B-depleted human serum supplemented with a mixture of control mouse IgG (0.8 μg) and 500 ng of purified human factor B protein; Control mouse IgG 1.6 μg, factor B-depleted human serum supplemented with a mixture of control mouse IgG (1.6 μg) and 500 ng of purified human factor B protein; Control mouse IgG 2.4 μg, factor B-depleted human serum supplemented with a mixture of control mouse IgG (2.4 μg) and 500 ng of purified human factor B protein.

FIG. 25 shows analysis of human factor B3 protein. Panel A shows silver staining of affinity purified human factor B3 protein: Lane 1, molecular weight marker; Lane 2, eluted sample from the first fraction; Lane 3, eluted sample from combination of the second and the third fractions. Panel B shows a Western blot analysis for human factor B3 protein and the lane assignment is the same as in Panel A.

FIG. 26 shows a hemolytic activity assay to assess alternative complement pathway activity. Y-axis displays the relative hemolytic activity as measured by the hemoglobin level released to the supernatant after lysis of erythrocytes. X-axis from left to right: Positive control with 100% lysis, RBC lysed in water; Blank, the RBCs were incubated in isotonic saline (no red blood cell lysis); Wild type factor B protein (50 ng, 100 ng, 200 ng, and 500 ng), factor B depleted human serum supplemented with 50 ng, 100 ng, 200 ng, and 500 ng wild type human factor B (Quidel); Wild type factor B protein plus factor B3 protein, factor B depleted human serum supplemented with a mixture of 40 μl of affinity purified mutant factor B3 plus 0 ng, 200 ng, or 500 ng of wild type human factor B from Quidel.

FIG. 27 shows GFP expression and C3 staining in mouse retinas. FIGS. 27A & 27B: GFP vector was administered as described in Example 27. Two weeks later, flat mounts were prepared and examined with a fluorescent microscope. FIGS. 27C & 27D: Null and hfB3 vectors were administered as described in Example 27. Two weeks later, laser photocoagulation was performed near the center of the retinas. Twenty hours later, the retinas were harvested, stained for C3 deposition, and examined with a fluorescent microscope.

FIG. 28 shows the vector titer in each of the elution fractions from a Sephacryl S 500-HR column. For this study, the culture medium from which the vector was purified was not supplemented with FBS. Details are described in Example 32.

FIG. 29 depicts the following plasmids. FIG. 29A is pAVTrGP038 (SEQ ID NO:19. FIG. 29B is pAVTrREV039 (SEQ ID NO:20). FIG. 29C is pAVTrGP64-040 (SEQ ID NO:21). FIG. 29D is pAVT001 (SEQ ID NO:22). FIG. 29E is pAVTGFP006 (SEQ ID NO:23).

### DETAILED DESCRIPTION OF THE INVENTION

The practice of the present invention will employ, unless otherwise indicated, conventional techniques of cell biology, molecular biology, cell culture, virology and the like which are in the skill of one in the art. These techniques are fully disclosed in current literature, for example, Sambrook, Fritsch and Maniatis eds., “Molecular Cloning, A Laboratory Manual”, 2nd Ed., Cold Spring Harbor Laboratory Press (1989); Celis J. E. “Cell Biology, A Laboratory Handbook” Academic Press, Inc. (1994) and Bahnson et al., J. of Virol. Methods, 54:131-143 (1995).

The term “complement-mediated” refers to a process or disease that involves complement. Typically, a “complement-mediated” disease or condition is one wherein complement activity is one of the underlying causes of the disease or condition and wherein inhibition or blocking of the complement activity lessens the extent of the disease or condition. Examples of numerous complement-mediated diseases or conditions are described herein.

The term “complement protein” or “complement pathway component” is a protein of the complement system or a receptor thereof. Complement proteins are a group of about 35 interacting proteins and glycoproteins found in all vertebrates. The complement proteins can be soluble or on the cell-surface. (Sim and Tsiftsoglou, Biochemical Society Transactions (2004) 32(1):21-27) In addition, there are regulatory membrane proteins that protect host cells from accidental or undesirable complement attack. A complement protein can be one that functions in the classical pathway, for example, C2 or one that functions in the alternative pathway, for example, Factor B. At least 6 complement proteins are proteases. For example, proteins included in the following exemplary list are complement proteins: C1q, C1r, C1s, C2-9, Factor B, Factor D, Factor H, Factor I, CR1, CR2, CR3, CR4, properdin, C1 (Inh), C4bp, MCP, DAF, CD59 (MIRL) and HRF.

The term wildtype (or wild-type), which is used interchangeably with native, as used herein relates to a naturally occurring protein encoded by a mammalian genome, a naturally occurring nucleic acid, a naturally occurring individual or animal and so on.

“Complement protein variant”, “complement protein mutant” or “complement protein analog” are used interchangeably and refer to a structural derivative of the parent protein that does not necessarily retain all of the properties of the native (naturally-occurring) parent protein or has at least one altered property as compared to the native parent protein. An analog or variant is produced by replacing, substituting, deleting, and/or adding amino acids with regard to the native amino acid sequence of the protein. The substitutions or insertions typically involve naturally occurring amino acids, but may also include synthetic or unconventional amino acids as well. In some embodiments, an analog or variant is produced by mutating a protein, e.g., mutating a nucleic acid encoding it. An analog will typically retain at least 70%, at least 75%, at least 80%, at least 85%, at least 90%, at least 95%, at least 96%, at least 97%, at least 98%, at least 99%, at least 99.5% of the native parent protein's amino acid sequence (e.g., have that percent amino acid sequence identity with respect to the naturally occurring parent protein as determined over the length of the entire parent protein or, in certain embodiments, over a specific domain or portion of the parent protein). Analogs also include fragments of full length analogs that comprise a portion of the amino acid sequence (for example, at least 30, 50, 70, 100, 150, 200, 300, 400, 500, 600 or 700 amino acids or comprise one or a subset of the domains of the analog) and either retain one or more biological activities of the parent protein or a full length analog or inhibit one or more of these biological activities.

The term “corresponds” when referring to an amino acid in a particular protein refers to the particular amino acid in that particular protein and also to an amino acid in a related or similar protein that provides a similar function to the protein. For example, an amino acid in a human complement factor B may be found to correspond with an amino acid in a murine complement factor B or in a human allelic variant of factor B, usually determined by aligning the two amino acid sequences. For example, one skilled in the art can align two related sequences, such as SEQ ID NO:2 and 16, to determine corresponding amino acids, e.g., using a BLAST program. Also, corresponding amino acids can be determined, e.g., by aligning motifs (e.g., a protease cleavage motif) within related or unrelated proteins. Such an alignment may also be used to derive consensus sequences of target protein or domains thereof.

As used herein, the term “gene” typically refers to a coding region for a protein. However, in some contexts herein it will be clear that the term “gene” is also referring to elements (e.g., regulatory elements) operatively linked to a coding region such as promoters, enhancers, splice sites (acceptors and/or donors), polyadenylation signals, introns, 5′ untranslated regions, 3′ untranslated regions, etc.

As used herein, “gene of interest”, sometimes referred to as a transgene, is a heterologous gene or a foreign gene, relative to the source of the nucleic acid vector or vector construct. Thus, for example, in the case of a BIV vector, a gene of interest is generally not a BIV gene. In some embodiments, a gene of interest is a therapeutic gene.

The term “packaging sequence” is a sequence necessary for packaging viral nucleic acids into virions, virus particles or virus-like particles. In BIV, for example, a packaging sequence(s) is generally located in the region between the 5′ major splice donor and the upstream region of the gag gene. Vectors according to the invention may include packaging sequences corresponding to other viruses, e.g., lentiviruses such as HIV-1, HIV-2 or SIV. Packaging sequences may include as many as 1000 nucleotides or a few as 50 nucleotides. The size of a packaging sequence region can be easily determined by one of ordinary skill in the art.

The term “pharmaceutically acceptable” means approved by a regulatory agency of the Federal or a state government or listed in the U.S. Pharmacopeia or other generally recognized pharmacopeia for use in humans.

A “therapeutic benefit” is not necessarily a cure for a particular disease or condition (including any disease or condition described herein), but rather, encompasses a result which most typically includes alleviation of the disease or condition, elimination of the disease or condition, reduction of one or more symptoms associated with the disease or condition, prevention or alleviation of a secondary disease or condition resulting from the occurrence of a primary disease or condition, diminishing the likelihood of developing a condition or disease, diminishing the severity of a disease or condition, changing the character of a disease or condition, shortening the course of a disease or condition, slowing or preventing the progression or worsening of a disease or condition, and/or prevention of the disease or condition.

“Transformed” is meant to include any means in which an exogenous, heterologous or foreign nucleic acid is introduced into a cell. That can occur by using a nucleic acid per se, a plasmid (transfection), a virus (infection or transduction), a synthetic carrier molecule and so on, as known in the art. A cell that is transformed is one which is treated in any of a variety of ways to carry a foreign nucleic acid of interest. Such cells can be somatic cells, stem cells that are not embryonic stem cells, and embryonic stem cells.

A “vector construct” or “vector sequence” or “transfer vector” refers to an assembly which is capable of directing the expression of a nucleotide sequence, a transgene, a protein or a therapeutic expression product. In some embodiments of the invention, vector construct can include a 5′ sequence (comprising an operably linked promoter region) which is capable of initiating transcription; a DNA segment from a viral genome; and/or a packaging sequence from a virus such as a lentivirus or retrovirus. In one embodiment, the present invention provides a BIV vector construct comprising: a DNA segment from a BIV genome, a packaging sequence for packaging RNA into virions, a first promoter operably linked to the DNA segment, and a transgene operably linked to a second promoter. In one embodiment, a packaging sequence of the BIV vector construct is a BIV packaging sequence.

### Target Molecules

“Target molecules” are molecules, which can be involved in a pathway, which themselves or their activity can be modulated resulting in modulation of the pathway. A target molecule may not necessarily be involved directly in a pathway, e.g., it may have an activity that affects another, second molecule that in turn affects a molecule directly involved in the pathway. In some embodiments of the invention, the quantity and/or activity of a target molecule may be increased, decreased or maintained. This may be accomplished by, but is not limited to, modulating expression of the target molecule or destabilizing or eliminating at least some of the target molecule, sequestering the molecule and/or altering one or more biological activities of the target molecule. In some embodiments, modulating expression of the target molecule is accomplished by transforming a cell (e.g., in vitro or in vivo) with at least one nucleic acid coding for the target molecule when the target molecule is a protein or nucleic acid. In some embodiments, a target protein can be destabilized or its activity abrogated or reduced by utilizing a binding molecule that destabilizes or reduces the activity of the target protein. In some embodiments, a binding molecule is utilized that binds a target protein resulting in destabilization of the target protein. In some embodiments, a protease that cleaves the target protein can be used to destabilize or eliminate at least a part of the target molecules. In some embodiments, a protease cleaves a target molecule at 1, 2, 3, 4, 5, 6, 7, 8, 9, 10, or more sites within the target molecule. Some embodiments of the invention inhibit a target molecule(s). Inhibitors of the invention include, but are not limited to, binding molecules such as antibody molecules (as well as homologues, analogues and modified or derived forms thereof) including immunoglobulin fragments such as Fab, F(ab′)₂ and Fᵥ, small molecules, peptides, oligonucleotides, aptamers, peptidomimetics, and organic compounds.

The complement system can mediate a chain reaction of proteolysis and assembly of protein complexes, e.g., that result in the elimination of invading microorganisms. Three activation pathways (the classical, lectin, and alternative pathways) and a lytic pathway regulate these events.

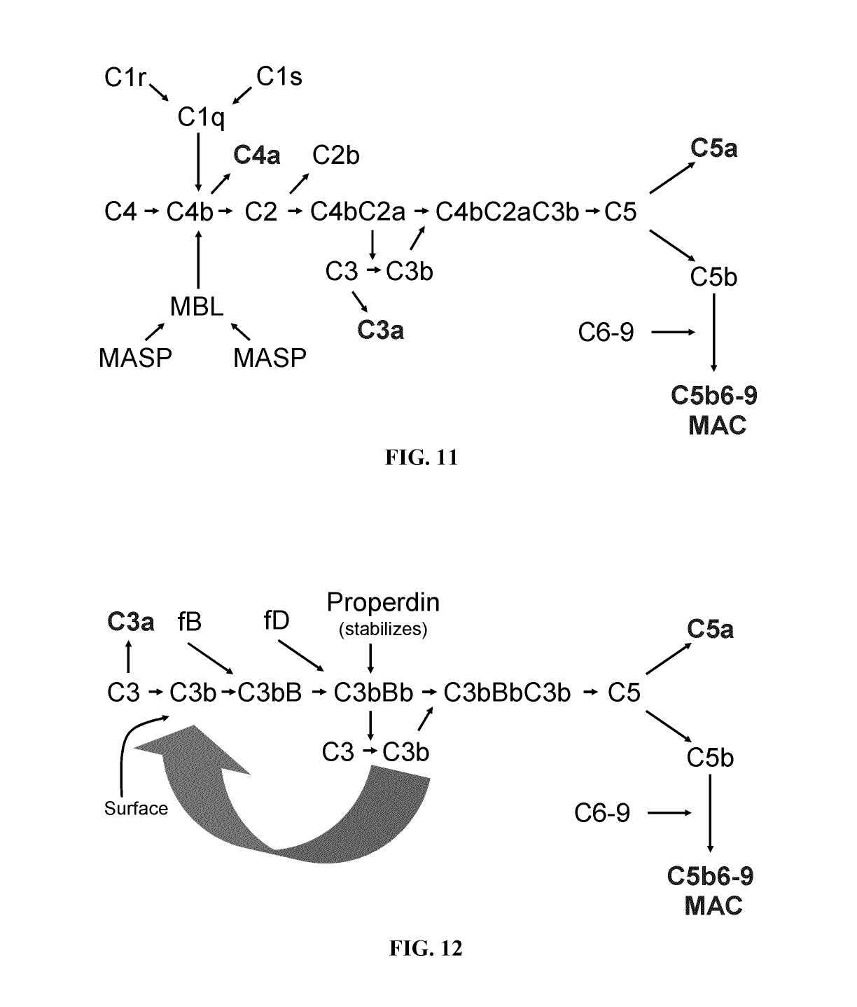

In the classical pathway as shown in FIG. 11, C1q, a collagenous subcomponent of the first component (C1), binds to immunoglobulins within immune complexes, and its associated serine proteases, C1r and C1s, become activated. This complement cascade is initiated by the subsequent cleavage of C4 and C2, followed by C3 activation. The resulting C3b fragment not only acts as an opsonin but also leads to the membrane attack complex (MAC) formation in the lytic pathway. In innate immunity, a complex composed of a recognition molecule (lectin) and serine proteases, termed the mannose-binding lectin (MBL)-associated serine protease (MASP), activates C4 and C2 upon binding to carbohydrates on the surface of microorganisms via the lectin pathway. This binding occurs in the absence of immunoglobulins. Recognition molecules of the lectin pathway found in jawed vertebrates are MBLs and ficolins, both of which are characterized by the presence of a collagen-like domain, like C1q, and a carbohydrate binding domain having a common binding specificity for GlcNAc. MASPs and C1r/C1s share the same domain organization and form a subfamily of serine proteases.

The lectin complement pathway in innate immunity is closely related to the classical complement pathway in adaptive immunity, e.g., with respect to the structures and functions of their components. Both pathways are typically initiated by complexes consisting of collagenous proteins and serine proteases of the mannose-binding lectin (MBL)-associated serine protease (MASP)/C1r/C1s family. It has been speculated that the classical pathway emerged after the lectin pathway.

Activation of the alternative complement pathway, shown in FIG. 12, begins when C3b (or C3i) binds to a cell and other surface components, e.g., of microbes. C3b can also bind to IgG antibodies. Alternative pathway protein Factor B then combines with the C3b to form C3bB. Factor D then splits the bound Factor B into Bb and Ba, forming C3bBb. Properdin then binds to the Bb to form C3bBbP that functions as a C3 convertase capable of enzymatically splitting typically hundreds of molecules of C3 into C3a and C3b. The alternative complement pathway is now activated. Some of the C3b subsequently binds to some of the C3bBb to form C3bBbC3b, a C5 convertase capable of splitting molecules of C5 into C5a and C5b

Since C3b is free in the plasma, it can bind to either a host cell or pathogen surface. To prevent complement activation from proceeding on the host cell, there are several different kinds of regulatory proteins that disrupt the complement activation process. Complement Receptor 1 (CR1 or CD35) and DAF (also known as CD55) compete with Factor B in binding with C3b on the cell surface and can even remove Bb from an already formed C3bBb complex. The formation of a C3 convertase can also be prevented when a plasma protease called Factor I cleaves C3b into its inactive form, iC3b. Factor I works with C3b-binding protein cofactors such as CR1 and Membrane Cofactor of Proteolysis (MCP or CD46). Another complement regulatory protein is Factor H which either competes with factor B, displaces Bb from the convertase, acts as a cofactor for Factor I, or preferentially binds to C3b bound to vertebrate cells.

Target molecules include, but are not limited to, components of immunological pathways or variants thereof, such as components of the classical complement pathway; components of the alternative complement pathway or components of a lectin complement pathway. In some embodiments, more than one target molecules may be modulated and/or acted on. In some embodiments, a component of the alternative complement pathway is selected from the group consisting of C3, C3a, C3b, C3bB, fB (factor B), fD (factor D), C3bBb, C3bBbC3b, C5, C5a, C5b, C6, C7, C8, C9, C5b6-9, MAC (membrane attack complex) and fragments thereof and components thereof. In some embodiments, a component of the classical complement pathway is selected from the group consisting of C1r, C1q, C1s, C4, C4b, C4a, MBL, MASP, C2, C2b, C4bC2a, C3, C3b, C3a, C4bC2aC3b, C5, C5a, C5b, C6, C7, C8, C9, C5b6-9, and MAC.

Target molecules that initiate or facilitate inflammation would benefit from reduced expression and/or activity, and include, properdin, TGFβ, complement factor B (human, accession no. NP 001701, Kavanaugh et al., Mol. Imm. 43(7)856, 2006; mouse, accession no. NP 032224m, Bora et al., J. Imm. 177(3)1872, 2006), complement factor D and other factors of the complement system, including, C2, C3, C4, C5, C6, C7, C8 and C9. Thus, a delivery means of interest can express, for example, a specific antigen-binding polypeptide, a soluble receptor, an inhibitory nucleic acid, a ribozyme, an aptamer, a catalytic antibody, a molecule carrying amino acid substitutions that interfere with function, such as preventing docking to a receptor, or making such binding irreversible, removing enzyme activity and so on. Alternatively, a molecule which could compete with a target molecule, such as a receptor, but which is ineffective in activating or encouraging inflammation, can be used as a competitive inhibitor to attenuate inflammation. Also, a complement factor with altered or no (devoid) activity, which may be one or more particular activities or functions of any one molecule, can be used in the practice of the instant invention. Altered means an activity or function other than that obtained under normal, ambient condition, such as operating at a higher activity or presenting with a higher level of activity, or a lower level of activity. Other means of dampening expression or activity as known in the art can be practiced.

Complement factors often are polyfunctional. Thus, molecules may be considered to have plural domains. For example, a complement factor may have a domain carrying a recognition and binding function or activity; a domain that is recognized by another molecule, which site or a region adjacent thereto, can be reacted, changed or cleaved; a domain that may have a biological activity, such as an enzymatic activity, such as a protease activity; and so on. A biological activity of a complement factor can be diminished or negated when any one or more of said domain functions is altered, and alteration of one domain may or may not have an impact on the normal functioning of another domain. Altering domains of a protein can be accomplished by, for example, substituting, inserting and/or deleting amino acids in the domain and testing for the desired characteristic. Even if the location of the domain is not known a systematic approach can be used to locate and obtain variants with altered function.

### Pathway Modulators and Molecules of the Invention

A number of molecules can be targeted in the treatment of complement mediated disease such as those related to ocular disease. With a goal of mitigating or ameliorating inflammation, or not allowing inflammation to continue, the instant invention, in part, relates to methods of having local or systemic delivery of a molecule to achieve that result. Thus in some embodiments of the invention, a molecule can be one which inhibits expression of or activity of a target molecule that initiates, contributes to or facilitates inflammation, or one that enhances the expression of or activity of a target molecule that dampens inflammation.

In some embodiments, the invention contemplates modulating a pathway via modulation of the activity of any one or more components of the pathway as described herein and using any of the methods for modulating activity of a pathway component as described herein.

Immunological pathways and processes can contribute to the progression, maintenance, and/or inhibition of a condition (e.g., a disease) in an animal. The present invention provides, in part, methods of modulating (e.g., enhancing, increasing, inhibiting, decreasing or generally maintaining) an immunological pathway, e.g., in vitro or in vivo. Some embodiments of the invention can be used to study an immunological pathway, to study associated disease states, to develop treatments for a disease state(s), to create disease states in an animal (e.g., to develop a disease model in an animal such as a mouse or rat), or for screening drugs.

In some embodiments, a target molecule is a component of a complement pathway, e.g., as described herein. Some embodiments of the invention involve modulating a classical complement pathway; an alternative complement pathway or a lectin complement pathway.

Some embodiments of the invention reduce, increase or prevent inflammation and/or complement activity. Some embodiments of the invention modulate complement function or activity. Because complement is comprised of a number of factors which act in a synchronized manner, and a number of molecules and factors not classically identified as complement which regulate or control the presence and activity of a complement factor, any one of such complement factors or regulatory molecules of complement can serve as an entry point or as a target molecule of the instant invention as means to impact complement activity and function, such as enhancing or reducing complement activity as compared to baseline.

Inhibition of a complement pathway according to the present invention can be accomplished, for example, by directly affecting the expression (e.g., transcription and/or translation) or biological activity of a protein in the complement pathway, or by directly affecting the ability of a protein to bind to a protein in the complement pathway or to otherwise contribute (positively or negatively) to the activation of complement via the alternative pathway. In some embodiments, expression of a protein refers to either the transcription of the protein or the translation of the protein. Therefore, some methods of the invention can inhibit the transcription and/or the translation of a protein (e.g., in an animal) that naturally expresses the protein (e.g., by administering an agent that inhibits the expression of the proteins and/or genetically modifying an animal to have reduced protein expression). In another embodiment, inhibition of a complement pathway is defined herein as any measurable (detectable) reduction (e.g., decrease, downregulation, or inhibition) of the activity of the pathway, such as by any measurable reduction in the expression and/or biological activity of a protein within the alternative complement pathway. In yet another embodiment, the invention relates to enhancing expression and/or function of a factor that attenuates complement activity, such as complement factor H, DAF and so on.

In some embodiments, vectors of interest can express known inhibitors of complement, such as C1 esterase inhibitor (Kirschfink & Molines, Expert Opin. Pharmacother. 2, 1073, 2001) or compstatin (Nilsson et al., Blood, 92, 1661, 1998). Other target molecules that modulate the complement system and which are alternative targets of interest include HtrA1 (Oka et al. Development 131, 1041, 2003; which inhibits TGFβ signaling, a known stimulus of C3 and factor B expression) and CRP.

In some embodiments, a target molecule's activity is diminished, decreased, increased, enhanced or maintained. This can be accomplished utilizing various techniques. In some embodiments, a binding molecule (e.g., a protein, ligand, receptor or antibody) binds a target molecule. In some embodiments, 1, 2, 3, 4, 5, 6, 7, 8, 9, 10 or more binding molecules bind a target molecule. In some embodiments, a binding molecule binds a target protein and inhibits a target protein by, for example, blocking, partially blocking or competing for binding of a site on a target molecule involved in an activity of the target molecule; by sequestering a target molecule; by competing for binding of a target molecule with a ligand (e.g. a native ligand); by causing a target molecule to be degraded (e.g., macrophage targeted degradation utilizing a binding protein comprising a macrophage binding portion, such as an Fc portion, etc.).

As discussed herein in one approach in the practice of the instant invention, the coding sequences of a target molecule(s) (e.g., which participate in inflammation pathways or regulate the same), whether a nucleic acid or protein, can be altered and/or manipulated to yield less operative or inoperative expression products; to yield dominant negative expression products; to yield expression products that are more operative or with a greater activity; or to yield molecules where particular functions thereof are manipulated to have reduced or negated activity. Target molecules which have a binding or docking function, an enzymatic function and so on, such as those with domains or segmented portions with plural functions are suitable targets for selective diminution of an activity. For example, a target molecule can be altered not to bind to the natural receptor, or to bind irreversibly thereto. A target molecule can be manipulated so that the enzymatic activity is lost or diminished. Such changes can be made using materials and methods known in the art, for example, to introduce amino acid substitutions, such as, by site directed mutagenesis. Some disorders which are amenable to treatment by the instant invention may be caused or have as a symptom, inflammation, which as known in the art, is characterized by release or activation of inflammatory cytokines, vascular leakage, leukocyte infiltration, and/or tissue damage. Thus, for the purposes of the instant invention, a disease which presents with inflammation is one which has inflammation as an etiology or one which has as a symptom or manifestation during the course of the disease, a feature of inflammation.

In another embodiment, molecules with binding ability can be used to entrap and to sequester target molecules to prevent them from interacting and carrying out their normal biological function. Thus, for example, soluble complement receptors or particular molecules that bind inducers of inflammation can be delivered or expressed locally using a vector construct of interest, e.g., to minimize having the bound molecules exerting their pro-inflammatory activities.

For example, target molecules that would dampen inflammation would benefit from enhanced expression and/or activity, and include complement receptor 1 (CR1, also known as CD35, binds to C3b, a soluble form of CR1 is described in Weisman et al., Science 249, 146, 1990); C4 binding protein and binding portions thereof; clusterin, S protein and homologous restriction factor (HRF), all three of which inhibit MAC formation; complement receptor 1-related protein/gene y (Crry); complement factor I (breaks down C3b); complement factor H (which inhibits binding of factor B to 3b); DAF (also known as CD55, dissociates C3 convertase); membrane cofactor protein (MCP, also known as CD46, is a cofactor for complement factor I); the membrane inhibitor of reactive lysis (also known as MIP or CD59) prevents formation of the membrane attack complex (MAC); and FHL-1, for example. Thus, a delivery means of interest can introduce into cells (e.g., eye cells) an additional copy or an expressed copy of a nucleic acid expressing a protein such as those noted hereinabove; or can introduce a means to enhance expression of an endogenous coding sequence, such as, by inserting an operably linked enhancer or inducible promoter, see, for example, U.S. Pat. Nos. 5,272,071 and 6,303,379, for example. Other means and methods for obtaining expression can be practiced as known in the art. The present invention also contemplates the delivery of proteins. Therefore, any discussion of expressing a protein or peptide also contemplates the direct delivery of the protein itself, for example, locally and/or systemically.

In accordance with the present invention, complement protein analogs are provided which are structurally modified as compared to the naturally-occurring complement proteins, and which thereby have a functional modification in one or more properties such as proteolytic activity; stability; binding affinity; target specificity; susceptibility to regulatory proteins; susceptibility to proteolysis; and cofactor requirements. Modification of these properties in a complement protein analog can directly or indirectly affect their complement-mediated activity. These analogs can be used to modulate the complement system. A complement protein's proteolytic activity, stability, binding affinity for a target, susceptibility to regulatory proteins and/or susceptibility to proteolysis may be increased or decreased. Substrate specificity may be broadened or narrowed and/or a requirement for cofactor may be made more or less stringent or abolished. In some embodiments, modified complement proteins are produced by mutations in regions that control the above-mentioned properties.

In some embodiments, the invention is directed to analogs and/or inhibitors of, for example, C1r, C1s, Factor B, Factor D, Factor I, C3b and/or C2. The invention also provides methods and encoding polynucleotides for preparing these analogs, amino acid sequences encompassing the mutations, pharmaceutical compositions of these analogs as therapeutic agents in the treatment of complement related disorders, and diagnostic methods using these analogs as reagents; for example, as standards for competitive ELISA of complement proteins in serum or tissue samples and the like.

Mutations to modify a complement protein(s) includes one or more amino acid mutations (e.g., substitutions) such as in a short consensus repeat(s) (SCR), a von Willebrand Factor (vWF) domain(s) and/or a protease domain(s) or in any other region(s) that binds or associates with substrates, regulatory proteins or cofactors. An amino acid substitution comprises substituting at least one amino acid up to an entire domain or more than one domain (such as several SCRs); or a combination of the above. In addition to substitutions, additions and deletions of one or more amino acid residues or domains may be accomplished.

In some embodiments, a protease domain of a member of the complement protease family is substituted with the protease domain of either (a) a second member of the complement family, or (b) a member of the serine protease superfamily. An example of (a) is the substitution of the protease domain of Factor I with the protease domain of Factor B. An example of (b) is the substitution with the protease domain of chymotrypsin or elastase. Such substitutions will alter substrate specificity of the complement protease. For example, the substrate specificity of a C3 convertase may be altered such that the C3 convertase is able to cleave a toxin instead of its normal substrate. In some variations, an analog will lack substantial protease activity or have decreased protease activity but will bind to a complement component, e.g., with detectable binding affinity.

As discussed herein, the invention provides, inter alia, methods of inhibiting complement activity using complement protein analogs, such as factor B analogs. As demonstrated in some of the examples below, in some cases a native factor B from one species may have activity in a complement reaction/pathway from another species. Therefore, the present invention also include complement protein analogs from one species for inhibiting complement activity in another species.

In some embodiments, a complement protein analog may comprise glycosylation patterns which are distinct from glycosylation patterns on a naturally-occurring complement component, or may lack glycosylation altogether. Carbohydrates may be added to and/or removed from polypeptide analogs comprising glycosylation site sequences for N- and/or O-linked glycosylation in vitro, such as with a canine pancreatic microsome system (e.g., see Mueckler and Lodish (1986) Cell 44: 629 and Walter, P. (1983) Meth. Enzymol. 96:84) or the like. A complement protein analog of the invention may be produced comprising adding or deleting/mutating an amino acid sequence corresponding to a glycosylation site, e.g., changing the glycosylation pattern/status of a protein can change functional characteristics of a protein. For example, fb2 and fb3 (factor B analogs) comprise an N285D substitution which removes an N-glycosylation site. The loss of the N-glycosylation site alters the characteristics of the protein. The same effect may be achieved by producing the protein in a cell that has an altered glycosylation pattern or that does not glycosylate this N285. For example, an fB protein or analog may be produced in an E. coli cell that does not glycosylate the N285. In some embodiments, a protein comprising the sequence of either fb2 or fb3, except that the corresponding amino acid 285 is an asparagine, is produced in a cell (e.g., an E. coli) that does not glycosylate amino acid 285.

In some embodiments, a composition or molecule of the invention is PEGylated, e.g., see Roberts et al., Advanced Drug Delivery Reviews 54(4): 459-476 (2002); Veronese, Biomaterials 22(5): 405-417 (2001); Fee and AlstineChemical Engineering Science 61(3): 924-939 (2006); Kodera et al., Progress in Polymer Science 23(7): 1233-1271 (1998); Morar, Biopharm International 19(4):34 (2006); and Veronese and Pasut, Drug Discovery Today 10(21): 1451-1458 (2005).

In certain embodiments, either the carboxy-terminus or the amino-terminus, or both, are chemically modified. Amino-terminal modifications such as acylation (e.g., acetylation) or alkylation (e.g., methylation) and carboxy-terminal modifications such as amidation, as well as other terminal modifications, including cyclization, may be incorporated into various embodiments of the invention. Certain amino-terminal and/or carboxy-terminal modifications and/or peptide extensions (such as fused to a heterologous polypeptide, such as albumin, immunoglobulin or portion thereof, such as an immunoglobulin Fc domain) to the core sequence can provide advantageous physical, chemical, biochemical, and pharmacological properties, such as: enhanced stability, increased potency and/or efficacy, resistance to serum proteases, desirable pharmacokinetic properties, and others.

The invention further provides analogs which are fragments of a complement protein (including fragments of complement protein analogs) that contain at least a 10, 20, 50, 100, 200, 300, 500, or 600 amino acid portion of the target protein and/or comprises one, 2 or 3 domains of the protein and have one or more biological activities of the wild type complement protein or analog and/or acts as an inhibitor of an aspect of the complement system (either the classical pathway, alternative pathway or both).

Analogs of the invention can be prepared by various techniques, including but not limited to, chemical synthesis or by expression of the recombinant analog.

For exemplary purposes, factor B is presented as an example of a target molecule whose activity can be modulated, e.g., inhibited. Also, for exemplary purposes, specific analogs of Factor B are described herein. Factor B can be manipulated in a number of ways, e.g., to inhibit or reduce activation of the alternative pathway. For example, a factor B binding molecule, such as an antibody-derived molecule or an aptamer, can bind and/or sequester factor B, e.g., so the molecule cannot contribute to forming the convertase. An inhibitory molecule, such as an RNAi molecule, a ribozyme or a catalytic antibody can be expressed locally to prevent expression of or to destroy factor B. In some embodiments, particular sites in factor B can be altered, for example, by site directed mutagenesis, so that the molecule no longer fully functions properly. In some embodiments, the enzyme portion or domain, (the protease, which is a serine protease) of the molecule can be altered so that the molecule no longer has enzymatic activity or has reduced enzymatic activity (e.g., reduced by at least 2 fold, 5 fold, 10 fold, 50 fold or 100 fold), such as by altering the residue corresponding to amino acid 740 of human factor B (as depicted in SEQ ID NO:2) from D to another amino acid such as N, A, E, S, Y, or G. The numbering of particular factor B amino acids herein relates to the entire polypeptide including the signal peptide and is reflected in SEQ ID NO:2. Other sites in factor B that can be altered include: 1) the binding site for properdin (the properdin binding domain) such that binding occurs with lower affinity (for example, such as 2 fold, 5 fold, 10 fold, 50 fold or 100 fold reduced affinity as compared to the wild type factor B) or with greater affinity (such as 2 fold, 5 fold, 10 fold, 50 fold or 100 fold increased affinity as compared to the wild type factor B); 2) the binding site for C3b (the C3b binding domain) such that binding occurs with lower affinity (such as 2 fold, 5 fold, 10 fold, 50 fold or 100 fold reduced affinity as compared to the wild type factor B) or with greater affinity (such as 2 fold, 5 fold, 10 fold, 50 fold or 100 fold increased affinity as compared to the wild type factor B, for example, this may be achieved by substituting the amino acid corresponding to position 279 and/or position 285 of SEQ ID NO:2 with other amino acids, for example, wherein the amino acid at the position corresponding to position 279 is substituted with G, A, or N, and/or the amino acid at the position corresponding to position 285 is substituted with D or A); 3) the site acted on by factor D such that factor D has reduced ability to cleave or no longer cleaves factor B to form Bb (for example, at the factor D cleavage site, at least one of the amino acids at the positions corresponding to position 258, 259 or 260 of SEQ ID NO:2, for example, can be altered to A or, a combination of 1, 2, and/or 3 above. Because factor B has a central role in complement activation, factor B is an attractive target molecule.

A “von Willebrand Factor (vWF)” domain (also called the A-type domain) averages about 200 amino acids. It is found 3 times in vWF and once in Factor B, C2, CR3 (Mac-1), CR4 and other proteins (reviewed by Columbatti and Bonaldo (1991) Blood 77:2305). The overall sequence similarities among the vWF domains typically range from 18-64%.

The invention also provides modified human Factor B which exhibits increased, decreased, or undetectable complement-mediated cell lysis activity.

Some modified factor B analogs of the invention comprise one or more of the amino acid alterations discussed herein and additionally have one or more additional amino acid substitutions, insertions or alterations (e.g., at least or no more than 1, 2, 5, 8, 10, 15 20, 50, 100, or 200 alterations), which analogs retain the increased binding to C3b and/or factor D or other biological activity of the factor B analogs discussed herein, which mediates inhibition of the complement pathway. Such analogs may have at least 99.9%, 99%, 98%, 95%, 90%, 85%, 80%, 75% or 70% amino acid sequence identity with wild type factor B, for example, the amino acid sequence of SEQ ID NO:2 and retain the increased binding to C3b and/or factor D.

Some factor B analogs of the invention may have increased binding to C3b and/or factor D by a factor of 2 fold, 4 fold, 5 fold, 10 fold, 20 fold, 50 fold, 100 fold, 500 fold, 1000 fold as compared to binding of wild type factor B to C3b and/or factor D.

Some embodiments of the invention are directed to polynucleotides and host cells (or host multicellular organisms) useful in the production of a modified complement pathway component(s), e.g., fB3. Methods of isolating and testing of complement-mediated activity of these modified complement pathway component(s) are also provided. Some aspects of the invention are directed to pharmaceutical compositions wherein these modified complement pathway component(s) are active ingredients in therapeutic and/or prophylactic contexts. Some embodiments of the invention are also directed to methods of treating complement-mediated disorders using a therapeutically effective amount of a modified complement protein.

Some embodiments of the invention utilize a binding molecule (e.g., an antibody or fragment thereof) that binds a target molecule. In some embodiments, a binding molecule inhibits the activity of a target molecule, such as one involved in a complement related pathway.

In some embodiments, a binding molecule that binds factor B and/or factor D is utilized, e.g., see U.S. Patent Application No. 20050260198 and PCT Publication No. WO0021559. In some embodiments, a binding molecule binds factor B and binds within the third short consensus repeat domain.

Some embodiments of the invention may utilize a peptide, protein or other molecule (e.g., an antibody or an aptamer) that binds a component of a complement related pathway. In some embodiments, a peptide or other molecule binds a component and blocks or competes for binding by another component. In some embodiments, a peptide or other molecule binds a component and blocks or inhibits the component's activity (e.g., blocks an enzyme's catalytic domain) and/or blocks a site to be acted upon by, e.g., another component. In these embodiments a site to be acted upon may be a cleavage site or another site that is a substrate for an enzymatic activity (e.g., a phosphorylation site or dephosphorylation site). In some embodiments, a peptide is utilized that mimics the binding properties of factor B, but lacks the ability to activate C3, e.g., see PCT Publication No. WO0021559. In some embodiments, peptides or proteins described herein can be used or a nucleic acid or vector can be used to express them. In some embodiments, multiple peptides acting on multiple sites and/or components are utilized.

The invention includes (i) molecules that bind to both factors C3b and D e.g., fB3, a bispecific antibody, etc; (ii) complement protein analogs with increased binding (as compared to their native form) to both factors C3b and D; (iii) complement protein analogs with increased binding (as compared to their native form) to factor D; and (iv) complement protein analogs with increased binding (as compared to their native form) to C3bB complex. The invention also includes methods of inhibiting a complement pathway using the molecules of the invention, such as i-iv, above. In some embodiments, a molecule that binds to both factors C3b and D is not a wild-type fB. In some embodiments, a molecule that binds to both factors C3b and D is not fB1, fB2 or fB3.

In some embodiments, increased binding is increased by about 1.5 to about 10,000, about 10 to about 10,000, about 100 to about 10,000, about 1,000 to about 10,000, about 1.5 to about 1,000, about 1.5 to about 100, about 1.5 to about 10, about 2 to about 5, about 2 to about 10, about 5 to about 10, about 5 to about 20, about 10 to about 20, about 10 to about 30, about 20 to about 30, about 30 to about 50, about 50 to about 100, about 100 to about 500, about 500 to about 1,000, about 1,000 to about 5,000, or about 5,000 to about 10,000 fold. In some embodiments, increased binding is increased by greater than 1.5, 2, 3, 4, 5, 10, 50, 100, 500, 1000, 5000 or 10,000 fold. In some embodiments, increased binding can be measured by immunoprecipitation, e.g., as compared to the wild type protein. As an example for (i) above, binding could be measured by immunoprecipitation of the protein with a binding molecule for C3b and then detecting D in the immunoprecipitate, e.g., using an immunoassay such as an ELISA or Western, for example, with increased binding demonstrated as a band of increased intensity in a Western.

In some embodiments, a binding molecule binds to an epitope in the third

SCR domain of factor B selected from: (a) an epitope of factor B that includes at least a portion of human factor B corresponding to the portion comprising from about position 164 to about position 210 of SEQ ID NO:2, or equivalent positions thereto in a non-human factor B sequence; (b) an epitope of factor B that includes at least a portion of human factor B comprising the amino acid sequence corresponding to from about position 164 to about position 166 of SEQ ID NO:2, or equivalent positions thereto in a non-human factor B sequence; (c) an epitope of factor B that includes at least a portion of human factor B corresponding to the amino acid sequence comprising from about position 207 to about position 210 of SEQ ID NO:2, or equivalent positions thereto in a non-human factor B sequence; or (d) an epitope of factor B that includes at least a portion of human factor B comprising amino acids corresponding to any one or more of the following positions or their equivalent positions in a non-human factor B sequence: 164, 165, 166, 207, 209, or 210 of SEQ ID NO:2. In yet another aspect, the antibody or antigen binding fragment thereof selectively binds to an epitope in the third SCR domain of factor B comprising amino acids corresponding to one or more of the following amino acid positions or their equivalent positions in a non-human factor B sequence: 162, 164, 166, 207, 210, 214, 215, and 217 of SEQ ID NO:2. In another aspect, the antibody or antigen binding fragment thereof selectively binds to an epitope in the third SCR domain of factor B comprising amino acids corresponding to the following amino acid positions or their equivalent positions in a non-human factor B sequence: 162, 164, 166, 207, 210, 214, 215, and 217 of SEQ ID NO:2. In yet another aspect, the antibody or antigen binding fragment thereof selectively binds to an epitope in the third SCR domain of factor B consisting of amino acids corresponding to the following amino acid positions or their equivalent positions in a non-human factor B sequence: 162, 164, 166, 207, 210, 214, 215, and 217 of SEQ ID NO:2. The antibody or antigen-binding fragment can bind to a non-linear epitope within the three-dimensional structure of a portion of the third SCR domain of factor B, wherein the portion is defined by at least the amino acid positions corresponding to positions 162-217 of SEQ ID NO:2 or equivalent positions in a non-human factor B sequence.

In some embodiments, a binding molecule binds factor B and inhibits, prevents, reduces and/or ablates formation of a C3bBb complex. In some embodiments, a binding molecule binds factor B and inhibits, prevents, reduces and/or ablates cleavage of factor B by factor D. In some embodiments, a binding molecule competitively inhibits binding of the monoclonal antibody 1379 (produced by the hybridoma bearing ATCC Deposit No. PTA-6230, American Type Culture Collection, P.O. Box 1549, Manassas, Va. 20108) to human factor B. In some embodiments, a binding molecule is the monoclonal antibody 1379 (ATCC Deposit No. PTA-6230), a humanized or chimeric form of monoclonal antibody 1379, or any antigen binding fragment of antibody 1379 or a humanized form thereof, for example, an antibody comprising CDR1, CDR2, and CDR3 of the heavy chain variable domain of antibody 1379 and/or CDR1, CDR2, and CDR3 of the light chain variable domain (optionally with 1, 2, 3, 5, 10, 12, 15, or 20 amino acid deletions, insertions and/or substitutions in said CDRs which improve binding affinity or other kinetic parameter); or an antibody, preferably a humanized or human antibody, that competes for binding with monoclonal antibody 1379 as determined, for example, by ELISA or other immunoassay.

In some embodiments of the invention, activity of factor D is modulated (e.g., inhibited). In some embodiments, this is done in combination with modulation of another pathway component's activity (e.g., factor B's, C3b's and/or C3bB's activity). Serum concentrations of factor D are believed to be relatively low. In some embodiments, factor D can be a target molecule, e.g., for an antibody, aptamer, an inhibitory analog of a pathway component or soluble receptor sequestering strategy. Local expression of a factor D “inhibiting” molecule, such as a neutralizing antibody or aptamer thereto, into the space between RPE cells and Bruch's membrane is obtainable by the practice of the instant invention. In some embodiments, a factor D ‘inhibiting” molecule such as a protein can be used (e.g., by injection to the eye). For example, U.S. Pat. No. 6,956,107 relates to factor D inhibitors. In some embodiments, an antibody or factor D binding fragment thereof is utilized, such as the monoclonal antibody 166-32 (Accession No. HB-12476, ATCC) a humanized or chimeric form of monoclonal antibody 166-32, or any antigen binding fragment of antibody 166-32 or a humanized form thereof, for example, an antibody comprising CDR1, CDR2, and CDR3 of the heavy chain variable domain of antibody 166-32 and/or CDR1, CDR2, and CDR3 of the light chain variable domain (optionally with 1, 2, 3, 5, 10, 12, 15, or 20 amino acid deletions, insertions and/or substitutions in said CDRs which improve binding affinity or other kinetic parameter); or an antibody, preferably a humanized or human antibody, that competes for binding with monoclonal antibody 166-32 as determined, for example, by ELISA or other immunoassay.

Another component of the alternative pathway is CFH. In some embodiments of the invention, activity of CFH is modulated (e.g., inhibited). In some embodiments, this is done in combination w/modulation of another pathway component's activity (e.g., factor B's, factor D's, C3b's and/or C3bB's activity). For example, tyrosine at position 402 of CFH is associated with a low risk for AMD whereas a histidine at that position is associated with high risk. The Y402H polymorphism located in SCR7 of CFH is also found in the CFH splice variant, FHL-1 (Estaller et al., 1991; Sim et al., 1993). CFH and FHL-1 display similar complement regulatory functions (Zipfel & Sherka, 1999). CFH functions as an important inhibitor to prevent uncontrolled complement activation. The identified tyr402his polymorphism is potentially associated with inflammation in the eye. Thus, a therapeutic coding region can be a nucleic acid encoding tyrosine at position 402 of a CFH or FHL-1 molecule. Cells transformed to carry and to express said molecule will have a beneficial, therapeutic and/or prophylactic effect in the eye, e.g., with regards to inflammation.

It is understood that when introduction of a nucleic acid encoding a protein is discussed, that the invention also contemplates the introduction of the protein itself. It is understood that when introduction of a protein discussed, that the invention also contemplates the introduction of a nucleic acid encoding the protein. In some embodiments, both a protein and a nucleic acid encoding it are introduced.

Additionally, a nucleic acid or protein of the invention can be delivered or administered to an animal via a cell, e.g., as cell therapy. For example, if a particular protein(s) is to be administered or delivered, this can be accomplished by administering or delivering a cell(s) expressing the protein(s). In some embodiments, the protein(s) is expressed from the cell via a regulatable, inducible and/or repressible promoter. In some embodiments, encapsulated cells are delivered to an animal that express a protein(s) and/or nucleic acid(s) of interest, e.g., see PCT Publication No. WO07078922. Cells to be administered to an animal can be autologous, allogeneic or xenogeneic. In some embodiments, autologous cells are manipulated ex vivo to cause them to produce a molecule of the invention and then the cells are introduced back to the animal. In some embodiments, cells are administered locally (e.g., in a joint, intravitreal, intraretinal, etc.) or systemically (e.g., i.v.).

In some embodiments, target cells are mammalian cells such as primate cells, and human cells. In some embodiments, target cells are cells of the eye, such as RPE cells, retinal cells, or pluripotential cells. Target cells can be in vitro, ex vivo or in vivo. In some embodiments, a gene delivery system contemplated herein can result in stable integration of a gene or coding region of interest in a host cell genome. In some embodiments, a cell is a stem cell. Stem cells include, but are not limited to, pluripotent stem cells, totipotent stem cells, hematopoietic stem cells, cancer stem cells and embryonic stem cells. In some embodiments, pluripotential cells contemplated herein are not those for propagating a living entity from a zygote or blastomere. The instant invention also contemplates the use of a partially undifferentiated cell for implantation into the eye of a patient in need of treatment, e.g., to regenerate cells of the eye.

Non-limiting examples of a gene of interest or a therapeutic gene include nucleic acids encoding expression products that enhance expression of an inflammation inhibitor, such as, DAF, that reduce expression of an inflammation inducer or facilitator and so on.

In some embodiments, a molecule of the present invention is an aptamer. Aptamers are nucleic acid sequences that, similar to antibodies, bind to a target molecule. The technology provides nucleic acid molecules, each having a unique sequence, which have the property of binding specifically to a desired target compound or molecule, but not necessarily by complementary base pairing of a single strand to another, instead resembling the reaction of a ligand to its receptor or partner or an antigen to an antibody. A nucleic acid molecule can be a specific ligand of a given target compound or molecule. Nucleic acids have sufficient capacity for forming a variety of two-dimensional and three-dimensional structures and have sufficient chemical versatility to act as ligands or binders (form specific binding pairs) with virtually any chemical compound, whether monomeric or polymeric. Molecules of any size can serve as targets, see, for example, U.S. Pat. Nos. 5,567,588; 5,270,163; and 5,756,291. Aptamers can be used similarly as described for antibodies or binding molecules as described herein. In some embodiments, an aptamer of the invention is a thioaptamer (e.g., see Volk et al. Annals of the New York Academy of Sciences 1082 (1), 116-119 (2006).

In some embodiments, a molecule or binding molecule of the invention is an antibody mimic such as an ADNECTIN™, also e.g., see U.S. Pat. No. 7,115,396. ADNECTINS™ are a class of targeted biologics that are derived from fibronectin.

Generally, in vitro selection techniques for identifying aptamers involve having a pool of DNA molecules that contain at least some region that is randomized or mutagenized. Thus, an oligonucleotide pool for aptamer selection might contain a region of 20-100 randomized nucleotides flanked by about 15-25 base regions of defined sequence useful, for example, for the binding of PCR primers. The oligonucleotide pool can be amplified using standard PCR techniques, although any means that provides amplification of the nucleic acid sequences can be employed. The pool can then be in vitro transcribed to produce RNA transcripts. The RNA transcripts may then be subjected to a selection scheme to isolate nucleic acids that bind specifically to another molecule, the ligand (e.g., a protein or any target molecule). The RNA molecules which bind the ligand can then be reverse transcribed and amplified. The selected sequences can be exposed to additional selection steps. The cDNA thereof then can be amplified, cloned, and sequenced to further characterize the candidate aptamers for the target ligand. Once an aptamer sequence has been successfully identified, the aptamer may be further optimized by additional selection steps or can be modified by mutagenesis to obtain molecules, with, for example, higher specificity. It may be beneficial for the aptamer to be selected for ligand binding in the presence of salt concentrations and temperatures which mimic normal physiological conditions.

As with many of the nucleic acids used herein, the nucleic acid of interest can be a DNA or an RNA, and may be a single-stranded molecule, or may be partial or fully double-stranded. In some embodiments, a nucleic acid may be, but is not limited to, DNA, RNA, miRNA or siRNA. In some embodiments, a nucleic acid encodes a protein. In some embodiments, a nucleic acid does not code for a protein. In some embodiments, a nucleic acid inhibits expression of a protein from a second nucleic acid, e.g., a mRNA.

An aptamer, as with any therapeutic nucleic acid, can bind to a target protein, or can bind to a target nucleic acid, whether in a coding region, or in a non-coding region, such as an intron or an upstream/downstream regulatory sequence.

There are a number of ways to modify a nucleic acid to obtain beneficial properties associated therewith, without diminishing the desired nucleic acid binding property thereof. These are known to those of skill in the art and include PEGylation, sulfur backbone modifications and methylation.

A “stabilized nucleic acid molecule” shall mean a nucleic acid molecule that is relatively resistant to in vivo degradation (e.g., via an exonuclease or endonuclease). Stabilization can be a function of length and/or secondary structure. Stabilization can be obtained by controlling, for example, secondary structure which can stabilize a molecule. For example, if the 3′ end of a nucleic acid molecule is complementarily to an upstream region, that portion can fold back and form a “stem loop” structure which stabilizes the molecule.

“Ribozyme” refers to a nucleic acid capable of cleaving a specific nucleic acid sequence. Within some embodiments, a ribozyme should be understood to refer to RNA molecules that contain anti-sense sequences for specific recognition, and an RNA-cleaving enzymatic activity, see, for example, U.S. Pat. No. 6,770,633.

Antisense oligonucleotides generally are small oligonucleotides complementary to a part of a gene to impact expression of that gene. Gene expression can be inhibited through hybridization of an oligonucleotide to a specific gene or messenger RNA (mRNA) thereof. In some cases, a therapeutic strategy can be applied to dampen expression of one or several genes believed to initiate or to accelerate inflammation, see, for example, U.S. Pat. No. 6,822,087 and WO 2006/062716.

A “small interfering RNA” or “short interfering RNA” or “siRNA” or “short hairpin RNA” or “shRNA” are forms of RNA interference (RNAi). An interfering RNA can be a double-stranded RNA or partially double-stranded RNA molecule that is complementary to a target nucleic acid sequence, for example, VEGF. Micro interfering RNA's (miRNA) also fall in this category. A double-stranded RNA molecule is formed by the complementary pairing between a first RNA portion and a second RNA portion within the molecule. The length of each portion generally is less than 30 nucleotides in length (e.g., 29, 28, 27, 26, 25, 24, 23, 22, 21, 20, 19, 18, 17, 16, 15, 14, 13, 12, 11, or 10 nucleotides). In some embodiments, the length of each portion is 19 to 25 nucleotides in length. In some siRNA molecules, the complementary first and second portions of the RNA molecule are the “stem” of a hairpin structure. The two portions can be joined by a linking sequence, which can form the “loop” in the hairpin structure. The linking sequence can vary in length. In some embodiments, the linking sequence can be 5, 6, 7, 8, 9, 10, 11, 12 or 13 nucleotides in length. Linking sequences can be used to join the first and second portions, and are known in the art. The first and second portions are complementary but may not be completely symmetrical, as the hairpin structure may contain 3′ or 5′ overhang nucleotides (e.g., a 1, 2, 3, 4, or 5 nucleotide overhang). The RNA molecules of the invention can be expressed from a vector or produced chemically or synthetically.

miRNA's are small RNAs that regulate gene expression following transcription through interaction with homologous mRNAs. miRNA's can control expression of genes by binding to complementary sites in target mRNAs from protein coding genes. miRNA's are processed from larger double-stranded precursor molecules. The precursor molecules are often hairpin structures of about 70 nucleotides in length, with 25 or more nucleotides that are base-paired in the hairpin. The RNAse III-like enzymes, Drosha and Dicer, cleave the precursor to produce an miRNA. miRNA's generally are single-stranded and incorporate into a protein complex, termed an RNA-induced silencing complex (RISC) or miRNP. The RNA-protein complex targets a complementary mRNA. miRNA's inhibit translation or direct cleavage of target mRNAs. (Brennecke et al., Genome Biology 4:228 (2003); Kim et al., Mol. Cells 19:1-15 (2005)).

RNAi-mediated suppression of nucleic acid expression is relatively specific. Some base pair mismatch between the RNAi molecule and the targeted nucleic acid can be tolerated without abolishing the action of RNA interference, e.g., see WO 2006/062716. An RNAi of the invention generally does not elicit significant anti-viral responses.

There are schemes for designing siRNAs (see, e.g., Elbashire et al., 2001, Nature, 411:494-8; Amarzguioui et al., 2004, Biochem. Biophys. Res. Commun., 316(4):1050-8; and Reynolds et al., 2004, Nat. Biotech., 22(3):326-30) known in the art. Details for making siRNA's can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs of the invention can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

Triplex molecules refer to single DNA strands that target duplex DNA, forming co-linear triplexes by binding to the major groove, and thereby preventing or altering transcription (see, e.g., U.S. Pat. No. 5,176,996).

A number of target-binding molecules can be expressed by recombinant means. For example, single chain antibodies, domain antibodies, receptors and the like, and the nucleic acids encoding same, can be used as therapeutic genes to make therapeutic proteins in the practice of the instant invention.

An “antibody” refers to an intact immunoglobulin, or to an antigen-binding portion thereof that competes with the intact antibody for specific binding, that is, the fragment retains cognate antigen-binding ability. In some embodiments, antigen-binding portions may be produced by recombinant DNA techniques or by enzymatic or chemical cleavage of intact antibodies. Antigen-binding portions include, inter alia, F_{ab}, F_{ab′}, F_{(ab′)2}, Fᵥ, dAb, and complementarity determining region (CDR) fragments, single-chain antibodies (scFᵥ), chimeric antibodies, diabodies and polypeptides that contain at least a portion of an immunoglobulin that is sufficient to confer specific antigen binding to the polypeptide. An F_{ab} fragment is a monovalent fragment consisting of the V_{L}, V_{H}, C_{L} and C_{H}1 domains; an F_{(ab′)2} fragment is a bivalent fragment comprising two F_{ab} fragments linked by a disulfide bridge at the hinge region; an F_{d} fragment consists of V_{H} and C_{H}1 domains; an Fᵥ fragment consists of V_{L} and V_{H} domains of a single arm of an antibody; and a dAb fragment (Ward et al., Nature 341:544-546, 1989) consists of a V_{H} domain. A single-chain antibody (scFᵥ or scAb) is an antibody derivative in which V_{L} and V_{H} regions are paired to form a monovalent molecule via a synthetic linker that enables the V regions to be made as a single protein chain (Bird et al., Science 242:423-426, 1988 and Huston et al., Proc. Natl. Acad. Sci. USA 85:5879-5883, 1988). Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently. As known in the art, a single CDR can confer antigen binding ability and specificity on a polypeptide carrying same. A molecule that specifically binds is one that carries the requisite focused reactivity to recognize and to bind the epitope to which the original antibody was generated, the cognate antigen, from a plurality of other molecules using standards for identifying and assessing specificity and cross reactivity as known in the immunology arts. Any of the antibodies or fragments thereof that retain at least some of their binding can be utilized in the present invention. Various methods are known in the art for preparing, purifying, administering and/or utilizing antibodies, e.g., see U.S. Pat. No. 6,884,879.

Chimeric antibodies are molecules containing different portions which are derived from different animal species, such as those having a variable region derived from a murine mAb and a human immunoglobulin constant region. Chimeric antibodies are primarily made and used to reduce immunogenicity. Chimeric antibodies and methods for their production are known in the art (Morrison et al., Proc. Natl. Acad. Sci. USA 81:6851-6855 (1984); Boulianne et al., Nature 312:643-646 (1984); Europe Patent Application 125023; Neuberger et al., Nature 314:268-270 (1985); Europe Patent Application 171496; Europe Patent Application 184187; Sahagan et al., J. Immunol. 137:1066-1074 (1986); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow & Lane Antibodies: a Laboratory Manual Cold Spring Harbor Laboratory (1988)).

The term “humanized immunoglobulin” as used herein refers to an immunoglobulin comprising portions of immunoglobulins of different origin, wherein at least one portion is of human origin. For example, the humanized antibody can comprise portions derived from an immunoglobulin of nonhuman origin with the requisite specificity (e.g., from a mouse) and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin of the present invention is an immunoglobulin containing one or more immunoglobulin chains comprising a CDR derived from an antibody of nonhuman origin and a framework region derived from a light and/or heavy chain of human origin (e.g., CDR-grafted antibodies with or without framework changes to improve antibody stability and/or binding affinity and, optionally, one or more changes in one or more CDRs to improve antibody stability and/or, preferably, binding affinity). Chimeric or CDR-grafted single chain antibodies are also encompassed by the term humanized immunoglobulin, see, e.g., Cabilly et al., U.S. Pat. No. 4,816,567; Boss et al., U.S. Pat. No. 4,816,397; Neuberger et al., WO 86/01533; Winter, U.S. Pat. No. 5,225,539; Padlan et al., Europe Patent Application No. 0,519,596, Ladner et al., U.S. Pat. No. 4,946,778; Huston, U.S. Pat. No. 5,476,786; Studnicka et al., U.S. Pat. No. 5,766,886; Queen et al., U.S. Pat. No. 7,022,500 and Bird et al., Science, 242:423-426 (1988).

Catalytic antibodies are those that can effect a chemical reaction or change, such as cleavage of a peptide bond, in the bound antigen. Catalytic antibodies can be induced by immunizing an animal with a transition state analogue (TSA) rendered immunogenic (Pollack et al., J. Am. Chem. Soc. (1988) 110:8713, Jackson et al., PNAS (1988) 85:4953, Shokat et al., Chem. Int. Ed. Engl. (1988) 27:1172) and are capable of catalyzing different types of chemical reactions, see, for example, U.S. Pat. Nos. 5,401,641; 6,590,080; 7,109,291 and 7,205,136. In some embodiments, a catalytic antibody causes destabilization of a target protein. In some embodiments, a catalytic antibody acts as a protease that cleaves a target molecule, e.g., factor B, factor D, factor Bb, factor C3 and/or factor C3b.

Also, camelid antibodies that naturally lack a light chain can be used. Structures known as nanobodies and domain antibodies can be used, including polypeptides comprising a single CDR of an antibody known to bind the cognate antigen so long as the antigen, determinant or epitope binding ability is retained.

Single chain antibody (“SCA”), and other forms of recombinantly produced binding molecules are useful, e.g., for gene delivery. The relevant coding sequences of a particular antibody, and antigen-binding portions thereof, can be isolated and cloned for preparation of polypeptides with the requisite cognate antigen binding ability. Methods of making these fragments are known in the art (see, for example, Harlow & Lane, Antibodies: A Laboratory Manual, Cold Spring Harbor Laboratory, New York, 1988).

In some embodiments of the invention, antibody sequences are humanized or human, e.g., to reduce the risk of generating an immune response thereto. Various steps can be taken, as known in the art, to modify the amino acids of an antigen-binding polypeptide or any therapeutic protein of interest to retain the binding activity while making that molecule less antigenic, by substituting amino acids at particular sites of interest, as determined as a design choice.

In some embodiments, amino acid substitutions include, but are not limited to, those which: (1) reduce susceptibility to proteolysis, (2) reduce susceptibility to oxidation, (3) alter binding affinity for forming protein complexes, (4) alter (e.g., increase or decrease) binding affinities, and (5) reduce immunogenicity.

The present invention also includes the use of analogs. Analogs can include various muteins of a sequence other than the naturally-occurring peptide sequence. For example, single or multiple amino acid substitutions (e.g., conservative amino acid substitutions) may be made in the naturally-occurring sequence (e.g., in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991). In some embodiments, a non-conservative substitution is used.

In the context of gene therapy, alterations will be reflected at the level of the encoding nucleic acid. Thus, modifications also can be made to the nucleic acid to enhance expression. For example, certain codons may be preferred by a particular host cell. Thus, recoding can occur where certain codons are preferred in, for example, mammalian expression systems and cells. Recoding of nucleic acids is a design choice available to the artisan.

Methods for controlling transcription and/or translation can be used in the practice of the instant invention. In some embodiments, these methods are amenable to local gene delivery, for example, to eye cells. For example, one can use peptide-nucleic acid oligomers (PNA's) (Eglon et al. Nature 365, 566, 1993) or nucleic acid binding polypeptides, such as transcription factors, which can be designed to bind any genomic DNA sequence through the incorporation of engineered zinc fingers. Such engineered transcription factors can be designed to either up regulate or down regulate any endogenous gene.

Various methods for gene therapy and gene transfer are known and any can be used to practice the instant invention. General reviews of the methods of gene transfer include Goldspiel et al., Clin. Pharm. 12:488-505 (1993); Wu & Wu, Biotherapy 3:87-95 (1991); Tolstoshev, Ann. Rev. Pharmacol. Toxicol. 32:573-596 (1993); Mulligan, Science 260:926-932 (1993); Morgan & Anderson, Ann. Rev. Biochem. 62:191-217 (1993); and May, TIBTECH 11(5):155-215 (1993). Methods of recombinant DNA technology are known and reference can be made to Ausubel et al. (eds.), Current Protocols in Molecular Biology, John Wiley & Sons, NY (1993); and Kriegler, Gene Transfer and Expression, A Laboratory Manual, Stockton Press, NY (1990).

In one aspect, an expression vector or vector construct encodes an antibody derivative, such as a single chain antibody. In some embodiments, an expression vector of interest expresses an antibody derivative in a human eye cell.

Some embodiments of the invention involve delivery of a vector construct into an animal, e.g., a human. Delivery of a vector or even a protein into a human may be either direct, in which case the human is directly exposed to the vector or protein, such as by injection (e.g., into the eye such as intravitreally or subretinally), or indirect, in which case, cells are first transformed with the vector in vitro, and then the transformed cells are transplanted into the patient. In some embodiments, these transformed cells are autologous. In some embodiments, these transformed cells allogeneic. Transferring a nucleic acid comprised of a coding region to cells in tissue culture can be by any method, such as, electroporation, microinjection, cell fusion, chromosome-mediated gene transfer, microcell-mediated gene transfer, spheroplast fusion, lipofection, microparticle bombardment, calcium phosphate mediated transfection, viral infection and so on. Optionally, a selectable marker also can be introduced into the cells. If a selectable marker is utilized, the cells can be then placed under selection, e.g., to enhance expression and/or to isolate those cells that express the transferred coding region (see, e.g., Loeffler & Behr, Meth. Enzymol. 217:599-618 (1993); Cohen et al., Meth. Enzymol. 217:618-644 (1993); and Cline, Pharmac. Ther. 29:69-92 (1985)).

Recombinant cells (e.g., autologous or allogeneic cells transformed in vitro) can be delivered to a patient by various methods known in the art. For example, cells can be encapsulated prior to administration, as known in the art. In some embodiments, when encapsulated, the cells are not autologous. In some embodiments, recombinant blood cells (e.g., hematopoietic stem and/or progenitor cells) are administered intravenously. In some embodiments, eye cells and/or pluripotential cells can be injected directly into the eye. The amount of cells needed depends on the desired effect, the animal's state, etc., and can be determined by one skilled in the art practicing methods\known in the art.

An amount of a composition, such as comprising a protein, nucleic acid or vector particle of the invention that will be effective in the treatment, inhibition and prevention of a disease, pathway or disorder, e.g., associated with blinding ocular disease, can be determined by standard clinical techniques. In addition, in vitro assays may optionally be employed to help identify optimal dosage ranges. The precise dose to be employed in a formulation will also depend on the route of administration, and the seriousness of the disease or disorder. In some embodiments, this should be decided according to the judgment of the practitioner and/or the individual circumstances of the patient. Effective doses may be extrapolated from dose-response curves derived from in vitro or animal model test systems.

Some molecules of interest need not be delivered by a gene transfer means, and may be administered by any drug delivery means known in the art, such as by injection, drops and so on. Thus, an aptamer, a spiegelmer, an antibody-type or antibody-derived molecule, a complement regulator protein, a dominant negative complement protein, a complement binding peptide or protein, a peptide nucleic acid (PNA), and so on, which, for example, dampen, neutralize, reduce expression of and so on of, for example, a complement factor or complement regulatory molecule, are included in the present invention and can be formulated as known in the art. Thus, a molecule of interest may be lyophilized for reconstitution when used, or may be presented in liquid form containing pharmaceutically acceptable diluents, such as water, saline or buffer, and can contain optional buffers, preservatives, stabilizers and the like. A formulation may be stable at room temperature, reduced or refrigerator temperatures or may be frozen. A formulation can also take other forms, such as being tableted or stored in a capsule or depot and so on.

### Complement Pathways and a Disease Model for an Etiology of AMD in Humans

This section provides background information on the complement system as well as a novel disease model to facilitate an understanding of the instant invention. There are three pathways of complement activation, the classical pathway, the alternative pathway, and the lectin pathway. Examples 8 and 9 describe examples of proteins of the instant invention. In some embodiments, these proteins can attenuate the alternative pathway of complement activation. However, based on the way all three complement pathways intersect, these proteins will diminish inflammation caused by any of the three complement pathways and thereby provide therapy for any illness whose etiology involves, at least in part, complement activation. These include, but are not limited to, early AMD, wet AMD, and Geographic Atrophy.

Complement pathways are a part of the immune system known as the innate immune system that provides immediate protection from infection prior to activation of the humoral and cell mediated branches of the immune system. These pathways are composed of approximately 35 factors. They are activated and inactivated through cascading reactions that exhibit high order kinetics but are remarkably well-regulated. The alternative complement pathway, in particular, has evolved to cycle up with great rapidity through a positive feedback loop. The inventors are not aware of any developed therapeutic complement inhibitors that are both efficacious and safe for the treatment of chronic diseases possibly because persistent systemic blockade or inhibition of complement activity may predispose a patient to infection.

Proteins of the invention and/or the vectors that express them may advantageously be used for systemic administration to a mammal and/or to treat chronic diseases. For example, complement protein analogs, such as factor B or factor D analogs of the invention inhibit the complement pathway by competing with the binding of the native protein. This can allow attenuation of complement activity as opposed to complete blockade of the pathway. Therefore, it may be possible to downregulate complement activity to a level that is therapeutic (e.g., alleviates some symptoms or their severity) without completely blocking complement activity. Thus, avoiding or decreasing the risks associated with blockage of complement activity, such as increased risk of infection. Therefore, the present invention provides methods for treating a complement related disease (e.g., a chronic disease) by systemic administration (e.g., i.v., intraperitoneal or oral) of a protein or composition of the invention.

Complement-mediated activity not specifically directed at the offending infectious agent can cause significant collateral damage to normal cells. To protect itself from this collateral damage, several components of the complement system are specifically designed to rein in complement activity and protect nearby normal cells. These complement inhibitors are usually found either in the fluid (plasma) phase or as integral membrane proteins on normal cells. One complement factor in particular, CFH, is a major inhibitor of the alternative pathway that normally circulates at high levels in plasma but has the capability of binding to cell membranes, intercellular matrix components, and some plasma proteins.

FIGS. 11 and 12 outline complement pathways, note that they intersect at C3b.

Table One outlines some of the regulators of complement activation.

TABLE 1
Regulators of Complement Activation

| Protein | Pathway | Function |
|---|---|---|
| C1inh | Classical | Blocks initiation |
| C4bp | Classical | Decay acceleration and CFI cofactor |
| DAF | Both | Decay acceleration of C3 convertases |
| MCP | Both | Cofactor for CFI (for fluid phase C3b) |
| CR1 | Both | Decay acceleration and CFI cofactor |
| CFH | Alternative | Decay acceleration, CFI cofactor, and |
| | | MAC inhibitor |
| CFI | Both | Degrades C3b and C4b |
| CD59 | Both | Inhibits MAC assembly |
| Clusterin | Both | Inhibits MAC assembly |
| S Protein | Both | Inhibits MAC assembly |
| HRF | Both | Inhibits MAC assembly |

The distribution of each inhibitor is relevant to its function. For example, DAF, MCP, CD59 and others are integral membrane proteins on the host cells. CFH, on the other hand, is in the fluid phase, but can bind to certain solid structures.

### Model to Describe One Etiology for AMD

Whereas the instant invention is not meant to be limited by a specific mechanistic model, the inventors speculate that the following model describes a major etiology for most cases of AMD. Many factors contribute to the development and course of AMD in an individual. The following model depicts one set of factors that can accelerate the course of the illness, e.g., in a majority of patients. Moreover, these factors can lead to a particularly early or rapid course in those individuals who, for a variety of reasons, are genotypically predisposed to this etiology.

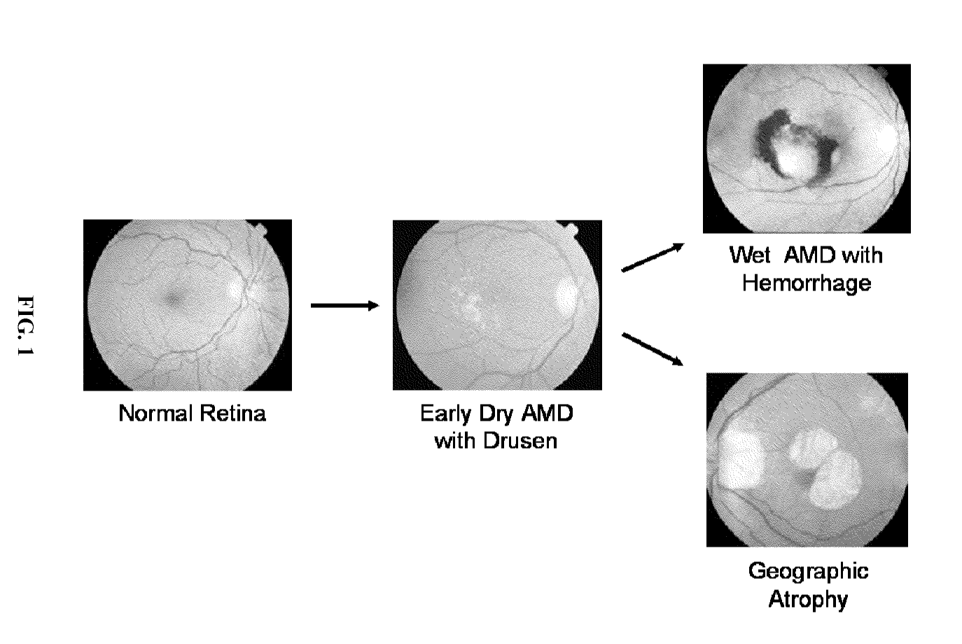

Early AMD is characterized by the accumulation of drusen between the RPE cells and the underlying membrane, termed Bruch's membrane (FIG. 1). Drusen contains many components including some complement factors as well as a plasma protein called C Reactive Protein (CRP), which is generally viewed as a marker for inflammation. CRP is a pentomeric protein that binds to various lipids and nuclear components. CRP also serves to: 1) bind to the Fc receptors on phagocytotic cells; 2) activate the early steps in the classical complement pathway; 3) up-regulate the membrane bound complement inhibitory factors DAF, MCP, and CD59; and 4) bind CFH (e.g., see Johnson et al. 2006, Black et al. 2004, Mold et al. 1999, and Li et al. 2004).

Not wishing to be bound by theory, the inventors speculate that, in early AMD, as drusen accumulates, CRP binds to and is immobilized in the drusen. The immobilized CRP would then serve to attract phagocytotic cells and stimulate them to engulf and to remove the drusen. CRP would do so through its ability to bind the Fc receptor and through generation of the biologically active fragments C4a and C3a. However, activation of the classical pathway to yield these fragments also yields C3b, which is then able to enter the alternative pathway and cycle up through the positive feedback loop. Thus, unless the alternative pathway is controlled, drusen may become a repository for increasing inflammation. Whereas CRP does up-regulate DAF, MCP, and CD59, these are cell surface proteins and may not be able to effectively control the alternative pathway inside the drusen. The inventors speculate that, to prevent runaway activation of the alternative pathway and significant inflammation, the CRP may bind and immobilize CFH within the drusen. According to this non-limiting model, if the CFH is not able to bind efficiently to CRP or if it is simply not effective in attenuating the alternative pathway, then there could be significant inflammation under the retina. This could lead to increased tissue damage with increased binding of CRP. Thus, the whole process would cycle up and eventually lead to progressive AMD including both end-stage courses, wet AMD and Geographic Atrophy (GA). Whether or not a patient develops wet AMD or GA would be determined by additional acquired and genetic factors including the balance of pro- and anti-angiogenic factors as well as the expression levels, e.g., of the matrix protease and TGF-β regulator HtrA1 (e.g., see Oka et al. 2004, Yang et al. 2006, and DeWan et al. 2006).

In this model, CRP is part of the natural clearing process for debris in the back of the eye. However, in carrying out this role, CRP can potentially provoke tissue injury from activation of the alternative complement pathway. Ineffective control of the alternative pathway, over the years, may lead to progressive AMD. The instant invention provides various embodiments which inhibit the alternative pathway.

Design of Proteins that can Attenuate Complement Activation

The instant invention includes proteins, e.g., that can be delivered as proteins and/or via gene transfer (e.g., vectors comprises of genes and/or coding regions that code for the protein(s)) to attenuate the alternative pathway of complement activation. These proteins may overcome hurdles that impede the development of complement inhibitors (e.g., for eye disease) including, for example: 1) avoiding long term systemic immune suppression; 2) achieving efficacy in the face of otherwise prohibitively high levels of complement factors in the blood; 3) achieving sufficient levels and distribution of the therapeutic protein in the proximity of the retina and Bruch's membrane for efficacy; 4) achieving activity of the therapeutic protein within drusen; 5) achieving sufficient duration of therapeutic delivery to treat a chronic disease; 6) achieving efficacy without interfering with the classical complement pathway activities in the back of the eye; and 7) avoiding or diminishing an immune reaction (e.g., a local immune reaction) to the therapeutic.

The inventors have determined that attenuating the positive feedback loop in the alternative pathway is a means of down-regulating the entire pathway. Such attenuation could be achieved by interfering with the function and/or level of fB, fD, or properdin. In some embodiments, pathway attenuation could be achieved by up-regulating or normalizing the function of natural regulators such as DAF, MCP, CR1, or CFH. In some embodiments, expressing the extracellular domains thereof may enable penetration into the drusen.

The inventors have determined that a suitable means of attenuating the alternative pathway feedback loop is to interfere with complement factor B (fB) function or levels. Some embodiments of the invention use a dominant negative strategy for attenuating fB function. The following describes as examples three specific dominant negative fB moieties, each with unique attributes.

Complement factor B circulates as an inactive protease and functions via a two-step process. First, it binds factor C3b to form a transient complex. Second, it is cleaved by fD to yield Bb and to activate the fB serine protease. The C3bBb complex is then stabilized by properdin.

Nucleotide sequences for genes and coding regions encoding human factor B and other complement proteins, as well as the amino acid sequence of such proteins, are known in the art. For example, a gene encoding human factor B and other complement proteins is found in NCBI Database Accession No. NG_{—}000013. A coding sequence for factor B is found in NCBI Database Accession No. NM_{—}001710 and the amino acid sequence for factor B preproprotein is found in NCBI Database Accession No. NP_{—}001701 or P00751. NCBI Database Accession No. P00751 is a human preproprotein factor B sequence. Sequences from other animal species are also known in the art. By way of comparison, in the mouse factor B sequence (e.g., see NCBI Database Accession No. P04186), the third SCR domain is located at positions 160-217 of this 761 amino acid preprotein, and the mature murine factor B protein spans positions 23-761. The first 22 amino acids of mouse factor B is a signal sequence (SEQ ID NO:16).

Human factor B preprotein is a 764 amino acid protein (SEQ ID NO:2) with a signal peptide spanning amino acid positions 1-25. The mature chain of factor B corresponds to positions 26-764. The three SCR regions of human factor B are SCR1, also known as Sushi 1, spanning from about position 35 to about position 100, SCR2, also known as Sushi 2, spanning from about position 101 to about position 160 and SCR3, also known as Sushi 3, spanning from about position 163 to about position 220.

The first of the three dominant negatives, termed fB1, alters one amino acid in the fB protease site. This fB moiety binds C3b with normal affinity and kinetics, but when acted upon by fD and stabilized by properdin, does not function as a protease and does not form a C3 convertase e.g., a substation with N at an amino acid corresponding to position 740 of SEQ ID NO:2 (e.g., D740N).

The second dominant negative, termed fB2, alters the same amino acid as fB1, but in addition, alters two additional amino acids in the C3b binding domain (substitutions at amino acids corresponding to positions 279 and 285 of SEQ ID NO:2) to increase the binding affinity of fB2 to C3b, e.g., D279G, N285D and D740N changes. The N285D substitution removes a putative N-glycosylation site.

The third dominant negative, termed fB3, combines the mutations that increase C3b binding from fB2 with a mutation that knocks out the binding site for cleavage by factor D, particularly with substitutions at positions corresponding to residues 258, 259 and 260 of SEQ ID NO:2 as well as substitutions at 279 and 285, e.g., K258A, R259A, K260A, D279G and N285D changes. Cleavage by factor D of wild type fB activates the fB protease. Thus, fB3, with its five amino acid changes, efficiently binds C3b but has minimal protease activity.

FB1, fB2 and fB3 are examples of fB analogs that can be used in the practice of the invention, but the invention is not limited to these specific analogs. Some embodiments of the invention include any fB analog that inhibits a complement pathway. In some embodiments, an fB analog is not fB1. In some embodiments, an fB analog is not fB2. In some embodiments, an fB analog is not fB3. In some embodiments, an fB analog comprises one or more mutations of amino acids corresponding to one or more of the following amino acids in SEQ ID NO:2: amino acid 258, 259, 260, 279, 285, 739, 740, 741, 742, 743, 744, 745 and 746. These one or more mutations can be a substitution or deletion of the amino acid or an addition of at least one amino acid next to or within 1, 2, 3, 4, 5, 6, 7, 8, 9 or 10 amino acids. In some embodiments, this addition disrupts, changes, enhances or inhibits the function of the listed amino acid, e.g., disrupts its role (i) in cleavage of another protein (e.g., 740), (ii) as a site of cleavage by another protein (e.g., 258, 259 and/or 260), or (iii) its role in binding another protein (e.g., 279 or 285).

Some embodiments of the invention comprise a substitution of the amino acid corresponding to one or more of the 258, 259 and/or 260 amino acids with an amino acid selected from the group consisting of alanine, glycine, valine, leucine and isoleucine. Some embodiments of the invention comprise a deletion of the amino acid corresponding to one, two or three of the following amino acids: 258, 259 and/or 260. Some embodiments of the invention comprise at least one addition of 1, 2, 3, 4, 5, 6, 7, 8, 9, 10 or more amino acids immediately next to an amino corresponding to the 258, 259 and/or 260 amino acids.

Some embodiments of the invention comprise a substitution of the amino acid corresponding to position 739 of SEQ ID NO:2 with an alanine. Some embodiments of the invention comprise a substitution of the amino acid corresponding to position 739 of SEQ ID NO:2 with an amino acid selected from the group consisting of alanine, glycine, valine, leucine and isoleucine. Some embodiments of the invention comprise deletion the amino acid corresponding to the 739 amino acid.

Some embodiments of the invention comprise a substitution of the amino acid corresponding to position 740 of SEQ ID NO:2 with an amino acid selected from the group consisting of glutamic acid, asparagine, alanine, serine, glycine and tyrosine. Some embodiments of the invention comprise a substitution of the amino acid corresponding to position 740 with an amino acid selected from the group consisting of valine, leucine, isoleucine, threonine, cysteine, methionine, aspartic acid, glutamine, phenylalanine, tyrosine, tryptophan, glutamic acid, asparagine, alanine, serine, glycine and tyrosine. Some embodiments of the invention comprise a deletion of the amino acid corresponding to the 740 amino acid.

Some embodiments of the invention comprise a substitution of the amino acid corresponding to position 741 of SEQ ID NO:2 with an amino acid selected from the group consisting of tryptophan and alanine Some embodiments of the invention comprise a substitution of the 741 amino acid with an amino acid selected from the group consisting of alanine, glycine, valine, leucine and isoleucine. Some embodiments of the invention comprise a substitution of the 741 amino acid with an amino acid selected from the group consisting of tryptophan, tyrosine and phenylalanine. Some embodiments of the invention comprise a deletion of the 741 amino acid.

Some embodiments of the invention comprise a substitution of the amino acid corresponding to position 742 of SEQ ID NO:2 with a glutamine. Some embodiments of the invention comprise a substitution of the 742 amino acid with an amino acid selected from the group consisting of glutamine, glutamic acid, asparagine, and aspartic acid. Some embodiments of the invention comprise a deletion of the 742 amino acid.

Some embodiments of the invention comprise a substitution of the amino acids corresponding to positions 743 and/or 745 of SEQ ID NO:2 with a phenylalanine. Some embodiments of the invention comprise a substitution of the 743 and/or 745 amino acid with an amino acid selected from the group consisting of phenylalanine, tyrosine and tryptophan. Some embodiments of the invention comprise a deletion of one or more of the 743, 744 and/or 745 amino acids.

Some embodiments of the invention comprise a substitution of the amino acid corresponding to position 746 of SEQ ID NO:2 with an amino acid selected from the group consisting of tryptophan and alanine. Some embodiments of the invention comprise a substitution of the 746 amino acid with an amino acid selected from the group consisting of alanine, glycine, valine, leucine and isoleucine. Some embodiments of the invention comprise a substitution of the 746 amino acid with an amino acid selected from the group consisting of tryptophan, tyrosine and phenylalanine. Some embodiments of the invention comprise a deletion of the 746 amino acid.

Some embodiments of the invention comprise the insertion or substitution of 1, 2, 3, 4, 5, 6, 7, 8, 9, 10 or more amino acids immediately next to or in place of positions 739, 740, 741, 742, 743, 744, 745 and/or 746 amino acids.

Some embodiments of the invention comprise a substitution of the amino acid corresponding to position 279 of SEQ ID NO:2 with an amino acid selected from the group consisting of glycine, alanine and asparagine. Some embodiments of the invention comprise a substitution of the 279 amino acid with an amino acid selected from the group consisting of glycine, alanine, valine, leucine and isoleucine. Some embodiments of the invention comprise a substitution of the 279 amino acid with an amino acid selected from the group consisting of aspartic acid, asparagine, glutamic acid and glutamine. Some embodiments of the invention comprise a deletion of the 279 amino acid. Some embodiments of the invention comprise the insertion or substitution of 1, 2, 3, 4, 5, 6, 7, 8, 9, or more amino acids immediately next to or in place of 279.

Some embodiments of the invention comprise a substitution of the amino acid corresponding to position 285 of SEQ ID NO:2 with an amino acid selected from the group consisting of alanine and aspartic acid. Some embodiments of the invention comprise a substitution of the 285 amino acid with an amino acid selected from the group consisting of glycine, alanine, valine, leucine and isoleucine. Some embodiments of the invention comprise a substitution of the 285 amino acid with an amino acid selected from the group consisting of aspartic acid, asparagine, glutamic acid and glutamine. Some embodiments of the invention comprise a deletion of the N285 amino acid. Some embodiments of the invention comprise the insertion or substitution of 1, 2, 3, 4, 5, 6, 7, 8, 9, 10 or more amino acids immediately next to or in place of 285.

Some embodiments of the invention comprise a substitution of the one or more of the amino acids corresponding to positions 279, 282, 283, 284 and 285 of SEQ ID NO:2. In some embodiments, these amino acids are replaced with glycine, isoleucine, proline, histidine and aspartic acid, respectively.

Some embodiments of the invention comprise mutations of the amino acids corresponding to 258, 259, 260, 279 and 285 as described herein.

In specific embodiments, factor B analogs can be used in methods of the invention that comprise the amino acid sequence of one of SEQ ID NO:4, 6 or 8 (optionally without any signal sequence contained therein).

Analogs of the invention include factor B analogs that comprise a combination of the substitutions discussed herein and retain one or more of the attributes of the fB1, fB2 and/or fB3 analogs or any other fB analog discussed herein. The invention further provides analogs that are fragments (for example comprising one or more domains of a factor B protein having one or more of the amino acid alterations set forth herein) of these analogs that have one or more of the attributes of the analogs discussed above. In addition, the analogs may comprise additional amino acid substitutions, deletions or insertions (for example, conservative amino acid substitutions, truncations of the N-terminus or C-terminus, etc.) such that the analog is at least 99.5%, 99%, 98%, 95%, 90%, 85%, 80%, 75% or 75% identity.

In some embodiments, a dominant negative fB moiety is produced or delivered at levels that approximate or exceed the levels of native fB and C3b found locally, e.g., in the retina. In this regard, it is noteworthy that the high levels of fB and C3 in the plasma, which are 200 and 1,000 μg/ml, respectively, may not reflect the local levels of fB and C3b in other areas, such as the retina. In some embodiments, a dominant negative fB moiety is produced or delivered at levels that are from about 1% to about 100,000%; about 1% to about 10%; about 1% to about 20%; about 1% to about 30%; about 1% to about 40%; about 1% to about 50%; about 1% to about 60%; about 1% to about 70%; about 1% to about 80%; about 1% to about 90%; about 1% to about 100%; 90% to about 100%; 80% to about 100%; 70% to about 100%; 60% to about 100%; 50% to about 100%; 40% to about 100%; 30% to about 100%; 20% to about 100%; 10% to about 100%; 10% to about 20%; about 20% to about 30%; about 30% to about 40%; about 40% to about 50%; about 50% to about 60%; about 60% to about 70%; about 70% to about 80%; 80% to about 90%; about 100% to about 125%; about 100% to about 150%; about 100% to about 175%; about 100% to about 200%; about 100% to about 250%; about 100% to about 300%; about 100% to about 400%; about 100% to about 500%; about 100% to about 700%; about 100% to about 850%; about 100% to about 1000%; about 200% to about 300%; about 300% to about 400%; about 400% to about 500%; about 500% to about 600%; about 600% to about 700%; about 700% to about 800%; about 800% to about 900%; about 900% to about 1000%; about 250% to about 500%; about 500% to about 750%; about 750% to about 1000%; about 1000% to about 2000%; about 2000% to about 3000%; about 3000% to about 4000%; about 4000% to about 5000%; about 5000% to about 6000%; about 6000% to about 7000%; about 7000% to about 8000%; about 8000% to about 9000%; about 9000% to about 10,000%; about 10,000% to about 20,00%; about 10,000% to about 50,000%; or about 50,000% to about 100,000% of the levels of native fB and C3b found locally, e.g., in the retina of an animal such as a human.

To perform rodent experiments, analogous mutations for fB1, fB2, and fB3 were introduced into a mouse fB to yield mfB1, mfB2, and mfB3. At the amino acid level, human and mouse fB have 83% sequence identity. The amino acids that were modified in the human fB are completely conserved in the mouse fB. Despite this sequence identity, complement factors generally function in a species-specific manner. All in vitro studies are performed with both human and mouse specific assays, and the in vivo rodent studies are performed initially with the human and mouse fB mutants.

Exemplary procedures for generating eight cDNAs (human wild type fB and the three dominant negatives as well as the four analogous murine sequences) and their incorporation into vectors are detailed below in Example 8.

The inventors have concluded that another suitable means of attenuating the alternative pathway feedback loop is to interfere with complement factor D (fD) function or levels. Some embodiments of the invention use a dominant negative strategy for attenuating fD function. The following describes as examples of factor D analogs for inhibiting alternative complement pathway activity.

Complement factor D (fD) is a protease that cleaves and activates fB in the C3bB complex (FIG. 12). Factor D is typically present in plasma at low levels of approximately 2 ug/ml and serves as a catalyst in the alternative pathway. That is, a single fD moiety binds C3bB, cleaves fB to form the complex C3bBb, dissociates from the complex, and then goes on to repeat these steps. A dominant negative version of fD, which does not have proteolytic activity, would not be expected to inhibit the Alternative Pathway. In the presence of wild type fD, the dominant negative would simply bind and release, leaving the C3bB complex to be cleaved by the wild type fD. However, the discovery outlined in Example 21 showed that, in the absence of fB cleavage, fD does not release or at least releases from the complex at a lower frequency/rate. Therefore, it is possible that, in some embodiments, a dominant negative fD would, in fact, function as an inhibitor of the alternative pathway. In some embodiments, a dominant negative fD would bind C3bB and not dissociate or dissociate at a slower rate than wild-type fD. The complex would remain unavailable for fB cleavage by wild type fD and would not continue through the process of complement activation.

A wild-type human factor D is provided in SEQ ID NO:27. This is a fD preprotein with a signal peptide spanning amino acid positions 1-20.

In some embodiments, a fD analog binds the C3bB complex but does not cleave the fB or has a reduced ability to cleave fB. In some embodiments, a fD analog of the invention comprises additional amino acids to the N-terminus as compared to a wild-type fD. For example, it has been shown that there is a variant of fD with two additional amino acids, Gly-Arg, at the N-terminus, which circulates in the plasma at less than 1% of the level of wild type fD. This variant has no protease activity and can only be activated by a very high concentration of trypsin (Yamauchi et al., 1994 J. Immunol. 152(7):3645-53). This dominant negative variant should compete with wild type fD by binding to C3bB and preventing it from becoming a functional C3bBb, a C3 convertase. Therefore, the present invention provides fD analogs comprising one or more additional amino acids on the N-terminus as compared to wild-type fD, wherein the fD analog has a reduced or ablated ability or rate of cleaving fB. In some embodiments, this fD analog comprises more than two additional N-terminus amino acids. In some embodiments, this fD analog comprises 2, 3, 4, 5, 6, 7, 8, 9 or 10 additional N-terminus amino acids. In some embodiments, the additional N-terminus amino acids comprise glycine and arginine.

In some embodiments, a factor D analog has a mutation in the catalytic domain of the wild-type factor D. Three amino acids of fD are believed to be important components for the serine protease catalytic domain of fD. These three amino acids correspond to amino acids 66, 114, and 208 of SEQ ID NO:27, which also corresponds to amino acids 57, 102, and 195 as described in Volanakis & Narayana et al. (1996 Protein Science 5:553-564). In some embodiments, substitutions in one, two or all of these three amino acids can diminish or eliminate the serine protease activity but still enable binding to C3B. In some embodiments, the amino acid corresponding to the amino acid at position 66 of SEQ ID NO:27 is substituted with at least one neutral amino acid, at least one negatively charged amino acid or at least one nonpolar amino acid. In some embodiments, the amino acid corresponding to the amino acid at position 114 of SEQ ID NO:27 is substituted with at least one charged amino acid or at least one nonpolar amino acid. In some embodiments, the amino acid corresponding to the amino acid at position 208 is substituted with at least one charged amino acid or at least one nonpolar amino acid. In some embodiments, the amino acid corresponding to the amino acid at position 66, 114, or 208 of SEQ ID NO:27 is substituted with at least one amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

These fD analogs will compete with wild type fD by binding to C3bB and prevent or slow the rate at which it becomes a functional C3 convertase. Therefore, some embodiments of the invention include a fD analog comprising one or more mutations of amino acids corresponding to amino acids His66, Asp114, and Ser208 of SEQ ID NO:27. These three amino acids also correspond to His57, Asp102, and Ser195 amino acids using the numbering as used in Volanakis & Narayana et al. (1996 Protein Science 5:553-564). These one or more mutations can be a substitution or deletion of the amino acid or an addition of at least one amino acid next to or within 1, 2, 3, 4, 5, 6, 7, 8, 9 or 10 amino acids. Some embodiments of the invention include fD analogs with a mutation resulting in a change to the fD serine protease catalytic domain, wherein the fD analog still has the capability to bind C3B, but the fD analog has decreased ability to cleave fB. Analogs of the invention include factor D analogs that comprise a combination of the substitutions discussed above and retain one or more of the attributes of the fD analogs discussed above (such as decreased ability to cleave fB). The invention further provides analogs that are fragments (for example comprising one or more domains of a factor D protein having one or more of the amino acid alterations set forth herein) of these analogs that have one or more of the attributes of the analogs discussed herein. In addition, the analogs may comprise additional amino acid substitutions, deletions or insertions (for example, conservative amino acid substitutions, truncations of the N-terminus or C-terminus, etc.) such that the analog is at least 99.5%, 99%, 98%, 95%, 90%, 85%, 80%, 75% or 75% identity.

fD analogs as described herein can be used as a stand-alone therapeutic to block alternative pathway activation or in combination with other inhibitors of the alternative complement pathway, such as fD analogs as described herein. fD analogs as described herein, can be used as described for or in place of uses for fB as described herein.

Factor H, Factor H-like 1, MCP, DAF and CD59 typically act by inhibiting complement activity. Some embodiments of the invention include using Factor H, Factor H-like 1, CR1, MCP, DAF, and/or CD59 (alone or in combinations) to regulate complement activity. These proteins can be used in their protein form or be delivered via nucleic acids that encode the proteins, e.g., using a vector. In some embodiments, a MCP, DAF or CD59 is a soluble form or soluble fragment (e.g., wherein the transmembrane region is missing or replaced) capable of modulating (e.g., inhibiting) complement activity.

### Modulating Complement Related Pathways or Proteins Related to Various Complement-Associated Conditions

In some embodiments, the present invention provides compositions and methods for modulating, regulating, inhibiting and/or enhancing a complement-related, complement-associated pathway(s). Complement-related pathways include, but are not limited to, the classical and lectin complement pathways and the alternative complement pathway. In some cases, a complement-related pathway may play a role in a particular condition, disease or diseases. Therefore, some embodiments of the invention provide methods of regulating, modifying, curing, inhibiting, preventing, ameliorating, slowing progression of and/or treating a disease state mediated by one or more complement-related pathways. Such disease states or conditions include, but are not limited to, drusen formation, macular degeneration, AMD, dry eye, corneal ulcers, atherosclerosis, diabetic retinopathy, vitreoretinopathy (Grisanti et al. Invest. Opthalmol. Vis. Sci. 32:2711-2717), corneal inflammation, airway hyperresponsiveness, immune-related diseases, autoimmune-related diseases, lupus nephritis, systemic lupus erythematosus (SLE), arthritis (e.g., rheumatoid arthritis), rheumatologic diseases, anti-phospholipid antibody syndrome, intestinal and renal I/R injury, asthma, atypical hemolytic-uremic syndrome, Type II membranoproliferative glomerulonephritis, non-proliferative glomerulonephritis, fetal loss (e.g., spontaneous fetal loss), glaucoma, uveitis, ocular hypertension, brain injury (e.g., traumatic brain injury), stroke (e.g., see Arumugam et al. PNAS 93(12):5872-6 (1996)), post-traumatic organ damage, post infarction organ damage (e.g., cardiac, neurological), vasculitis, ischemic-reperfusion injury, cerebrovascular accident, Alzheimer's disease, transplant rejection (e.g., xeno and allo), infections, sepsis, septic shock, Sjögren's syndrome, myasthenia gravis, antibody-mediated skin diseases, all antibody-mediated organ-specific diseases (including Type I and Type II diabetes mellitus, thyroiditis, idiopathic thrombocytopenic purpura and hemolytic anemia, and neuropathies), multiple sclerosis, cardiopulmonary bypass injury, polyarteritis nodosa, Henoch Schonlein purpura, serum sickness, Goodpasture's disease, systemic necrotizing vasculitis, post streptococcal glomerulonephritis, idiopathic pulmonary fibrosis (usual interstitial pneumonitis), membranous glomerulonephritis, myocarditis (e.g., autoimmune myocarditis) (Kaya et al. Nat. Immunol. 2001; 2(8):739-45), myocardial infarction, muscular dystrophy (e.g., associated with dystrophin-deficiency), acute shock lung syndrome, adult respiratory distress syndrome, reperfusion, rejection and/or or a complement mediated disease.

The formation of drusen in the eye can be associated with various diseases such as macular degeneration. In some cases, drusen formation and/or its association with a disease has been implicated to be related to complement activity (e.g., see FIG. 1). Some embodiments of the invention provide compositions and methods for modulating, regulating, inhibiting, reducing, retarding and/or reversing the formation or growth of drusen in an animal, such as a human. For example, compositions or molecules of the invention may be delivered to drusen (e.g., by direct injection into drusen (intradrusen injection) or intravitreal injection). Some embodiments of the invention can be utilized to slow the progression of macular degeneration, e.g., via inhibiting drusen formation.

Atherosclerosis has been shown to typically involve complement related pathways, e.g., see Niculescu et al. Immunologic Research, 30(1):73-80(8) (2004) and Niculescu and Horea, immunologic Research 30(1):73-80 (2004). Complement activation and C5b-9 deposition typically occurs both in human and experimental atherosclerosis. C5b-9 may be responsible for cell lysis, and sublytic assembly of C5b-9 induces smooth muscle cell (SMC) and endothelial cell (EC) activation and proliferation. Complement C6 deficiency has a protective effect on diet-induced atherosclerosis, suggesting that C5b-9 assembly is required for, or at least plays a significant role, in the progression of atherosclerotic lesions, e.g., see Niculescu and Horea, Immunologic Research 30(1):73-80 (2004). Some embodiments of the invention may be used to inhibit the formation of C5b-9 and/or inhibit atherosclerosis. This can be done by inhibiting the formation directly or inhibiting a step in a pathway that thereby inhibits the formation and/or activation of C5b-9. In some embodiments, a factor B variant(s) as described herein is administered to a site or potential site of atherosclerosis. This factor B variant(s) inhibits a pathway (e.g., the classical and/or alternative complement pathway) which in turn inhibits the formation or activation of C5b-9 or another complement pathway related compound involved in atherosclerosis. There may be other complement related proteins involved in atherosclerosis whose formation and/or activation may be inhibited or blocked in a similar manner.

Airway hyperresponsiveness (AHR) is characteristic of various diseases including, but not limited to, asthma (e.g., allergic asthma). AHR has been shown to typically involve complement related pathways, e.g., see Taube et al., 2006 PNAS 103(21):8084-8089; Park et al., American Journal of Respiratory and Critical Care Medicine 169:726-732, (2004); Thurman and Holers, J Immunology 176:1305-1310 (2006) and U.S. Patent Publication No. 20050260198. Park et al. showed that Crry-Ig administered by intraperitoneal injection had an effect on AHR. Some embodiments of the invention provide compositions and methods for modulating, regulating, inhibiting, reducing, activating and/or increasing AHR in an animal, such as a human. Specific AHR related diseases that may be treated, alleviated, inhibited and/or ameliorated include, but are not limited to, asthma, chronic obstructive pulmonary disease (COPD), allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma, cough variant asthma, parasitic lung disease, respiratory syncytial virus (RSV) infection, parainfluenza virus (PIV) infection, rhinovirus (RV) infection, Hantaan virus (e.g., four-corners strain) and adenovirus infection

Immune-related diseases such as autoimmune-related diseases, HLA-B27 associated inflammatory diseases, lupus nephritis and systemic lupus erythematosus (SLE) have been shown to typically involve complement related pathways, e.g., see Thurman and Holers, J Immunology 176:1305-1310 (2006). Lupus nephritis is one complication of SLE. It is related to the autoimmune process of lupus, where the immune system produces antibodies (antinuclear antibody and others) against body components. Complexes of these antibodies and complement typically accumulate in the kidneys and result in an inflammatory response. Some embodiments of the invention provide methods and compositions for regulating, modifying, curing, inhibiting, preventing, ameliorating and/or treating an immune-related disease, e.g., involving or related to a complement pathway such as SLE.

Arthritis has been shown to typically involve complement related pathways, e.g., see Thurman and Holers, J Immunology 176:1305-1310 (2006) and Banda et al. J. Immunol. 177(3):1904-12 (2006). The alternative complement pathway plays a significant role in the induction of arthritis and the alternative complement pathway may even be required. Some embodiments of the invention provide methods and compositions for regulating, modifying, curing, inhibiting, preventing, ameliorating and/or treating arthritis, e.g., rheumatoid arthritis or inflammatory arthritis.

Glaucoma is a group of diseases of the optic nerve involving loss of retinal ganglion cells in a characteristic pattern of optic neuropathy. Approximately 25% of glaucoma patients with retinal ganglion cell loss have normal ocular pressure. Ocular hypertension (OHT) is a significant risk factor for developing glaucoma and lowering it via pharmaceuticals or surgery is currently the mainstay of glaucoma treatment. Ocular hypertension and glaucoma have been shown to typically involve complement related pathways, e.g., see Khalyfa et al., Molecular Vision, 13:293-308 (2007); Stasi et al. IOVS 47(3):1024-1029 (2007); and Kuehn et al., Experimental Eye Research 83:620-628 (2006). Expression and/or the presence of C1q and C3 have been shown to be higher in retinae subjected to OHT. Some embodiments of the invention provide methods and compositions for regulating, modifying, curing, inhibiting, preventing, ameliorating and/or treating glaucoma.

Uveitis has been shown to typically be associated with the complement pathway, e.g., see Mondino and Rao, Investigative Ophthalmology & Visual Science 24:380-384 (1983) and Jha et al. Molecular Immunology 44:3901-3908 (2007). Mondino and Rao found that mean values of all tested complement components in aqueous humor to serum measurements were increased in patients with a history of previous eye surgeries and were highest in patients with anterior uveitis. Some embodiments of the invention provide methods and compositions for regulating, modifying, curing, inhibiting, preventing, ameliorating and/or treating uveitis.

Diabetic retinopathy is one of the leading causes of vision loss in middle-aged individuals. Activation of the complement system is believed to play an important role in the pathogenesis of diabetic retinopathy (e.g., see Jha et al. Molecular Immunology 44:3901-3908 (2007)). Some embodiments of the invention provide methods and compositions for regulating, modifying, curing, inhibiting, preventing, ameliorating and/or treating diabetic retinopathy.

Proliferative vitreoretinopathy (PV) is one of the most common complications of retinal detachment. PV has been linked to complement activity, e.g., see Grisante et al. Invest Opthalmol V is Sci. 1991; 32(10):2711-7 and Grisante et al. Ophthalmologe. 1992; 89(1):50-4. Some embodiments of the invention provide methods and compositions for regulating, modifying, curing, inhibiting, preventing, ameliorating and/or treating PV.

Anti-phospholipid antibody syndrome, intestinal and renal ischemic reperfusion I/R injury, atypical hemolytic-uremic syndrome, Type II membranoproliferative glomerulonephritis, and fetal loss (e.g., spontaneous fetal loss), have been shown to typically involve complement related pathways, e.g., see Thurman and Holers, J Immunology 176:1305-1310 (2006).

Brain injury (e.g., traumatic brain injury) has been shown to typically involve complement related pathways, e.g., see Leinhase et al., J Neuroinflammation 4:13 (2007) and BMC Neurosci. 7:55 (2006). Leinhase 2006, showed that after experimental traumatic brain injury in wild-type (fB+/+) mice, there was a time-dependent systemic complement activation. In contrast, the extent of systemic complement activation was significantly attenuated in fB−/− mice. Some embodiments of the invention provide methods and compositions for regulating, modifying, curing, inhibiting, preventing, ameliorating and/or treating neuronal cell death, traumatic neural injury (e.g. brain), complement-mediated neuroinflammation and/or neuropathology.

Ischemia-reperfusion injury can cause increases in the production of or oxidation of various potentially harmful compounds produced by cells and tissues, which can lead to oxidative damage to or death of cells and tissues. For example, renal ischemia-reperfusion injury can result in histological damage to the kidneys, including kidney tubular damage and changes characteristic of acute tubular necrosis. The resultant renal dysfunction permits the accumulation of nitrogenous wastes ordinarily excreted by the kidney, such as serum urea nitrogen (SUN). Ischemia-reperfusion may also cause injury to remote organs, such as the lung. Some embodiments of the invention utilize modulators, such as inhibitors, of a complement pathway (e.g., inhibitors of factor B activity), e.g., when administered to an animal that has, or is at risk of experiencing or developing, ischemia-reperfusion. In some embodiments, these modulators, prevent, reduce or inhibit at least one symptom of injury due to ischemia-reperfusion. Other types of ischemia-reperfusion injury, that can be prevented or reduced using methods and compositions of the invention, include, but are not limited to, cardiac ischemia-reperfusion injury such as myocardial infarction or coronary bypass surgery, central nervous system ischemia-reperfusion injury, ischemia-reperfusion injury of the limbs or digits, ischemia-reperfusion of internal organs such as the lung, liver or intestine, or ischemia-reperfusion injury of any transplanted organ or tissue. See, e.g., PCT Publication No. WO03/061765 which discusses myocardial infarction and complement pathways.

Inflammation is a major etiologic determinant of myocardial infarction (Ridker, 2007 Nutr. Rev. 65(12 Pt 2):S253-9). It has also been shown that delivery (e.g., intracoronary) of bone marrow (stem) cells leads to an improvement in systolic function after acute myocardial infarction (Wollert, 2008, Curr. Opin. Pharmacol. January 31 [Epub]). Also, bone marrow stem cells can regenerate infarcted myocardium (Orlic et al. 2003 Pediatr. Translpant. 7 Suppl 3:86-88). Mesenchymal stem cells have been shown to provide a cardiac protective effect in ischemic heart disease (Guo et al. 2007 Inflammation 30(3-4):97-104). In the present invention, delivery of the stem cells can be by any means, such as intracoronary injection, injection directly into myocardium (e.g., into diseased and/or healthy myocardium (e.g., adjacent to the injured area)). In some embodiments, a mammal is treated with cytokines to mobilize their bone marrow stem cells in the circulation allowing the stem cells to traffic to the myocardial infarct.

Various stem cells have been used in vivo for various applications. One issue with the use of stem cells in vivo is the lower than desired survival and/or seeding of the stem cells, e.g., in the area of interest. The inventors believe that one significant reason for low seeding and survival of stems cells can be inflammation at the site. Therefore, the present invention provides a method of treatment and/or a method of improving stem cell survival and/or seeding, the methods comprising administering a composition of the invention before, during and/or after administration or mobilization of stem cells. In some embodiments, complement inhibitors of the invention act as anti-inflammatory agents that will create a favorable environment for stem cells to home in and survive in the area of desired seeding (e.g., damaged heart) and therefore repair or replace the damaged tissue. Stem cells may be administered in a solution that also contains a molecule of the present invention, such as a fB analog or a fD analog. Stem cells may be, but are not limited to, hematopoietic stem cells, embryonic stem cells, mesenchymal stem cells, neural stem cells, mammary stem cells, olfactory stem cells, pancreatic islet stem cells, totipotent stem cells, multipotent stem cells or pluripotent stem cells. The stem cells may be autologous, allogeneic, or syngeneic.

It appears that complement activity is involved in muscular dystrophy (e.g., associated with dystrophin-deficiency). For example, see PCT Publication No. WO2007130031, Spuler & Engel 1998 Neurology 50:41-46, and Selcen et al. 2001 Neurology 56:1472-1481. Therefore, some embodiments of the invention provide methods and compositions for regulating, modifying, curing, inhibiting, preventing, ameliorating and/or treating muscular dystrophy.

Complement activity may contribute to corneal inflammation. Therefore, some embodiments of the invention provide methods and compositions for regulating, modifying, curing, inhibiting, preventing, ameliorating and/or treating corneal inflammation, e.g., after surgery. In some embodiments, a molecule of the invention is administered via eye drops or as otherwise described herein.

Some embodiments of the invention provide methods for enhancing the efficacy of post-coronary or peripheral artery bypass grafting or angioplasty. In some embodiments, a vector of the invention encoding a protein of the invention (e.g., fB1 and/or fB3) is used to transduce cells of a blood vessel (e.g., endothelial cells). In some embodiments, cells of a blood vessel are transduced prior to implantation in an animal. In some embodiments, cells of a blood vessel are in vivo.

Alleviating pain and suffering and inflammation in postoperative patients is an area of special focus in clinical medicine, especially with the growing number of out-patient operations performed each year. Compositions of the present invention can be utilized to inhibit inflammation, e.g., by inhibiting a complement activity. Therefore, compositions of the invention can be used to reduce inflammation, e.g., in postoperative patients. In some embodiments, a composition of the invention is delivered locally (e.g., perioperative delivery) to a site of surgery to inhibit inflammation, which in some cases will reduce pain and suffering. In some embodiments, the composition of the invention is administered in a solution, e.g., in a physiologic electrolyte carrier fluid. In some embodiments, the composition is delivered via perioperative delivery directly to a surgical site of an irrigation solution containing the composition. In some embodiments, due to the local perioperative delivery method of the present invention, a desired therapeutic effect may be achieved with lower doses of agents than are necessary when employing other methods of delivery, such as intravenous, intramuscular, subcutaneous and oral. In some embodiments, when used perioperatively, the solution will result in a clinically significant decrease in operative site pain and/or inflammation, thereby allowing a decrease in the patient's postoperative analgesic (e.g., opiate) requirement and, where appropriate, allowing earlier patient mobilization of the operative site. In some embodiments, no extra effort on the part of the surgeon and operating room personnel is required to use the present solution relative to conventional irrigation fluids. In some embodiments, a composition of the invention is used (e.g., in irrigation fluid) for arthroscopy, cardiovascular and general vascular therapeutic and diagnostic procedures, urologic procedures, general surgical wounds and wounds in general. Compositions of the invention may be delivered by, but not limited to, injection (e.g., via syringe), via irrigation fluid, as part of a bandage over a wound, or in a topical application such as a solution, cream, gel or the like.

In another embodiment, a composition or molecule of the invention is administered in combination with LUCENTIS® or a molecule(s) that binds VEGF or that inhibits angiogenesis. LUCENTIS® is used to treat wet AMD. Some embodiments of the invention can also be used to treat wet AMD. Therefore, the present invention provides methods and compositions for treating wet AMD comprising administering LUCENTIS® and a composition of the invention, wherein they can be administered separately or together. Additionally, intraocular inflammation is one of the most common adverse reactions reported after administration of LUCENTIS®, e.g., see the “Full Prescribing Information” for LUCENTIS®. Therefore, the present invention provides a method for inhibiting or reducing intraocular inflammation (e.g., resulting from the administration of LUCENTIS®) comprising administering a molecule or composition of the invention prior to, at the same time, and/or after the administration of LUCENTIS®.

Complement pathways contributing to and/or causing a disease can be modulated, regulated, inhibited and/or activated using various methods and/or compositions that are part of the present invention. Some embodiments of the invention utilize a protein(s) to modulate a pathway.

Administration to an Animal and/or a Cell

Compositions and methods are provided herein relating to regulating a complement related pathway. This can be done in vitro, ex vivo, or in vivo. Administration to an animal (e.g., to a site of interest) can be accomplished any number of ways, e.g., as described herein or known in the art. In some embodiments, a protein(s) is administered “indirectly” through the administration of a nucleic acid(s) that encodes the protein(s). In some embodiments, a protein itself is administered to an animal. One of skill in the art is aware of delivery methods that are compatible with delivering a composition(s) of the invention to a desired site in an animal.

In some embodiments, compositions (e.g., comprising an analog(s)) can be administered locally or systemically. Useful routes of administration are described herein and known in the art. Methods of introduction or administration include, but are not limited to, intradermal, intramuscular, intraperitoneal, intravenous, subcutaneous, intranasal, intratracheal, topical, inhaled, transdermal, rectal, parenteral routes, epidural, intracranial, into the brain, intraventricular, subdural, intraarticular, intrathecal, intracardiac, intracoronary, intravitreal, subretinal, intraanterior chamber of the eye, particular, locally on the cornea, subconjunctival, subtenon injection, by applying eyedrops, oral routes, via balloon catheter, via stent or any combinations thereof. In some embodiments, a composition or molecule of the invention is administered to a drusen, e.g., by injecting directly into a drusen. Systemic administration may be, but is not limited to, by injection or by transmucosal and/or transdermal delivery. In some embodiments, a composition of the invention may be initially directed to a site other that a site of, for example, disease. For example regarding AHR which occurs in the lungs of an animal, an intraperitoneal injection of a protein or nucleic acid of the invention may result in a change in AHR in the lungs, e.g., see Park et al., American Journal of Respiratory and Critical Care Medicine 169:726-732, (2004). In some embodiments, a dosage level and/or mode of administration of a composition may depend on the nature of the composition, the nature of a condition(s) to be treated, and/or a history of an individual patient. In some embodiments, cells expressing a protein of the invention are administered. These cells can be a cell line, xenogeneic, allogeneic or autologous.

In some embodiments, e.g., comprising administration to the eye, the molecule or vector of the invention is administered about once every week, month, 2 months, 3 months, 6 months, 9 months, year, 18 months, 2 years, 30 months, 3 years, 5 years, 10 years or as needed. In some embodiments, e.g., comprising administration to the eye, the molecule or vector of the invention is administered from about every 1 to 4 weeks, about every 4 to 8 weeks, about every 1 to 4 months, about every 3 to 6 months, about every 4 to 8 months, about every 6 to 12 months, about every 9 to 15 months, about every 12 to 18 months, about every 15 to 21 months, about every 18 to 24 months, about every 1 to 2 years, about every 1.5 to 3 years, about every 2 to 4 years, about every 3 to 5 years, about every 5 to 7 years, about every 7 to 10 years or about every 10 to 20 years.

In some embodiments, e.g., comprising administration to the eye, the molecule or vector of the invention is administered 1, 2, 3, 4, 5, 6, 7, 8, 9, 10 or more times to a patient in their lifetime. In some embodiments, e.g., comprising administration to the eye, a lentiviral vector of the invention is administered 1, 2, 3, 4, 5, 6, 7, 8, 9, 10 or more times to a patient in their lifetime.

In some embodiments, an anti-inflammatory may be delivered in combination with a molecule or vector (e.g., fB3) of the invention. An anti-inflammatory may be delivered prior to, concurrently with, and/or after administration of a molecule or vector of the invention. In some embodiments, an anti-inflammatory is administered in the same solution and/or same syringe as a molecule or vector of the invention. In some embodiments, a molecule or vector of the invention and an anti-inflammatory are co-administered to the eye, e.g., as described herein.

Many anti-inflammatory drugs are known in the art and include, but are not limited to, dexamethasone, dexamethasone sodium metasulfobenzoate, dexamethasone sodium phosphate, fluorometholone, bromfenac, pranoprofen, RESTASIS™, a cyclosporine ophthalmic emulsion, naproxen, glucocorticoids, ketorolac, ibuprofen, tolmetin, non-steroidal anti-inflammatory drugs, steroidal anti-inflammatory drugs, diclofenac, flurbiprofen, indomethacin, and suprofen.

Some embodiments of the invention include proteins that inhibit complement activity and/or vectors that code for a protein that inhibits complement activity. Some embodiments of the invention include administration of both a protein and a vector encoding it or encoding another protein of the invention. A protein of the invention may be delivered prior to, concurrently with, and/or after administration of a vector of the invention. In some embodiments, a protein of the invention is administered in the same solution and/or same syringe as a vector of the invention. In some embodiments, a protein of the invention and a vector of the invention are co-administered to the eye, e.g., as described herein.

### Nucleic Acids

To ensure local and long term expression of a nucleic acid of interest, some embodiments of the instant invention contemplate the transformation of a cell using a nucleic acid or vector. The instant invention is not to be construed as limited to any one particular nucleic delivery method, and any available nucleic acid delivery vehicle with either an in vivo or in vitro nucleic acid delivery strategy, or the use of manipulated cells (such as the technology of Neurotech, Lincoln, R I, e.g., see U.S. Pat. Nos. 6,231,879; 6,262,034; 6,264,941; 6,303,136; 6,322,804; 6,436,427; 6,878,544) as well as nucleic acids encoding a therapeutic protein per se (e.g., “naked DNA”), can be used in the practice of the instant invention. Various delivery vehicles, such as vectors, can be use with the present invention. For example, lentiviruses, adenoviral vectors (see, for example, U.S. Pat. No. 7,045,344), AAV vectors (see, for example, U.S. Pat. No. 7,105,345), plasmids (see, for example, U.S. Pat. No. 6,936,465), other viral vectors (for example, Herpes, U.S. Pat. Nos. 5,830,727 and 6,040,172; Hepatitis D, U.S. Pat. No. 5,225,347; and EBV, U.S. Pat. No. 6,521,449), amphitrophic lipids, cationic polymers, such as polyethyleneimine (PEI) and polylysine, dendrimers, such as combburst molecules and starburst molecules, nonionic lipids, anionic lipids, vesicles, liposomes and other synthetic nucleic acid means of gene delivery (see, for example, U.S. Pat. Nos. 6,958,325 and 7,098,030; and Langer, Science 249:1527-1533 (1990); Treat et al., in “Liposomes” in “The Therapy of Infectious Disease and Cancer”; and Lopez-Berestein & Fidler (eds.), Liss, New York, pp. 317-327 and 353-365 (1989); “naked” nucleic acids and so on can be used in the practice of the instant invention. Solely for the purpose of exemplification, some discussions herein will focus on some particular vector types, including lentiviral vectors such as those developed and obtained from bovine immunodeficiency virus (BIV), a lentivirus distantly related to HIV.

A vector is a means by which a nucleic acid of interest (e.g., a therapeutic nucleic acid, e.g., that can encode a therapeutic protein) is introduced into a target cell of interest. A vector is typically constructed or obtained from a starting material, such as a nucleic acid capable of carrying a foreign gene or transgene and which is capable of entering into and being expressed in a target cell. Suitable starting materials from which a vector can be obtained include transposons, plasmids, viruses, PCR products, cDNAs, mRNAs and so on, as known in the art. Methods for obtaining or constructing a vector of interest include, but are not limited to, standard gene manipulation techniques, sequencing reactions, restriction enzymes, polymerase, PCR, PCR soeing, ligations, recombinase reactions (e.g., Invitrogen's GATEWAY® technology) other enzymes active on nucleic acids, bacteria and virus propagation materials and methods, chemicals and reagents, site directed mutagenesis protocols and so on, as known in the art, see, for example, the Maniatis et al. text, “Molecular Cloning.”

A wide variety of nucleotide sequences generally referred to as transgenes may be carried by a vector of the present invention, in addition to an optional marker gene, which may be used, for example, as a selection means or for enhancing expression. In some embodiments, a foreign nucleotide sequence should be of sufficient size to allow production of viable virus particles. For example, certain virus particles will only package nucleic acids of a particular size range. A non-exhaustive list of these transgenes (heterologous genes) includes sequences which encode proteins, such as single chain antibodies, antibodies and various antigen-binding forms thereof, ribozymes, inhibitory RNA molecules such as siRNA, catalytic antibodies, as well as antisense sequences, for example.

A protein may be a therapeutic protein or a protein that impacts a therapeutic protein. Further, a protein may be an entire protein or a functionally active fragment thereof. The protein may be, for example, one that participates in or regulates inflammation, e.g., wherein the protein is a therapeutic protein or may be a protein that regulates inflammation by acting on a complement factor, for example, or acting on a gene expressing a complement factor or a regulator thereof, or acting on another gene capable of regulating elements responsible for inflammation.

In some embodiments, expressed sequences will be operably linked to a promoter. In some embodiments, a transgene insert will be operably linked to a second promoter.

With respect to constructs as disclosed herein, the choice of promoter is well within the skill of one in the art and extends to any eukaryotic, prokaryotic or viral promoter capable of directing gene transcription in a target or host cell transformed with a construct using a first promoter (e.g., functional in the producer cell) or construct using a second promoter (e.g., functional in the ultimate target cell) according to the invention. A promoter may be a tissue specific promoter, a cell specific promoter, an inducible promoter, a repressible promoter, a synthetic promoter or a hybrid promoter, for example. More than one promoter may be placed in a construct of the invention. Examples of promoters useful in the constructs of the invention include, but are not limited to, a phage lambda (PL) promoter; an SV40 early promoter; a herpes simplex viral (HSV) promoter; a cytomegalovirus (CMV) promoter, such as the human CMV immediate early promoter; a tetracycline-controlled trans-activator-responsive promoter (tet) system; a long terminal repeat (LTR) promoter, such as a MoMLV LTR, BIV LTR or an HIV LTR; a U3 region promoter of Moloney murine sarcoma virus; a Granzyme A promoter; a regulatory sequence(s) of the metallothionein gene; a CD34 promoter; a CD8 promoter; a thymidine kinase (TK) promoter; a B19 parvovirus promoter; a PGK promoter; a glucocorticoid promoter; a heat shock protein (HSP) promoter, such as HSP65 and HSP70 promoters; an immunoglobulin promoter; an MMTV promoter; a Rous sarcoma virus (RSV) promoter; a lac promoter; a CaMV 35S promoter; and a nopaline synthetase promoter. Numerous promoters are available from commercial sources, such as, Stratagene (La Jolla, Calif.) and Invitrogen (Carlsbad, Calif.).

In some embodiments, promoters include the promoter region of LTRs, such as a 5′ LTR promoter of HIV or BIV. In some embodiments, promoters include CMV promoters and PGK promoters. In some embodiments, a promoter is an MND promoter (Robbins et al., 1997, J. Virol. 71:9466-9474), or an MNC promoter, which is a derivative of the MND promoter in which the LTR enhancers are combined with a minimal CMV promoter (Haberman et al., J. Virol. 74(18):8732-8739, 2000).

Heterologous introns are known and non-limiting examples include a human β-globin gene intron. In some embodiments, a vector of the invention comprises an intron, e.g., as part of the gene coding for a protein or transgene of interest. In some embodiments, introns used in some constructs of the invention may be obtained from an SV40 virus or a human insulin gene. In some embodiments, in retroviral constructs, an intron will be located upstream of gag and/or pol coding region(s).

Signal sequences or leader sequences are known and can be used in expression constructs, e.g., to express proteins of the invention such as an fB analog or fD analog. Signal sequences are translated in frame as a peptide attached to the amino-terminal end of a polypeptide of choice, the secretory signal sequence will cause the secretion of the polypeptide by interacting with the machinery of the host cell. As part of the secretory process, this secretory signal sequence will be cleaved off. The human placental alkaline phosphatase secretory signal sequence is an example of a signal sequence. The present invention is not limited by specific secretory signal sequences and others are known to those skilled in the art. The term “signal sequence” also refers to a nucleic acid sequence encoding the secretory peptide. If a signal sequence is included, it can either be the native sequence, homologous sequence, or a heterologous sequence.

Expression of a viral gene or a transgene typically involves an adequate promoter being operably linked to the coding nucleic acid sequence. The terms “operably linked” or “operatively linked” are interchangeable and refer to an arrangement of elements in a construct wherein the components are configured so as to perform their usual, expected or stated function. A promoter or other control elements need not be contiguous with the coding sequence. For example, there may be intervening residues between a promoter or control elements and the coding region so long as the functional relationship is maintained.

As disclosed herein, one or more constructs according to the invention may further include a polyadenylation signal (polyA) that is positioned 3′ of a coding sequence. A polyA tail may be of any size which is sufficient to promote stability, e.g., in the cytoplasm. A polyA signal may be derived from a lentivirus such as BIV, HIV and SIV. However, a polyA sequence may be derived from other cells or viruses as well, such as from SV40. A number of polyA sites are known and can be used as a design choice, or a synthetic polyA can be used, as known in the art.

In some embodiments, a therapeutic gene may be one that expresses a complement factor or regulator thereof, or that antagonizes production or function of an element that contributes to inflammation, such as a ribozyme, catalytic antibody, inhibitory RNA, antisense molecule and so on.

### Viral Vectors

The present invention is not limited to a particular viral vector. Viral vectors include, but are not limited to, retroviral vectors, lentiviral vectors, adenoviral vectors (see, for example, U.S. Pat. No. 7,045,344), AAV vectors (see, for example, U.S. Pat. No. 7,105,345), Herpes viral vectors (e.g., U.S. Pat. Nos. 5,830,727 and 6,040,172), Hepatitis D viral vectors (e.g., U.S. Pat. No. 5,225,347), SV40 vectors and EBV vectors (e.g., U.S. Pat. No. 6,521,449).

Virions of the invention (e.g., BIV-based vectors) may be administered in vivo or in vitro to cells (e.g., mammalian cells). Vectors (viral and nonviral) can be used to transduce or transform cells including, but not limited to, undifferentiated cells, differentiated cells, somatic cells, primitive cells and/or stem cells. In some embodiments, stem cells are intended for administration to a human and not for implantation in a suitably pseudopregnant woman for differentiation and development into an infant.

In some embodiments, a virion encodes a heterologous (as compared to the virus and/or to the target cell) coding region or transgene. In some embodiments, the heterologous coding region encodes a therapeutic product. Some virions produced according to the invention are, but are not limited to, recombinant particles, recombinant virus particles, recombinant BIV particles, recombinant vector particles or virions. A “recombinant particle or virion” refers to a virus particle that contains a viral based vector nucleic acid. In some instances, a vector construct may be contained in a particle derived from viruses (e.g., other than BIV), for example, retroviruses or lentiviruses, such as FIV, HIV, SIV, BIV, and EIAV, of which type strains are publicly available to serve as starting materials from which vectors of interest can be obtained.

Generally, the concentration of some viral particles of the invention can be increased by, first, collecting the virus particles via centrifugation, (e.g., of the virus-containing medium), and then removing the supernatant. In some embodiments, most or essentially all of the supernatant is removed and the virus particles are suspended in a smaller volume. Some embodiments, comprise concentrating the virus 10-fold by suspending the collected virus in a volume of liquid 10-fold smaller than the original volume before centrifugation. In some cases, this can result in 3-fold to 10-fold increase in transduction efficiency. As the person skilled in the art will readily appreciate, a further concentration of the virus can result in even higher increases in transduction efficiency. Other forms of concentration that can be used alone or in combination with others described herein include, but are not limited to, tangential flow purification, diafiltration, ion exchange chromatography, affinity chromatography, size exclusion chromatography, immunoaffinity chromatography, reverse phase chromatography, heparin sepharose affinity chromatography and other known forms of separation and concentration. In some embodiments, viral particles are concentrated from about 1.5 to about 1000 fold; about 2 to about 90 fold; about 2 to about 80 fold; about 2 to about 70 fold; about 2 to about 60 fold; about 2 to about 50 fold; about 2 to about 40 fold; about 2 to about 30 fold; about 2 to about 20 fold; about 2 to about 15 fold; about 2 to about 10 fold; about 2 to about 7 fold; about 2 to about 5 fold; about 5 to about 500 fold; about 10 to about 500 fold; about 50 to about 500 fold; about 100 to about 500 fold; about 200 to about 500 fold; about 300 to about 500 fold; about 400 to about 500 fold; about 450 to about 500 fold; about 500 to about 1000 fold; about 600 to about 1000 fold; about 700 to about 1000 fold; about 800 to about 1000 fold; about 900 to about 1000 fold; about 10 to about 200 fold; about 50 to about 300 fold; about 50 to about 150 fold; about 50 to about 100 fold; about 100 to about 200 fold; about 100 to about 300 fold; or about 300 to about 500 fold.

BIV vectors are presented herein as an exemplary vector type and as an example of nucleic acid delivery methods/compositions and as an example of a retroviral or lentiviral vector that can be utilized in the present invention. However, the present invention is not limited to BIV vectors or even lentiviral or retroviral vectors. BIV vectors and the systems that produce them are but some examples of vectors that can be used in accordance with the current invention. Other vectors can be used with the present invention. For features described herein for BIV vectors, corresponding features can be designed into other vectors such as other lentiviruses and HIV viruses and in some cases other retroviruses. Therefore, BIV is provided as an exemplary embodiment.

BIV is not known to cause human disease. Nevertheless, BIV vectors do transduce a variety of human cells, including cells of the eye, such as RPE cells. In some embodiments of the invention, a vector contains only the minimal of BIV elements required for transfer of the therapeutic gene (Molina et al., 2004). Essentially all of the viral genes, accounting for more than 90% of the BIV genome, can be removed in such vectors. Features of some BIV vector systems include, but are not limited to, high titers, e.g., at least 10⁶ and up to 3×10⁹ or more transducing units/ml; efficient gene transfer in vitro in a broad spectrum of human cells; efficient gene transfer to retinal cells in vivo; extensive safety features that in some embodiments equal or exceed those of other lentiviral vector systems; and technology for scale-up and manufacturing. Examples of BIV systems are described, for example, in Matukonis et al., 2002; Molina et al., 2002; 2004; U.S. Pat. Nos. 6,864,085, 7,125,712 and 7,153,512.

A number of different combinations of DNA constructs can be used to obtain BIV particles carrying a viral-based genome housing a therapeutic gene of interest. In one system, four DNA components are used for BIV vector production. These include the expression construct encoding a BIV vector sequence; an expression construct encoding a BIV rev sequence; an expression construct encoding a BIV gag/pol sequence; and an expression construct encoding an envelope. In some embodiments, an envelope coding region is not derived from BIV. An expression construct encoding a vector sequence may generate RNA carrying a desired transgene that is packaged into vector/viral particles. Expression constructs for gag/pol and envelope produce the BIV capsid proteins that form the vector particle. An expression construct for rev produces a protein which, inter alia, assists with transport of vector RNA out of the cell nucleus. Rev can be placed on the gag/pol and/or env constructs. In some embodiments, rev is not part of a gag, pol, or env expression construct. In some embodiments, a cell line(s) carrying one or more BIV genes integrated into the host cell line genome can be used, e.g., to minimize the number of transformation events at the time the BIV vector is introduced. In some embodiments, a cell line which incorporates all of the components necessary to generate the vector can be used to eliminate the need for transformation at the time vector is prepared. Some methods of the invention for generating vector particles involve co-transformation of the expression constructs into cells in tissue culture. After packaging of the vector RNA and assembly of the particles in the cytoplasm, vector particles bud through the cell membrane, acquiring a lipid bilayer coat, and accumulate in the tissue culture medium from which they may optionally be purified and/or optionally concentrated. Methods of collecting virions produced by transformed cells are described, for example, in Rigg et al., Virology 218:290-295 (1996).

In some embodiments, for example regarding lentiviral or retroviral vectors, a portion of a gag gene may be incorporated into a DNA segment from a viral genome. As an example a BIV gag coding sequence is typically approximately 1431 nucleotides. In some embodiments, a portion of a gag coding region used in a vector of interest will include no more than about 102 nucleotides of the gag coding region. In some embodiments, a portion of a gag coding region used in a vector of interest will include between from about 76 to about 500, about 76 to about 200, about 76 to about 102, about 76 to about 100, about 76 to about 95, about 76 to about 90, about 76 to about 85, about 76 to about 80, about 80 to about 90, or about 90 to about 100 nucleotides of the gag coding region. In some embodiments, this RNA sequence can enhance packaging of the vector RNA into the vector particles. In some embodiments, a DNA segment may comprise a gene or coding region for a protein selected from the group consisting of vif, vpw, vpy, tat, vpu, vpr, nef, tmx, or rev such as from BIV, HIV or another lentivirus may be used to enhance gene transfer.

A BIV vector construct may further comprise one or more regulatory elements such as an RNA transport element (e.g., a rev response element (RRE)), a constitutive transport element (CTE), such as a Mason-Pfizer Monkey Virus CTE or an Avian Leukemia Virus CTE, sequences that enhance translation, signal sequences, copy number control elements, integration compatible sequences, termination sequences, mRNA leader sequences, a scaffold attachment region (SAR), e.g., of human origin, a polypurine tract, generally upstream of the 3′ LTR, a posttranscriptional regulatory element, such as that of a woodchuck hepatitis virus (WPRE; Zufferey et al., Virol. 73(4):2886-92 (1999)), an internal ribosome entry site (IRES), a ribosome binding site (RBS), enhancers and other regulatory sequences as known in the art. In some embodiments, a transgene (e.g., operably linked to a second promoter) is located downstream of a putative BIV RRE. An RRE may be from a lentivirus other than BIV. As with most genetic elements included in a construct of interest, said elements are configured and joined in a fashion that results in operable expression products, that is, the elements are operably linked. The elements can be synthesized, purchased, and/or subcloned from other nucleic acids or from natural sources and so on.

In some embodiments, a retroviral or lentiviral vector construct of the invention is a self-inactivating vector. For example, when an LTR is present, a portion of the U3 region of the 3′ LTR of the BIV vector construct may be deleted or replaced by a heterologous sequence. In such a situation, a transgene may be operably linked to an internal promoter. In some embodiments, the U3 element may further contain a sequence that enhances polyadenylation. For example, a portion of the U3 region of the 3′ LTR can be replaced with the SV40 late polyadenylation signal enhancer element (e.g., see Sehet et al., Mol. Cell Biol., 12:5386-5393 (1992)).

A “packaging construct,” also sometimes referred to as a helper construct, refers to an assembly which is capable of directing expression of at least a gag and/or pol coding region and a promoter operably linked thereto and optionally, a polyadenylation sequence located downstream of the nucleotide sequence encoding the gag and/or pol. The polyadenylation sequence can be, for example, derived from Simian virus 40 (SV40) or a bovine growth hormone gene. Numerous polyadenylation signals are known in the art.

A packaging construct may include other coding regions in addition to the specific genes mentioned above. Other genes include vif, vpw, vpy, tat and rev genes. In some embodiments, a rev gene can be obtained from BIV or from a different lentivirus, such as, from HIV. Constructs can also include a sufficient number of nucleotides corresponding to a functional tat gene.

In some embodiments, a splice site, such as the major splice donor site, may be inactivated or eliminated to reduce or eliminate aberrant splicing. Sequence changes, such as to the packaging sequence and/or the splice donor site, may be accomplished by standard techniques as known in the art.

Some embodiments of the invention also provide a minimal retroviral or lentiviral packaging construct. In some embodiments, a construct comprises a promoter operatively linked to a gag/pol coding sequence and a polyadenylation signal at the 3′ end of the gag/pol coding sequence. In some embodiments, the packaging construct comprises a heterologous intron upstream (i.e., 5′) of the gag/pol coding sequence. In addition, a packaging construct may contain an RNA transport element. In some embodiments, this element may be a lentiviral RRE, a BIV RRE, or it may be a CTE as described herein. In some embodiments, a packaging construct may also contain a Rev coding sequence. In some embodiments, a gag/pol coding region can be altered without changing the amino acid sequence, but eliminates the need for an RRE, e.g., by recoding a gag/pol coding region, such as with optimized codons.

In some embodiments, a virus cell surface protein expression construct of the invention, an expression construct carrying an envelope coding sequence, includes a VSV-G env coding region. In some embodiments, VSV-G protein is a desirable env gene because VSV-G confers broad host range on a recombinant virus. However, in some cases a VSV-G env can have deleterious effects on a host cell. Thus in some embodiments, when a coding region such as that for VSV-G env is used, a controlled gene expression mechanism is employed, such as an inducible promoter system, so that VSV-G expression can be regulated to minimize host cell toxicity when VSV-G expression is not required. For example, the tetracycline-regulatable gene expression system of Gossen & Bujard (Proc. Natl. Acad. Sci. (1992) 89:5547-5551) can be employed to provide for inducible expression of VSV-G. In some embodiments, a tet/VP16 transactivator may be present on a first vector and the VSV-G coding sequence may be cloned downstream from a promoter controlled by tet operator sequences on another vector. Other non-limiting examples of regulatable expression systems are described in PCT Publications WO 01/30843 and WO 02/06463.

In some embodiments, an envelope-encoding construct of the invention includes an LCMV mutant env coding region (Beyer, et al., J. Virol., 76:1488-1495, 2002), a Thogoto envelope (e.g., see PCT Publication No. WO03066810) and/or a baculovirus gp64 env coding region (Monsma et al., J. Virol. 70(7):4607-4616, 1996). In one embodiment, an LCMV mutant env and/or the gp64 env coding region is constitutively expressed. In another embodiment, an LCMV mutant env and/or a gp64 env coding region is expressed from an inducible promoter. Inducible promoter systems and constitutive promoter systems are known in the art (see e.g., WO03066810) and some are also described herein. Other envelopes can be used in the practice of the invention and include, but are not limited to, a 4070A env, a murine leukemia viruses (MuLV) env, a Moloney murine leukemia virus env, an amphotropic env, a xenotropic env, an ecotropic env, a polytropic env, a GP120 env from an HIV, a HTLV I env, HTLV II env, hepatitis B virus (HBV) env, influenza env such as HA, a Lyssavirus glycoprotein (GP), an alphavirus GP, a Ross River virus GP, a Semliki Forest virus GP, a Sindbis GP, a Filovirus GP, an Ebola virus (e.g., Zaire strain) env, a Marburg virus env, a gammoretrovirus GP, and an EBV env, also see, e.g., Reiser, Gene Therapy 7:910-913 (2000) and Cronin et al., Curr Gene Ther 5(4):387-398 (2005). These envelopes are not limited to the use in BIV or lentiviral vectors, but can be used with any appropriate or compatible enveloped virus or enveloped vector.

In some embodiments, a viral vector of the invention comprises a decay accelerating factor (DAF). For example, an enveloped viral vector includes a DAF on the viral membrane. In some embodiments, a DAF is a wild-type DAF. In some embodiments, a DAF is part of a fusion protein with an envelope protein, e.g., see Guibinga et al. Mol Ther. 2005 11(4):645-51. The invention also includes a BIV producer cell that expressed a DAF.

Some vector constructs (e.g., BIV constructs) according to the invention may be used to transform virtually any cell line or host cell, which will serve as the packaging cell or the producer cell. Such cells can be prokaryotic or eukaryotic host cells. The cells can be bacterial, yeast, insect and so on. Transformation generally is by transfection or transduction. Transfection is the transformation of target or host cells with isolated DNA genome, such as a plasmid, see, for example, Kriegler, M., Gene Transfer and Expression: A Laboratory Manual, W.H. Freeman & Company NY (1990). Reference is made to commercially available kits, such as CalPhos kit, (Clontech Inc. Palo Alto, Calif.) and Profection kit, (Promega, Madison Wis.). Also, see Kotani et al., Human Gene Ther. 5:19-28 (1994) and Forstell et al., J. Virol. Methods 60:171-178 (1996) for examples of methods of spinoculation. In some embodiments, cells are mammalian cells, such as primate cells or human cells. Examples include, but are not limited to, human embryonic kidney cells (e.g., 293 or 293T), EREp rabbit cells, Cf2Th (ATCC No. CRL 1430), CHO, SW480, CV-1, the human T cell line CEM-SS, Jurkat, the MDCK and D17 dog cell line, HT1090, LINA, WES and a murine cell line such as NIH3T3. A cell line or cell culture denotes eukaryotic cells grown or maintained in vitro. It is understood that descendants of a cell may not be completely identical (e.g., morphologically, genotypically or phenotypically) to the parent cell.

Because a packaging cell and/or producer cell may be human, and target cells can be human, various elements of a vector construct of interest may be selected to provide optimized expression in the specific host and target cells, e.g., human derived promoters, recoding of the coding region with human optimized codons, etc.

Once a packaging cell and/or producer cell is transfected or transduced with a vector construct of the invention, genes will be expressed and new virions will be made by the cell. As a result, virions may be collected and used to infect or to transduce a target cell, thereby transferring the gene or genes of interest in the vector to the target cell. Methods of transduction using virus include direct co-culture of cells (Bregni et al., Blood 80:1418-1422 (1992)) or culturing with viral supernatant alone or with appropriate growth factors (Xu et al., Exp. Hemat., 22:223-230 (1994). In some embodiments, viral virions are purified (partially or nearly completely) and/or concentrated.

Some embodiments of the present invention are also concerned with establishing stable packaging cell lines and producer cell lines for making virus. Mutations in the active site of a respective lentiviral protease can enable the construction of lentiviral packaging vectors which are useful to establish stable packaging cell lines for the production of lentiviral vectors, e.g., see U.S. Pat. No. 7,070,993. In brief, the catalytic center of HIV protease includes a three amino acid motif, Asp-Thr-Gly (e.g., see Konvalinka, J. et al., J. Virol. 69:7180-7186, 1995). These three amino acids are typically conserved among HIV, BIV, EIAV, FIV and SIV isolates documented so far (Korber B et al., Science (1998) 280:5371). A mutation altering the Thr residue (corresponding to amino acid number 26 from the start of the protease gene in HIV isolate HXB2) to, for example, a Ser, yields a functional protease which can enable sustained, constitutive expression in a host cell.

Accordingly, in one embodiment, the invention provides for a mutation of the Thr to Ser in the Asp-Thr-Gly motif of a protease, e.g. a BIV or HIV protease. Some embodiments of the invention employ a BIV-based stable packaging cell line, for BIV-based lentiviral vector production, expressing BIV gag/pol with this point mutant in the protease coding region. Such a stable packaging cell line can allow for the production of a BIV lentiviral vector producing cell line that generates high titers of viral particles.

A mutation “corresponding to” a T→S substitution in the encoded lentiviral protease may be either the T→S substitution itself or a substitution having an equivalent biologic effect at the same or analogous motif in the protease. One skilled in the art can readily evaluate other substitutions to see if they result in an equivalent biologic effect. “Equivalent biologic effect” means a substitution resulting in constitutive and sustained expression in the host cell, retaining a similar level of viral protease activity as the T→S substitution or the wildtype protease, Konvalinka et al., J. Virol. 69:7180-7186, 1995. “Viral protease activity” may be measured as described in Konvalinka J. et al., J. Virol. 69:7180-7186, 1995. Activities and cytotoxicities are “similar” within the meaning of the invention when the difference between that measured for the T→S substitution and with another amino acid at that site under essentially the same experimental conditions is less than 2-fold, less than 1.5-fold or even less than 1.2-fold. In some embodiments, a substitution at the same or analogous motif in the protease, when used in a production system, will produce similar viral particle yields, e.g. within about 3-fold, within about 4-fold, within about 5-fold, within about 2-fold, within about 1.5-fold or within about 1.2-fold difference as compared to a production system using the corresponding wild-type protease, for example in a transient transfection system. The fold difference may be less and/or more than the yields with the corresponding wild-type protease.

In some embodiments of the invention, safety features have been engineered into a lentiviral vector system, such as HIV or BIV. One of the major directives in developing a virus-based vector system can be to eliminate or minimize the possibility that the cells, which produce the vector, can generate a “live” virus, e.g., one that can propagate outside of producer cells or can propagate in the target cells. To that end, some vector systems (e.g., BIV) of the present invention incorporate safety features that equal or exceed those of other currently available vector systems, e.g., other lentiviral vectors. Some features that can be incorporated into a BIV, retroviral or lentiviral production system of the invention include, but are not limited to, gag and/or RRE sequences in the vector being minimized; the gag ATG in the vector being mutated; a vector backbone can be self-inactivating (SIN); a tat gene can be eliminated from the system; and/or all or some accessory genes can be eliminated. In some embodiments, a virus-based vector system is used which is designed to minimize, reduce or eliminate the chances that the viral vector will propagate or replicate in the target cells. In some embodiments, a virus-based vector system is used which is designed to allow a viral vector to propagate, spread and/or replicate in the target cells. This includes controlled replication which may limit the spread of the virus, e.g., limited to one round of replication in the target cell such as the initial transformation results in the transformed cells producing viral vector particles which in turn are “released” and transform a second population of cells. In some embodiments, this second population of cells does not produce viral vector particles. A replication competent viral vector or limited replication competent viral vector can result in an increased quantity of transformed cells.

A BIV vector system and its use for nucleic acid delivery (e.g., ocular delivery), can contribute to an excellent safety profile. For example, BIV does not cause human disease; typically BIV vectors do not share significant sequence identity with the human pathogen, HIV; BIV is the most distant virus from HIV in the lentivirus phylogenetic tree; and HIV does not package a BIV vector into viral particles. Thus, infection with HIV will not mobilize and spread a therapeutic BIV vector.

### Proteins of the Invention

The invention provides methods of expressing and producing proteins of the invention, e.g., an antibody or protein analog as described herein. The invention also provides isolated nucleic acid encoding a protein(s) of the invention, vectors and host cells comprising the nucleic acid, and recombinant techniques for the production of the proteins(s).

For recombinant production of a protein, a nucleic acid encoding it may be inserted into a vector (e.g., replicable) for further cloning (e.g., amplification of the DNA) or for expression. In some embodiments, a sequence coding for a protein of the invention may be a coding sequence containing codons optimized for the cell that it is expressed in. In another embodiment, a protein may be produced by homologous recombination, e.g., as described in U.S. Pat. No. 5,204,244.

Many vectors are available. Vector components generally include, but are not limited to, one or more of the following: a signal sequence, an origin of replication, one or more marker genes, an enhancer element, a promoter, and a transcription termination sequence, e.g., as described in U.S. Pat. No. 5,534,615.

Suitable host cells for cloning or expressing a coding region in a vector are prokaryote, yeast, or higher eukaryote cells. Suitable prokaryotes for this purpose include, but are not limited to, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P), Pseudomonas such as P. aeruginosa, and Streptomyces. In some embodiments, an E. coli cloning host is E. coli 294 (e.g., ATCC 31,446), although other strains such as E. coli B, E. coli X1776 (e.g., ATCC 31,537), and E. coli W3110 (e.g., ATCC 27,325) may be suitable. These examples are illustrative rather than limiting.

In addition to prokaryotes, eukaryotic microbes such as filamentous fungi or yeast are suitable cloning or expression hosts. Saccharomyces cerevisiae, or common baker's yeast, is commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (e.g., ATCC 12,424), K. bulgaricus (e.g., ATCC 16,045), K. wickeramii (e.g., ATCC 24,178), K. waltii (e.g., ATCC 56,500), K. drosophilarum (e.g., ATCC 36,906), K. thermotolerans, and K. marxiamis; yarrowia (e.g., EP402,226); Pichia pastoris (e.g., EP183,070); Candida; Trichoderma reesia (e.g., EP244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified and can be used for expressing proteins. A variety of viral strains for transfection can be used for protein expression and are publicly available, e.g., the L-1 variant of Autographa californica NPV and the Bm-5 strain of Bombyx mori NPV, and such viruses may be used as the virus herein according to the present invention, for example, for transfection of Spodoptera frugiperda cells. Plant cell cultures of cotton, corn, potato, soybean, petunia, tomato, and tobacco can also be utilized as hosts.

Some embodiments of the invention utilize vertebrate cells, and propagation of vertebrate cells in culture (tissue culture) can be a routine procedure. Examples of useful mammalian host cell lines are monkey kidney CVI line transformed by SV40 (e.g., COS-7, ATCC CRL 1651); human embryonic kidney line (e.g., 293 or 293T cells including either cell line subcloned for growth in suspension culture, Graham et al., J. Gen Virol. 36:59 (1977) such as 293 Freestyle (Invitrogen, Carlsbad, Calif.)) or 293FT; baby hamster kidney cells (e.g., BHK, ATCC CCL 10); Chinese hamster ovary cells; Chinese hamster ovary cells/-DHFR (e.g., CHO, Urlaub et al., Proc. Natl. Acad. Sci. USA 77:4216 (1980)); mouse sertoli cells (e.g., TM4, Mather, Biol. Reprod. 23:243-251 (1980)); monkey kidney cells (e.g., CVI ATCC CCL 70); African green monkey kidney cells (e.g., VERO-76, ATCC CRL-1587); human cervical carcinoma cells (e.g., HELA, ATCC CCL 2); canine kidney cells (e.g., MOCK, ATCC CCL 34); CF2TH cells; buffalo rat liver cells (e.g., BRL 3A, ATCC CRL 1442); human lung cells (e.g., W138, ATCC CCL 75); human liver cells (e.g., Hep G2, HB 8065); mouse mammary tumor (e.g., MMT 060562, ATCC CCL51); TRI cells (Mather et al., Annals N.Y. Acad. Sci. 383:44-68 (1983)); MRC 5 cells; FS4 cells; and a human hepatoma line (Hep G2).

Host cells are typically transformed with the expression or cloning vectors for protein production and cultured in conventional nutrient media modified as appropriate for inducing promoters, selecting transformants, amplifying the genes encoding the desired sequences or for downstream purification and/or concentration procedures.

In some instances, a host cell may be modified to decrease or eliminate expression of an endogenous protein. For example, if a factor B analog is to be produced in a particular host cell (e.g., a CHO cell), then the host cell could be modified so as expression of the host cell's native factor B (e.g., hamster factor B), is reduced or eliminated. Therefore, the invention provides a method of producing a complement protein analog comprising reducing or eliminating the expression of the corresponding native complement protein in the host cell. Methods for reducing, eliminating or knocking out expression of a host cell protein are known in the art. For example, a protein's expression level may be reduced or eliminated by engineering the host cell to express inhibitory RNA (e.g., RNAi) specific for the RNA coding for the protein. For example, Clontech (Mountain View, Calif.) sells various vectors and kits, such as those referred to as part of the KNOCKOUT™ RNAi Systems, for knocking down expression of proteins in a host cell. Other methods include gene targeting by homologous recombination which allows the introduction of specific mutations into any cloned gene, e.g., see Current Protocols in Molecular Biology, John Wiley & Sons, Inc., 1994-1998, Sections 9.16 and 9.17. This can be used to knockout the gene expressing the host cell protein.

Another method which may be utilized to reduce expression of an endogenous protein, involves using a targeted transcription factor that represses expression of the endogenous protein. For example, a repressor domain from a transcription factor may be attached or fused to a DNA binding domain such as a zinc finger polypeptide. One skilled in the art can design zinc finger polypeptides that bind specific DNA sequences, e.g., see U.S. Pat. Nos. 6,140,081; and 7,067,617; and U.S. Published Patent Applications 20060078880; 20040224385; and 20070213269. One skilled in the art can associate designed zinc finger polypeptides with a transcriptional repressor domain (e.g., a KRAB (Krüppel-associated box) domain). Examples of such molecules and techniques are described in Beerli et al. (Proc Natl Acad Sci USA. 2000 97(4): 1495-1500) and U.S. Published Patent Application 20070020627. In some embodiments of the invention, a host cell would be transduced with a vector expressing the transcriptional repressor. This approach has an advantage over knocking out the gene of interest using homologous recombination because, in most cases, a host cell will be diploid and it would be desirable to knock out both gene copies. Whereas, expression of a transcriptional repressor should repress expression of both gene copies.

The expression of particular endogenous protein may also be reduced using compounds that will directly or indirectly reduce the expression of the particular endogenous protein. Using fB as an example, various compounds can be used to reduce the expression of endogenous fB expression. For example, fB expression has been shown to be inhibited by histamine (Falus & Meretey, Immunology 1987 60:547-551 and Falus & Meretey, Mol Immunol 1988 25(11):1093-97), sodium butyrate (Andoh et al. Clin Exp Immuno 1999 118:23-29), a glucocorticoid such as dexamethasone (Dauchel et al. Eur J Immunol 1990 20(8):1669-75), platelet derived growth factor (Circolo et al. 1990 The Journal of Biol Chem 265(9):5066-5071), epidermal growth factor (Circolo et al. 1990), and fibroblast growth factor (Circolo et al. 1990). A host cell of the invention may be cultured in the presence of any one or combination of these molecules to reduce the endogenous expression of fB and possibly fD. This is useful for the production of fB analogs or fD analogs in a host cell. Therefore, in some embodiments of the invention, a host cell expressing an fB analog or fD analog is cultured in the presence of any one or more compounds selected from the group consisting of a histamine, a sodium butyrate, a glucocorticoid (e.g., dexamethasone), a platelet derived growth factor, an epidermal growth factor, or a fibroblast growth factor.

Various compounds and proteins have been shown to upregulate or maintain expression of fB. For example, fB expression has been shown to be upregulated or maintained by TNF (Andoh et al. Clin Exp Immuno 1999 118:23-29), estrogen (Sheng-Hsiang et al. Biology of Reproduction 2002 66:322-332), Interleukin-1 (Dauchel et at. Eur J Immunol 1990 20(8):1669-75), dexamethasone (Lappin & Whaley, Biochem J 1991 280:117-123), prednisolone (Lappin & Whaley 1991), cortical (Lappin & Whaley 1991), and Interferon-gamma (Huang et al. 2001 Eur J Immunol 31:3676-3686). A host cell of the invention may be cultured in the absence of any one or combination of these molecules to reduce the endogenous expression of fB and possibly fD. Additionally, a host cell may be cultured in the presence of an inhibitor of any one or more of these compounds. This can be useful for the production of fB analogs or fD analogs in a host cell. Therefore, in some embodiments of the invention, a host cell expressing an fB analog or fD analog is cultured in the presence of any one or more compounds that inhibit a compound selected from the group consisting of a TNF, estrogen, interleukin-1, dexamethasone, prednisolone, cortical, and interferon-gamma. In some embodiments, expression by the host cell of one or more of these compounds is reduced, e.g., using methods as described herein. Examples of inhibitors of estrogen include, but are not limited to, tamoxifen. Inhibitors also include antibodies that bind and reduce the activity of the compound. For example, various antibodies that bind and inactivate TNF are know in the art.

Host cells used to produce proteins of the invention may be cultured in a variety of media. Commercially available media such as Ham's F10 (e.g., Sigma), Minimal Essential Medium ((MEM), (e.g., Sigma), RPM1-1640 (e.g., Sigma), and Dulbecco's Modified Eagle's Medium ((DMEM), e.g., Sigma) can be suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. No. 30,985 may be used as culture media for the host cells. In some embodiments, medium can be completely defined (e.g., CD-CHO medium (Invitrogen)), serum-free or serum containing Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, or will be within the skill of the ordinarily skilled artisan to develop.

When using recombinant techniques, a protein can be produced intracellularly, in the periplasmic space and/or can be directly secreted into the medium. In some embodiments, if a protein is produced intracellularly, as a first step, the particulate debris, either host cells or lysed fragments, is removed, for example, by centrifugation or ultrafiltration. Carter et al., Bio/Technology 10:163-167 (1992) describe a procedure for isolating a protein (an antibody) which is secreted to the periplasmic space of E. coli. In some embodiments, a protein is secreted into a medium. In some embodiments, supernatant from such expression systems are generally first concentrated, e.g., using a commercially available protein concentration filter, for example, an Amicon or Millipore Pellicon ultrafiltration unit. In some embodiments, a protease inhibitor such as PMSF may be included in any of the foregoing steps to inhibit proteolysis and/or antibiotics may be included to prevent the growth of adventitious contaminants.

A protein composition prepared from cells can be purified using, for example, hydroxylapatite chromatography, gel electrophoresis, dialysis, size exclusion chromatography, affinity chromatography, immunoaffinity chromatography, tangential flow purification, diafiltration, ion exchange chromatography, reverse phase chromatography, heparin sepharose affinity chromatography and other known forms of separation and concentration.

When the protein is an antibody, protein A can be utilized for purification. The suitability of protein A as an affinity ligand can depend on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-12 (1983)). Protein G is recommended for all mouse isotypes and for human γ 3 (Guss et al., EMBO J. 5:1567-1575 (1986)). In some embodiments, a matrix to which an affinity ligand is attached is agarose, but other matrices are available. Mechanically stable matrices such as controlled pore glass or poly(styrenedivinyl)benzene can allow for faster flow rates and shorter processing times than can be achieved with agarose. Where the antibody comprises a C_{H}3 domain, the BAKERBOND ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), SDS-PAGE, and ammonium sulfate precipitation are also available depending on the protein or antibody to be recovered.

Following any preliminary purification step(s), a mixture comprising the protein of interest and contaminants, if any, may be subjected to low pH hydrophobic interaction chromatography, e.g., using an elution buffer at a pH between about 2.5-4.5, in some cases performed at low salt concentrations (e.g., from about 0-0.25M salt) or other procedures for further purification.

### Ocular Delivery or Therapy

The choice of an ocular gene therapy use is relevant to the safety of the therapy. Vectors that achieve sustained expression generally do so by integrating their transgene payloads into a target cell genomic DNA. An integration event could cause a local disruption in the target cell DNA known as “insertional mutagenesis.” In some embodiments, a BIV vector can specifically target a cell type(s), for example, RPE cells. RPE cells generally do not undergo division; RPE cells very rarely give rise to tumors; in some cases only a limited number of RPE cells need be transduced; and transduced RPE cells will remain localized at the injection site, which in some cases can be repeatedly visualized via non-invasive methods, e.g., opthalmoscopy. In some embodiments, a non-invasive method for visualization is opthalmoscopy and RPE cells (e.g., transduced RPE cells) can be eliminated via laser treatment. Thus, RPE cells as target cells for producing products (e.g., therapeutic products such as proteins or siRNA) are particularly well suited for genetic modification. Finally, a vector can be engineered to achieve site-directed integration. One means for obtaining such directed integration is through the use of particular nucleic acid binding molecules, such as a molecule containing a zinc finger motif e.g. associated with an integrase. Another way to address this issue is to use a vector such as AAV which, for the most part, remains episomal.

FIG. 1 is an example of the progression of AMD. Early AMD is typically characterized by drusen, small deposits between the retinal pigment epithelial (RPE) layer and Bruch's membrane. Drusen can act to block the flow of nutrients, oxygen, and/or waste between the retina and the underlying choroidal capillary bed. Importantly, drusen contains many inflammatory factors and complement factors. The course of AMD primarily proceeds in one of two directions. In wet AMD, new blood vessels grow out of the choroid by a process known as neovascularization. The new vessels are defective. They leak and bleed, which rapidly leads to blindness. The second disease course is Geographic Atrophy. In this case, the retina, RPE layer, and choroid all die leading to expanding areas in the back of the eye with no retina. The expanding areas are associated with blind spots, which eventually can knock out central vision and lead to blindness. Importantly, some embodiments of the instant invention will treat all stages of AMD; that is, early AMD and both forms of late AMD. The instant invention can be used as a general treatment of AMD.

### Compositions, Formulations and Preparations

Some embodiments of the invention provide compositions, e.g., pharmaceutical compositions such as for therapeutic uses. In some embodiments, a composition comprises a complement analog(s) as described herein. In some embodiments, a “therapeutically effective amount” or appropriate dosage is determined by comparing the in vitro activity of the naturally occurring protein with that of the analog and/or comparing the in vitro activity of the naturally occurring protein with the in vivo activity of the naturally occurring protein (e.g., in an animal model), then calculating or extrapolating the expected and/or desired in vivo activity of the analog, in some cases adjusting for any differences in half-life.

Formulations (e.g., for injection) are generally, but not necessarily, biocompatible solutions of the active ingredient, e.g., comprising Hank's solution or Ringer's solution. Formulations for transdermal or transmucosal administration generally include, but are not limited, penetrants such as fusidic acid or bile salts in combination with detergents or surface-active agents. In some embodiments, formulations can be manufactured as aerosols, suppositories, or patches. In some embodiments, oral administration may not be favored for protein or peptide active ingredients; however, this type of composition may be suitably formulated, e.g., in an enteric coated form, in a depot, in a capsule and so on, so as to be protected from the digestive enzymes, so that oral administration can also be employed. Some formulations of the invention comprise balanced salt solution (Alcon Laboratories, Inc., Fort Worth, Tex.) or balanced salt solution plus (Alcon Laboratories, Inc.). In some embodiments, a formulation comprises one or more of the following: citrate, NaCl (e.g., 0.64%), potassium chloride (KCl) (e.g., 0.075%), calcium chloride dihydrate (CaCl₂.2H₂O) (e.g., 0.048%), magnesium chloride hexahydrate (MgCl₂.6H₂O) (e.g., 0.03%), sodium acetate trihydrate (CH₃CO₂Na.3H₂O) (e.g., 0.39%), sodium citrate dihydrate (C₆H₅O₇Na₃.2H₂O) (e.g., 0.17%), and sodium hydroxide and/or hydrochloric acid (to adjust pH) and water. The preceding list includes some molecules that are listed as particular hydrates, e.g., dihydrate, trihydrate, hexahydrate, etc. It is understood that various hydrates of these compounds can be used in the present invention and the invention is not limited to these particular hydrate forms of the listed molecules. In some embodiments, a formulation comprises one or more of the following: NaCl, monobasic phosphate monohydrate, dibasic sodium phosphate heptahydrate and hydrochloric acid and/or sodium hydroxide to adjust pH and water. In some embodiments, a formulation comprises one or more of the following: histidine (e.g., about 10 mM), α, α-trehalose dehydrate (e.g., about 10% or about 50 mM), MgCl₂ (e.g., about 10 mM), a polysorbate such as polysorbate 20 (e.g., about 0.01%) and NaCl₂ (e.g., about 0.1%). In some embodiments, a formulation comprises or consists of a molecule(s) of the present invention, 10 mM histidine, 10 mM MgCl₂, 50 mM trehalose and 0.01% polysorbate 20. In some embodiments, a formulation comprises or consists of a molecule(s) of the present invention, 1.0% NaCl and 10 mM MgCl₂. In some embodiments, a formulation or composition is at a pH of about 5.5. In some embodiments, a formulation or composition is at a pH of between from about 5.0 to 9.0, about 5.0 to 5.5, about 5.3 to 5.7, about 5.5 to 6.0, about 5.8 to 6.2, about 6.0 to 6.5, about 6.3 to 6.7, about 6.5 to 7.0, about 6.8 to 7.2, about 7.0 to 7.5, about 7.3 to 7.7, about 7.5 to 8.0, about 7.8 to 8.2, about 8.0 to 8.5, about 8.3 to 8.7 and about 8.5 to 9.0, whatever is suitable to retain the biological activity and stability of the active ingredient(s).

Examples of suitable formulations and formulatory methods for a desired mode of administration may be found in Remington's Pharmaceutical Sciences, latest edition, Mack Publishing Co., Easton, Pa. and in U.S. Pat. No. 7,208,577. Dosage levels and precise formulations may also be determined by routine optimization procedures as is generally known in the art.

Some embodiments of the invention provide pharmaceutical compositions of complement analogs (e.g., factor B analogs). In some embodiments, a therapeutically effective amount or appropriate dosage is determined by, e.g., comparing the in vitro activity of the naturally occurring protein with that of the analog, comparing the in vitro activity of the naturally occurring protein with the in vivo activity of the naturally occurring protein, then calculating the expected in vivo activity of the analog, adjusting for any measured differences in half-life. In some embodiments, a therapeutically effective amount is determined based on previous studies, such as clinical trials. In some embodiments, it is determined by varying the dosage, e.g., in a patient, until a desired effect is achieved or a therapeutic benefit is achieved. Guidance as to particular dosages and methods of delivery is provided in the literature; see, for example, U.S. Pat. Nos. 4,657,760; 5,206,344; or 5,225,212.

In some embodiments, a composition for use in vivo contains a “carrier” or a “pharmaceutically acceptable carrier”. The term “carrier” refers to a diluent, adjuvant, excipient, or vehicle with which the vector of interest is administered. The term “carrier’ includes, but is not limited to, either solid or liquid material, which may be inorganic or organic and of synthetic or natural origin, with which an active component(s) of the composition is mixed or formulated to facilitate administration to a subject. Any other materials customarily employed in formulating pharmaceuticals may be suitable. Solid carriers include, but are not limited to, natural and synthetic cloisonne silicates, for example natural silicates such as diatomaceous earths; magnesium silicates, for example talcs; magnesium aluminum silicates, for example attapulgites and vermiculites; aluminum silicates, for example kaolinites, montmorillonites, and micas; calcium carbonate; calcium sulfate; synthetic hydrated silicone oxides and synthetic calcium or aluminum silicates; elements such as carbon or sulfur; natural and synthetic resins such as polyvinyl alcohol; and waxes such as paraffin and beeswax. Examples of suitable liquid carriers include water; aqueous solutions containing oxygenated organic compounds such as ethanol and oils, including those of petroleum, animal, vegetable or synthetic origin, such as peanut oil (e.g., hypoallergenic), soybean oil, mineral oil, sesame oil and the like. In some embodiments, water, physiological saline, dextrose and glycerol solutions or a buffer can be a carrier. In some embodiments, a composition comprises one or more pharmaceutically acceptable carriers such as saline, phosphate buffered saline, and/or a controlled release formulation. Buffers and other materials normally present in pharmaceutical preparations, such as flavoring, odoring, coloring and suspending agents, can also be present. Pharmaceutical carriers can differ from typical solutions and suspensions in that they are specifically prepared for use in vivo to exclude and/or minimize the amount or availability of substances that may be harmful to the host to whom the composition is administered (e.g., removal of bacterial toxins).

In general, water, a suitable oil(s), saline, aqueous dextrose (glucose), and related sugar solutions and glycols such as propylene glycol or polyethylene glycols are typically suitable carriers for parenteral solutions. In some embodiments, solutions for parenteral administration contain a water soluble salt of the active ingredient, suitable stabilizing agents, and if desirable or necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical excipients include, but are not limited to, starch, glucose, lactose, sucrose, gelatin, antibiotics, preservatives, malt, rice, flour, chalk, silica gel, sodium stearate, glycerol monostearate, talc, sodium chloride, dried skim milk, glycerol, propylene, glycol, water, ethanol and the like. A composition, if desired, can also contain wetting and/or emulsifying agents, and/or pH buffering agents. Where necessary, a composition may also include a solubilizing agent and/or a local anesthetic such as lignocaine to ease pain at the site of the injection.

Suitable pharmaceutical carriers are described in Remington's Pharmaceutical Sciences, Mack Publishing Co., a standard reference text in this field. Some embodiments of the invention include the use of 1, 2, 3, 4, 5, 6, 7, 8, 9, 10, 11, 12, 13, 14, 15 or more carriers and/or excipients.

Active ingredients may also be entrapped in microcapsules prepared, for example, by coascervation techniques or by interfacial polymerization, for example, hydroxymethylcellulose or gelatin microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences, Mack Publishing Co.

Stabilizers can be used in the present invention. Stabilizers refer to a broad category of excipients which can range in function from a bulking agent to an additive which solubilizes an agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc., organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (e.g., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose, glucose; disaccharides, such as lactose, maltose and sucrose; trisaccharides such as raffinose; polysaccharides such as dextran and so on. Stabilizers are typically present in the range from 0.1 to 10,000 w/w per part of active agent (e.g., an fB analog such as fb3 or an antibody or fragment thereof that binds fB).

Disintegrants may be included in the formulation of a therapeutic into a solid dosage form. Materials used as disintegrates include, but are not limited to, starch including the commercial disintegrant based on starch, Explotab. Sodium starch glycolate, Amberlite, sodium carboxymethylcellulose, ultramylopectin, sodium alginate, gelatin, orange peel, carboxymethyl cellulose, natural sponge and bentonite may all be used. Another form of the disintegrants is the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold an agent together to form a tablet and include materials from natural products such as acacia, tragacanth, starch and gelatin. Others include methyl cellulose (MC), ethyl cellulose (EC), carboxymethyl cellulose (CMC) and other celluloses. Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall and/or can be added to the formulation, and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, e.g., Carbowax 4000 and 6000 and so on.

Glidants that might improve the flow properties of the agent during formulation and aid rearrangement during compression might be added. Glidants may include starch, talc, pyrogenic silica and hydrated silicoaluminate.

To aid dissolution of an agent into an aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Nonionic detergents that could be included in the formulation as surfactants include, but are not limited to, lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose, carboxymethyl cellulose and any of the pluronic detergents such as Pluronic F68 and/or Pluronic F127 (e.g., see Strappe et al. European Journal of Pharmaceutics and Biopharmaceutics 61:126-133 (2005)). Surfactants could be present in the formulation of a protein or derivative either alone or as a mixture in different ratios.

Additives which potentially enhance uptake of a protein (or derivative) include, but are not limited to, fatty acids, oleic acid, linoleic acid and linolenic acid.

In some embodiments, a controlled release formulation may be desirable. An agent (e.g., a protein) could be incorporated into an inert matrix which permits release by either diffusion, swelling or leaching mechanisms, e.g., gums and cellulosic compounds. Slowly degenerating matrices may also be incorporated into a formulation. Another form of a controlled release is by a method based on the Oros therapeutic system (Alza Corp.), e.g., a drug is enclosed in a semipermeable membrane which allows water to enter and push drug out through a single small opening due to osmotic effects. Entric coatings have a delayed release effect, but may also have a sustained release effect.

Other coatings may be used for the formulation. These include a variety of sugars which could be applied in a coating pan. An agent could also be given in a film coated tablet and the materials used in this instance can be divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide an optimum film coating. Film coating may be carried out in a pan coater or in a fluidized bed or by compression coating.

In some embodiments, nucleic acids and particles of the invention can be formulated as neutral or salt forms. Pharmaceutically acceptable salts include, but are not limited to, those formed with anions such as those derived from hydrochloric, phosphoric, acetic, citric, oxalic and/or tartaric acids, etc., and those formed with cations such as those derived from sodium, potassium, ammonium, calcium, ferric hydroxides, isopropylamine, triethylamine, 2-ethylamino ethanol, histidine, procaine, etc. Other stabilizing agents include proteins, such as albumin, emulsifiers, such as, Pluronics, Tweens and so on. In addition, amino acids, saccharides, such as trehalose, mannose, sucrose and other compounds with stabilizing properties can be included as known in the art.

Also contemplated herein is pulmonary delivery of an agent or protein (or derivative thereof) of the present invention. A protein (derivative) is delivered to the lungs of a mammal while inhaling and in some embodiments traverses across the lung epithelial lining to the blood stream. (e.g., see Adjei et al., Pharmaceutical Research 7: 565-569 (1990); Adjei et al., International Journal of Pharmaceutics 63: 135-144 (1990); Braquet et al., Journal of Cardiovascular Pharmacology 13 (suppl. 5):s.143-146 (1989); Hubbard et al., Annals of Internal Medicine 3:206-212 (1989); Smith et al., J. Clin. Invest. 84:1145-1146 (1989); Oswein et al., Proceedings of Symposium on Respiratory Drug Delivery II, Keystone, Colo., March, 1990; Debs et al., The Journal of Immunology 140:3482-3488 (1988) and Platz et al., U.S. Pat. No. 5,284,656).

Contemplated for use in the practice of this invention are a wide range of mechanical devices designed for pulmonary delivery of therapeutic products, including but not limited to nebulizers, metered dose inhalers, and powder inhalers.

Some specific examples of commercially available devices suitable for the practice of some embodiments of the invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

In some embodiments, these devices use formulations suitable for the dispensing of protein. Typically, each formulation is specific to the type of device employed and may involve the use of an appropriate propellant material, in addition to diluents, adjuvants and/or carriers useful in therapy.

In some embodiments, a protein is prepared in particulate form. In some embodiments, this particulate form has an average particle size of less than 10 μm (or microns), most preferably 0.5 to 5 μm, for delivery to the distal lung.

Carriers can include carbohydrates such as trehalose, mannitol, glutathione, xylitol, sucrose, lactose, and sorbitol. Other ingredients for use in formulations may include, for example, DPPC, DOPE, DSPC and DOPC. Natural or synthetic surfactants may be used. Polyethylene glycol may be used (even apart from its use in derivatizing a protein). Dextrans, such as cyclodextran, may be used. In some embodiments, cyclodextrin, tertiary amines and/or beta-cyclodextrin may be used. Bile salts and other related enhancers may be used. Cellulose and cellulose derivatives may be used. Amino acids may be used, such as use in a buffer formulation. Also, the use of liposomes, microcapsules or microspheres, inclusion complexes, or other types of carriers is contemplated.

Formulations suitable for use with a nebulizer (e.g., jet or ultrasonic) will typically comprise protein dissolved in water, in some embodiments, at a concentration of about 0.1 to about 25 mg of biologically active protein per mL of solution. A formulation may also include a buffer and/or a simple sugar (e.g., for protein stabilization and regulation of osmotic pressure). A nebulizer formulation may also contain a surfactant, to reduce or prevent surface induced aggregation of a protein(s) caused by atomization of the solution in forming the aerosol.

Formulations for use with a metered-dose inhaler device will generally comprise a finely divided powder containing a protein or agent of the invention suspended in a propellant, e.g., with the aid of a surfactant. A propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

In some embodiments, formulations for dispensing from a powder inhaler device will comprise a finely divided dry powder containing an agent(s) or protein(s) of the invention and may also include a bulking agent, such as lactose, sorbitol, sucrose, mannitol, trehalose, or xylitol in amounts which facilitate dispersal of the powder from the device, e.g., 50 to 90% by weight of the formulation.

Nasal delivery of a protein is also contemplated. In some embodiments, nasal delivery allows the passage of the protein to the blood stream, e.g., directly after administering an agent(s) to the nose. In some embodiments, this is accomplished without the necessity for deposition or minimal deposition of the agent(s) in the lung. Formulations for nasal delivery include those with dextran or cyclodextran. Delivery via transport across other mucus membranes is also contemplated.

In some embodiments, the formulation of an agent such as a protein will be such that between about 0.10 μg/kg/day and 10 mg/kg/day will yield a desired (e.g., therapeutic) effect. Methods and routes of administration are described herein. Compositions and formulations of the invention may be administered to an animal by, for example, infusion (e.g., slow infusion) or bolus injection. A molecule or vector of interest may be administered by infusion (e.g., slow infusion) or bolus injection, by absorption through epithelial or mucocutaneous linings and may be administered together with other biologically active agents. Administration can be systemic (e.g., I.V.) or local.

In some embodiments, administration is by ocular injection. Various types of ocular injections are described herein. In some embodiments, an ocular injection of a protein of the invention is between about 0.05 mg to about 10 mg, about 0.1 mg to about 10 mg, about 0.5 mg to about 10 mg, about 1 mg to about 10 mg, about 5 mg to about 10 mg, about 0.05 mg to about 5 mg, about 0.5 mg to about 3 mg, about 0.5 mg to about 1 mg, about 0.05 mg to about 0.5 mg, about 0.05 mg to about 0.1 mg, about 0.1 mg to about 5 mg, about 1 mg to about 5 mg, about 1 mg to about 3 mg, and 0.5 to about 2 mg of the protein per injection. In some embodiments, an ocular injection of between from about 5 ul to about 150 ul, about 25 ul to about 150 ul, about 50 ul to about 150 ul, about 100 ul to about 150 ul, about 5 ul to about 100 ul, about 50 ul to about 150 ul, about 25 ul to about 150 ul, about 25 ul to about 100 ul, or about 35 ul to about 70 ul is performed. In some embodiments, an ocular injection of about 50 ul is performed.

Some formulations of the invention can then be manufactured as aerosols, suppositories, eye drops or patches.

In some embodiments, ingredients are supplied either separately or mixed together in unit dosage form, for example, as a dry lyophilized powder or water free concentrate which may be in a hermetically sealed container such as an ampoule or sachet typically indicating the quantity of active agent. When a pharmaceutical composition is administered by injection, an ampoule of sterile water for injection or saline can be provided, e.g., so that the ingredients may be mixed prior to administration. Ingredients also can be supplied in frozen form or in liquid form. Various ingredients (e.g., inert ingredients) to stabilize the active ingredients and/or to enhance shelf life can be included, e.g., as known in the art.

To prolong the serum circulation in vivo of some composition of the invention (e.g., an fb3 or an antibody), various techniques can be used. For example, inert polymer molecules, such as high molecular weight polyethylene glycol (PEG), can be attached (e.g., to an antibody) with or without a multifunctional linker either through site-specific conjugation of the PEG (e.g., to the N-terminus or to the C terminus of an antibody) or via epsilon amino groups present on lysine residues. In some embodiments, linear or branched polymer derivatization that results in minimal loss of biological activity can be used. The degree of conjugation can be closely monitored by SDS-PAGE and mass spectrometry to ensure proper conjugation of PEG molecules to the composition (e.g., a protein such as an antibody or an fB analog). In some embodiments, unreacted PEG can be separated from PEG conjugates by size-exclusion and/or by ion exchange chromatography. PEG-derivatized compositions can be tested for activity as well as for in vivo efficacy using methods known to those of skilled in the art.

### Articles of Manufacture

In some embodiments, an article of manufacture contains materials, e.g., useful for the treatment of the disorders or diseases as described herein. In some embodiments, an article of manufacture comprises a container and a label. Suitable containers include, for example, bottles, vials, syringes, and test tubes. Containers may be formed from a variety of materials such as glass or plastic. A container may hold a composition of the invention, e.g., which is effective for treating a condition. A container may have an access port (such as a sterile port), for example, the container may be an intravenous solution bag or a vial having a stopper pierceable by a hypodermic injection needle. In some embodiments, a container may have an access port, e.g., having a sealing means that can be traversed to allow removal of the contents such as a pierceable and/or pliable material. An active agent in the composition can be any of those described herein. In some embodiments, a label on or associated with the container indicates that the composition is used for treating a condition of choice. In some embodiments, an article of manufacture may comprise a second container comprising a pharmaceutically-acceptable buffer, such as phosphate-buffered saline, Ringer's solution and/or dextrose solution. In some embodiments, an article of manufacture comprises a container of water. It may further include other materials desirable from a commercial and user standpoint, including other buffers, diluents, filters, needles, syringes, and package inserts with instructions for use.

### Transgenic Animals

Some embodiments of the invention provide a transgenic animal (e.g., nonhuman) expressing a variant or mutant of at least one complement pathway component as described herein. Methods for making a transgenic animal are known in the art. Some embodiments of the invention provide a transgenic animal expressing a nucleic acid and/or protein of the invention, e.g. a factor B analog such as fB3. In some embodiment, a transgenic animal (such as a mouse) will also comprise a mutation, deletion or disruption in the Fas gene, e.g., see Macmicking et al. Cell. 81:641-650 (1995).

The instant invention now will be exemplified in the following non-limiting examples.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

The inventors, inter alia, have determined that gene delivery provides an efficacious means of achieving sustained and continuous delivery of therapeutics to the eye. Embodiments of the invention will achieve sufficient gene transfer, sufficient gene expression, appropriate timing and distribution of expression and of the expressed protein therapeutic, negligible systemic distribution of the expressed therapeutic protein, appropriate biological activity of the expressed protein therapeutic, and/or an absence of or diminished immune response. The examples will outline vector delivery with BIV-based vectors, derived from a bovine lentivirus and in some embodiments in combination with proteins that attenuate complement activation (e.g., alternative pathway complement activation). One purpose is to treat and/or study age related macular degeneration (AMD) as well as both types of end stage AMD, Geographic Atrophy and wet AMD. These diseases are the leading causes of blindness in the developed world and affect more than 25% of people over the age of 65. The inventors chose the alternative pathway of complement activation as a therapeutic target based on their conclusion that this pathway is an important and general underlying cause for all forms of AMD. This conclusion is consistent with the statistical association of certain polymorphisms in the gene encoding the complement inhibitory protein, Complement factor H, with an increased risk for the development of all forms of AMD (Klein et al. 2005, Haines et al. 2005, Edwards et al. 2005, Hageman et al. 2005, Li et al. 2006, Maller et al. 2006, Magnusson et al. 2006, Sepp et al. 2006, and Postel et al. 2006).

Example one outlines some production methods for BIV vectors by a process that can be utilized for most if not all lentiviral vectors. Examples two through seven demonstrate the efficiency with which BIV vectors genetically modify animal retinal cells in vivo and primary human retinal cells in vitro. These examples also include two studies of efficacy in mouse models relevant to the treatment of human eye disease. Example eight describes novel therapeutic proteins that attenuate activation of a complement pathway by impeding a positive feedback loop. These include three dominant negative variants of complement factor B designated fB1, fB2, and fB3. Example nine describes the generation of antibodies to inhibit a complement pathway. Examples ten and eleven discuss in vitro evaluations of complement-inhibiting proteins. Example twelve outlines the strategy for an in vivo evaluation of vectors in a mouse model relevant to complement-mediated AMD. Example thirteen utilizes a vector system that is not based on a lentivirus to deliver proteins. Example 14 is included to demonstrate that the instant invention has utility for diseases or conditions other than those of the eye. Examples 15 through 18 provide additional details and data to demonstrate that dominant negative factor B moieties inhibit human and other non-human complement pathways. Example 19 provides data on the cleavage of dominant negative factor B moieties by factor D. Example 20 examines the affinity with which dominant negative factor B variants form a complex with complement factor C3b. Example 21 shows that the dominant negative variant, fB3 forms a stable complex with complement factor D. Examples 22 through 24 demonstrate that antibodies against complement factors B and D can inhibit a complement pathway. Examples 25 and 26 provide data to verify the biological activity of purified human fB3 protein and describe the generation of cell lines for the production of human fB3. Examples 27 and 28 describe the evaluation of a BIV vector encoding human fB3 in a mouse laser injury model of complement activation as well as the evaluation of human fB3 protein injection in the same animal model. Example 29 provides a detailed and scaleable protocol for the concentration and purification of BIV vectors. Examples 30 through 32 provide modifications to the protocol in Example 29 for vector production.

### Example One

### Production of BIV Vectors

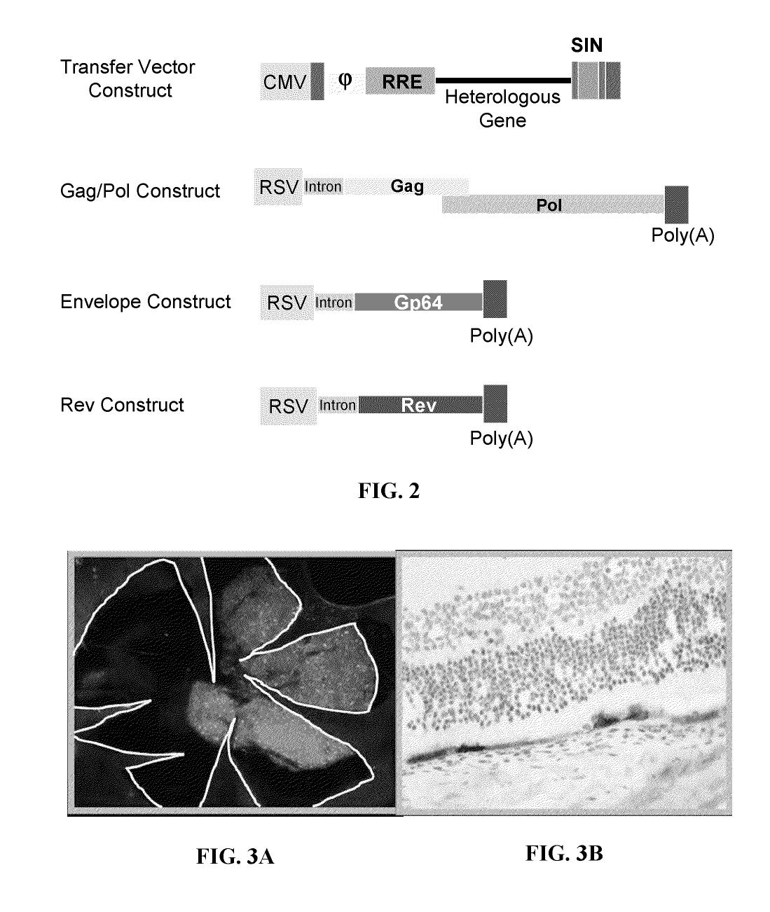

Some general production methods for BIV vectors are described in the literature, e.g., see Matukonis et al., 2002; Molina et al., 2004 as well as in U.S. Pat. No. 6,864,085 and PCT Publ. No. WO 03/066810. In some methods, four components can be used for vector production. These components, e.g., shown in FIG. 2, include: 1) the BIV transfer vector construct; 2) an expression construct encoding the BIV gag/pol polyproteins; 3) an expression construct encoding an envelope protein such as the VSV-G protein or baculovirus gp64 envelope protein; and 4) an expression construct encoding the BIV rev protein. The transfer vector construct contains the heterologous (therapeutic) gene and generates an RNA transcript that is packaged into the vector particles. The gag/pol and envelope constructs produce the capsid proteins that form the vector particle. The rev construct produces a protein that is required to transport the vector RNA out of the cell nucleus.

To generate vector, cells in culture are co-transfected with the four expression constructs via calcium phosphate co-precipitation. One to three days later, after assembly of the particles in the cytoplasm and packaging of the vector RNA, the vector particles bud through the cell membrane, acquire a lipid bilayer coat, and accumulate in the tissue culture medium from which they can be purified and/or concentrated.

Specifically, 1×10⁷ 293T (ATCC) or 293 FT (Invitrogen) cells are seeded into 150 mm dishes in DMEM plus 10% FBS and incubated at 37° C. in a 5% CO₂ incubator. The cells are transfected the following day when the plates are approximately 85-90% confluence. Typically, 45 μg of the transfer vector construct, 45 μg of the gag/pol construct, 15 μg of the envelope construct (which, in this example, encodes the baculovirus gp64 envelope protein), and 30 μg of the rev expression construct are used for each dish. Methods for calcium phosphate transfection are well known to those skilled in the art, and kits for calcium phosphate transfection are commercially available (e.g. Promega). After 24 hr, the medium is aspirated and replaced with fresh medium plus 5 mM sodium butyrate. The viral vector supernatant is harvested 24 hours later, filtered through a 0.45 μm filter, and stored frozen in aliquots at −80° C. for in vitro use. Titers of vector in cell supernatants are generally 2−6×10⁶ transducing units per ml (tu/ml).

For in vivo use, viral vector can be readily purified and concentrated 100-fold with an ultracentrifugation procedure well known to those skilled in the art or with the following chromatographic method. Vector supernatant is incubated with 50 units/ml of Benzonase at 37° C. for 30 minutes and then filtered through a 0.2 μm aPES filter. Three hundred mls of vector supernatant are diluted 1:1 with loading buffer (2×PBS containing a total of 1M NaCl) and then loaded onto a Sartobind Q75 membrane adsorber unit (Sartorius) at a rate of 5 ml/min using a peristaltic pump. The Q75 membrane adsorber unit is then washed with 50-75 mls of wash buffer (1×PBS containing a total of 500 mM NaCl) at 5 ml/min. After the wash step, the vector is eluted as follows. The Q75 unit is disconnected from the peristaltic pump and carefully attached to a 5 ml syringe containing elution buffer (1×PBS containing a total of 1.3 M NaCl). At a rate of approximately 5 drops/min, three one ml fractions are collected with a fifteen-minute incubation between each fraction. The concentrated vector is eluted in fractions two and three. A further two-fold concentration along with a buffer exchange into a storage buffer is achieved via diafiltration with a Vivaspin20 (1 million dalton mwco) (Sartorius) centrifugation apparatus. The concentrated vector is placed into the Vivaspin20 unit, a dialysis cup is inserted, and 12 mls of appropriate storage buffer are added. The unit is centrifuged at 800 g for approximately 40 minutes or until the volume of concentrated vector is reduced to 0.75-1.0 ml. Concentrated vector is filter sterilized with a 0.2 μm PES syringe filter and then aliquoted and stored at −80° C. If the storage buffer contains protein, e.g. BSA, then the BSA is added after the diafiltration step but prior to sterile filtration. This procedure typically leads to about a 100-fold increase in vector titer typically with about a 30% yield. Unless otherwise noted, the storage buffer was PBS supplemented with 2.5 mM KCl and 1.0 mM MgCl₂.

In the following examples, when lentiviral vectors are used to transduce cells in culture, either Polybrene at 8 μg/ml or protamine at 1 or 5 μg/ml was added to the tissue culture medium. All of these polycation additions typically can be used interchangeably. Unless otherwise noted, polycations were not co-administered with the lentiviral vectors when they were injected directly into animals.

### Example Two

### Transduction of Rodent Retinal Cells In Vivo

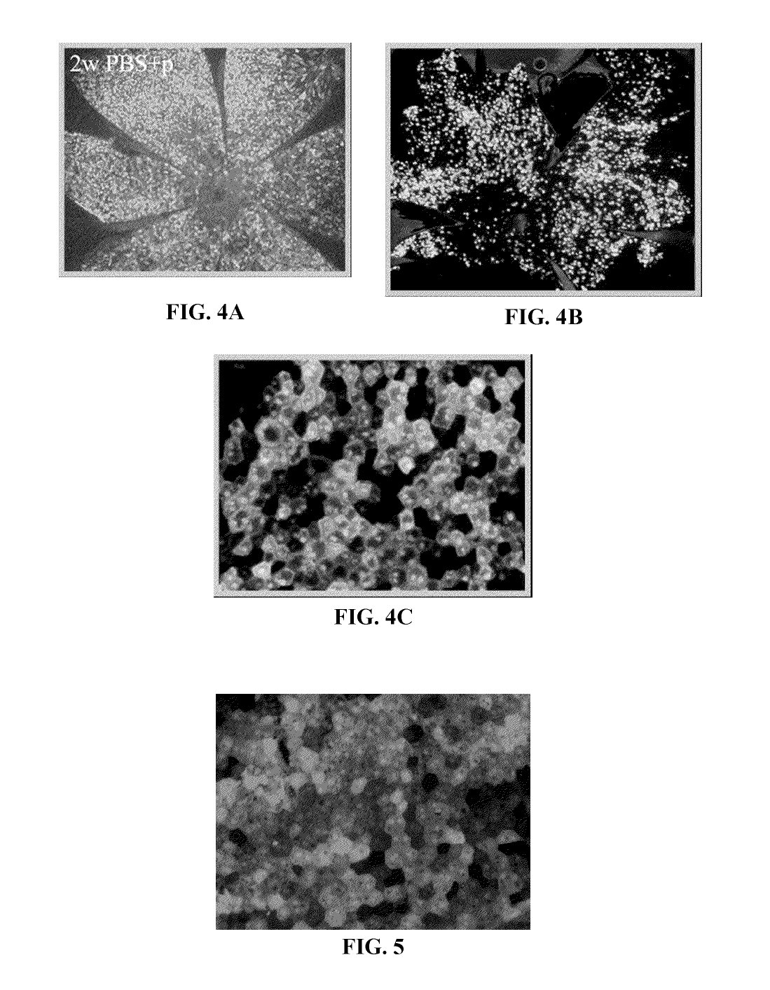

A BIV vector efficiently transduced ocular cells in vivo in both rats and mice. For the rat and the mouse studies shown in FIGS. 3 and 4A, the GFP vector was concentrated by ultracentrifugation and formulated in PBS supplemented with 2% BSA. Polybrene at 8 μg/ml was added to the vector at the time of injection. For the mouse study shown in FIGS. 4B and 4C, the vector was concentrated by the chromatographic method of Example 1 and formulated in PBS. Polybrene was not co-administered with the vector. One to three microliters of vector encoding Green Fluorescent Protein (GFP) with a titer of approximately 1×10⁸ to (transducing units)/ml were injected under the retinas, and the retinas were subsequently examined for GFP expression. The rats were followed for up to nine months and the mice were followed for up to five months. In both animal models, high level GFP expression was seen in the retinal cells at the injection site. Additionally, there was no noticeable loss of expression for the duration of each study. An example of GFP expression in the rat retina is shown in FIG. 3, and examples of GFP expression in mice retinas are shown in FIG. 4. Immunocytochemical analysis of retinal cross sections demonstrated that the vector expressed predominantly in retinal pigment epithelial (RPE) cells, the retinal cell type that underlies the photoreceptor layer.

### Example Three

### Transduction of Rabbit Retinal Cells In Vivo

A BIV vector encoding GFP was prepared by the chromatographic method of Example 1 and formulated in the PBS/KCl/MgCl₂ buffer of Example 1 supplemented with 50 mM trehalose and 0.1% BSA. New Zealand white rabbits, weighing approximately 3 kg each, were anesthetized, and the pupils were dilated with trapicamide 1% and AK-dilate 1%. A drop of alcaine 0.5% was instilled in each eye. A lid speculum was placed, and the eyelids and conjunctiva cul-de-sac were swabbed with povidone iodine. Under a Zeiss operating microscope, paracentesis was performed with 30 g needle to reduce intraocular pressure. A contact lens was placed over the cornea to facilitate viewing the retina. A 25 g needle (SurModics) was pushed through the conjunctiva and sclera 3-4 mm posterior to the limbus in the superior temporal quadrant at a steep angle to avoid the lens. A 39 g cannula was guided through the 25 g needle until slightly touching the retinal surface. A pulse of 100 μl of GFP vector solution, with a titer of 1×10⁸ tu/ml, was injected to create a retinotomy. A subretinal bleb was clearly formed. The needle and cannula were then slowly withdrawn from the eye, which resulted in a self-sealing sclerotomy. Following the surgery, animals received a subconjunctival injection of 0.5 ml Kenalog-G, and a corneal application of ointment containing neomycin, polymixin B, and dexamethasone (Alcon).

Four weeks later, the rabbits were sacrificed, the retinas were harvested, and retinal whole mounts were prepared. As shown in FIG. 5, there was robust expression of GFP in the RPE cells of the rabbit retina. Expression was also demonstrated in the neural retina in a variety of cell types (data not shown).

### Example Four

### Transduction of Monkey Retinal Cells In Vivo

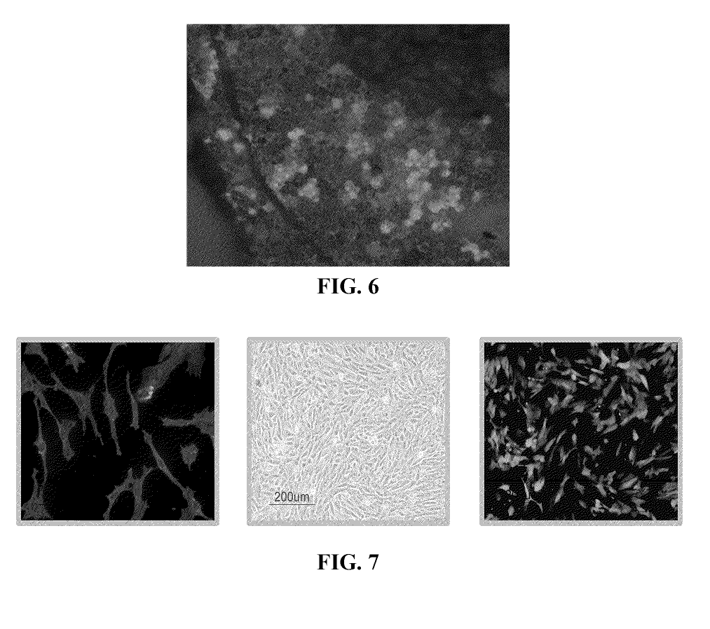

A BIV vector encoding GFP was prepared by the chromatographic method of Example 1 and formulated in HEPES buffered saline (20 mM HEPES pH 7.4, 130 mM NaCl, 1 mM MgCl₂, 50 mM trehalose, 0.1% BSA). The vector titer was 1.5×10⁸ tu/ml. Two cynomolgus monkeys each received 75 μl of the BIV GFP vector in one eye via subretinal injection with a surgical procedure similar to that described for the rabbits (Example 3). One monkey was sacrificed ten weeks later, and a flat mount of the retina was prepared. FIG. 6 shows expression of GFP in the RPE layer of the retina.

### Example Five

### Expression of GFP in Primary Human RPE Cells

Human eyes from a 75-year old female, a 73-year old male, and a 43-year old male were procured from the Lions Eye Bank of Oregon. The eyes were dissected by removing the anterior segment, vitreous, and neurosensory retina. The eyecups were rinsed with PBS and incubated in 2 ml of 0.05% trypsin-EDTA for 10-20 min depending on the condition of the RPE layer. RPE cells were gently scraped off with a spatula and collected in 15 ml conical tubes. The cells were centrifuged 5 min at 100 rpm and washed with PBS. Finally, RPE cells from each eye were resuspended in 1 ml of DMEM+15% FBS prior to seeding in a 12-well culture plate.

The RPE cells grew slowly and took 2-3 weeks to become confluent in the wells. To ensure that the pigmented cells were RPE, they were assayed for expression of the RPE-specific protein, RPE65, by immunofluorescent staining. As shown in FIG. 7, the cells exhibited strong staining with a monoclonal antibody to RPE65 (Novus Biologicals). In contrast, only background staining was observed in controls from which the primary antibody was omitted (data not shown).

The cells were transduced with 5 μl of BIV GFP vector in the presence of 2 μg/ml protamine. The vector was formulated in PBS and had a titer of 1×10⁸ tu/ml. As shown in FIG. 7, the cells exhibited marked GFP expression 48 hrs after transduction.

### Example Six

### The Inducible VEGF Model

As shown in the examples above, BIV vectors are able to transduce retinal cells in vivo in both small and large animal models including non-human primates. Moreover, a BIV vector can efficiently transduce primary human retinal cells in vitro. Additional studies in mice and rats in which the vector was administered via intravitreal, subtenon, and periocular injections as well as via application of the vector directly onto the cornea demonstrated that the vector can transduce other ocular cell types including corneal cells, conjunctival cells, and scleral fibroblasts (data not shown). These data support a gene transfer strategy to cells of the eye, e.g., to ameliorate or stabilize human ocular disorders.

Several studies were performed to verify that BIV vectors are capable of achieving efficacy in disease models relevant to the treatment of human eye disease. In the following two examples, the vectors encoded genes for anti-angiogenic factors to block or inhibit new blood vessel growth and leakage in mouse models of retinal neovascularization. Following injection into the mouse eyes, the vectors genetically modified the retinal cells. The modified cells then secreted the anti-angiogenic factor, which diffused throughout the entire eye and blocked and/or inhibited neovascularization.

A BIV vector encoding the anti-angiogenic protein endostatin (the endostatin cDNA was purchased from InvivoGen, catalog#pbla-hendo18) was evaluated in a very aggressive mouse model of ocular neovascularization (e.g., see Okamoto et al. 1997). The mice were engineered such that, when they were treated with doxycycline, their photoreceptors began to secrete VEGF. Within three days, the VEGF led to significant vascular leakage and new vessel formation. Within one week, the pathology was so severe that the retinas detached from the backs of the eyes.

In each animal, one eye was treated via subretinal injection with a BIV endostatin vector and the other eye was treated with a control vector that did not encode a therapeutic protein (Takahashi et al., The FASEB Journal, published online Mar. 28, 2003). Three weeks later, doxycycline was administered. Five days later, vascular leakage was evaluated in the live animals by fluorescein angiography.

In this evaluation, fluorescein is administered via intravenous injection, and vascular leakage in the retina is visualized by a diffuse fluorescence pattern in the back of the eye.

A separate cohort of 10 mice was sacrificed seven days after doxycycline treatment. Histological sections of the retinas were examined for the thickening that results from vascular leakage, and cross-sections of the entire eyes were examined for the severe consequence of retinal detachment.

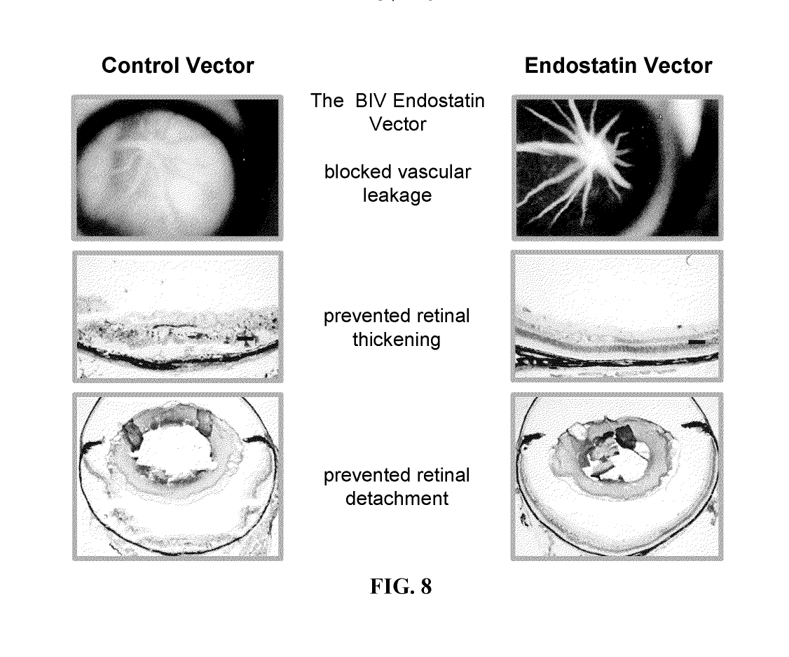

The results are depicted in FIG. 8 and are provided in Takahashi et al. (2003). Fluorescein angiography revealed extensive vascular leakage in the control eyes but normal or nearly normal vascular patterns in the endostatin vector-treated eyes. The histological evaluations confirmed these results. Retinas from the control eyes exhibited severe thickening from vascular leakage, whereas retinas from the endostatin vector-treated eyes appeared normal or minimally thickened. Finally, the control eyes suffered from partial or complete retinal detachment, whereas the endostatin vector-treated eyes demonstrated much less and, in some cases, no retinal detachment. Overall, the BIV endostatin vector protected the treated eyes in 80% of the mice. Moreover, the therapeutic benefit extended throughout each retina and was not limited to the injection site.

These data indicate that a BIV vector has the potential to prevent ocular neovascularization and leakage.

### Example Seven

### The Laser Injury Model

BIV vectors with two different transgenes were evaluated in the laser injury model of ocular neovascularization. This model, which is well accepted for the development of ocular therapeutics, uses a laser burn to the retina to stimulate neovascularization (e.g., see Gehlbach et al. Hum Gene Then 2003 14(2):129-41; Mori et al. Invest Ophthalmol Vis Sci. 2002 43(6):1994-2000; and Mori et al. J Cell Physiol. 2001 188(2):253-63). In brief, the burn creates a hole in the retina allowing new capillaries to grow into the retinal from the underlying choroidal capillary bed. The vessels are usually defective and are leaky.

In the first study, a BIV vector encoding the anti-angiogenic protein Pigment Epithelial-Derived Factor (PEDF) (the cDNA for PEDF was purchased from InvivoGen, catalog# pbla-hpedf) was administered via subretinal injection into rat eyes. In each rat, one eye received the PEDF vector and the other received a control BIV vector that did not encode a therapeutic transgene. Two weeks later, neovascularization was induced by laser injury. After an additional two weeks, the rats were treated with FITC-dextran, which outlines the new vessels. The retinas were harvested and examined for vessel structure. The results are shown in FIG. 9. As expected, the eyes treated with the control vector demonstrated pathological neovascularization. In contrast, the new capillaries in the eyes treated with the PEDF vector had an appearance that was characteristic of resolving neovascularization.

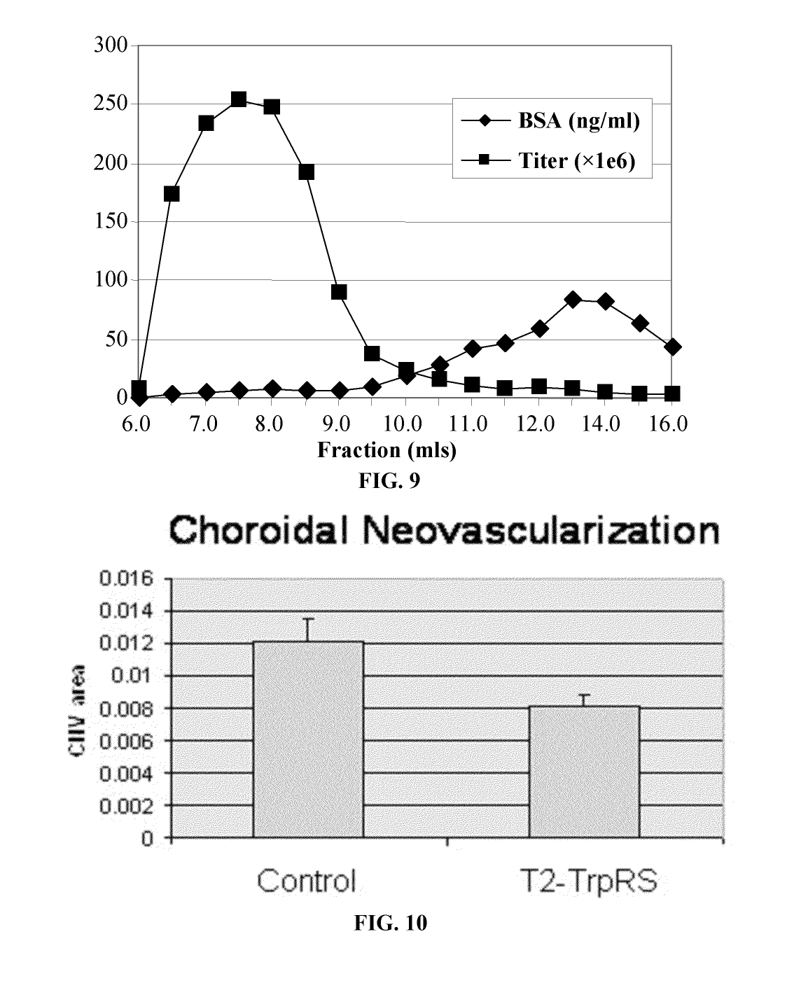

In the second study, a BIV vector encoding the anti-angiogenic protein T2-TrpRS (the cDNA for T2-TrpRS was purchased from InvivoGen, catalog# pbla-htrprs) was evaluated in mice with the laser injury model. One eye of each mouse received the T2-TrpRS vector, the other received the control vector. On the same day, neovascularization was induced by laser injury. Two weeks later, the size of the neovascular areas was evaluated with FITC-dextran and serial sectioning. The data in FIG. 10 show that the average neovascular area was significantly smaller in the eyes treated with the T2-TrpRS vector indicating that this vector was highly efficacious in inhibiting neovascularization.

The data from these PEDF and T2-TrpRS animal studies support, inter alia, the use of gene transfer for the amelioration and/or stabilization of eye disease.

### Example Eight

### Design of Therapeutic Proteins that can Attenuate Complement Activation

A wild type human fB cDNA with its native flanking sequences at both the 5′ and 3′ ends was obtained by PCR from a human liver cDNA library (Origene Technologies, Inc., Catalog# CH1005). The two primers used for the PCR were: 5′-CTAGCTAGCTCCTGCCCCAGGCCCAGCTTCTCTCC-3′ (Forward primer) (SEQ ID NO:17) and 5′-CTAGCTAGCTCAATCCCACGCCCCTGTCC-3′ (Reverse primer) (SEQ ID NO:18). Both primers contained Nhe I sites. The amplified PCR products were digested with Nhe I and ligated into a BIV transfer vector plasmid previously digested with Nhe I (see Example 15). In this construct, the MNC promoter is used to drive fB transcription. The sequences of the vector and fB were confirmed. The DNA sequence for a wild type human fB is shown in SEQ ID NO:1, and the corresponding amino acid sequence is shown in SEQ ID NO:2. The schematic illustration for the lentiviral transfer vector construct encoding fB is shown in FIG. 2. In this case, the section of the construct labeled Heterologous Gene is the fB sequence. It should be noted that the nucleic acid sequences of the Sequence Listing include flanking sequences.

Subsequently, three DNA sequences encoding human dominant negative fB mutants/analogs (fB1, fB2, and fB3) were directly synthesized (Geneart, Inc.). These three DNA sequences were identical to SEQ ID NO:1 with the exception of specific mutations in the fB coding region. At the amino acid level, fB1 contains the change D740N. The DNA and amino acid sequences of fB1 are shown in SEQ ID NOs:3 and 4, respectively. At the amino acid level, fB2 contains the changes D279G, N285D, and D740N. The DNA and amino acid sequences of fB2 are shown in SEQ ID NOs:5 and 6, respectively. At the amino acid level, fB3 contains the changes K258A, R259A, K260A, D279G, and N285D. The DNA and amino acid sequences of fB3 are shown in SEQ ID NOs:7 and 8, respectively. Each dominant negative human fB construct was subcloned into a BIV transfer vector plasmid as an Nhe I fragment. All of the constructs were sequenced to verify their integrity.

The three analogous mouse dominant negative fB mutants/analogs were also directly synthesized (Geneart, Inc.) and subcloned into a BIV transfer vector plasmid as NheI fragments. Mouse fB 1 contains a D737N change. The sequences are shown in SEQ ID NOs:9 and 10, respectively. Mouse fB2 contains D276G, N282D, and D737N changes. The sequences are shown in SEQ ID NOs:11 and 12, respectively. Mouse fB3 contains K255A, R256A, K257A, D276G, and N282D changes. The sequences are shown in SEQ ID NOs:13 and 14, respectively. The mouse wild type fB was obtained by reverse engineering mfB1. Specifically, the N at position 737 was converted to a D by site-directed mutagenesis with the Quick Change kit (Stratagene Inc.). The sequences for mouse wild type fB are shown in SEQ ID NOs:15 and 16, respectively. All of the constructs were sequenced to verify their integrity.

Herein, human factor B wild type and mutants/analogs are designated as such with the letter h (e.g. hfB1) and the mouse analogues are designated with an m (e.g. mfB1).

Lentiviral vector preps were generated as outlined in Example 1, and the vectors were evaluated in vitro for expression. Vector supernatants were used to transduce ARPE cells, a human retinal pigment epithelial cell line (ATCC, CRL-2302). Transduction of cells with BIV vectors has been described previously (e.g., Matukonis et al., 2002; Molina et al., 2004). Briefly, the ARPE cells were plated in 6-well plates at a density of 1×10⁵ cells per well. The following day, the cells were transduced with 3 ml of vector supernatant in the presence of 5 μg/ml protamine sulfate (Sigma) at 37° C. in a 5% CO₂ incubator. Five hours later, the medium was replaced with fresh cell culture medium (DMEM with 10% FBS). The transduced cells were cultured for 72 hours at which time the culture medium was subjected to SDS-PAGE and Western-blot analysis for fB expression.

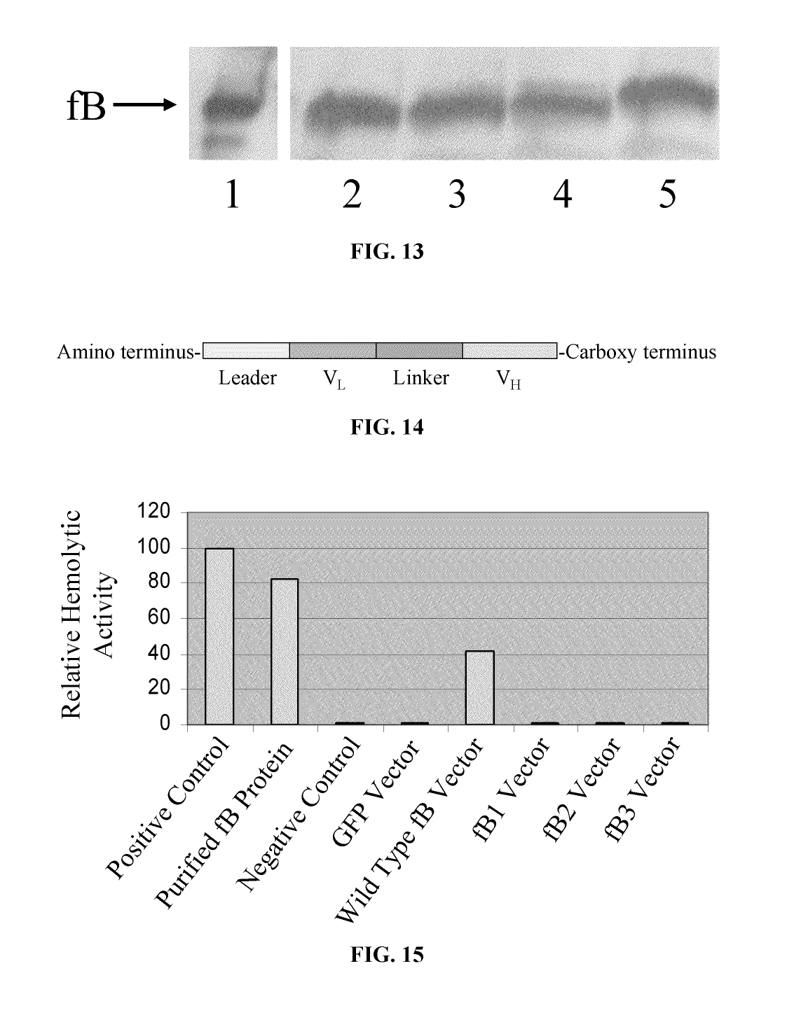

Forty μl of cell culture medium was mixed with 10 μl of 5×SDS-sample buffer and heated to 95° C. for 3 minutes. The sample was then separated on a 7.5% SDS-polyacrylamide gel. The separated proteins were transferred onto a nitrocellulose membrane, which was probed with a goat-anti human fB serum (Nordic Immunological Laboratories, Catalog# GAHu/PFB). The membrane was then incubated with biotinylated rabbit anti-goat IgG (Vector Laboratories, Catalog# BA-5000) followed by an avidin-biotinylated alkaline phosphatase complex. Finally, the membrane was incubated with alkaline phosphatase substrate (Vector Laboratories) to visualize the bands. Representative data for human wild type fB, fB1, fB2, and fB3 are shown in FIG. 13. fB proteins with the correct molecular weight were secreted from the vector-transduced cells at levels of 2.5 μg/ml. Similar data were obtained with vectors encoding the mouse wild type and dominant negative fB proteins (data not shown). These data indicate that BIV lentiviral vectors can mediate efficient expression of fB proteins in retinal cells and support the potential for clinical application.

### Example Nine

### Binding Molecule Strategy to Inhibit Complement fB Activity

A second strategy to inhibit the alternative complement pathway involves generating antibodies (e.g., monoclonal) or fragments thereof that neutralize or inhibit fB activity. Monoclonal antibodies are routinely generated in mice, rabbits, and chickens. For this example, the inventors used rabbits. The use of rabbits optimizes the likelihood of obtaining a monoclonal antibody that cross-reacts with mouse fB for rodent studies. Obviously this simplifies studies in rodents, but is not a requirement for inhibiting human fB. An exemplary strategy further involves cloning antibody sequences from the hybridoma, e.g., via RT-PCR, to generate a recombinant single chain antibody. Techniques for preparing single chain antibodies are well known to those skilled in the art (Carolina et al. 1994). If necessary, the antibody can be further optimized for therapeutic use by humanizing the framework sequences, again by procedures well known to those skilled in the art (Adams et al. 2005). Additionally, binding properties of the antibody or antibody fragment can be altered, for examples see U.S. Pat. Nos. 7,175,996 and 6,656,467. Finally, an antibody(s) (e.g., a single chain antibody that neutralizes fB can be encoded in the vector and delivered via gene transfer. In some embodiments, an antibody or fragment thereof can be delivered.

The process of generating rabbit monoclonal antibodies against human fB is as follows. Purified (>95%) plasma-derived human fB is obtained from Quidel (San Diego, Calif.). Each rabbit is immunized up to three times with a total of 2.5 mg of human fB. Six weeks to three months after the initiation of the immunization protocol, serum samples from the rabbits are checked by ELISA for antibody titers against fB. The serum samples are also assayed for neutralizing titers in the hemolytic assay (see Example Eleven). Both human-specific and mouse-specific hemolytic assays are performed to reveal antibodies that cross-react with human and mouse fB. Rabbits with high titer serum are used for monoclonal antibody generation. The spleens are removed for cell fusion. Hybridomas are isolated and monoclonal antibodies from each are screened by ELISA and for human and mouse fB neutralizing titers. The hybridomas that secrete monoclonal antibodies with the highest neutralizing titers are subcloned one to three times to insure the stability of antibody production. With each subcloning, the clones are screened for neutralizing titers. The best clones are then chosen for RT-PCR amplification of the antibody sequences to produce recombinant single chain antibodies. Briefly, primers are designed that flank the antibody variable regions, which confer antigen binding specificity. The variable regions are then PCR amplified and the resulting DNA sequences are used to construct single chain antibodies with the general structure shown in FIG. 14. The recombinant single chain antibodies are re-evaluated to insure that they still exhibit fB binding and neutralizing activities. The single chain antibodies are then subcloned into the transfer vector construct of FIG. 2 to generate BIV vectors for in vitro and in vivo evaluations. A single chain antibody chosen for clinical application in humans can be further modified to humanize the framework regions. Finally, if necessary, directed evolution techniques are employed to further increase the affinity of the antibody for fB and to further improve the efficacy of fB neutralization (e.g., see Broder et al. 2000). These techniques include, but are not limited to, CDR grafting, framework shuffling, and resurfacing technologies.

In some embodiments, individual CDRs are PCR amplified from the hybridomas and combined into DNA constructs that encode polypeptides with antigen-binding capacity. Although such polypeptides do not have the structure of single chain antibodies, they efficiently neutralize fB and can serve as proteins (e.g., therapeutic) to be delivered via BIV vectors or delivered as proteins.

Whereas the antibodies in this example are generated against plasma-derived human fB, equally effective antibodies can also be generated from recombinant fB or even from manufactured fB polypeptides (e.g., 20 amino acid epitopes). Finally, an identical or similar strategy can be used to generate antibodies that neutralize any component(s) of the complement pathway. Complement factor D (fD) is an excellent target for an antibody strategy to attenuate the alternative complement pathway since fD is found in plasma and in the eye at very low levels (approximately 1-2 μg/ml) and mediates a rate limiting step in the alternative pathway (Volanakis & Narayana et al. 1996 Protein Science 5:553-564). Moreover, recombinant single chain antibodies that neutralize fD can be used in combination with the technologies described above that attenuate fB activity.

The steps of rabbit immunization, hybridoma generation, subcloning, and antibody collection can be performed by commercial entities such as Genesis Biotech, Inc. in Taiwan. Additionally, numerous companies provide the service of generating mouse monoclonal antibodies (e.g. Covance or Charles River Laboratories).

### Example Ten

### Hemolytic Assays to Evaluate Examples of fB Dominant Negatives

BIV vectors encoding the human wild type fB and the three dominant negatives were used to transduce ARPE cells, and the functional activities of the secreted fB proteins were evaluated with a hemolytic assay of the alternative complement pathway. This assay utilizes rabbit erythrocytes, which spontaneously activate the human and mouse alternative pathways (Sohn et al. 2000). First, a cell suspension of unsensitized rabbit erythrocytes (Erab) at 2×10⁸ cells/ml was prepared in gelatiniveronal buffered saline plus Mg⁺⁺ and EGTA, pH 7.35 (GVB-EGTA). Second, 20 μl of fB-depleted human serum (Quidel) was diluted 1:5 with GVB-EGTA containing: (1) no additive; (2) 500 ng of plasma-derived human fB (Quidel); or (3) 40 μl of culture medium from ARPE cells that had been transduced with vectors encoding either GFP, wild type human fB, fB1, fB2, or fB3. The culture media had been concentrated ten-fold with an Amicon Ultra Centrifugal Filter (Millipore, catalog# UFC803008). Then 100 μl of each serum mixture was added to 100 μl of Erab and incubated for 60 min at 37° C. in a shaker water bath. Ice-cold NaCl (0.15 M) was used to stop the reactions. The tubes were centrifuged at 1250 g for 10 minutes at 4° C. to pellet the cells, and the OD₄₀₅ of each supernatant was determined. For the positive control, distilled water was added to the Erab suspension, which resulted in osmotic lysis of 100% of the cells.

The data are presented in FIG. 15. When added to the fB-depleted serum, 500 ng of purified plasma-derived human fB yielded 80% hemolysis (compared to the 100% obtained through osmotic lysis). The fB-depleted serum without the addition of any fB yielded no hemolysis. Tissue culture media from cells transduced with a GFP vector did not restore the hemolytic capacity of the serum. In contrast, tissue culture media from cells transduced with the wild type human fB vector yielded 40% hemolysis. As expected, tissue culture media from cells transduced with vectors encoding each of the three human dominant negative fB moieties did not restore the hemolytic capacity of the serum. These data verify that vector-derived human wild type fB is biologically active and that the dominant negatives do not activate the alternative complement pathway.

### Example Eleven

### Use of Hemolytic Assays to Evaluate Proteins that Inhibit a Complement Pathway

Competition assays can be used to evaluate the potency of vectors encoding dominant negative fB moieties. fB dominant negative proteins are prepared in tissue culture by vector transduction of ARPE cells. The fB proteins are secreted into the tissue culture medium and quantified by Western analysis as shown in Example Eight. Competition assays are performed in which varying ratios of a dominant negative fB and wild type fB are added to the hemolytic assay. The potency of each dominant negative fB is determined by its ability to compete with the wild type fB protein and attenuate the wild type protein's capacity to reconstitute the hemolytic activity of fB-depleted serum. Vectors encoding the proteins can be also evaluated in animal models and in some cases ones with a desired potency (e.g., the most potent) are further developed, such as for studying complement pathways and/or for application in animal (e.g., humans).

As noted herein, fB1 is believed to bind C3b with normal affinity and kinetics, but when acted upon by fD and stabilized by properdin, fB1 does not function as a protease and does not form a C3 convertase. fB2 has an increased binding affinity for C3b while inactivating the protease function. fB3 has an increased binding affinity for C3b but cannot be cleaved by factor D and should therefore have minimal protease activity. fB1, fB2, and fB3 are tested in the competition assay with wild type fB.

The potency of the anti-fB antibodies is evaluated in a similar hemolytic assay, as described in Example Ten, using fB-depleted serum. Specifically, the assay measures the potency with which dilutions/concentrations of each antibody block or inhibit the capacity of purified fB to reconstitute hemolytic activity. The initial in vitro evaluations of the anti-fB antibodies in the hemolytic assay are performed with antibody proteins without using a vector for the antibody's production. That is, purified antibody is added to the assay to evaluate the potency with which each antibody diminishes hemolysis. Subsequent in vitro evaluations with the hemolytic assays are performed with tissue culture media from ARPE cells that are transduced with vectors encoding single chain versions of the antibodies, e.g., of the most potent antibodies.

### Example Twelve

### In Vivo Analyses of Vectors Encoding fB Dominant Negatives and fB Neutralizing Antibodies

In vivo evaluations in mice are performed initially with the mouse fB dominant negatives and with those antibodies that neutralize mouse fB. The human fB dominant negatives are also evaluated in vivo in mice, although species specificity may interfere with the reliability of these evaluations.

A mouse model used to evaluate the vectors encoding the potential therapeutic proteins is the laser injury model (Campochiaro and Hackett 2003). As described in Example Seven, a laser pulse is used to create a hole in Bruch's membrane through which new blood vessels grow from the choroidal capillary bed. The extent of blood vessel growth is quantified by FITC-dextran infusion one to two weeks after the laser treatment. Interestingly, the new blood vessel growth is dependent upon activation of the alternative complement pathway. Recent data demonstrate that when the alternative pathway is inhibited or blocked in mice, laser-induced neovascularization is substantially diminished (Bora, N. et al. 2006, Bora, P. et al. 2006, and Bora, N. et al. 2007). Therefore, this model provides a facile method for assessing the effectiveness with which the gene transfer vectors of the instant invention inhibit complement activation in vivo. Since the model assays complement inhibition, it has predictive value for the development of therapeutics to treat human diseases whose etiology involves complement activation including all forms of AMD (early dry AMD, wet AMD, and Geographic Atrophy). Finally, it is noteworthy that the complement components C3a and C5a found in human drusen have been shown to induce VEGF expression in vitro suggesting that the mechanism of neovascularization in mice may be very similar to that in humans (Nozaki et al. 2006).

Briefly, vectors encoding fB dominant negatives or a control vector that encodes an irrelevant protein (e.g., does not encode a therapeutic protein) are injected via subretinal and/or intravitreal injection into mice, e.g., neonatal (p5) or adolescent (approximately 6 week old) C57Bl/6 mice. When the animals are approximately eight weeks old, laser injury is performed generating three spots per retina. Many different laser injury procedures are known to those skilled in the art. The inventors typically use an Iris Oculight SLX laser that contains a red diode and emits light with a wavelength of 810 nm. The laser parameters are typically set for a beam diameter of 75 μm, energy level of 100 mwatts, and pulse duration of 100 msec. Seven days after the laser injury, the animals are perfused with FITC-dextran, the retinas are harvested, and the extent of neovascularization is determined by confocal microscopy.

To test an antibody strategy, laser injury is performed in adolescent C57Bl/6 mice. The same day, the animals are treated with an intravitreal injection of monoclonal anti-fB antibody. In each case, the negative control cohort is treated with an irrelevant antibody.

As with the in vitro analyses, typically, but not necessarily always, hybridomas expressing the most potent antibodies are used to generate recombinant single chain antibodies. These single chain antibodies are then encoded in a gene transfer vector and tested in the mice in a manner identical to that described for the fB dominant negatives. For human applications, a recombinant single chain anti-fB antibodies can be optionally further modified to humanize the framework regions.

Optionally, directed evolution techniques are employed to further increase the affinity of the antibody for fB and to further improve the efficacy of fB neutralization (Broder et al. 2000).

### Example Thirteen

### Delivery of fB Dominant Negatives and Anti-fB Antibodies with a Vector System that is not Based on a Lentivirus

Whereas the previous examples have focused on lentiviral vector gene transfer systems, and, in particular, a BIV-based vector system, the proteins or therapeutics can be delivered to the eye via other vector systems. For example, dominant negative fB moieties and single chain antibodies described above are easily encoded in AAV vectors. The use of AAV vectors is quite facile and is well known to those skilled in the art (e.g., see Lu 2004; U.S. Pat. Nos. 7,037,713; 6,953,575; 6,897,063; 6,764,845; 6,759,050; 6,710,036; 6,610,290; 6,593,123; 6,582,692; 6,531,456; 6,416,992; 6,207,457; and 6,156,303). There are at least eight AAV serotypes with varying gene transfer efficiencies in vivo and varying onsets of expression. Most serotypes of AAV vectors work in the eye (e.g., see Aurricchio et al. 2001 and Yang et al. 2002). An AAV vector system is commercially available through Stratagene (La Jolla, Calif.) along with a detailed instruction manual. The steps of subcloning the therapeutic proteins into the vectors, generating the vector preps by transient transfection, and purifying the vector by density gradient ultracentrifugation or column chromatography are facile and well known to those skilled in the art. AAV vectors encoding the proteins, e.g., described in the previous Examples, are evaluated in vitro and injected into animal models via the same or similar procedures described for the BIV vectors.

### Example Fourteen

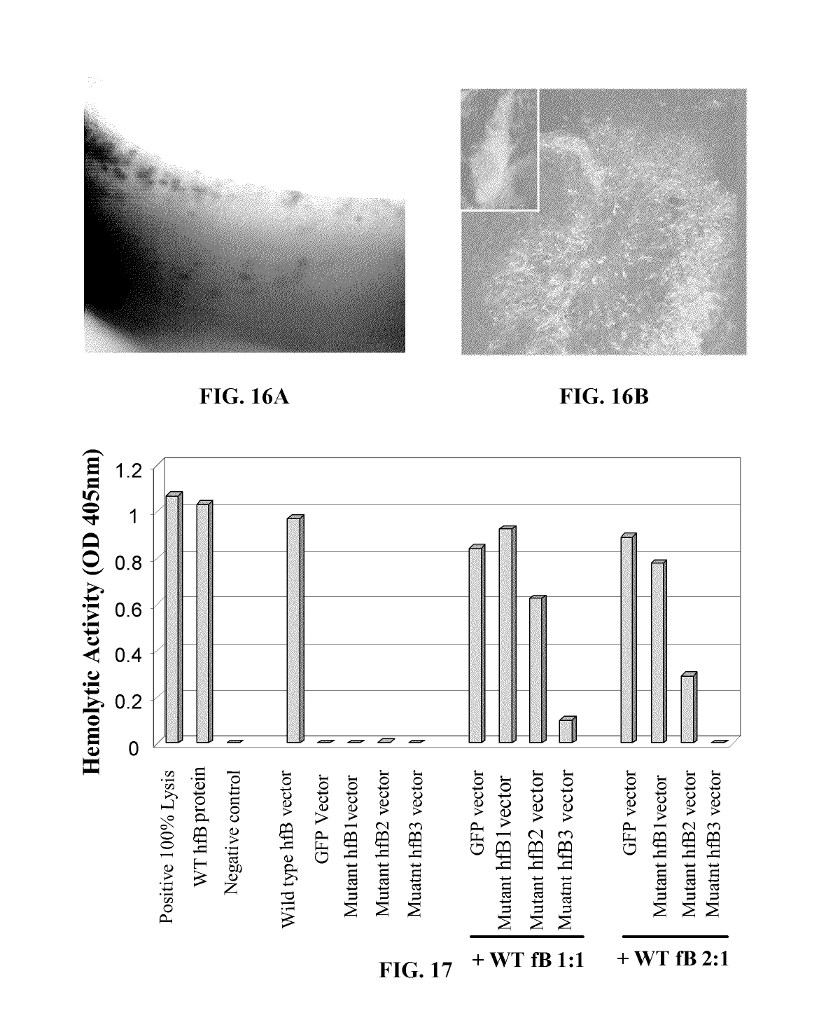

### Application of the Instant Invention to the Treatment of Human Diseases Such as Atherosclerotic Cardiovascular Disease

Whereas the previous Examples have focused on ocular diseases, it is noteworthy that many different diseases in humans have complement activation as an etiology. These include, among others, rheumatologic, neurologic, and cardiovascular diseases (e.g., see Niculescu and Rus 2004, Kardys et al. 2006, and Rus et al. 2006). Therefore, some vectors of the instant invention have immediate application to the inhibition, stabilization and/or treatment of diseases other than eye diseases, e.g., via direct injection of vectors or proteins of the invention into a diseased organ. In particular, atherosclerotic plaques may grow, at least in part, via the same etiology as that described for drusen in the model of AMD provided herein. Local expression and/or delivery of complement inhibitors will slow the development of the plaques and slow progression of the disease. In some embodiments to treat coronary artery or peripheral artery diseases, vector is administered to blood vessels. If a treatment involves angioplasty, a vector and/or protein can be administered via catheter, e.g., to a site of the angioplasty. If a treatment involves vascular grafting, a vector and/or protein can be infused through a vessel prior to grafting. In some embodiments, vector and/or protein can attenuate local inflammation and slow the recurrence or progression of atherosclerotic plaques. FIG. 16 provides support for non-ocular applications by demonstrating the efficiency with which lentiviral vectors transfer genes to blood vessels and brain.

FIG. 16 shows lentiviral vector gene transfer to rat aorta and mouse brain. FIG. 16A demonstrates transduction of a section of rat aorta. In this case, the vessel was infused with a lentiviral vector derived from HIV that encoded β-galactosidase. The β-galactosidase reporter protein was engineered to localize in the cell nucleus. The vessel was subsequently sectioned and stained for β-galactosidase, which produces a blue color. The blue nuclei shown in FIG. 16A indicate efficient gene transfer to endothelial and smooth muscle cells along the luminal surface and, to some extent, throughout the vessel wall. The generation and use of HIV vectors is well known to those skilled in the art, and an HIV vector system is commercially available from Invitrogen (Carlsbad, Calif.). FIG. 16B demonstrates gene transfer to mouse brain using a BIV GFP vector. One μl of vector was administered via stereotactic injection to the substantia nigra. Seventeen days later the mouse was sacrificed and the brain was sectioned. GFP expression is seen in both the neurons and glial cells. Interestingly, GFP expression is noted on both sides of the brain even though only one side was injected. The brain was stained for neurons with NeuN, shown in red. The inset shows, at high power, the yellow co-incidence of green and red staining verifying the transduction of neurons.

The combination of in vitro and in vivo analyses described above support, inter alia, a gene transfer strategy for the treatment of human eye diseases with gene transfer vectors encoding anti-inflammatory therapeutic proteins. The studies of this example also demonstrates the potential of the instant invention to treat other human diseases such as cardiovascular and neurologic diseases.

### Example Fifteen

### Inhibition of Human Alternative Complement Pathway Activity by Human Factor B Mutants

### Materials, Methods and Equipment

Beckman Allegra 6KR Centrifuge, C76 Water bath shaker (New Brunswick Scientific Classic Series), Fisher Vortex Genie 2, Molecular Device Spectra Max 190 microplate reader, Corning 96 well plate white, 14 ml polystyrene round bottom tubes (Fisher), Amico Ultra filter device with molecular weight cutoff 30K (Millipore).

### Reagents and Buffers

GVB⁺⁺ (Sigma) contained 5 mM Barbital buffer, 0.15 mM CaCl₂, 141 mM NaCl, 0.5 mM MgCl₂. Mg²⁺-EGTA buffer was prepared fresh for each use and contained 100 mM EGTA, 100 mM MgCl₂, GVB⁺⁺ and 5% Glucose. Rabbit Erythrocytes were purchase from Innovative Research. Human factor B depleted serum was purchased from Quidel (Catalog #A506).

Production of Human Factor B Wild Type Protein and Three Dominant Negative Human Mutant Proteins, fB1, fB2, and fB3 Encoded by BIV Vectors

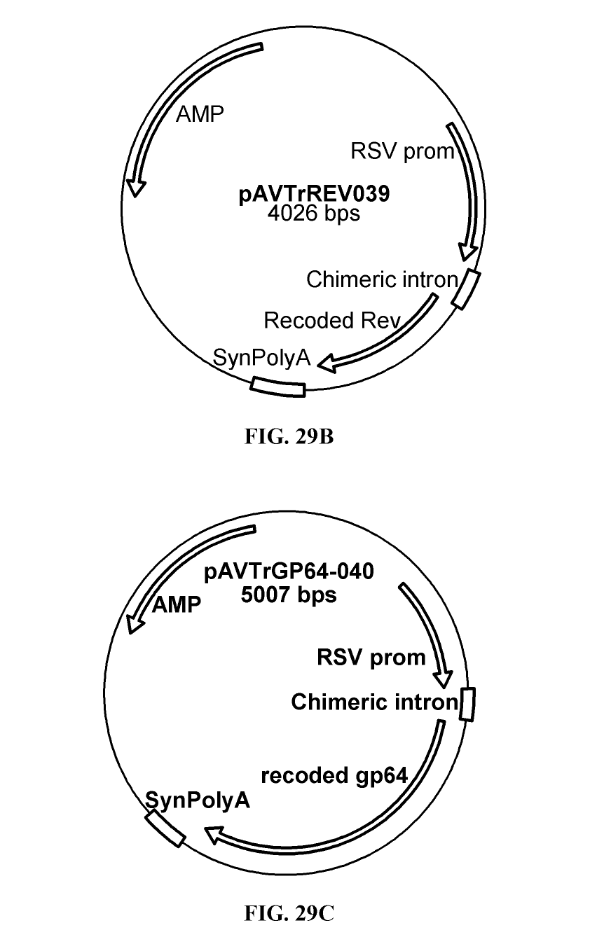

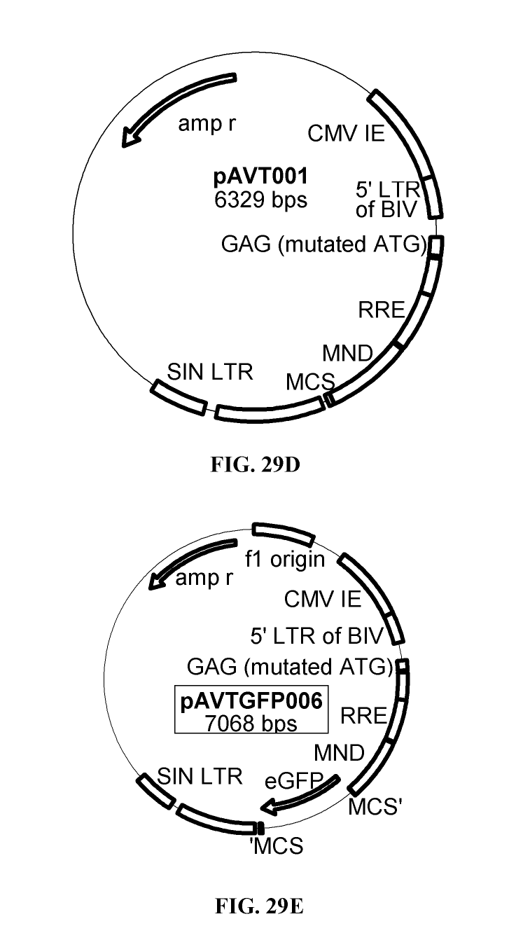

Plasmids were constructed for the production of BIV based vectors. pAVTrGP038 (SEQ ID NO:19; FIG. 29A) has an RSV promoter operatively linked to a BIV gag/pol coding region (that codes for a threonine to serine mutation in the DTGAD motif of the protease) followed by a synthetic polyA signal, e.g., see U.S. Pat. No. 7,070,993. The codons of the gag/pol coding sequence have also been optimized for expression, e.g., see Molina et al. Hum Gene Ther. 2004 15(9):865-77. pAVTrREV039 (SEQ ID NO:20; FIG. 29B) contains an RSV promoter operatively linked to a BIV rev coding region (that was recoded with optimal codons) followed by a synthetic polyA signal. pAVTrGP64-040 (SEQ ID NO:21; FIG. 29C) codes for a GP64 envelope. BIV-based transfer vectors coding for human wild-type factor B, fB1, fB2 and fB3 (SEQ ID NOs:2, 4, 6 and 8, respectively) were all prepared by cloning their respective coding regions (see the coding regions of SEQ ID NOs:1, 3, 5 and 7, respectively and Example 8) into the Nhe I site of pAVT001 (SEQ ID NO:22; FIG. 29D).

To generate BIV vector particles encoding wild type human factor B and three dominant negative human factor B mutants, 293FT cells were plated in 150-mm dishes at 1.1×10⁷ cells/dish. The following day the cells were transfected as described in Example 1 with 45 μg of the BIV-based packaging construct, pAVTrGP038, 45 μg of the BIV-based transfer vector construct encoding wild type human factor B protein, fB1, fB2, or fB3, 30 of Rev expression construct, pAVTrREV039 and 15 μg of GP64 envelope expression construct, pAVTrGP64-040. A control BIV vector encoding eGFP was similarly prepared using pAVTGFP006 (SEQ ID NO:23; FIG. 29E). Thirty-six hours post-transfection, the vector supernatants were harvested and centrifuged at 2000 rpm for 10 min at 4° C. to clear cell debris.

To generate wild type human factor B and three dominant negative human factor B mutant proteins, 2×10⁵ ARPE cells or Cf2Th cells were transduced with 3 ml of tissue culture media containing the appropriate vector. As a control, cells were transduced with 3 ml of vector encoding eGFP. To enhance transduction, protamine sulfate was added to the wells at a final concentration of 8 μg/ml. After 6 hours, vector supernatant was aspirated and replaced with 3 ml of fresh cell DMEM culture medium containing 10% heat-inactivated fetal bovine serum. After the cells reached confluence, the medium was replaced with 1.5 ml of medium without phenol red containing 2% heat-inactivated fetal bovine serum. Ninety-six hours later, the cell culture medium was collected and centrifuged at 2000 rpm for 10 min to clear the cell debris and then filtered through a 0.2 μm filter. The harvested cell culture medium was then concentrated five fold with a Millipore Amico Ultra filter device with a molecular weight cutoff of 30K. The protein expression levels of the wild type human factor B and the three human dominant negative factor B mutants were assessed by Western blot and were found to be essentially equivalent, e.g., within 2-fold by visual observation. The concentrated media containing the wild type and the dominant negative mutant human factor B proteins were filter sterilized and stored as aliquots at −80° C. until use.

### Procedure for the Alternative Complement Pathway Hemolytic Activity Assay

The following assay is a competition assay that utilizes human serum that is depleted of factor B. This factor B-depleted serum, by itself, is devoid of detectable complement-mediated hemolytic activity. The addition of human wild type factor B to the assay reconstitutes the serum hemolytic activity. The concurrent addition of culture supernatant containing a dominant negative factor B protein ([3], fB2, or fB3) is performed to demonstrate whether a dominant negative competes with wild type factor B and attenuates the reconstitution of hemolytic activity.

One ml of rabbit erythrocyte suspension (Erab) was transferred into a 50 ml conical centrifuge tube and washed with 30 ml of freshly made cold Mg²⁺-EGTA buffer. The Erab were centrifuged in the cold Mg2⁺-EGTA buffer with a Beckman Allegra 6KR centrifuge at 1200 rpm at 4° C. for 5 min. with the brake turned off. The Erab were washed 2 more times and resuspended in 2 ml of ice-cold Mg²⁺-EGTA buffer. Cell counts were performed with a hemocytometer. It should be noted that the EGTA functions to inhibit the classic complement pathway without affecting the alternative complement pathway.

The first arm of the study was designed to demonstrate that fB1, fB2, and fB3, by themselves, do not reconstitute the hemolytic activity. The second arm of the study was designed to show that fB1, fB2, and fB3 block the capacity of wild type fB to reconstitute hemolytic activity. The hemolytic reaction mixture was set up on ice in 14 ml polystyrene round bottom tubes. For the first arm of the study, 40 μl of culture medium containing wild type fB, fB1, fB2, or fB3, prepared as described above, was added to each tube. For the second arm of the study, 40 μl of a mixture of culture media containing wild type factor B and either fB1, fB2, or fB3 at the indicated ratios was added to each tube. Then, 50 μl of 25-fold diluted human factor B-depleted human serum was added to each tube. The factor B-depleted human serum was diluted in the freshly made ice-cold Mg²⁺-EGTA buffer. The tubes were vortexed thoroughly with a Fisher Vortex Genie 2 device. Then, 10 μl of Mg²⁺-EGTA washed Erab containing 5×10⁷ erythrocytes was added to each tube followed by gentle mixing without vortexing. The tubes were incubated in a 37° C. water bath with orbital shaking at 110 rpm per min for 40 min. Then, each tube was placed on ice and 150 μl of ice-cold 0.9% saline was added to stop the reaction. The tubes were gently mixed and centrifuged at 2000 rpm in a Beckman Centrifuge for 5 min at 4° C. with the brake turned off. Without disturbing the pellet, 180 μl of supernatant was transferred into a flat-bottom 96-well plate and the OD 405 was determined in a 96 well microplate reader.

Additionally, two control tubes were prepared to establish the OD readings for 100% and 0% Erab lysis. For 100% lysis, the tube contained 40 μl of culture medium from cells transduced with the GFP vector and 10 μl of Mg²⁺-EGTA washed Erab containing 5×10⁷ erythrocytes. After the 37° C. incubation in the orbital shaker, 200 μl of ice-cold water was added to osmotically lyse the red blood cells. For the 0% lysis (blank), the tube contained 90 μl of culture medium from cells transduced with the GFP vector and 10 μl of Mg²⁺-EGTA washed Erab containing 5×10⁷ erythrocytes. After the 37° C. incubation in the orbital shaker, 150 μl of ice-cold 0.9% saline was added to prevent red blood cell lysis.

### Results

As noted, human wild type fB and three human dominant negative fB moieties were made in tissue culture medium from BIV vector-transduced cells. The potencies with which the fB1, fB2, and fB3-containing culture supernatants competed with the wild type fB to inhibit alternative complement pathway hemolytic activity are shown in FIG. 17. The data indicate, inter alia, that: (1) human wild type fB encoded by a vector can functionally substitute for endogenous human complement factor B and did reconstitute the alternative pathway hemolytic activity in factor B-depleted human serum; (2) human fB1, fB2, and fB3 did not, by themselves, function like wild type fB and did not reconstitute the alternative pathway hemolytic activity in factor B-depleted human serum; (3) fB1, at the ratios shown, demonstrated no significant inhibitory activity in blocking alternative complement pathway activity (it is noteworthy that, at a higher fB1 to wild type fB ratio of 1:6, fB 1 did demonstrate inhibitory activity (data not shown)); (4) fB2 displayed some inhibitory activity; and (5) fB3 demonstrated potent inhibition of alternative complement pathway activity. When mixed at a 1 to 1 ratio with wild type fB, fB3 inhibited the hemolytic activity by approximately 90%. At a 2 to 1 ratio, fB3 completely inhibited alternative pathway complement activity (FIG. 17).

### Example Sixteen

### Inhibition of Mouse Alternative Complement Pathway Activity by Mouse Factor B Mutants and Human Factor B Mutants

### Materials and Methods

The equipment, reagents and buffers were essentially the same as described in Example 15 except fresh normal mouse serum was purchased from Innovative Research or freshly drawn from mice.

In addition to BIV vectors encoding human wild type factor B and three human factor B mutants, BIV vectors encoding the wild type mouse factor B and three mouse factor B mutants were also prepared as described in Example 15. The human factor B mutants are designated hfB1, hfB2, and hfB3, and the mouse factor B mutants are designated mfB1, mfB2, and mfB3. The vectors were then used to transduce ARPE cells or Cf2Th cells to produce mouse wild type factor B protein and mouse mutant factor B proteins as described in Example 15. Expression levels of wild type mouse factor B and three mouse factor B mutants were assessed by Western blot analysis and were found to be essentially equivalent, e.g., within 2-fold by visual observation.

### Alternative Complement Pathway Hemolytic Activity Assay

There is no commercially available complement factor B-depleted mouse serum. We found that, for alternative complement pathway activation in mouse serum, factor B is a rate limiting factor. Therefore, by diluting the serum appropriately, which for this study was four fold, the assay could be carried out similarly to the study in Example 15. A competition assay was set up by mixing diluted whole mouse serum with culture medium from the cells transduced with BIV vectors encoding GFP or mfB1, mfB2, or mfB3 respectively at 1 to 1 or at 1 to 2 ratios by volume. In parallel, the three human factor B mutants were also used in this study to determine if the human dominant factor B mutants can compete with the endogenous wild type mouse factor B; that is, to determine if the human factor B mutant(s) could be evaluated in an appropriate mouse model. (In this regard, it is noteworthy that complement factors frequently function in a species specific manner (e.g., see Horstmann et al. J Immunol (1985) 134:11401-4) and will not support complement activation in serum from a different species.)

Hemolytic activity reactions were set up in 14 ml polystyrene round bottom tubes on ice. 40 μl of the mixtures described above were added to each tube along with 50 μl of freshly made ice-cold Mg²⁺-EGTA buffer. The tubes were mixed by vortexing thoroughly with a Fisher Vortex Genie 2 device. Then, 10 μl of Mg²⁺-EGTA washed Erab containing 5×10⁷ erythrocytes were added to each tube followed by gentle mixing without vortexing. The tubes were then incubated in a 39° C. water bath with orbital shaking at 110 rpm for 1 hour. Note these incubation conditions were optimized for the assay with mouse serum and differed from those used for the assay with human serum. The tubes were then returned to ice and 150 μl of ice-cold 0.9% saline was added to stop the reaction. After gentle mixing, the tubes were centrifuged at 2000 rpm with a Beckman Centrifuge for 5 min at 4° C. with the brake turned off. Without disturbing the pellet, 180 μl of each supernatant was transferred into a flat-bottom 96-well plate and the OD 405 was determined in a microplate reader. The 100% lysis and the negative control (blank) samples were set up as described in Example 15.

### Results

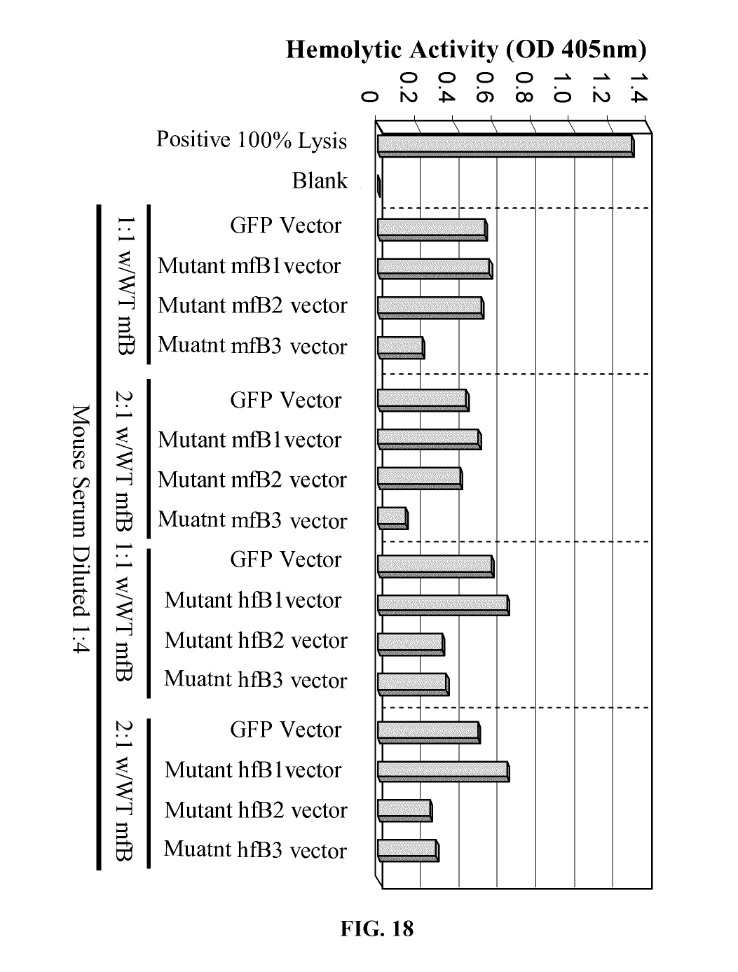

The potencies with which the mouse and human factor B mutants competed with endogenous mouse factor B to inhibit the alternative complement pathway in mouse serum are shown in FIG. 18. The data indicate, inter alia, that: (1) mfB3 inhibited the mouse alternative complement pathway while mfB1 and mfB2 displayed less inhibitory activity in this study; (2) surprisingly, hfB2 and hfB3 inhibited the mouse alternative complement pathway while hfB 1 did not inhibit the mouse alternative complement pathway in this study. This result, which was not expected due to expected species specificity of complement factors, enables the testing of hfB3 in vivo in mouse models.

### Example Seventeen

### Inhibition of Human Alternative Complement Pathway Activity by Mouse Factor B Mutants

### Materials and Methods

The equipment, reagents and buffers were essentially the same as described in Examples 15 and 16.

The generation of BIV vectors encoding mouse wild type factor B and dominant negative factor B mutants was essentially the same as described in Examples 15 and 16. Production of the wild type mouse factor B and dominant negative factor B mutants from BIV vector-transduced cells was essentially the same as described in Examples 15 and 16. The hemolytic assay was performed with human factor B-depleted serum according to the procedure in Example 15.

### Results

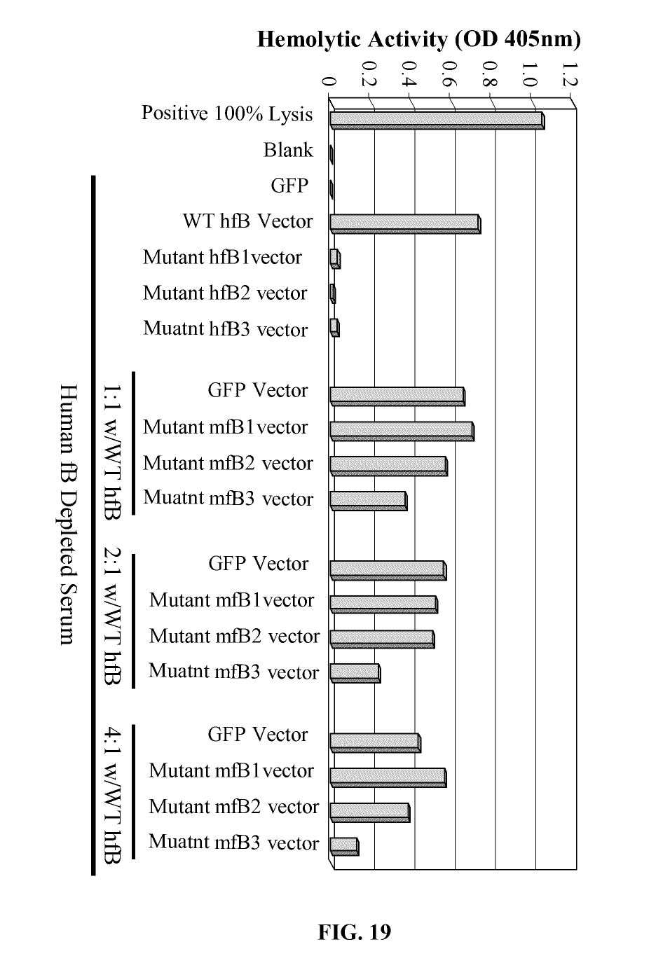

Examples 15 and 16 show, inter alia, that the human dominant negative mutant factors B2 and B3 can inhibit both the human and mouse alternative complement pathways and that mouse dominant negative mutant factor B3 can inhibit mouse alternative complement pathway. This Example is designed to determine if the mouse factor B mutants are capable of inhibiting the human alternative complement pathway. As shown in FIG. 19, the data indicate that: (1) mfB3 inhibited the human alternative complement pathway while (2) mfB1 and mfB2 did not show substantial inhibitory activity in this study. Also included were wild type human factor B and the human factor B mutants as assay controls.

### Example Eighteen

### Inhibition of Porcine Alternative Complement Pathway Activity by Human Factor B Mutants

### Materials and Methods

The equipment, reagents and buffers were essentially the same as described in Example 15 except that fresh porcine serum was drawn from Yucatan Mini-pigs.

The generation of BIV vectors encoding human wild type factor B and the human dominant negative factor B mutants was essentially as described in Example 15. The production of wild type human factor B and the human factor B mutants from BIV vector-transduced cells was essentially the same as described in Example 15.

The porcine alternative complement pathway hemolytic activity assay was essentially the same as described in Example 16 except that diluted fresh porcine serum was used instead of the mouse serum. The serum dilutions in this study were 1:2, 1:4, and 1:6.

### Results

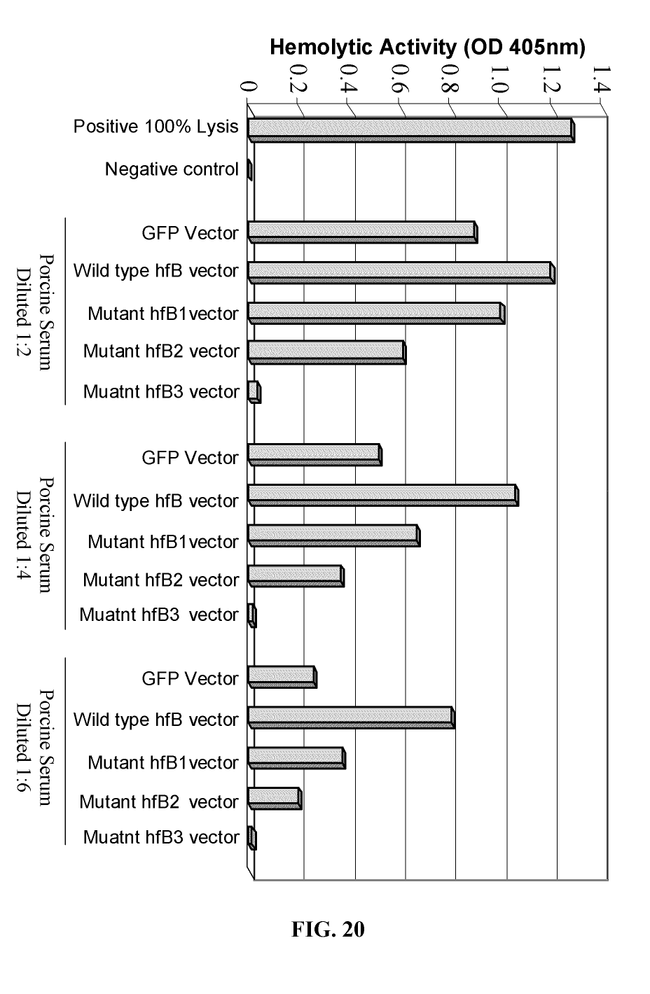

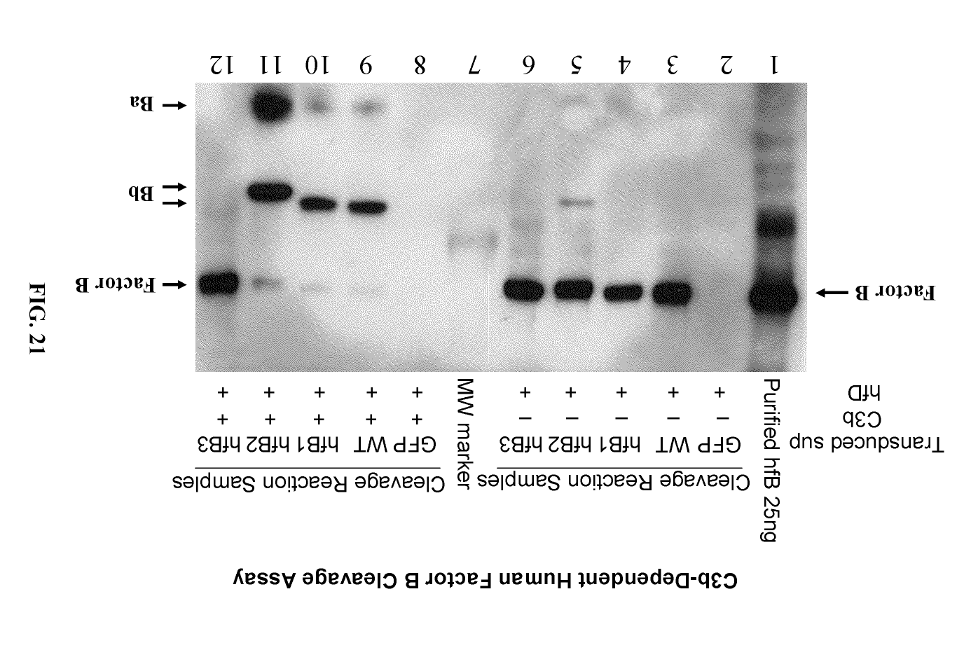

Pigs are useful as a large animal ocular model since the size and vasculature of pig eyes are similar to those of human eyes. We determined if the human dominant negative factor B mutants could inhibit the pig alternative complement pathway and thereby potentially allow future in vivo modeling in pigs. As shown in FIG. 20, the results indicate that hfB3 efficiently inhibited the pig alternative complement pathway. hfB2 also demonstrated inhibitory activity, although hfB2 was not as potent as hfB3.

As evidenced in FIG. 20, wild type human factor B appears to be functional in the pig alternative complement pathway. Specifically, as the pig serum was diluted, the overall potency of hemolytic activity declined. However, at each dilution, the addition of human wild type complement factor B boosted the hemolytic activity. In addition to the observation that human wild type factor B has biological activity in pig serum, this finding also supports the assay supposition that factor B is the limiting compound for porcine and probably human complement-mediated hemolytic activity, at least under these experimental conditions. The results further strengthen the strategies of the present invention, inter alia, that blocking or decreasing factor B function will potently inhibit the alternative complement pathway.

### Example Nineteen

### C3b Dependent Factor B Cleavage

### Reagents and Buffers and Materials and Methods

Purified human complement factor D protein and purified human complement factor C3b protein were purchased from Quidel (Catalog numbers A409 and A413, respectively). Anti-human complement factor B polyclonal antibodies were purchased from Quidel (Catalog# A311).

The VECTASTAIN ABC-Amp Western Blotting Immunodetection Kit was purchased from Vector Laboratories (Catalog# AK-6000). Phosphate buffer solution (PB) contained 8 mM Na₂HPO₄, 2 mM NaH₂PO₄, pH 7.4

The generation of BIV vectors encoding the human factor B wild type protein and three human dominant-negative mutant proteins was essentially as described in Example 15 except the factor B proteins harvested from the BIV transduced cells were used without any further concentration step.

### C3b Dependent Factor B Cleavage Assay

Wild type human factor B protein and three human factor B mutant proteins from transduced supernatant, at a final concentration of approximately 500 ng/ml, were incubated with human factor D, at a final concentration of 200 ng/ml, and human factor C3b, at a final concentration of 2000 ng/ml, in PB plus 25 mM NaCl and 10 mM MgCl₂ (PB+) for 30 min at 37° C. The amount of each component in the reaction mixture is listed in Table two below. As a control, one set of reactions was performed without C3b.

Cleavage of wild type factor B by factor D is C3b dependent. Upon binding of factor B to C3b, factor B undergoes a conformation change, exposing a factor D cleavage site and allowing factor D to cleave factor B (93 Kda) to Bb (63 Kda) and Ba (30 Kda), thereby activating the C3 convertase, C3bBb. Factor B cleavage by factor D can not occur, or occurs at very minimal levels, in the absence of C3b. It should be noted that the concentration of each BIV-encoded human factor B protein from transduced-cell supernatant was estimated to be approximately 10 μg/ml based on Western Blot analysis.

After the reaction, 25 μl reaction mixture was mixed with reducing protein sample buffer incubated at 90° C. for 30 min, cooled, and subjected to SDS-PAGE electrophoresis in a 7.5% Tris-HCl PAGE gel (Bio-RAD). The gel was blotted onto a nitrocellulose membrane via a semi-gel transferring system. The membrane was then probed with a 1:8000 dilution of goat anti-human factor B polyclonal antibody (Quidel, Catalog# 8000), followed by a 1:5000 dilution of rabbit anti-goat biotinylated IgG(H+L) antibody (Vector Laboratories, Catalog# BA-5000). Detection was performed with the VECTASTAIN ABC-Amp Western Blotting Immunodetection Kit (Vector Laboratories).

TABLE 2

| | Samples (μl) | | | | | | | | | |
|---|---|---|---|---|---|---|---|---|---|---|
| | GFP | | hfB WT | | hfB1 | | hfB2 | | hfB3 | |
| Transduced | 25 | | 25 | | 25 | | 25 | | 25 | |
| Supernatant | | | | | | | | | | |
| hfD protein | 1 | | 1 | | 1 | | 1 | | 1 | |
| (0.1 mg/ml) | | | | | | | | | | |
| hfC3b protein | 0 | 1 | 0 | 1 | 0 | 1 | 0 | 1 | 0 | 1 |
| (1.0 mg/ml) | | | | | | | | | | |
| 10 mM PB+ | 474 | 473 | 474 | 473 | 474 | 473 | 474 | 473 | 474 | 473 |

| | | | | | |
|---|---|---|---|---|---|
| Total Volume | 500 μl | 500 μl | 500 μl | 500 μl | 500 μl |

Mix, 30 mins at 37° C.

### Results

The wild type human complement factor B and three factor B mutants were examined for cleavage by factor D. As shown in FIG. 21, in the absence of C3b, there was no efficient cleavage of wild type factor B or any of the three factor B mutants. In the presence of C3b, factor D efficiently cleaved wild type factor B. It also cleaved fB1 and fB2 to varying degrees. However, there was little or no or cleavage of fB3, verifying that the mutation introduced into the factor D cleavage site of fB3 effectively blocked factor D cleavage. The significance of this finding is that, in the absence of this proteolytic cleavage, the C3 convertase, C3bBb, cannot be formed and activation of the alternative complement pathway will be blocked or inhibited. It should be noted that, in FIG. 21, the Bb from fB2 was smaller in molecular mass than Bb from wild type factor B or fB 1 because the mutation engineered into fB2 removed an N-glycosylation site.

### Example Twenty

### Binding of Factor B to C3b

### Reagents and Buffers and Materials and Methods

Purified human complement factor D protein and purified human complement C3b protein were purchased from Quidel (Catalog# A409 and A413, respectively). Anti-human complement factor B and C3 polyclonal antibodies were purchased from Quidel (Catalog# A311 and A413). Biotinylated rabbit anti-goat IgG Fc Fragment Antibody was purchased from Jackson ImmunoLaboratory (Catalog#305-065-046).

Phosphate buffer solution (PB) contained 8 mM Na₂HPO₄, 2 mM NaH₂PO₄, pH 7.4. Wash buffer contained 20 mM Tris-HCL pH 8.0, 0.15 M NaCl, 1% NP-40, 2 mM EDTA. Phenylmethanesulfonyl fluoride (PMSF) was purchased from Sigma (Cat# P7626). Proteinase Inhibitor Cocktail was purchased from Roche (Cat#11836170001). Protein A Agarose Beads were purchased from Invitrogen (Cat#15918-014). Normal Rabbit IgG was purchased from R & D Systems (Cat# AB-105-C). VECTASTAIN ABC-Amp Western Blotting Immunodetection Kit was purchased from Vector Laboratories. The generation of BIV vectors encoding the human factor B wild type protein and the three human dominant-negative mutant proteins was essentially as described in Example 15 except the factor B proteins harvested from the BIV transduced cells were used without any further concentration step.

### Assay for Binding of Factor B to C3b

Wild type human factor B protein and three human factor B mutant proteins from transduced supernatants, at a final concentration of approximately 500 ng/ml, were incubated with human factor D, at a final concentration of 200 ng/ml, and human factor C3b, at a final concentration of 2000 ng/ml, in PB plus 25 mM NaCl and 10 mM MgCl₂ (PB+) for 30 min at 37° C. The amount of each component in the reaction mixture is listed in Table three below. As a control, one set of reactions was performed without C3b.

TABLE 3

| | Samples (ul) | | | | | | | | | |
|---|---|---|---|---|---|---|---|---|---|---|
| | GFP | | hfB WT | | hfB1 | | hfB2 | | hfB3 | |
| Transduced | 25 | | 25 | | 25 | | 25 | | 25 | |
| supernatant | | | | | | | | | | |
| hfD protein | 1 | | 1 | | 1 | | 1 | | 1 | |
| (0.1 mg/ml) | | | | | | | | | | |
| hfC3b | 1 | | 1 | | 1 | | 1 | | 1 | |
| (1 mg/ml) | | | | | | | | | | |
| 10 mM PB+ | 473 | | 473 | | 473 | | 473 | | 473 | |
| Total | 500 | μl | 500 | μl | 500 | μl | 500 | μl | 500 | μl |
| Volume | | | | | | | | | | |

Mix, 30 mins at 37° C.

After the cleavage assay, the reaction tubes were put on ice and the samples were subjected to immunoprecipitation. Each sample (500 μl) was mixed with 1 ml of ice-cold wash buffer containing PMSF and the proteinase inhibitor cocktail tablet. Then 2 μl of normal Rabbit IgG (1 mg/ml) were added, the tubes were rocked at 4° C. for 1 hr, and 100 μl Protein A beads were added. After mixing, the tubes were again rocked at 4° C. for 1 hr and then centrifuged at 14,000 rpm for 5 min. Each supernatant was transferred to a new tube, 2 μl of anti-human complement factor B polyclonal antibody was added, and the tubes were rocked overnight at 4° C. Then 100 μl Protein A beads in ice cold wash buffer were added to each tube and the tubes were rocked at 4° C. for 1 hr. The tubes were then centrifuged at 14,000 rpm for 1 min, the supernatants were discarded, and, without disturbing the pellet, the beads were washed 3 times with ice-cold wash buffer plus PMSF and the protease inhibitor cocktail tablet. The beads were then resuspended and centrifuged at 14,000 rpm for 1 min. Finally, the beads were washed with 1 ml of 500 mM MgCl₂ buffer to remove any proteins that had bound nonspecifically. After an additional spin at 14,000 rpm for 1 min, the supernatant was discarded, 100 μl of the reducing protein sample buffer was added, and the beads were mixed well by vortexing. The beads were then heated to 95° C. for 3 min to release the proteins. The beads were removed by centrifugation at 14,000 rpm for 2 min and 90 μl of the supernatants were transferred into new tubes.

Forty μl of each sample were loaded onto a 7.5% Tris-HCl PAGE gel (Bio-RAD) and subjected to SDS-PAGE followed by Western Blot analysis. The gel was blotted onto a nitrocellulose membrane via a semi-gel transferring system. The membrane was probed with a 1:5000 dilution of goat anti-human factor C3 polyclonal antibody followed by a 1:20,000 dilution of rabbit anti-goat biotinylated IgG Fc Fragment antibody. The protein band was visualized with the VECTASTAIN ABC-Amp Western Blotting Immunodetection Kit (Vector Laboratories).

### Results

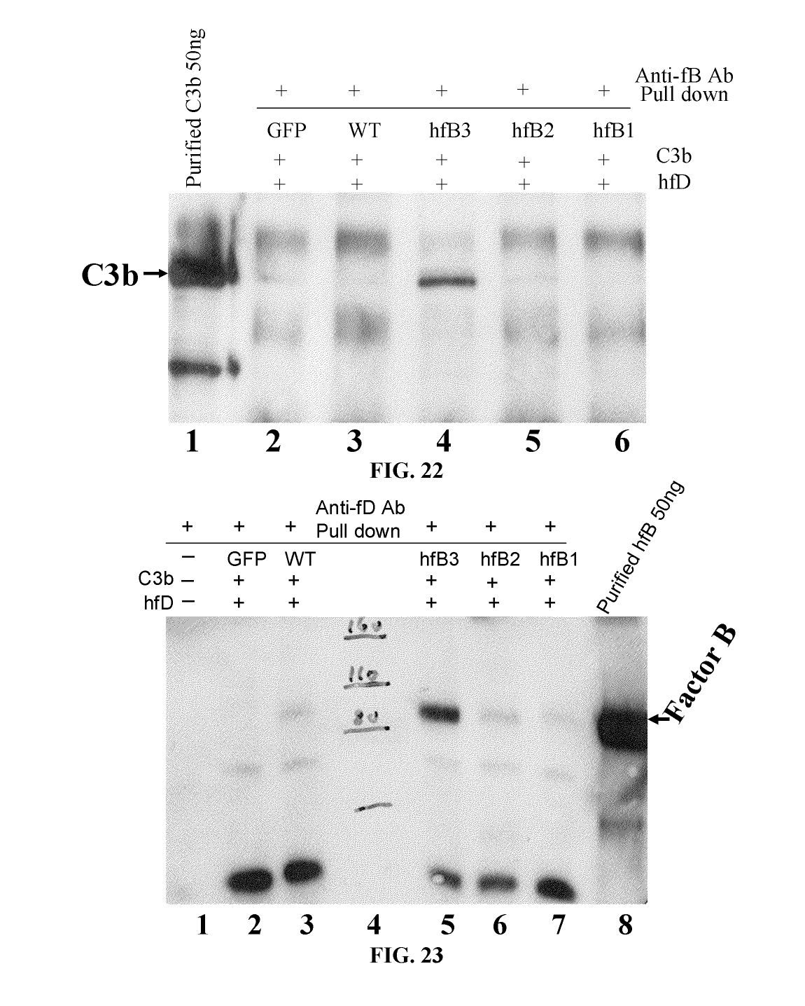

This experiment examined the C3b binding characteristics of the human factor B mutants compared to the wild type factor B. As shown in FIG. 22, an in vitro binding assay was performed using C3b, factor B and factor D. The reaction complex was then immunoprecipitated with a polyclonal anti-factor B antiserum, and the complex was evaluated by Western analysis with a C3b polyclonal antibody probe under denaturing conditions. The extent of C3b immunoprecipitation was greatest with fB3 (FIG. 22, Lane 4). Significantly smaller amounts of C3b were immunoprecipitated with fB2, and even small amounts of C3b were immunoprecipitated with wild type factor B and fB1 (FIG. 22, Lanes 3, 5, and 6). It is noteworthy that, in this study, the amount of immunoprecipitated C3b was anticipated to be small since the complex with wild type factor B is short-lived with a known half-life of less than two minutes. The observation that the binding of C3b to fB3 was significantly greater than the binding of C3b to fB2 is quite surprising since the amino acid changes designed to improve C3b binding were identical in both fB2 and fB3. The reason that the particular combination of mutations in fB3 was so effective to result in tighter binding to C3b remains unknown. Most importantly, the strong binding of fB3 to C3b may be one explanation for the finding that fB3 is the most potent of the factor B dominant negatives at inhibiting the alternative complement pathway. Specifically, sustained binding of fB3 to C3b would lead to sustained sequestration of C3b in a nonfunctional C3 convertase.

### Example Twenty-One

### Binding of Factor D to the C3bB Complex

### Materials and Methods

The reagents and buffers were essentially the same as described in Example 20 except that polyclonal goat anti-human factor D antiserum was purchased from Quidel (R&D Systems, catalog# AF-1824).

The generation of BIV vectors encoding the human wild type factor B protein and three dominant negative human factor B proteins was essentially the same as described as in Example 15.

The assay for the binding of factor D to the C3bB complex was essentially the same as the assay for the binding of fB to C3b described in Example 20 except that the reaction complex was immunoprecipitated with anti-factor D antiserum and the Western blot was probed with polyclonal goat anti-human factor B antiserum.

### Results

Examples 19 and 20 showed that the human fB3 is resistant to cleavage by factor D and that it binds to C3b more tightly than either wild type factor B, fB1, or fB2. This Example was designed to evaluate the binding characteristics of factor D to different C3bB complexes, with each different C3bB complex having a different human dominant negative factor B analog. Factor D is normally found at very low levels and functions as a catalyst to cleave factor B in the C3bB complex to Ba and Bb. Therefore, very little factor D would be expected to be bound to the C3bB complex at any point in time. As expected, and as shown in FIG. 23, only small amounts of factor B were co-precipitated with factor D for the wild type factor B, fB1, and fB2. Interestingly, and very unexpectedly, with fB3, much more co-precipitated with the factor D (FIG. 23, Lane 5). Thus, when C3b complexes with fB3, it appears factor D binds much more tightly to the complex. The mechanism by which this sustained binding occurs remains unknown. Most importantly, the sustained factor D binding provides a further explanation for why fB3 is more potent than fB1 or fB2 at inhibiting the alternate complement pathway. Specifically, after complexing with C3b, fB3 can achieve sustained binding of factor D and thereby sequester factor D. Since factor D is only available in small amounts and factor D protein is called upon to mediate many proteolytic cleavages, removal of factor D from the alternative complement pathway would serve to have a potent effect at blocking the pathway.

The data in this example and the previous ones demonstrate that, while all three dominant negative factor B moieties function to attenuate the alternative complement pathway, fB3 unexpectedly stood out as the most potent. Furthermore, mechanistic studies indicate that the unique potency of fB3 may be due to two attributes: 1) its tight binding to C3b and 2) its tight binding to factor D. Thus, upon entering the alternative complement pathway, fB3 achieves sustained binding to both C3b and factor D and removes both of these components from the pathway without forming a functional C3 convertase (or C5 convertase). The end result is a blockade of the positive feedback amplification loop of the alternative complement pathway and potent inhibition of the pathway.

Not wishing to be bound by theory, the finding that factor D binds tightly to a complex of C3b and fB3 may be related to its inability to cleave fB3. This supports the use of a dominant negative factor D (e.g., as described herein), designed to have debilitated protease activity, as an additional means of efficiently inhibiting the alternative complement pathway. Due to its inability to cleave factor B, such dominant negative factor D would be expected to bind a C3bB complex tighter than wild type factor D. The bound dominant negative factor D would prevent wild type factor D from entering the complex, cleaving the factor B, and activating the C3 convertase. The structure of factor D has been characterized (Volanakis J E and Narayana S V L, 1996, Protein Science 5:553-564) allowing the design of a dominant negative with debilitated protease activity. Finally, a dominant negative factor D moiety may be used alone or in combination with a dominant negative factor B moiety.

### Example Twenty-Two

### Inhibition of the Human Alternative Complement Pathway by Anti-human Factor B Monoclonal Antibodies

### Materials and Methods

The equipment, reagents, and buffers were essentially the same as described in Example 15 except that the mouse monoclonal antibody against human factor B was purchased from Quidel (Catalog# A227) and isotype matched control mouse IgG was purchased from eBioscience (Catalog#14-4714).

### Alternative Complement Pathway Hemolytic Activity Assay

Rabbit red blood cells were handled in the same way as described in Example 15. Before hemolytic reaction, 12.5 ng/μl of purified recombinant human factor B protein was pre-incubated with anti-human factor B monoclonal antibody at 1:0.5, 1:1, 1:2, and 1:3 molar ratios in Mg-EGTA buffer in each tube with 40 μl of total volume for 30 min at 4° C. Then, 50 μl of human factor B depleted serum diluted 25 fold in freshly prepared ice cold Mg-EGTA buffer was added into each tube. For the positive control tube, 500 ng of purified human complement factor B protein was prepared in 50 μl of the 25-fold diluted human factor B depleted serum and the volume was increased to 90 μl with Mg²⁺-EGTA buffer. Then 10 μl of Mg²⁺-EGTA washed Erab containing 5×10⁷ erythrocytes was added to each tube. The reactions were gently mixed without a vortex. The tubes were placed in a 37° C. water bath with orbital shaking at 110 rpm for 40 min. After 40 min, the tubes were placed back on ice and 150 μl of ice cold 0.9% saline was added to stop the reaction. Each tube was gently mixed and then centrifuged at 2000 rpm for 5 min at 4° C. in a Beckman centrifuge with the brake turned off. Without disturbing the pellet, 180 μl of each supernatant was gently transferred to a flat-bottom 96-well plate, and the OD 405 was determined in a microplate reader.

### Results

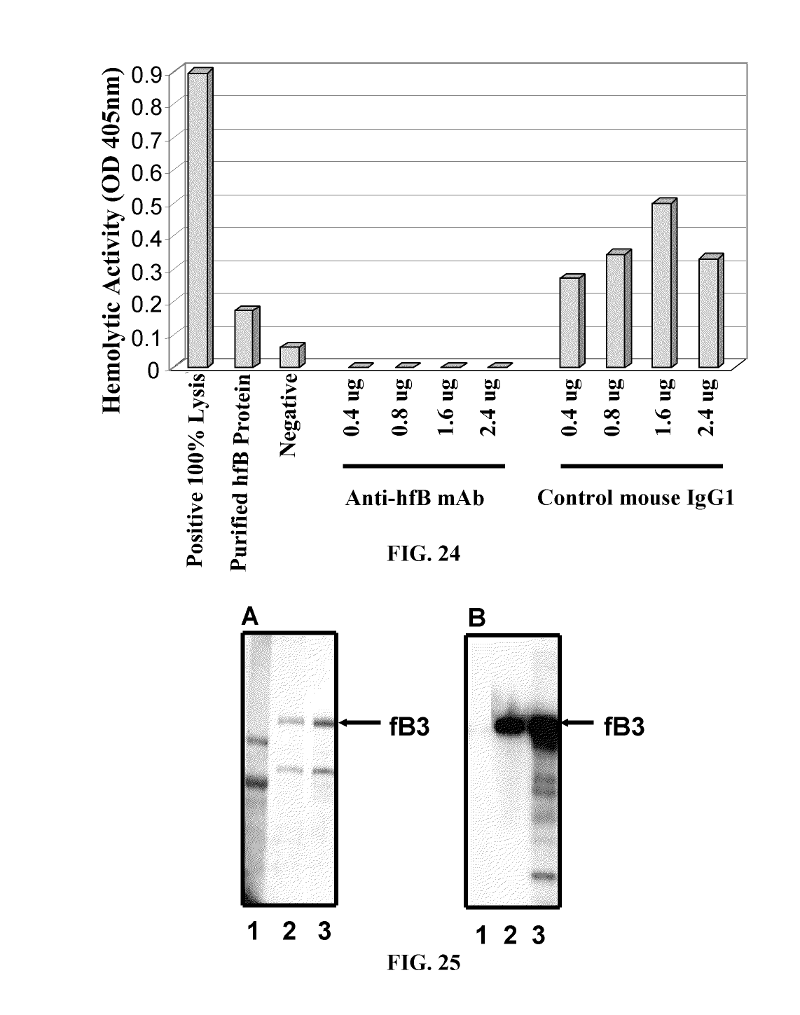

Another aspect of the invention for inhibiting the alternative complement pathway is to use a binding molecule, such as a monoclonal antibody (mAb) or binding fragment thereof against a component(s) of the pathway including, but not limited to, factor B, factor D, C3b, C3 convertase, etc. A binding molecule could be either delivered directly (e.g., to the retina) or expressed by a gene transfer vector (such as a lentiviral vector). A monoclonal antibody could be delivered as a fragment such as a Fab fragment or a single chain antibody. This experiment examined if a binding molecule, such as a monoclonal antibody, could effectively inhibit the alternative pathway. As shown in FIG. 24, an anti-human factor B mAb essentially shut down alternative complement pathway activity at all four doses tested. The inhibition was specific as the control mouse IgG did not inhibit or block the pathway activity at similar doses.

### Example Twenty-Three

### Inhibition of the Human Alternative Complement Pathway Activity by Anti-Human Complement Factor D

### Materials and Methods

The equipment, reagents and buffers were essentially the same as described in Example 15 except that the monoclonal anti-human factor D antibody was purchased from Affinity Bioreagents (Golden, Colo., Catalog# GAU008-01-02) and the isotype matched mouse control IgG was obtained from eBioscience (Catalog#14-4732). The alternative complement pathway activity hemolytic assay was essentially the same as described in Example 22 except the anti-factor D mAb was used instead of the anti-factor B mAb.

### Results

Factor D represents an important component of the alternative complement pathway, and is therefore an ideal target to inhibit the pathway. This experiment was designed to show that a binding protein, such as a mAb, against human factor D could inhibit the pathway. As shown in Table Four, anti-factor D mAb inhibited alternative complement pathway hemolytic activity in a dose-dependent manner. The inhibition is factor D specific as the isotype matched control mouse IgG did not score any inhibition.

TABLE 4
Inhibition of human alternative complement pathway
activity by an anti-human factor D monoclonal antibody.

| | OD |
|---|---|
| Sample | 405 nm |
| Anti-hfD mAb 5 ng + | 0.373 |
| Purified hfB protein 0.5 ug | |
| Anti-hfD mAb 10 ng + | 0.432 |
| Purified hfB protein 0.5 ug | |
| Anti-hfD mAb 20 ng + | 0.369 |
| Purified hfB protein 0.5 ug | |
| Anti-hfD mAb 30 ng + | 0.235 |
| Purified hfB protein 0.5 ug | |
| Anti-hfD mAb 40 ng + | 0.227 |
| Purified hfB protein 0.5 ug | |
| Anti-hfD mAb 50 ng + | 0.165 |
| Purified hfB protein 0.5 ug | |
| Anti-hfD mAb 60 ng + | 0.126 |
| Purified hfB protein 0.5 ug | |
| Anti-hfD mAb 70 ng + | 0.103 |
| Purified hfB protein 0.5 ug | |
| Anti-hfD mAb 80 ng + | 0.078 |
| Purified hfB protein 0.5 ug | |
| Anti-hfD mAb 90 ng + | 0.058 |
| Purified hfB protein 0.5 ug | |
| Anti-hfD mAb 100 ng + | 0.061 |
| Purified hfB protein 0.5 ug | |
| Anti-hfD mAb 150 ng + | 0.035 |
| Purified hfB protein 0.5 ug | |
| Anti-hfD mAb 200 ng + | 0.029 |
| Purified hfB protein 0.5 ug | |
| Anti-hfD mAb 400 ng + | 0.022 |
| Purified hfB protein 0.5 ug | |
| Anti-hfD mAb 800 ng + | 0.015 |
| Purified hfB protein 0.5 ug | |
| Anti-hfD mAb 1600 ng + | 0.014 |
| Purified hfB protein 0.5 ug | |
| Anti-hfD mAb 2400 ng + | 0.049 |
| Purified hfB protein 0.5 ug | |
| Positive (100% Lysis) | 1.453 |
| Negative Control | 0 |
| Purified hfB Protein 0.5 ug | 0.399 |
| Control IgG 5 ng + | 0.428 |
| Purified hfB protein 0.5 ug | |
| Control IgG 10 ng + | 0.361 |
| Purified hfB protein 0.5 ug | |
| Control IgG 20 ng + | 0.441 |
| Purified hfB protein 0.5 ug | |
| Control IgG 30 ng + | 0.392 |
| Purified hfB protein 0.5 ug | |
| Control IgG 40 ng + | 0.426 |
| Purified hfB protein 0.5 ug | |
| Control IgG 50 ng + | 0.430 |
| Purified hfB protein 0.5 ug | |
| Control IgG 60 ng + | 0.416 |
| Purified hfB protein 0.5 ug | |
| Control IgG 70 ng + | 0.395 |
| Purified hfB protein 0.5 ug | |
| Control IgG 80 ng + | 0.387 |
| Purified hfB protein 0.5 ug | |
| Control IgG 90 ng + | 0.347 |
| Purified hfB protein 0.5 ug | |
| Control IgG 100 ng + | 0.397 |
| Purified hfB protein 0.5 ug | |
| Control IgG 150 ng + | 0.426 |
| Purified hfB protein 0.5 ug | |
| Control IgG 200 ng + | 0.347 |
| Purified hfB protein 0.5 ug | |
| Control IgG 400 ng + | 0.506 |
| Purified hfB protein 0.5 ug | |
| Control IgG 800 ng + | 0.538 |
| Purified hfB protein 0.5 ug | |
| Control IgG 1600 ng + | 0.618 |
| Purified hfB protein 0.5 ug | |
| Control IgG 2400 ng + | 0.588 |
| Purified hfB protein 0.5 ug | |

Activity of the alternate complement pathway was assessed with a hemolytic assay. The relative hemolytic activity is measured by the amount of hemoglobin released into the supernatant after lysis of rabbit erythrocytes. Positive control with 100% lysis, RBC lysed in water; Purified hfB protein, factor B-depleted human serum supplemented with 500 ng of purified human factor B protein; Negative control, the erythrocytes were incubated in isotonic saline (no red blood cell lysis); Factor B-depleted human serum supplemented with a mixture of 500 ng of purified human factor B protein and either anti-human factor D mAb (from 5 ng to 2400 ng) or control mouse IgG (from 5 ng to 2400 ng).

### Example Twenty-Four

### Inhibition of the Human and Mouse Alternative Complement Pathways by Rabbit Anti-Factor B Monoclonal Antibodies

### Materials and Methods

The alternative complement pathway hemolytic assay was essentially the same as described in Example 22 except that the rabbit anti-factor B mAb described in this Example was used instead of mouse anti-factor B mAb.

We generated mAbs in rabbits to increase the likelihood that antibodies generated against human factor B would cross-react with mouse factor B thereby enabling animal modeling in mice. Purified human factor B was purchased from Quidel (Catalog# A408) and used as an antigen to immunize rabbits. The antibodies were generated by Genesis Biotech, Inc. (Taiwan) according to their standard procedure. Two rabbits were immunized, spleen cells were fused with a fusion partner, hybridomas were identified, and mAb secretion was screened by ELISA. Twenty hybridoma supernatants were further analyzed for their ability to block the alternative complement pathway with the hemolytic assay described in Example 22. Lyophilized hybridoma medium (from 1 ml of hybridoma culture medium) was dissolved in a total of 250 μl of 1×PBS per vial. The PBS re-suspended hybridoma solution was stored in aliquots at −80° C.

Prior to the hemolytic reaction, 20 μl of each hybridoma culture medium was pre-incubated with 500 ng of purified human factor B protein in Mg-EGTA buffer in a total of 40 μl for 30 min at 4° C. For the antibody positive control, 500 ng of purified human factor B protein was pre-incubated with 400 or 800 ng of anti-human factor B monoclonal antibody (Quidel) in Mg-EGTA buffer in 40 μl for 30 min at 4° C. Then, 50 μl of human factor B depleted serum diluted 25 fold in freshly prepared ice cold Mg-EGTA buffer was added into each tube. For the positive control tube, 500 ng of purified human complement factor B protein was prepared in 50 μl of the 25-fold diluted human factor B depleted serum and the volume was increased to 90 μl with Mg²⁺-EGTA buffer. Then 10 μl of Mg²⁺-EGTA washed Erab containing 5×10⁷ erythrocytes was added to each tube. The reactions were gently mixed without a vortex. The tubes were placed in a 37° C. water bath with orbital shaking at 110 rpm for 40 min. After 40 min, the tubes were placed back on ice and 150 μl of ice cold 0.9% saline was added to stop the reaction. Each tube was gently mixed and then centrifuged at 2000 rpm for 5 min at 4° C. in a Beckman centrifuge with the brake turned off. Without disturbing the pellet, 180 μl of each supernatant was gently transferred to a flat-bottom 96-well plate, and the OD 405 was determined in a microplate reader.

To examine the inhibition of the 20 rabbit mAbs against mouse alternative complement pathway activity, the same experiment was performed except it was done with four-fold diluted whole mouse serum instead of factor B-depleted human serum.

### Results

Human and mouse factor B are approximately 83% homologous at the amino acid level. It would be desirable to generate a mAb that can be used for both animal modeling in rodents and for therapy in human. As shown in Tables 5 and 6, twenty positive rabbit hybridomas were generated. Monoclonal antibodies produced by some of these clones inhibited both the human (Table 5) and mouse (Table 6) alternative complement pathways. These positive hybridomas can be used as sources to generated single chain antibodies, which can then be humanized, e.g., for human therapies. Such single chain antibodies can be delivered either via injection of protein or with a vector encoding the single chain antibody. Alternatively, a rabbit mAb can be humanized and delivered as an Fab fragment or as a whole protein, e.g., for therapeutic uses. The strategy in this example can be used to generate rabbit monoclonal antibodies with therapeutic utility against other alternative complement pathway critical components, e.g. factor D, C3b, or C3 convertase.

TABLE 5
Inhibition of Complement Activity using an anti-hfB mAb

| | OD |
|---|---|
| Sample | 405 nm |
| Hybridoma No. 7 + | 0.036 |
| Purified hfB protein 0.5 ug | |
| Hybridoma No. 9 + | 0.112 |
| Purified hfB protein 0.5 ug | |
| Hybridoma No. 11 + | 0.09 |
| Purified hfB protein 0.5 ug | |
| Hybridoma No. 13 + | 0.071 |
| Purified hfB protein 0.5 ug | |
| Hybridoma No. 16 + | 0.203 |
| Purified hfB protein 0.5 ug | |
| Hybridoma No. 18 + | 0.061 |
| Purified hfB protein 0.5 ug | |
| Hybridoma No. 19 + | 0.112 |
| Purified hfB protein 0.5 ug | |
| Hybridoma No. 21 + | 0.262 |
| Purified hfB protein 0.5 ug | |
| Hybridoma No. 22 + | 0.164 |
| Purified hfB protein 0.5 ug | |
| Hybridoma No. 23 + | 0.142 |
| Purified hfB protein 0.5 ug | |
| Positive | 1.146 |
| (100% Lysis) | |
| Negative Control | 0 |
| Purified hfB Protein 0.5 ug | 0.388 |
| Anti hfB mAb 0.4 ug + | 0.051 |
| Purified hfB Protein 0.5 ug | |
| Anti hfB mAb 0.8 ug + | 0 |
| Purified hfB Protein 0.5 ug | |
| Hybridoma No. 25 + | 0.135 |
| Purified hfB protein 0.5 ug | |
| Hybridoma No. 27 + | 0.067 |
| Purified hfB protein 0.5 ug | |
| Hybridoma No. 28 + | 0.037 |
| Purified hfB protein 0.5 ug | |
| Hybridoma No. 34 + | 0.103 |
| Purified hfB protein 0.5 ug | |
| Hybridoma No. 36 + | 0.056 |
| Purified hfB protein 0.5 ug | |
| Hybridoma No. 37 + | 0.2 |
| Purified hfB protein 0.5 ug | |
| Hybridoma No. 38 + | 0.041 |
| Purified hfB protein 0.5 ug | |
| Hybridoma No. 41 + | 0.125 |
| Purified hfB protein 0.5 ug | |
| Hybridoma No. 43 + | 0.093 |
| Purified hfB protein 0.5 ug | |
| Hybridoma No. 44 + | 0.085 |
| Purified hfB protein 0.5 ug | |

TABLE 6
Inhibition of Alternative Complement Pathway
Activity with an anti-hfB mAb

| | OD |
|---|---|
| Sample | 405 nm |
| Hybridoma No. 7 + | 0.143 |
| Mouse serum 50 ul (1:6 Diluted) | |
| Hybridoma No. 9 + | 0.05 |
| Mouse serum 50 ul (1:6 Diluted) | |
| Hybridoma No. 11 + | 0.095 |
| Mouse serum 50 ul (1:6 Diluted) | |
| Hybridoma No. 13 + | 0.055 |
| Mouse serum 50 ul (1:6 Diluted) | |
| Hybridoma No. 16 + | 0.074 |
| Mouse serum 50 ul (1:6 Diluted) | |
| Hybridoma No. 18 + | 0.107 |
| Mouse serum 50 ul (1:6 Diluted) | |
| Hybridoma No. 19 + | 0.073 |
| Mouse serum 50 ul (1:6 Diluted) | |
| Hybridoma No. 21 + | 0.22 |
| Mouse serum 50 ul (1:6 Diluted) | |
| Hybridoma No. 22 + | 0.077 |
| Mouse serum 50 ul (1:6 Diluted) | |
| Hybridoma No. 23 + | 0.165 |
| Mouse serum 50 ul (1:6 Diluted) | |
| Positive | 1.164 |
| (100% Lysis) | |
| Negative Control | 0 |
| Mouse serum 50 ul | 0.981 |
| (1:6 Diluted) | |
| Anti hfB mAb 0.4 ug + | 0.074 |
| Mouse serum 50 ul (1:6 Diluted) | |
| Hybridoma No. 25 + | 0.073 |
| Mouse serum 50 ul (1:6 Diluted) | |
| Hybridoma No. 27 + | 0.131 |
| Mouse serum 50 ul (1:6 Diluted) | |
| Hybridoma No. 28 + | 0.134 |
| Mouse serum 50 ul (1:6 Diluted) | |
| Hybridoma No. 34 + | 0.112 |
| Mouse serum 50 ul (1:6 Diluted) | |
| Hybridoma No. 36 + | 0.143 |
| Mouse serum 50 ul (1:6 Diluted) | |
| Hybridoma No. 37 + | 0.065 |
| Mouse serum 50 ul (1:6 Diluted) | |
| Hybridoma No. 38 + | 0.075 |
| Mouse serum 50 ul (1:6 Diluted) | |
| Hybridoma No. 41 + | 0.067 |
| Mouse serum 50 ul (1:6 Diluted) | |
| Hybridoma No. 43 + | 0.09 |
| Mouse serum 50 ul (1:6 Diluted) | |
| Hybridoma No. 44 + | 0.104 |
| Mouse serum 50 ul (1:6 Diluted) | |

### Example Twenty-Five

### Purified Human fB3 Protein Inhibits the Alternative Complement Pathway

Some embodiments of the present invention include delivering a protein that inhibits or blocks a pathway such as the alternative complement pathway. For exemplary purposes, this example describes the use of fB3 protein, e.g., to inhibit the alternative complement pathway.

fB3 protein, as described herein, could be made in mammalian cells, in bacteria, in yeast, in insect, or in other living organisms. This study was designed to determine if we could purify fB3 protein from cell culture medium and still preserve its biological activity. Cf2Th cells were transduced with a BIV vector encoding human fB3 (see Example 15). The transduced cells were maintained in DMEM medium containing 2% FBS. Seventy-two hours post transduction, the cell culture medium was harvested and cleared of cell debris by filtering through a 0.2 μm sterile filter. The cleared cell culture medium was loaded onto an affinity column (PIERCE, Catalog#44894) conjugated with a monoclonal antibody against human factor B (R&D Systems, Catalog# MAB2739). The column was washed and the sample was eluted according to the manufacture's instructions. The eluted fractions were evaluated by SDS-PAGE with a silver stain. The fractions were also examined by Western blot analysis to verify the identity of protein. As shown in FIGS. 25A and 25B, the affinity column yielded relatively pure fB3 of the expected size. FIG. 25A shows silver staining of affinity purified human factor B3 protein: Lane 1, molecular weight marker; Lane 2, eluted sample from the first fraction; Lane 3, eluted sample from combination of the second and the third fractions. FIG. 25B shows a Western blot analysis for human factor B3 protein and the lane assignment is the same as in Panel A.

Furthermore, there is no obvious degradation product, suggesting that the purification process was well tolerated by the mutant factor B3. There is an additional band at a molecular size equivalent to BSA at approximately 65 Kda (FIG. 25, Panel A, lanes 2 and 3).

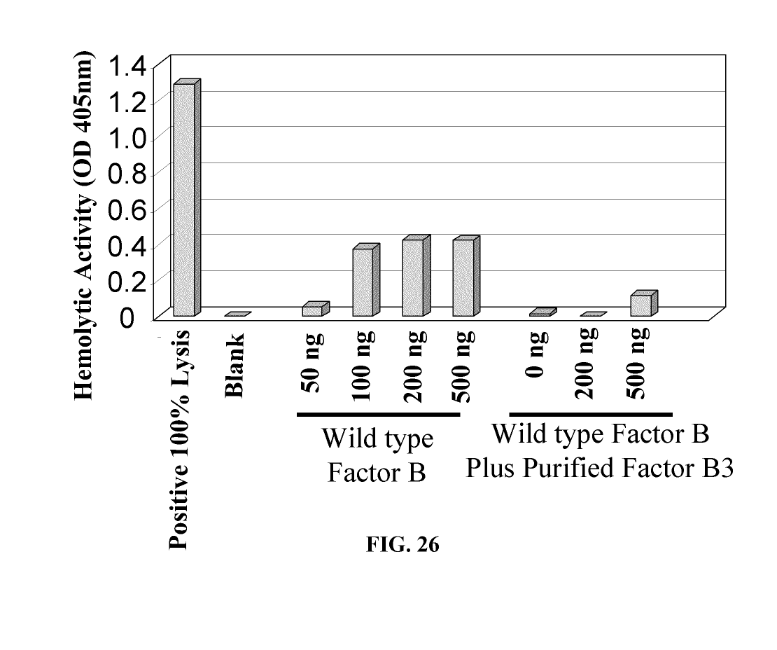

To determine if the purified mutant factor B3 was biologically active, the alternative complement pathway hemolytic assay was performed as described in Example 15. As shown in FIG. 26, the affinity purified human fB3 protein efficiently inhibited the human alternative complement pathway, showing that the purification process did not cause any obvious damage to the protein's biological function. The fB3 protein can also be purified by other means or in combination with other means, e.g. ion-exchange chromatography, size exclusion chromatography, ammonia sulfate precipitation, HPLC.

### Example Twenty-Six

### Cell Lines that Constitutively Express Human fB3

To manufacture and purify fB3 more readily, we generated a cell line that constitutively expresses fB3 in serum-free, suspension culture. Specifically, 293 Freestyle cells (Invitrogen) were transduced with a BIV vector encoding fB3 (e.g., see Example 15). The cells were grown in 293 Freestyle Serum-Free Medium (Invitrogen) in suspension cell culture. The tissue culture medium was subjected to SDS-PAGE and Western blot analyses as described in Example 25. fB3 protein was found to be expressed and secreted into the tissue culture medium (data not shown). The biological activity of the fB3 was evaluated with the hemolytic assay as described in Example 25 and the fB3 was shown to be biologically active (data not shown). fB3 protein has also been expressed from CHO cells and Cf2Th cells.

fB3 protein or other proteins described herein could be further modified to increase potency, e.g., for therapeutic purposes. For example, a protein could be conjugated to polyethylene glycol (PEGylated) to increase its half-life in vivo; could be delivered by a device that releases the protein when the device is implanted in vivo; could be made as a fusion protein to increase its potency or half-life in vivo; could be mixed with a carrier to increase its distribution in vivo; could be delivered via a cell that expresses the said protein; could be further modified to improve its pharmacokinetics profile; and/or could be truncated to make a variant that still preserves its biological function.

### Example Twenty-Seven

### A BIV Vector Encoding Human FB3 Inhibits Retinal Inflammation In Vivo

This example evaluated a BIV vector encoding human fB3 in a mouse model of laser injury to the retina. In this aggressive model of inflammation, a laser is used to burn the retina resulting, within hours, in a rapid activation of the alternative complement pathway.

In the study, each mouse received a subretinal injection at the periphery of the retina with a vector encoding either fB3 or no transgene. Two weeks later, three laser burns were made near the central part of the retina. Twenty hours later, the retinas were harvested and stained for deposition of complement factor C3 or Membrane Attack Complex (MAC), which are markers of complement activation.

To demonstrate the size and location of the vector injection, a separate cohort of mice received an injection of a vector encoding GFP. This cohort was not subject to laser injury.

### Methods

Vectors encoding GFP, human fB3, or no transgene (null) were prepared and concentrated via anion exchange chromatography as described in Example 15. The storage buffer was PBS plus 1 mM MgCl₂, 2.5 mM KCl, and 0.1% BSA. The vector was stored at −80° C. until use.

The GFP vector was titered as follows: On day 1, 2×10⁵ Cf2TH cells were seeded per well in a 6 well plate in 3 mls of DMEM supplemented with 2 mM glutamine, Pen/Strep, and 10% FBS (complete DMEM). The cells were incubated overnight at 37° C. in 8.5% CO₂. The following day, the medium was replaced with 1.5 mls of complete DMEM plus 8 ug/ml Polybrene. Vector (0.2 or 1 ul) was added to each well and the plates were incubated for 15-18 hours. The medium was then replaced with 3 mls of fresh complete DMEM. After an additional 49-52 hours, the cells were subjected to flow cytometry, and the percentage of cells that were fluorescent (i.e. more fluorescent than untransduced cells) was assessed. The titer was mathematically determined from the vector input volume, the number of cells in the well, and the percentage of fluorescent cells.

To determine the titer of the hfB3 and null vectors, all three vectors were used to transduce cells as above. However, instead of flow cytometry, the transduced cells were subjected to Real-Time PCR to determine their vector DNA copy number. DNA was prepared from the cells by routine procedures. PCR primers were designed to amplify the RRE region of the BIV vectors:

(SEQ ID NO: 24)

Probe:
5′-FAM-ACACCACCATCCCTCCGCATCCGA-BHQ-1-3′

(SEQ ID NO: 25)

Sense Primer:
5′-TGGGTTTGTGGTAGTAAATGACAC-3′

(SEQ ID NO: 26)

Anti-Sense Primer:
5′-TGGTTCACGAGCGTTGTAGC-3′

PCR amplification was performed with an IQ5 Multicolor Real-Time PCR Detection system (BioRad) with the following conditions: 100 nM probe, 600 nM sense primer, 600 nM anti-sense primer, and 1× SuperMix (BioRad). Reactions were incubated at 95° C. for 3 min followed by 40 cycles of 95° for 10 sec and 55° for 30 sec. The titer of the hFB3 and null vectors in transducing units (tu)/ml was determined by comparing their vector DNA copy numbers to that of the GFP vector.

For this study, the titers in tu/ml were as follows: GFP vector, 9×10⁷; hfB3 vector, 3.2×10⁷; and null vector, 7.3×10⁷.

The subretinal injection procedure was as follows. Each mouse (C57BL/6) received ketamine (100 ug/gm) via intramuscular injection. Dosages were adjusted to achieve a deep plane of anesthesia. The eyes were treated with topical 0.55 praparacaine immediately prior to the procedure. Under anesthesia, the eye was gently protruded manually. A small incision was made through the sclera just posterior to the limbus with the edge of a 30 gauge needle. A 33 gauge blunt-tipped needle was inserted tangentially toward the posterior pole of the eye and placed between the retina and retinal pigment epithelial layers. 0.5 to 1.0 ul of vector suspension was injected, and the success of the injection was verified by observing the retina lift from the surface at the injection site. (It is noteworthy that the injected fluid was absorbed and the retina was reattached within one day.) The needle was withdrawn and pressure maintained to prevent back leakage. The animals were observed until awake and ambulatory before being returned to their cages.

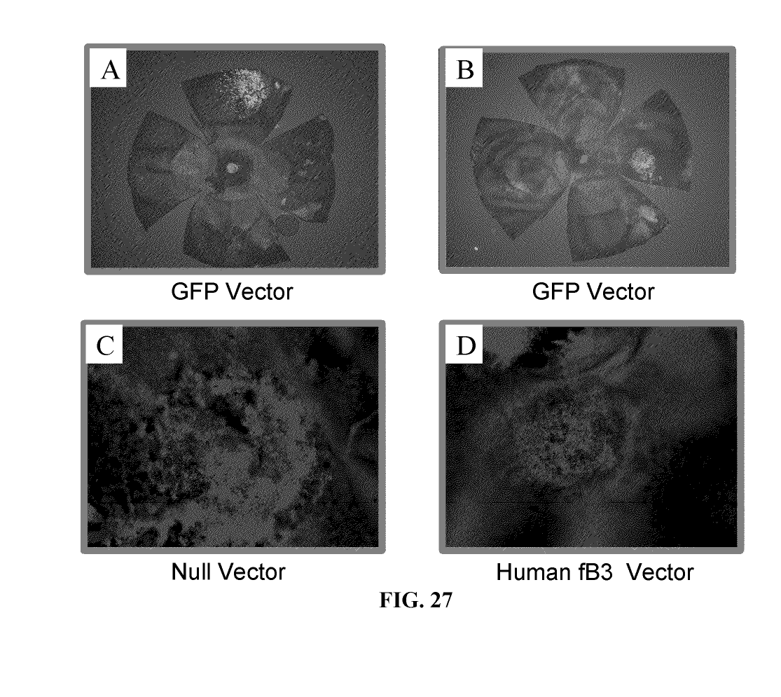

Two weeks after vector injection, retinas from the animals that received the GFP vector were harvested; retinal flat mounts were prepared; and GFP expression was observed (FIGS. 27A & 27B).

Two weeks after vector injection, the eyes that received the hfB3 and null vectors were subjected to laser injury as follows. Laser photocoagulation was performed using a diode laser (810 nm, OcuLight Six from IRIS Medical) and a Zeiss slit lamp system with a handheld cover glass as a contact lens. The laser parameters were set at 100 mW intensity, 75 micron spot size, 0.1 sec duration, and single pulse. Three burns (at 3, 12, and 9 o'clock) were made in each eye with each spot at 2 to 3 disc diameters from the optic nerve. The success of the burn procedure; that is, the rupture of Bruch's membrane, was verified by the identification of bubble formation at the site of the burn. These laser parameters had been previously optimized to consistently provide for a successful burn without causing excessive damage; that is, hemorrhage at the burn site.

Twenty hours after the laser injury, the retinas were harvested and stained for deposition of complement factor C3 or MAC at the burn sites. Animals were sacrificed by overdose of ketamine and xylazine, and the eyes were immediately enucleated. After overnight fixation in 4% paraformaldehyde at 4° C., each eye was carefully dissected under a Nikon dissecting microscope with removal of the anterior segment and vitreous. The neurosensory retina was carefully separated from the RPE layer, and the remaining RPE-choroid-sclera complex was cut radially to form a flat mount. The complexes were subjected to immunohistochemical staining.

The immunohistochemical staining procedure for C3 deposition was as follows. (The MAC staining procedure was done similarly.) Each RPE-choroid-sclera complex flat mount was washed for 5 minutes with TBS (Tris buffered saline: Tris/Tris-HCl 25 mM, NaCl 0.13 M, KCl 0.0027 M, pH 7.4±0.13; Fisher Scientific, Catalog #BP2471-100) times to remove the paraformaldehyde. Each complex was then blocked with 2% BSA and 1% Triton X 100 in TBS for 1 hr. The complexes were subsequently washed for 5 minutes in TBS 3 times. The complexes were then blocked with 10% Normal Rabbit Serum (NRS) and 1% Triton in TBS for 2 hours at room temperature followed by 3 five minute washes in TBS. The complexes were then incubated overnight at 4° C. in the primary antibody (Polyclonal Goat anti-human C3 from Calbiochem, cat. #204869) diluted 1:100 in TBS plus 10% NRS and 0.5% Triton. The following morning the complexes were washed 3 times for 10 minutes each with TBS plus 0.2% Tween 20 followed by 1 wash for 10 minutes with TBS. The complexes were then incubated for 2 hours at room temperature with the secondary antibody (Alexa fluor 594 conjugated Rabbit anti-goat IgG from Invitrogen, cat. # A-11080) diluted 1:300 in TBS. The complexes were washed 3 times for 10 minutes each with TBS plus 0.2% Tween 20 followed by 1 wash for 5 minutes with TBS. The complexes were mounted on slides under coverslips with Vectashield mounting medium. The complexes were then examined by fluorescence microscopy. Control stains omitted the primary antibody. It is noteworthy that, although the primary antibody was generated against human C3, it also stained mouse C3.

### Results

FIGS. 27A & 27B show GFP staining from two representative retinal flat mounts. In each case, the injection transduced an area of the peripheral retina. FIGS. 27C & 27D show C3 staining of a representative laser burn from an eye treated with the null vector and an eye treated with the hfB3-encoding vector. The area and intensity of C3 staining was substantially greater in the null vector treated eyes than in the hfB3 vector treated eyes. The results with the MAC staining were similar to those with C3 staining (data not shown).

### CONCLUSIONS

The vector encoding hfB3 was effective at inhibiting complement activation in this laser injury model. The results were particularly impressive for at least two reasons. First, this is a very aggressive model of complement activation in which the complement activation results from an acute burn. Second, efficacy was achieved with a very small number of transduced cells that were located distant from the laser injury sites. Efficacy in this relevant animal model of ocular inflammation predicts efficacy in the treatment of human disease.

### Example Twenty-Eight

### Evaluation of Human FB3 Protein Delivery as a Means of Blocking Complement Activation In Vivo

This experiment is designed to demonstrate that hfB3 protein, when delivered by direct intraocular injection is able to inhibit complement activation. The study is performed similarly to the one in the example above. In this case, human fB3 protein, e.g., as prepared by the procedure in Examples 25 or 26, is administered via intravitreal and/or subretinal injection. The intravitreal injection procedure differs from the subretinal injection procedure only in that the needle is placed in the vitreous rather than beneath the retina. In each case, the injected volume is approximately 1 μl with concentrations of fB3 ranging from 1 ng/μl to 20 μg/μl. Control eyes are injected with formulation without hfB3 protein. Laser burns are performed immediately prior to the injections. Twenty hours after the laser injury the retinas are harvested and stained for C3 or MAC and compared to injections of formulation alone.

### Example Twenty-Nine

### Vector Concentration and Purification Procedure: Scale-Up Using a Sartobind SingleSep Mini Q Membrane Adsorber Capsule

The following is a detailed procedure for scaling up the purification process for BIV-based lentiviral vectors.

Reagents and Materials
a. Benzonase, ultrapure, Sigma Cat# E8263 or equivalent
b. 1×PBS pH 7.4, Invitrogen, Cat#10010-023 or equivalent
c. 10×PBS, pH 7.4, Invitrogen, Cat#70011-044 or equivalent
d. Distilled Water, sterile, DNase, RNase-free, Invitrogen Cat#10977015 or equivalent
e. 5M NaCl, Cambrex Cat#51202 or equivalent
f. Sartobind SingleSep Mini Q, Sartorius Cat#921EXQ42D4-00
g. Vivaspin 20, 1 million MWCO, Sartorius Cat# VS2061
h. Diafiltration cups for Vivaspin 20, Sartorius Cat# VSA005
i. Nalgene Tubing, 180PVC, FDA/USPVI, ⅛″ID×¼″ OD s 1/16″ wall or equivalent
j. EGTA, BioChemika Ultra >99%, Sigma Cat#03778 or equivalent
k. Storage bottles, disposable, various sizes., Corning or equivalent
l. Centrifuge tubes, 250 ml, Corning
m. Centrifuge tube, 50 ml, Falcon
n. MF75 aPES 0.2 μm filter unit, 1 liter, Nalgene Item#567-0020 or equivalent
o. 0.2 μm PES, 26 mm, sterile syringe filter. Corning item#431229 or equivalent
p. Syringe, 3 ml, Becton-Dickinson, BD item#309585 or equivalent
q. Syringe, 60 ml, Becton-Dickinson, BD item#301627 or equivalent
r. Serological pipets, various sizes, sterile, individually wrapped
s. Ring stand and clamp
t. Peristaltic pump and pumphead, Masterflex L/S or equivalent
u. Centrifuge, Beckman Allegra 6KR with GH3.8 rotor or equivalent

### Sample Preparation

a. Start with 1250 mls of cell culture medium containing vector prepared by calcium phosphate plasmid transfection as described in Example 1 (unconcentrated vector).
b. Divide the unconcentrated vector into 250 ml centrifuge tubes.
c. Centrifuge for approximately 10 minutes at 2800 RPM, in Beckman Allegra 6KR centrifuge with GH3.8 rotor, to remove cell debris.
d. Place 600 mls of unconcentrated vector into each of two Corning 1 liter bottles.
e. To each bottle, add 12 mls of 500 mM EGTA (10 mM final concentration), pH 8.0 and 30,000 units of Benzonase (50 units/ml final concentration). Incubate at 37° C. for 30-40 minutes.
f. Filter each 600 mls of unconcentrated vector through a Nalgene 0.2 μm PES filter unit.

### Loading

a. Dilute the 1,200 mls of vector 1:1 with chilled Loading Buffer. Place on ice.
   Loading Buffer (2×PBS, 1.0 M NaCl):*

| | |
|---|---|
| 240.0 mls | 10X PBS, pH 7.4 |
| 165.6 mls | 5M NaCl |
| 794.4 mls | sterile, DNase and RNase - free water (chilled) |
| 1,200 mls | |

b. Place tubing in head of peristaltic pump as per manufacturer's directions.
c. Place end of tubing into vessel containing 1×PBS. Attach to SingleSep Mini Q and purge air from tubing and capsule unit using approximately 250 mls of 1×PBS. Make sure all air is removed from the Sartobind Mini Q Capsule.
d. Carefully place the end of the feed tube into the unconcentrated vector solution.
e. Pass sample solution through the unit at a rate of approximately 12 ml/min. Continue until a minimum of sample remains in the feed vessel. Do not draw air into the tube.

### Washing

a. Carefully remove feed tube from sample vessel and place into chilled Wash Buffer. Do not allow air to enter the SingleSep Mini Q unit.
b. Wash SingleSep Mini Q with approximately 200 mls of Wash Buffer at a rate of 12 ml/min.
   Wash Buffer (1×PBS, 500 mM NaCl):

| | |
|---|---|
| 40.0 mls | 10X PBS, pH 7.4 |
| 27.6 mls | 5M NaCl |
| 332.4 mls | sterile, DNase and RNase - free water (chilled) |
| 400 mls | total |

### Elution

a. Fill a 60 ml syringe with 40 mls of chilled Elution Buffer.
   Elution Buffer (1×PBS, 1.3 M NaCl):

| | |
|---|---|
| 20.0 mls | 10X PBS, pH 7.4 |
| 45.8 mls | 5M NaCl |
| 134.2 mls | sterile, DNase and RNase - free water (chilled) |
| 200 mls | total |

b. Attach to SingleSep Mini Q, making sure no air is introduced.
c. Holding the unit vertically, push through 9 ml of Elution Buffer, very slowly, drop by drop. Discard.
d. Allow to stand for 10-15 minutes.
e. Place fresh 50 ml centrifuge tube under SingleSep Mini Q and collect approximately 20 mls.

Further Concentration and Diafiltration
a. Pre-cool Beckman Allegra 6KR with GH3.8 rotor to 10° C.
b. Carefully place eluted vector into two Vivaspin 20 units (1 million MWCO). Centrifuge in the Beckman Allegra 6KR with GH3.8 rotor at 2200 RPM for 20 minutes, check volume and recentrifuge until approximately 2 mls remain (liquid is fully contained within the V of the device).
c. Place diafiltration cup into unit, push cup all the way down.
d. Add 12 mls Diafiltration Buffer (1×PBS supplemented with 1 mM MgCl₂ and 2.5 mM KCl) to diafiltration cup.
e. Centrifuge Vivaspin 20 in the Beckman Allegra 6KR with GH3.8 rotor at 2200 RPM for 15 minutes at 10° C.
f. Check volume remaining and respin as needed until vector volume is approximately 750 μl in each of the two Vivaspin 20 diafiltration units.
g. Remove the diafiltration cup. Collect the vector by pipeting up and down several times (about 10 times).
h. Pool the two diafiltrates to obtain a final volume of 1.5 ml of concentrated vector.
i. Sterile filter through a 0.2 μM PES syringe filter.
j. Store at ±80° C.

This procedure routinely yielded 2 mls of vector preparation with titers in the low to mid 10⁸ tu/ml range with less than 50 pg/ml plasmid DNA contamination, less than 4 pg/ml host cell DNA contamination, and undetectable (less than 0.1 ng/ml) Benzonase contamination. The procedure did not completely eliminate BSA. When the unconcentrated vector starting material contained 10% FBS, the BSA levels in the concentrated vector ranged from 7 to 21 μg/ml (BSA ELISA from Cygnus Technologies, Southport, N.C., Cat.# F030). This procedure is further scaleable to meet manufacturing requirements.

### Example Thirty

### Addition of a Size Exclusion Chromatography Polishing Step to the Vector Concentration and Purification Procedure of Example Twenty-Nine

The following is a procedure to further purify the vector of Example 29 and diminish the BSA level. Following the diafiltration step of Example 29, but prior to sterile filtration, the concentrated vector is applied to a Sephacryl S 500-HR column (Sigma).

Pack an empty 20 ml Econo-Pac Chromatography column (BioRad) with Sephacryl S 500-HR under gravity with a bed volume of 20 mls.

a. Equilibrate with five volumes of storage buffer (PBS supplemented with 2.5 mM KCl and 1.0 mM MgCl₂).

b. Carefully apply approximately 1.5 ml of concentrated vector to the top of the Sephacryl and allow the vector to enter the Sephracryl under gravity.

c. Add additional storage buffer to the top of the column, drain the column under gravity, and collect 0.5 to 1.0 ml fractions. Vector is found in the elution volume from 6.5 to 9.5 ml.

d. Pool the fractions containing vector, sterile filter, and store at ±80° C.

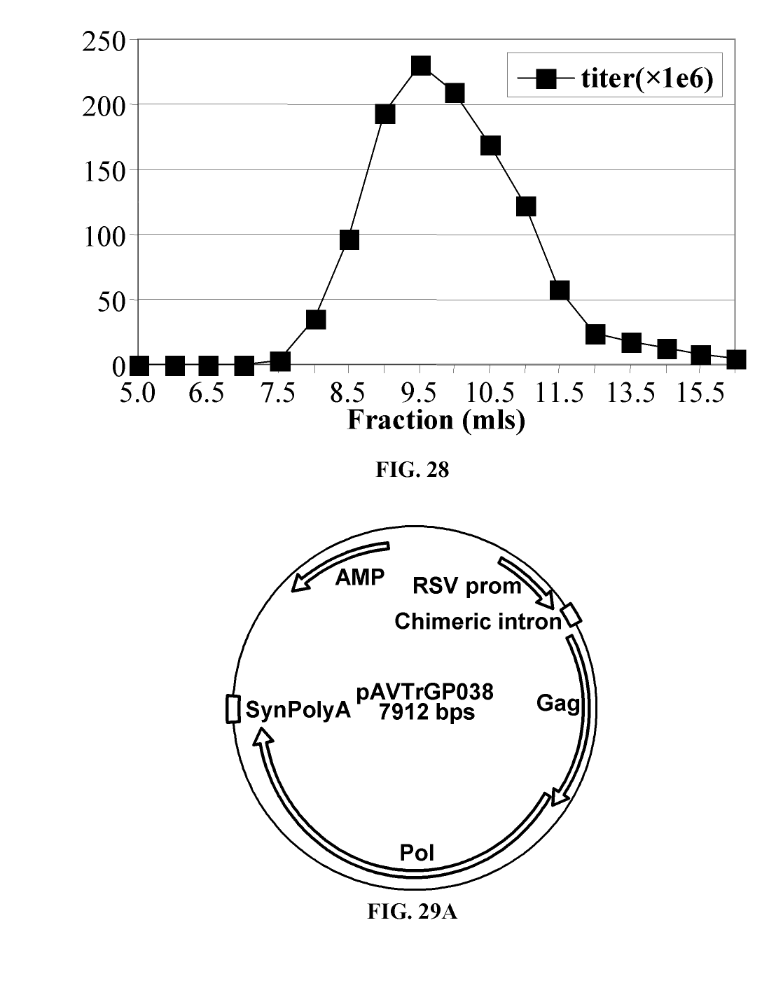

When compared to the diafiltrate that was applied to the column, the vector titer in the eluate was diluted two-fold and the yield was approximately 90%. In the study shown in FIG. 28, the unconcentrated vector starting material was in tissue culture medium containing 2% FBS (see Example 31, below). The BSA content of the diafiltrate that was applied to the column was 311 ng/ml. As shown in FIG. 9, the addition of this size exclusion chromatography step effectively separated the BSA from the vector. This procedure is further scaleable to meet manufacturing requirements.

### Example Thirty-One

### Collection of Vector in Low Serum or Serum-Free Defined Medium

The upstream process described in Example 1 involves calcium phosphate mediated transfection of four plasmids into 293, 293T, or 293FT cells followed by a media change with the addition of butyrate and then collection of the vector-containing medium. The examples above used, for the most part, tissue culture media containing 10% FBS. The inventors have found that it is possible to collect vector in media with less FBS or to use completely defined medium and eliminate the FBS all together without suffering any loss in titer. In this example, all of the cells were plated in DMEM plus 10% FBS. The next day, the media was changed to DMEM plus either 10% or 2% FBS. Three hours later the cells were transfected. Eighteen hours after transfection, the media was changed to DMEM plus either 10% FBS or 2% FBS each containing 5 mM sodium butyrate, or the medium was changed to CD CHO Medium (Invitrogen) without any FBS but containing 5 mM sodium butyrate. Twenty-four hours later the media was collected and the vector titer was determined. The results, shown in Table 7, indicate that calcium phosphate transfection can be performed in 2% FBS and high titer vector can be obtained in serum-free, completely defined medium. The inventors have also determined that the upstream processes of transfection and vector collection can be scaled up in Nunc Cell Factories with yields that approximate those achieved in tissue culture dishes (titers are within two-fold).

TABLE 7

| Transfection Media | Collection Media | Titer |
|---|---|---|
| DMEM plus 10% FBS | DMEM plus 10% FBS | 4 × 10⁶ tu/ml |
| DMEM plus 10% FBS | DMEM plus 2% FBS | 4 × 10⁶ tu/ml |
| DMEM plus 10% FBS | CD CHO Media (no FBS) | 5 × 10⁶ tu/ml |
| DMEM plus 2% FBS | CD CHO Media (no FBS) | 7 × 10⁶ tu/ml |

### Example Thirty-Two

### Combining the Vector Concentration and Purification Technologies of Examples 1 and 29 Through 31

293 FT cells (Invitrogen) were plated in DMEM plus 10% FBS as in Example 1. The following day, the media was changed on each plate to DMEM plus 2% FBS. Calcium phosphate transfection of the four vector-generating plasmids was performed as in Example 1. Eighteen hours later the media was changed to CD CHO Medium (Invitrogen) without any FBS but containing 5 mM sodium butyrate. Twenty-four hours later, the media was collected and subjected to the concentration and purification procedure of Example 29 in combination with the size exclusion chromatography procedure of Example 30. FIG. 28 shows the vector titer in each of the fractions from the Sephacryl column. There was no BSA detectable in any of the fractions from the Sephacryl column. The limit of detection for the BSA ELISA (Cygnus Technologies, Southport, N.C., Cat. # F030) was 500 pg/ml. This scaleable procedure provides highly purified, high titer vector.

### Example Thirty-Three

### Purification of Human fB3 by AKTA Purifier

Purification of soluble secreted fB3 from culture supernatant of cells (e.g., CHO or 293 cells) is conducted by a combination of anion exchange (IEXQ), hydrophobic interaction (HIC) and size exclusion chromatography (SEC) for capturing, intermediate purification and polishing steps. The fB3-containing cell culture supernatant is concentrated 40 to 50-fold on a 30 kDa cut-off ultrafiltration membrane and adjusted to pH 9.0 with 50 mM Tris-HCl before loading onto a pre-pack anion exchange column (HiTrap Capto Q, GE Healthcare). The column is previously equilibrated with buffer containing 50 mM Tris-HCl, pH 9.0 (buffer A, conductivity 5 mS/cm), at 60 ml/h linear flow rate and the effluent monitored by UV detection at 280 nm. After elution of unbound material, retained materials are eluted by mixing with buffer B (50 mM Tris-HCl and 1 M NaCl, pH 9.0) using a non-linear gradient to raise the conductivity of the mobile phase stepwise to 16 mS/cm (10% of buffer B for 10 CV), 34 mS/cm (27% of buffer B for 10 CV), 54 mS/cm (50% of buffer B for 5 CV) and then 101 mS/cm (100% of buffer B for 10 CV). Fractions from the anion-exchange column are analyzed by means of SDS-PAGE under reducing conditions and by ELISA. The majority of fB3 is detected in the material eluted at the second step (27% of buffer B) between conductivity 18-30 mS/cm which contains about 80% of total input fB3. These fractions will typically contain a main species (50-70% of total loading material) at about 93 kDa, corresponding to the completely reduced fB3. However, other protein contaminants may also be present within these fractions. Optionally, a hemolytic assay can be applied as a functional assay to determine if the fB3 purified under this condition retained its dominant-negative activity or not. Positive results will show that increasing doses of fB3 at this purification step can suppress alternative complement activation pathway-mediated hemolysis, indicating that the anion exchange purified fB3 maintains its dominant negative activity over wild-type fB.

To facilitate removal of protein contaminants, an intermediate step may be utilized. This intermediate step can use a hydrophobic interaction chromatography which purifies and separates proteins mainly based on differences in their surface hydrophobicity. The major fB3-containing fractions from anion exchange chromatography are pooled and adjusted to a final concentration 1.5 M ammonium sulfate and 50 mM phosphate buffer, pH 7.0 (conductivity 216 mS/cm) by adding 2 M ammonium sulfate and 50 mM phosphate buffer. The sample is then applied to a hydrophobic interaction column (e.g., HiTrap Phenyl HP, GE Healthcare) which is pre-equilibrated with 1.5 M ammonium sulfate and 50 mM phosphate buffer, pH 7.0 at the flow rate of 60 ml/h. After sample loading, the retained material is eluted by decreasing the ammonium sulfate concentration in a linear fashion (from 1.5 M to 0 M by 35 CV). The presence of fB3 in fractions can be determined, e.g., by SDS-PAGE, Western Blot and/or ELISA.

The fB3-containing peaks are then concentrated to a final volume of 1 ml and subjected to gel filtration on a Sephacryl 5300 16/26 HR column and equilibrated in PBS buffer (50 mM phosphate, 150 mM NaCl, pH 7.0) respectively. The elution of fB3 is performed at a constant linear flow rate of 30 cm/h and the effluent is monitored by UV detection at 280 nm. Purity of fractions can be confirmed, e.g., by SDS-PAGE using silver staining Activity can be performed using a hemolytic assay in the presence of wild-type fB.

Additionally, a PD-10 desalting column can be used to exchange fractions or pooled fractions of HIC-purified fB3 into GVB buffer. The hemolytic assay can be performed by adding increasing doses of HIC-purified fB3 to test for inhibitory activity.

### REFERENCES CITED

Adams, C. W., et al. (2005). Humanization of a recombinant monoclonal antibody to produce a therapeutic HER dimerization inhibitor, pertuzumab. Cancer Immunol. Immunotherapy 55, 717-727.
Age-Related Eye Disease Study (AREDS) Research Group. (2000). Risk factors associated with age-related macular degeneration. A case-control study in the age-related eye disease study: Age-Related Eye Disease Study Report Number 3. Ophthalmology, 107, 2224-2232.
AREDS Research Group. (2001). A randomized, placebo-controlled, clinical trial of high-dose supplementation with vitamins C and E and β carotene for age-related cataract and vision loss, AREDS report no. 9. Arch. Opthalmol. 119, 1439-1452.
Ambati, J., Anand, A., Fernandez, S., Sakurai, E., Lynn B. C., Kuziel. W. A., Rollins, B. J., and Ambati, B. K. (2003). An animal model of age-related macular degeneration in senescent Ccl-2 or Ccr-2 deficient mice. Nat. Med. 9, 1390-1397.
Anderson, D. H., Ozaki, S., Nealon, M., Neitz, J., Mullins, R. F., Hageman, G. S., and Johnson, L. V. (2001). Local cellular sources of apolipoprotein E in the human retina and retinal pigmented epithelium: implications for the process of drusen formation. Am. J. Opthalmol. 131, 767-781.
Ault, B. H. (2000). Factor H and the pathogenesis of renal diseases. Pediatr. Nephrol. 14, 145-1053.
Ault, B. H., Schmidt, B. Z., Fowler, N. L., Kaashtan, C. E., Ahmed, A. E., Vogt, B. A., and Colten, H. R. (1997). Human factor H deficiency. Mutations in framework cysteine residues and block in H protein secretion and intracellular catabolism. J. Biol. Chem. 272, 25168-25175.
Auricchio, A., et al. (2001). Exchange of surface proteins impacts on viral vector cellular specificity and transduction characteristics: the retina as a model. Hum. Mol. Genet. 10, 3075-3081.
Bradt, B. M., Kolb, W. P., and Cooper, N. R. (1998). Complement-dependent pro-inflammatory properties of the Alzheimers's disease β-peptide. J. Exp. Med. 188, 431-438.
Bennett, J., Duan, D., Engelhardt, J. F., and Maguire, A. M. (1997). Real-time, noninvasive in vivo assessment of adeno-associated virus-mediated retinal transduction. Invest. Opthalmol. Vis. Sci. 38, 2857-2863.
Bird, A. (1992). Pathophysiology of AMD. In Age-Related Macular Degeneration: Principles and Practice (Hampton, G., and Nelsen, P. T., eds.) Chap. 3, Raven Press, New York.
Black, S., Kushner, I. and Samols, D. (2004) C-reactive protein. J. Bio. Chem. 279, 48487-48490.
Blom, A. M., Kask, L., Ramesh, B., and Hillarp, A. (2003). Effects of zinc on factor I cofactor activity of C4b-binding protein and factor H. Arch. Biochem. Biophys. 418, 108-118.
Blumenkranz, M. S., Russell, S. R., Robey, M. G., Kott-Blumenkranz, R., and Penneys, N. (1986). Risk factors in age-related maculopathy complicated by choroidal neovascularization. Ophthalmology, 93, 552-558.
Broder, E. T. Midelfort, K. S., and Wittrup, K. D. (2000). Directed evolution of antibody fragments with monovalent femtomolar antigen-binding affinity. PNAS 97, 10701-10705.
Bora, P. S., et al. (2005) Role of complement and complement membrane attack complex in laser-induced choroidal neovascularization. J. Imm. 174, 491-497.
Bora, N. et al. (2006) Complement activation via alternative pathway is critical in the development of laser-induced choroidal neovascularization: role of factor B and factor H. J. Imm. 177:1872-1878.
Bora, N. S. et al. (2007) CD59, a complement regulatory protein, controls choroidal neovascularization in a mouse model of wet-type age-related macular degeneration. J. Imm. 178, 1783-1790.
Bressler, S. B., Maguire, M. G., Bressler, N. M., and Fine, S. L. (1990). Relationship of drusen abnormalities of the retinal pigment epithelium to the prognosis of neovascular macular degeneration. The Macular Photocoagulation Study Group. Arch. Opthalmol. 108, 1442-1447.
Campochiaro, P. A. & Hackett, S. F. (2003) Ocular neovascularization: a valuable model system. Oncogene 22, 6537-6548.
Carolina, R. et al. (1994) Mammalian expression and secretion of functional single chain Fv molecule. J. Biol. Chem. 269, 26267-26273.
Colville, D., Guymer, R., Sinclair, R. A., and Savige, J. (2003). Visual impairment caused by retinal abnormalities in mesangiocapillary (membranoproliferative) glomerulonephritis type II (“dense deposit disease”). Am. J. Kidney Dis. 42, E2-E5.
Connelly, S., Smith, T. A. G., Dhir, G., Gardner, J., Mehaffey, M., Zaret, K., McClelland, A., and Kaleko, M. (1995). In vivo Gene Delivery and Expression of Physiological Levels of Functional Human Factor VIII in Mice. Human Gene Therapy 6, 185-193.
Dahlback, K., Lofberg, H., and Dahlback, B. (1988). Immunohistochemical studies on vitronectin in elastic tissue disorders, cutaneous amyloidosis, lichen ruber planus and porphyria. Acta Derm. Venereol. 68, 107-115.
Dahlback, K., Wulf, H. and Dahlback, B. (1993). Vitronectin in mouse skin: immunohistochemical demonstration of its association with cutaneous amyloid. J. Invest. Dermatol. 100, 166-170.
DeWan, A. et al. (2006) HTRA1 promoter polymorphism in wet age-related macular degeneration. Science 314, 989.
Diaz-Guillen, M. A., Rodriguez de Cordoba, S., and Heine-Suner, D. (1999). A radiation hybrid map of complement factor H and factor H-related genes. Immunogenetics, 49, 549-552.
Edwards, A. O., Ritter, R., Abel, K. J., Manning, A., Panhuysen, C., and Farrer, L. A. (2005). Complement factor H polymorphism and age-related macular degeneration. Sciencexpress, 10 Mar. 2005.
Esparza-Gordillo, J., Soria, J. M., Buil, A., Almasy, L., Blangero, J., Fontcuberta, J. and Rodriguez de Cordoba, S. (2004). Genetic and environmental factors influencing the human factor H plasma levels. Immunogenetics, 56, 77-82.
Estaller, C., Schwaeble, W., Dierich, M., and Weiss, E. H. (1991). Human complement factor H: two factor H proteins are derived from alternatively spliced transcripts. Eur. J. Immunol. 21, 799-802.
Friese, M. A., Hellwage, J., Jokiranta, T. S., Meri, S., Peter, H. H., Eibel, H., and Zipfel, P. F. (1999). FHL-1/reconectin and factor H: two human complement regulators which are encoded by the same gene are differently expressed and regulated. Mol. Immunol. 36, 809-818.
Giannakis, E., Jokiranta, T. S., Male, D. A., Ranganathan, S., Ormsby, F. J., Fischetti, V. A., Mold, C., and Gordon, D. L. (2003). A common site within factor H SCR 7 responsible for binding heparin, C-reactive protein and streptococcal M protein. Eur. J. Immunol. 33, 962-969.
Gordon, D. L., Kaufman, R. M., Blackmore, T. K., Kwong, J., and Lublin, D. M. (1995). Identification of complement regulatory domains in human factor H. J. Immunol. 155, 348-356.
Gorin, M. B., Breitner, J. C., De Jong, P. T., Hageman, G. S., Klayer, C. C., Kuehn, M. H., and Seddon, J. M. (1999). The genetics of age-related macular degeneration. Mol. Vis. 5, 29.
Hageman, G. S., Luthert, P. J., Victor Chong, N. H., Johnson, L. V., Anderson, D. H., and Mullins, R. F. (2001). An integrated hypothesis that considers drusen as biomarkers of immune-mediated processes at the RPE-Bruch's membrane interface in aging and age-related macular degeneration. Prog. Retin, Eye Res. 20, 705-732.
Hageman, G. S., Mullins, R. F., Russell, S. R., Johnson, L. V., and Anderson, D. H. (1999). Vitronectin is a constituent of ocular drusen and the vitronectin gene is expressed in human retinal pigmented epithelial cells. FASEB J. 13, 477-484.
Hageman, G. S. et al. (2005) A common haplotype in the complement regulatory gene factor H(HF1/CFH) predisposes individuals to age-related macular degeneration. PNAS 102, 7227-7232.
Haines, J. L., Hauser, M. A., Schmidt, S., Scott, W. K., Olson, L. M., Gallins, P., Spencer, K. L., Kwan, S. Y., Noureddine, M., Gilbert, J. R., Schnetz-Boutaud, N., Agarwal, A., Postel, E. A., Pericak-Vance, M. A. (2005). Complement factor H variant increases the risk of age-related macular degeneration. Sciencexpress, 10 Mar. 2005.
Hamdi, H. K. and Kenney, C. (2003). Age-related macular degeneration: A new viewpoint. Frontiers in Bioscience, 8, 305-314.
Hegasy, G. A., Willhoeft, U., Majno, S. A., Seeberger, H., and Zipfel, P. F. (2003). Pig complement regulator factor H: molecular cloning and functional characterization. Immunogenetics 55, 462-471.
Hellwage, J., Skerka, C., Zipfel, P. F. (1997). Biochemical and functional characterization of the factor-H-related protein 4 (FHR-4). Immunopharmacology. 38, 149-157.
Hogasen, K., Jansen, J. H., Mollnes, T. E., Hovdenes J., Harboe, M (1995). Hereditary porcine membranoproliferative glomerulonephritis type II is caused by factor H deficiency. J. Clin. Invest. 95, 1054-1061.
Jansen, J. H., Hogasen, K., and Mollnes, T. E. (1993). Extensive complement activation in hereditary porcine membranoproliferative glomerulonephritis type II (porcine dense deposit disease). Am. J. Pathol. 143, 1356-1365.
Johnson, L. V., Leitner, W. P., Staples, M. K., and Anderson, D. H. (2001). Complement activation and inflammatory processes in drusen formation and age related macular degeneration. Exp. Eye Res. 73, 887-896.
Johnson, L. V., Leitner, W. P., Rivest, A. J., Staples, M. K., Radeke, M. J., and Anderson, D. H. (2002). The Alzheimer's AB-peptide is deposited at sites of complement activation in pathologic deposits associated with aging and age-related macular degeneration. Proc. Natl. Acad. Sci. USA, 99, 11830-11835.
Johnson, L. V., Ozaki, S., Staples, M. K., Erickson, P. A., and Anderson, D. H. (2000). A potential role for immune complex pathogenesis in drusen formation. Exp. Eye Res. 70, 441-449.
Johnson, P. T., Betts, K. E., Radeke, M. J., Hageman, G. S., Anderson, D. H. and Johnson, L. V. (2006) Individuals homozygous for the age-related macular degeneration risk-conferring variant of complement factor H have elevated levels of CRP in the choroid. PNAS 103, 17456-17461.
Jokiranta, T. S., Hellwage, J., Koistinen, V., Zipfel, P. F., and Meri, S. (2000). Each of the three binding sites on complement factor H interacts with a distinct site on C3b. J. Biol. Chem. 275, 27657-27662.
Kardys, I. et al. (2006) A common polymorphism in the complement factor H gene is associated with increased risk of myocardial infarction. J. Am. Coll. Cardiol. 47, 1568-1575.
Klein, R. J., Zeiss, C., Chew, E. Y., Tsai, J-Y., Sackler, R. S., Haynes, C., Henning, A. K., SanGiovanni, J. P., Mane, S. M., Mayne, S. T., Bracken, M. B., Ferris, F. L., Ott, J., Barnstable, C., and Hoh, J. (2005). Complement factor H polymorphism in age-related macular degeneration. Sciencexpress, 10 Mar. 2005.
Kuhn, S., Skerka, C., and Zipfel, P. F. (1995). Mapping of the complement regulatory domains in the human factor H-like protein 1 and in factor H1. J. Immunol. 155, 5663-56670.
Kuhn, S, and Zipfel, P. F. (1996). Mapping of the domains required for decay acceleration activity of the human factor H-like protein 1 and factor H. Eur. J. Immunol. 26, 2383-2387.
Li, M. et al. (2006) CFH haplotypes without the Y402H coding variant show strong association with susceptibility to age-related macular degeneration. Nature Genetics 38, 1049.
Li, S. et al. (2004) C-reactive protein upregulates complement-inhibitory factors in endothelial cells. Circulation 109, 833-836.
Lu, Y (2004) Recombinant adeno-associated virus as delivery vector for gene therapy—a review. Stem Cells Develop, 13, 133-145.
Magnusson, K. P. et al. (2006) CFH Y402H confers similar risk of soft drusen and both forms of advanced AMD. PLoS Med. 3, e5.
Maller, J., George, S., Purcell, S., Fagerness, J., Altshuler, D., Daly, M. J. and Seddon, J. M. (2006) Common variation in three genes, including a noncoding variant in CFH, strongly influences risk of age-related macular degeneration. Nature Genetics 38, 1055-1059.
Matukonis, M, Li, M., Molina, R., Paszkiet, B., Kaleko, M., and Luo, T. (2002). Development of second- and third-generation bovine immunodeficiency virus-based gene transfer systems. Hum. Gene Ther. 13, 1293-1303.
Mold, C., Gewurz, H., and Du Clos, T. W. (1999). Regulation of complement activation by C-reactive protein. Immunopharmacology, 42, 23-30.
Molina, R., Matukonis, M., Paszkiet, B., Zhang, J., Kaleko, M., and Luo, T. (2002). Mapping of the bovine immunodeficiency virus packaging signal and RRE and incorporation into a minimal gene transfer vector. Virology. 304, 10-23.
Molina, R. P., Ye, H. Q., Brady, J., Zhang, J., Zimmerman, H., Kaleko, M., and Luo, T. (2004). A Synthetic Rev-Independent Bovine Immunodeficiency Virus Based Packaging Construct. Hum. Gene Ther., 15, 65-877.
Muller-Eberhard, H. J. and Schreiber, R. D. (1980). Molecular biology and chemistry of the alternative pathway of complement. Adv. Immunol. 29, 1-53.
Mullins, R. F., Aptsiauri, N., and Hageman, G. S. (2001). Structure and composition of drusen associated with glomerulonephritis: implications for the role of complement activation in drusen biogenesis. Eye 15, 390-395.
Mullins, R. F., Russell, S. R., Anderson, D. H., and Hagemen, G. S. (2000). Drusen associated with aging and age-related macular degeneration contain proteins common to extracellular deposits associated with atherosclerosis, elastosis, amyloidosis, and dense deposit disease. The FASEB Journal, 14, 835-846.
Niculescu, F., Rus, H. G., Porutiu, D., Ghiurca, V., and Vlaicu, R. (1989). Immunoelectron-microscopic localization of S-protein/vitronectin in human atherosclerotic wall. Atherosclerosis, 78, 197-203.
Niculescu, F. and Rus, H. (2004) The role of complement activation in atherosclerosis. Imm. Res. 30, 73-80.
Nozaki, M. et al. (2006) Drusen complement components C3a and C5a promote choroidal neovascularization. PNAS 103, 2328-2333.
Ohno-Matsui, K., Hirose, A., Yamamoto, S., Saikia, J., Okamoto, N., Gehlback, P., Duh, E. J., Hackett, S. F., Chang, M., Bok, D., and Campochiaro, P. A. (2002). Inducible expression of vascular endothelial growth factor in photoreceptors of adult mice causes severe proliferative retinopathy and retinal detachment. Am. J. Pathol. 160, 711-719.
Oka et al. (2004) HtrA1 serine protease inhibits signaling mediated by Tgfβ family proteins. Develop. 131:1041-1053.
Okamoto, N., Tobe, T., Hackett, S. F., Ozaki, H., Vinores, M. A., LaRochelle, W., Zack, D. J. and Campochiaro, P. A. (1997) Transgenic mice with increased expression of vascular endothelial growth factor in the retina: a new model of intraretinal and subretinal neovascularization. Am. J. Pathol. 151, 281-291.
Pangburn, M. K., Schreiber, R. D., and Muller-Eberhard, H. J. (1977). Human complement C3b inactivator: isolation, characterization, and demonstration of an absolute requirement for the serum protein beta1H for cleavage of C3b and C4b in solution. J. Exp. Med. 146, 257-270.
Pauleikhoff, D., Barondes, M. J., Minassian, D., Chisholm, I. H., and Bird, A. C. (1990). Drusen as risk factors in age-related macular degeneration. Am. J. Opthalmol. 109, 38-43.
Penfold, K. L., Killingsworth, M. C., and Sarks, S. H. (1985). Senile macular degeneration: the involvement of immunocompetent cells. Graefe's Arch. Clin. Exp. Opthalmol. 223, 69-76.
Penfold, P. L., Provis, J. M., Furby, J. H., Gatenby, P. A., and Billson, F. A. (1990). Autoantibodies to retinal astrocytes associated with age-related macular degeneration. Graefes Arch. Clin. Exp. Opthalmol. 228, 270-274.
Pickering, M. C., Cook, H. T., Warren, J., Bygrave, A. E., Moss, J., Walport, M. J., and Botto, M. (2002). Uncontrolled C3 activation causes membranoproliferative glomerulonephritis in mice deficient in complement factor H. Nat. Genet. 31, 424-428.
Postel, E. A. et al. (2006). Complement factor H increases risk for atrophic age-related macular degeneration. Opthalmol. 113, 1504-1507.
Preissner, K. T. (1991). Structure and biological role of vitronectin. Ann. Rev. Cell Biol. 7, 275-310.
Provost, N., Le Meur, G., Weber, M., Mendes-Madeira, A., Podevin, G., Cherel, Y., Colle, M-A., Deschamps, J-Y., Moulier, P., and Rolling, F. (2005). Biodistribution of rAAV vectors following intraocular administration: Evidence for the presence and persistence of vector DNA in the optic nerve and the brain. Mol. Ther. 11, 275-283.
Rodriguez de Cordoba, S., Esparza-Gordillo, Goicoechea de Jorge, E., Lopez-Trascasa, M., Sanchez-Corral, P. (2004). The human complement factor H: functional roles, genetic variations and disease associations. Molecular Immunology, 41, 355-367.
Rus, H., Cudrici, C., Savid, S. and Niculescu, F. (2006) The complement system in central nervous system diseases. Autoimm. 39, 395-402.
Sanchez-Corral, P., Perez-Caballero, D., Huarte, O., Simckes, A. M., Goicoechea, E., Lopez-Trascasa, M., de Cordoba, S. R. (2002). Structural and functional characterization of factor H mutations associated with atypical hemolytic uremic syndrome. Am. J. Hum. Genet. 71, 1285-1295.
Seddon, J. M., Gensler, G., Milton, R. C., Klein, M. L., and Rifai, N. (2004). Association between C-reactive protein and age-related macular degeneration. JAMA 291, 704-710.
Sepp, T. et al. (2006) Complement factor H variant Y402H is a major risk determinant for Geographic Atrophy and choroidal neovascularization in smokers and nonsmokers. Invest. Opthalmol. 47, 536-540.
Sim, R. B., Kolble, K., McAleer, M. A., Dominguez, O., and Dee, V. M. (1993). Genetics and deficiencies of the soluble regulatory proteins of the complement system. Int. Rev. Immunol. 10, 65-86.
Sohn, J. H., Kaplan, H. J., Suk, H. J., Bora, P. S. and Bora, N. S. (2000) Complement regulatory activity of normal human intraocular fluid is mediated by MCP, DAF and CD59. Invest. Opthalmol. Vis. Sci. 41, 4195-4202.
Takahashi, K., Luo, T., Saishin, Y., Saishin, Y., Sung, J., Hackett, S., Brazzell, P. K., Kaleko, M., and Campochiaro, P. (2002). Sustained transduction of ocular cells with a bovine immunodeficiency viral vector. Hum. Gene Ther. 13, 1305-1316.
Takahashi, K., Saishin, Y., Saishin, Y., Lima e Silva, R., Oshima, Y., Oshima, S., Paszkiet, B., Zerby, D., Kadan, M., Liau, G., Kaleko, M., Connelly, S., Luo, T., and Campochiaro, P. A. (2003). Intraocular expression of endostatin reduces VEGF-induced retinal vascular permeability, neovascularization, and retinal detachment. FASEB, 17:896-899.
Thompson, R. A., and Winterborn, M. H. (1981). Hypocomplementaemia due to a genetic deficiency of beta 1H globulin. Clin. Exp. Immunol. 46, 110-119.
Vingerling, J. R., Dielemans, I., Bots, M. L., Hofman, A., Grobbee, D. E, and de Jong, P. T. (1995). Age-related macular degeneration is associated with atherosclerosis. The Rotterdam Study. Am. J. Epidemiol. 142, 404-409.
Volanakis, J. E., 1998. Overview of the complement system. Chapter 2. In The Human Complement System in Health and Disease. Edited by J. E. Volanakis, and M. M. Frank. Marcel Dekker, Inc., New York pp 9-32.
Weiler, J. M, Daha, M. R., Austen, K. F., and Fearon, D. T. (1976). Control of the amplification convertase of complement by the plasma protein beta1H. Proc. Natl. Acad. Sci. USA. 73, 3268-3272.
Whaley, K., and Ruddy, S. (1976). Modulation of the alternative complement pathways by beta 1 H globulin. J. Exp. Med. 144, 1147-1163.
Wistow, G., Bernstein, S. L., Wyatt, K., Fariss, R. N., Behal, A., Touchman, J. W., Bouffard, G., Smith, D., Peterson, K. (2002). Expressed sequence tag analysis of human RPE/choroid for the NEIBank Project: over 6000 non-redundant transcripts, novel genes and splice variants. Molecular Vision, 8, 205-220.
Wyatt, R. J., Julian, B. A., Weinstein, A., Rothfield, N. F., and McLean, R. H. (1982). Partial H (beta 1H) deficiency and glomerulonephritis in two families. J. Clin. Immunol. 2, 110-117.
Yang, G. S. et al. (2002) Virus-mediated transduction of murine retina with adeno-associated virus: effects of viral capsid and genome size. J. Virol. 76, 7651-7660.
Yang, Z. et al. (2006) A variant of the HTRA1 gene increases susceptibility to age-related macular degeneration. Science 314, 992.
Zipfel, P. F. (2001). Complement factor H: physiology and pathophysiology. Semin. Thromb Haemost. 27, 191-199.
Zipfel, P. F., and Skerka, C. (1999). FHL-1/rreconectin: a human complement and immune regulator with cell-adhesive function. Immunol. Today. 20, 135-140.
Zipfel, P. F., Skerka, C., Hellwage, J., Jokiranta, S. T., Meri, S., Brade, V., Kraiczy, P., Noris, M., and Remuzzi, G. (2002). Structure-function studies of the complement system. Biochemical Society Transactions, 30, 971-978.

It should be understood that various changes and modifications to the embodiments described herein will be apparent to those skilled in the art. Such changes and modifications can be made without departing from the spirit and scope of the present subject matter and without diminishing its intended advantages.

All publications, patents and patent applications mentioned in this specification are herein incorporated by reference in their entirety into the specification to the same extent as if each individual publication, patent or patent application was specifically and individually indicated to be incorporated herein by reference.

1-47. (canceled)

48. A method of treating a complement-mediated disease in a patient comprising administering to the patient a complement factor B analog treatment that inhibits or reduces complement activity, wherein said complement factor B analog has increased C3b binding affinity as compared to native complement factor B and the complement factor B analog has less activity in the alternative complement pathway as compared to native complement factor B.

49. The method of claim 48, wherein said complement factor B analog comprises a substitution corresponding to aspartic acid at 279 of SEQ ID NO:2, asparagine at 285 of SEQ ID NO:2 or both, wherein said substitution(s) increases C3b binding affinity as compared to the C3b binding affinity of native complement factor B.

50. The method of claim 49, wherein said aspartic acid is substituted with glycine, alanine or asparagine.

51. The method of claim 49, wherein said asparagine is substituted with glycine, alanine, or aspartic acid.

52. The method of claim 49, wherein said substitutions comprise replacing said aspartic acid with glycine and said asparagine with aspartic acid.

53. The method of claim 48, wherein said complement factor B analog comprises:
(i) a deletion corresponding to aspartic acid at 279 of SEQ ID NO:2, asparagine at 285 of SEQ ID NO:2 or both; or
(ii) an insertion next to said aspartic acid, said asparagine or both,
wherein said deletion(s) or insertion(s) increases C3b binding affinity as compared to the C3b binding affinity of native complement factor B.

54. The method of claim 48, wherein said complement factor B analog has an alteration in the active site of the serine protease domain that diminishes the activity of said complement factor B analog in the alternative complement pathway as compared to native complement factor B.

55. The method of claim 54, wherein said complement factor B analog comprises a substitution corresponding to aspartic acid at 740 of SEQ ID NO:2, wherein said substitution decreases the activity of said complement factor B analog in the alternative complement pathway as compared to native factor B.

56. The method of claim 55, wherein said aspartic acid is substituted with asparagine.

57. The method of claim 54, wherein said complement factor B analog comprises:
(i) a deletion corresponding to aspartic acid at 740 of SEQ ID NO:2; or
(ii) an insertion next to said aspartic acid,
wherein said deletion or insertion decreases activity of said complement factor B analog in the alternative complement pathway as compared to native complement factor B.

58. The method of claim 48, wherein said complement factor B analog has an alteration in its factor D cleavage site such that factor D has reduced ability to cleave the complement factor B analog as compared to the ability of factor D to cleave native complement factor B.

59. The method of claim 58, wherein said alteration comprises a substitution corresponding to one or more of lysine at 258, arginine at 259, or lysine at 260 of SEQ ID NO:2.

60. The method of claim 59, wherein said amino acids 258-260 of SEQ ID NO:2 are each substituted with alanine.

61. The method of claim 58, wherein said alteration in the factor D cleavage site comprises:
(i) a deletion corresponding to one or more of lysine at 258, arginine at 259, or lysine at 260 of SEQ ID NO:2; or
(ii) an insertion next to one or more of said lysine at 258, arginine at 259, or lysine at 260 of SEQ ID NO:2.

62. The method of claim 48, wherein said treatment comprises administering the complement factor B analog.

63. The method of claim 48, wherein said treatment comprises administering a vector that encodes the complement factor B analog.

64. The method of claim 63, wherein said vector is a retroviral vector, a lentiviral vector, an adenoviral vector, a Herpes viral vector, a Hepatitis viral vector, an SV40 vector, an EBV vector, an adeno-associated virus (AAV) vector or a nonviral vector.

65. The method of claim 64, wherein said lentivirus is HIV, EIAV, SIV, BIV or FIV.

66. The method of claim 63, wherein the vector is a viral vector and the viral vector comprises a decay accelerating factor.

67. The method of claim 48, wherein the administration is by intradermal injection, intramuscular injection, intraperitoneal injection, intravenous injection, subcutaneous injection, parenteral injection, epidural injection, intracranial injection, intraventricular injection, subdural injection, intraarticular injection, intrathecal injection, intracardiac injection, intracoronary injection, rectal infusion, intranasal application, intratracheal application, topical application, transdermal application or inhalation.

68. The method of claim 48, wherein the complement factor B analog comprises:
(i) amino acids 26-764 of SEQ ID NO:6 or
(ii) amino acids 26-764 of SEQ ID NO:8.

69. The method of claim 48, wherein the disease is myocardial infarction, stroke, ischemia reperfusion injury, traumatic organ injury, traumatic brain injury, arthritis or a disease of the eye.

70. The method of claim 69, wherein the disease of the eye is selected from the group consisting of macular degeneration, age-related macular degeneration (AMD), geographic atrophy, wet AMD, dry AMD, drusen formation, dry eye, diabetic retinopathy, vitreoretinopathy, corneal inflammation, uveitis, ocular hypertension or glaucoma.

71. The method of claim 69, wherein the disease is a disease of the eye and the complement factor B analog treatment is administered to the eye.

72. The method of claim 71, wherein the complement factor B analog treatment is delivered by intravitreal injection, subretinal injection, injection into the anterior chamber of the eye, injection or application locally to the cornea, subconjunctival injection, subtenon injection, or eye drops.

73. The method of claim 48, further comprising administering to the patient, prior to, concurrently with, or after the administration of the complement factor B analog treatment, another complement inhibiting factor or an anti-angiogenic factor.

74. The method of claim 73, where said complement inhibiting factor is selected from the group consisting of a Factor H, a Factor H-like 1, a Membrane Cofactor of Proteolysis (MCP), or a decay accelerating factor (DAF).

75. The method of claim 48, further comprising administering to the patient, prior to, concurrently with, or after the administration of the complement factor B analog treatment, an anti-inflammatory compound.

76. The method of claim 75, wherein the anti-inflammatory compound is selected from the group consisting of dexamethasone, dexamethasone sodium metasulfobenzoate, dexamethasone sodium phosphate, fluorometholone, bromfenac, pranoprofen, a cyclosporine ophthalmic emulsion, naproxen, glucocorticoids, ketorolac, ibuprofen, tolmetin, non-steroidal anti-inflammatory drugs, steroidal anti-inflammatory drugs, diclofenac, flurbiprofen, indomethacin, and suprofen.

77. A viral vector that encodes a complement factor B analog, the analog having increased C3b binding affinity as compared to native complement factor B and less activity in the alternative complement pathway as compared to native complement factor B.

78. A pharmaceutical composition comprising:
(i) a complement factor B analog with increased C3b binding affinity as compared to native complement factor B and less activity in the alternative complement pathway as compared to native complement factor B; and
(ii) a pharmaceutically acceptable carrier.

79. The pharmaceutical composition of claim 78, further comprising at least one ingredient selected from the group consisting of histidine, MgCl₂, trehalose, sucrose, a polysorbate, polysorbate 20, phosphate buffered saline, and NaCl.

80. A pharmaceutical composition comprising:
(i) a complement factor B analog having increased C3b binding affinity as compared to native complement factor B and which binds factor D more tightly than does native complement factor B; and
(ii) a pharmaceutically acceptable carrier.

81. A pharmaceutical composition comprising:
(i) a vector that encodes a complement factor B analog, the analog having increased C3b binding affinity as compared to native complement factor B and less activity in the alternative complement pathway as compared to native complement factor B; and
(ii) a pharmaceutically acceptable carrier.

82. A composition comprising a complement factor B analog with increased C3b binding affinity as compared to native complement factor B and less activity in the alternative complement pathway as compared to native complement factor B, wherein the composition is at least 95% pure with regards to total protein.

83. A complement factor B analog with increased C3b binding affinity as compared to native complement factor B and less activity in the alternative complement pathway as compared to native complement factor B, wherein the complement factor B analog is produced from CHO or 293 cells.

84. An isolated protein comprising:
(i) amino acids 26-764 of SEQ ID NO:4;
(ii) amino acids 26-764 of SEQ ID NO:6; or
(iii) amino acids 26-764 of SEQ ID NO:8.
